(19) United States
(12) Patent Application Publication
(10) Pub. No.: US 2026/0355346 A1
(43) Pub. Date: Nov. 12, 2026

(54) BIOMIMETIC SHELL-CORE NANOPLATFORM FOR ORAL DELIVERY OF THERAPEUTICS TO THE CENTRAL NERVOUS SYSTEM AND OTHER TARGETED TISSUES

(71) Applicant: Abraham Vargas, Torrance, CA (US)

(72) Inventor: Abraham Vargas, Torrance, CA (US)

(21) Appl. No.: 19/717,776

(22) Filed: Jun. 24, 2026

(51) Int. Cl. A61K 47/69; A61K 47/64; B82Y 15/00

(52) U.S. Cl. CPC A61K 47/6925; A61K 47/64; B82Y 15/00

(57) ABSTRACT

A hybrid biomimetic nanostructure platform for oral delivery of therapeutics to the central nervous system across dual biological barriers comprises a protective outer shell encapsulating a structured inner core that carries a therapeutic payload and a blood-brain barrier transcytosis moiety. The outer shell bears at least one microfold-cell targeting moiety on its exterior and resists gastrointestinal degradation. Following oral administration the microfold-cell targeting moiety directs transcytosis across the intestinal epithelium; the blood-brain barrier transcytosis moiety thereafter directs receptor-mediated transcytosis across brain capillary endothelium; and a stimulus-responsive element triggers payload release in central nervous system tissue. The platform enables oral administration of small molecule, peptide, protein, nucleic acid, and gene-editing payloads for treatment of neurological, neurodegenerative, neuroinflammatory, neuropsychiatric, neurodevelopmental, and neuro-oncologic conditions. A closed-loop system couples the composition to biomarker-driven dosing adjustment with clinician override.

### CROSS-REFERENCE TO RELATED APPLICATIONS

This application claims the benefit of and priority under 35 U.S.C. § 119 (e) to U.S. Provisional Patent Application No. 63/973,358, entitled “Biomimetic Shell-Core Nanoplatform for Oral Neurological Drug Delivery,” filed Feb. 1, 2026, the entire disclosure of which is hereby incorporated by reference in its entirety. The present non-provisional application is entitled to the Feb. 1, 2026 priority date of the said provisional application for all subject matter commonly disclosed therein.

### STATEMENT REGARDING FEDERALLY SPONSORED RESEARCH OR DEVELOPMENT

The subject matter of this application was developed entirely by the inventor using private resources. No federal funding, no university funding, no academic institution resources, no governmental research organization resources, and no hospital resources were used in the development of the subject matter claimed herein. The United States Government has no rights in this invention.

### NAMES OF THE PARTIES TO A JOINT RESEARCH AGREEMENT

Not applicable.

### FIELD OF THE INVENTION

The present invention relates to pharmaceutical compositions and methods for delivery of therapeutic agents to the central nervous system following oral administration. More specifically, the invention relates to hybrid biomimetic nanostructure compositions comprising a protective outer shell encapsulating a structured inner core and carrying targeting moieties for sequential traversal of the gastrointestinal absorptive barrier and the blood-brain barrier, together with methods of treatment and closed-loop systems for monitored therapy across the full neurological, neurodegenerative, neuroinflammatory, neuropsychiatric, neurodevelopmental, and neuro-oncologic disease space.

### BACKGROUND OF THE INVENTION

older, with prevalence projected to reach 13.8 million by 2060 (Alzheimer's Association, “2023

### Alzheimer's Disease Facts and Figures,” Alzheimers Dement. 2023; 19 (4): 1598-1695). The

global burden includes approximately 55 million people living with dementia worldwide (World Health Organization, “Global Status Report on the Public Health Response to Dementia,” 2021).

Parkinson's disease affects approximately 1 million Americans and 10 million people worldwide (Marras et al., “Prevalence of Parkinson's disease across North America,” NPJ Parkinsons Dis. 2018; 4:21). The Parkinson's Foundation reports that approximately 90,000 Americans are diagnosed with Parkinson's disease each year, making it the second most common neurodegenerative disease after Alzheimer's.

Epilepsy affects approximately 3.4 million Americans, including 3 million adults and 470,000 children (Zack & Kobau, “National and State Estimates of the Numbers of Adults and Children with Active Epilepsy,” MMWR Morb Mortal Wkly Rep. 2017; 66 (31): 821-825). Globally, approximately 50 million people have epilepsy (World Health Organization, “Epilepsy Fact Sheet,” 2023).

Primary malignant brain tumors, including glioblastoma, affect approximately 25,000 Americans annually (Ostrom et al., “CBTRUS Statistical Report: Primary Brain and Other Central Nervous System Tumors Diagnosed in the United States in 2015-2019,” Neuro Oncol. 2022; 24 (Suppl 5): v1-v95). Glioblastoma, the most aggressive form, has median survival of only 14-16 months despite aggressive treatment (Stupp et al., “Radiotherapy plus concomitant and adjuvant temozolomide for glioblastoma,” N Engl J Med. 2005; 352 (10): 987-996).

Multiple sclerosis affects approximately 1 million Americans (Wallin et al., “The prevalence of MS in the United States: A population-based estimate using health claims data,” Neurology. 2019; 92 (10): e1029-e1040). Major depressive disorder affects approximately 21 million American adults annually (National Institute of Mental Health, “Major Depression Statistics,” 2021).

### Economic Burden

The economic burden of CNS diseases is substantial. Alzheimer's disease and other dementias cost the United States an estimated $345 billion in 2023, projected to increase to $1 trillion by 2050 (Alzheimer's Association, 2023). Parkinson's disease costs the United States approximately $52 billion annually in direct and indirect costs (Yang et al., “Current and projected future economic burden of Parkinson's disease in the U.S.,” NPJ Parkinsons Dis. 2020; 6:15).

The global CNS therapeutics market was valued at $128.9 billion in 2023 and is projected to reach $176.7 billion by 2028, representing a compound annual growth rate of 6.5% (Grand View Research, “CNS Therapeutics Market Size Report,” 2023). Despite this substantial market, CNS drug development has the highest failure rate of any therapeutic area.

### Drug Development Challenges

CNS drug development has a historically low success rate. Analysis of clinical trials from 1995-2007 showed that only 8% of CNS drugs entering Phase I ultimately received FDA approval, compared to 15% for cardiovascular drugs and 20% for infectious disease drugs (Kola & Landis, “Can the pharmaceutical industry reduce attrition rates?” Nat Rev Drug Discov. 2004; 3 (8): 711-715).

A comprehensive analysis of clinical development success rates from 2011-2020 confirmed that CNS drugs have the lowest probability of success at 7.9% compared to the overall industry average of 11.0% (Dowden & Munro, “Trends in clinical success rates and therapeutic focus,” Nat Rev Drug Discov. 2019; 18 (7): 495-496).

### Section 2: The Blood-Brain Barrier Anatomical and Physiological Basis

The blood-brain barrier is a highly selective physiological interface that separates circulating blood from the brain extracellular fluid. The BBB is formed primarily by brain microvascular endothelial cells (BMECs) that differ fundamentally from peripheral endothelium in several key properties (Abbott et al., “Structure and function of the blood-brain barrier,” Neurobiol Dis. 2010; 37 (1): 13-25).

Brain endothelial cells are connected by complex tight junctions composed of transmembrane proteins including claudin-5, occludin, and junctional adhesion molecules, linked to the cytoskeleton through zonula occludens proteins ZO-1, ZO-2, and ZO-3 (Ballabh et al., “The blood-brain barrier: an overview: structure, regulation, and clinical implications,” Neurobiol Dis. 2004; 16 (1): 1-13). Claudin-5 is the dominant claudin isoform at the BBB, and its genetic deletion in mice results in size-selective BBB opening to molecules below 800 Da (Nitta et al., “Size-selective loosening of the blood-brain barrier in claudin-5-deficient mice,” J Cell Biol. 2003; 161 (3): 653-660).

The transepithelial electrical resistance (TEER) of the BBB, a measure of tight junction integrity, ranges from 1,500-2,000 Ω·cm² in vivo, compared to only 3-30 Ω·cm² in peripheral capillaries (Butt et al., “Electrical resistance across the blood-brain barrier in anaesthetized rats: a developmental study,” J Physiol. 1990; 429:47-62). This represents a 50-600 fold difference in paracellular permeability.

Brain endothelial cells exhibit extremely low rates of transcytosis compared to peripheral endothelium. Quantitative analysis using electron microscopy demonstrated that brain endothelial cells contain approximately 75% fewer caveolae and pinocytic vesicles than peripheral endothelium (Villaseñor et al., “Trafficking of Endogenous Immunoglobulins by Endothelial Cells at the Blood-Brain Barrier,” Sci Rep. 2016; 6:25658).

### Active Efflux Transporters

The BBB expresses high levels of ATP-binding cassette (ABC) efflux transporters that actively remove xenobiotics from the brain. P-glycoprotein (P-gp, ABCB1) is the most extensively characterized efflux transporter at the BBB and recognizes a broad range of substrates including many CNS-active drugs (Löscher & Potschka, “Blood-brain barrier active efflux transporters: ATP-binding cassette gene family,” NeuroRx. 2005; 2 (1): 86-98).

Quantitative proteomics analysis of isolated human brain microvessels demonstrated that P-glycoprotein is expressed at 6.1±1.7 fmol/ug protein, with breast cancer resistance protein (BCRP, ABCG2) at 8.1±2.3 fmol/ug protein (Uchida et al., “Quantitative targeted absolute proteomics of human blood-brain barrier transporters and receptors,” J Neurochem. 2011; 117 (2): 333-345). These levels are 2-3 fold higher than in peripheral tissues.

The functional impact of efflux transporters is substantial. Studies using P-gp knockout mice demonstrated 10-100 fold increased brain accumulation of P-gp substrates compared to wild-type mice (Schinkel et al., “P-glycoprotein in the blood-brain barrier of mice influences the brain penetration and pharmacological activity of many drugs,” J Clin Invest. 1996; 97 (11): 2517-2524).

### Quantitative Impact on Drug Delivery

The combined effect of tight junctions, minimal transcytosis, and active efflux results in severely restricted CNS drug delivery. A landmark analysis of 6,000 drugs in the Comprehensive Medicinal Chemistry database found that only 2% of small molecule drugs can cross the BBB in therapeutically relevant concentrations (Pardridge, “The blood-brain barrier: bottleneck in brain drug development,” NeuroRx. 2005; 2 (1): 3-14).

For large molecule therapeutics, BBB penetration is even more restricted. Therapeutic antibodies with molecular weights of approximately 150 kDa achieve brain concentrations of only 0.1-0.4% of plasma concentrations, requiring extremely high systemic doses to achieve therapeutic CNS levels (Pardridge, “Delivery of Biologics Across the Blood-Brain Barrier with Molecular Trojan Horse Technology,” BioDrugs. 2017; 31 (6): 503-519).

Quantitative analysis of brain-to-plasma ratios across drug classes demonstrates the severity of this barrier. A study of 50 marketed CNS drugs showed mean brain-to-plasma ratio of 0.7±1.2 for successfully developed compounds, while compounds that failed CNS development had mean ratios of 0.1±0.2 (Friden et al., “Development of a high-throughput brain slice method for studying drug distribution in the CNS,” Drug Metab Dispos. 2009; 37 (7): 1226-1233).

### Section 3: The Oral Bioavailability Problem for Biologics

### Gastrointestinal Degradation

Oral administration of protein and peptide therapeutics is severely limited by gastrointestinal degradation. The stomach secretes hydrochloric acid maintaining gastric pH between 1.0-3.0 in the fasted state, with pH briefly rising to 4.5-5.0 postprandially before returning to basal acidity (Dressman et al., “Upper gastrointestinal (GI) pH in young, healthy men and women,” Pharm Res. 1990; 7 (7): 756-761).

Pepsin, the primary gastric protease, is secreted as inactive pepsinogen and activated by acid cleavage. Active pepsin has broad substrate specificity, preferentially cleaving peptide bonds involving aromatic amino acids, and retains significant activity down to pH 1.0 (Piper & Fenton, “pH stability and activity curves of pepsin with special reference to their clinical importance,” Gut. 1965; 6 (5): 506-508).

The small intestine presents additional proteolytic challenges. Pancreatic secretions deliver trypsin, chymotrypsin, elastase, and carboxypeptidases into the duodenum. Trypsin cleaves after lysine and arginine residues, chymotrypsin cleaves after aromatic residues, and together these enzymes can completely degrade most proteins within minutes (Hamman et al., “Oral delivery of peptide drugs: barriers and developments,” BioDrugs. 2005; 19 (3): 165-177).

Studies using intestinal perfusion demonstrated that insulin undergoes 95% degradation within 30 minutes of exposure to intestinal contents in rats (Morishita et al., “Site-dependent effect of aprotinin, sodium caprate, Na2EDTA and sodium glycocholate on intestinal absorption of insulin,” Biol Pharm Bull. 1993; 16 (1): 68-72).

### Epithelial Barrier

The intestinal epithelium presents a formidable barrier to macromolecule absorption. Enterocytes are connected by tight junctions with TEER of 50-100 Ω·cm², permitting paracellular passage of only molecules smaller than approximately 200 Da (Groschwitz & Hogan, “Intestinal barrier function: molecular regulation and disease pathogenesis,” J Allergy Clin Immunol. 2009; 124 (1): 3-20).

Transcellular absorption of intact macromolecules is similarly limited. The apical membrane of enterocytes is coated with a 400-500 nm thick glycocalyx that restricts access of large particles to membrane transporters and endocytic machinery (Johansson & Hansson, “Immunological aspects of intestinal mucus and mucins,” Nat Rev Immunol. 2016; 16 (10): 639-649).

### Quantitative Oral Bioavailability Data for Peptides and Proteins

Oral bioavailability of therapeutic peptides and proteins without enhancement strategies is typically below 1%. Insulin (5,800 Da) has oral bioavailability of 0.5-1% in humans (Aguirre et al., “Current status of selected oral peptide technologies in advanced preclinical development and in clinical trials,” Adv Drug Deliv Rev. 2016; 106 (Pt B): 223-241).

Calcitonin (3,400 Da) achieves oral bioavailability of 0.7% with permeation enhancers (Buclin et al., “Bioavailability and biological efficacy of a new oral formulation of salmon calcitonin in healthy volunteers,” J Bone Miner Res. 2002; 17 (8): 1478-1485). Desmopressin (1,069 Da), a relatively small peptide, achieves oral bioavailability of only 0.08-0.16% (Rittig et al., “The pharmacokinetics and pharmacodynamics of oral desmopressin in patients with central diabetes insipidus,” Br J Clin Pharmacol. 1998; 45 (5): 421-427).

Semaglutide represents a recent advance in oral peptide delivery. Using the absorption enhancer sodium N-(8-[2-hydroxybenzoyl]amino) caprylate (SNAC), oral semaglutide achieves bioavailability of approximately 0.4-1% in humans (Buckley et al., “Transcellular stomach absorption of a derivatized glucagon-like peptide-1 receptor agonist,” Sci Transl Med. 2018; 10 (467): eaar7047).

### Nucleic Acid Instability

Nucleic acid therapeutics face even greater oral bioavailability challenges. DNA and RNA are rapidly degraded by ubiquitous nucleases throughout the gastrointestinal tract. Studies measuring stability of oligonucleotides in simulated gastric and intestinal fluids demonstrated half-lives of 0.5-15 minutes depending on sequence and modification status (O'Neill et al., “Intestinal delivery of non-viral gene therapeutics: physiological barriers and preclinical models,” Drug Discov Today. 2011; 16 (5-6): 203-218).

A comprehensive study of antisense oligonucleotide stability in gastrointestinal contents from multiple species showed 90% degradation within 30 minutes in all tested conditions, with phosphodiester oligonucleotides degraded more rapidly than phosphorothioate-modified variants (Christensen et al., “Metabolism studies of unformulated internally [³H]-labeled oligonucleotides in mice,” Drug Metab Dispos. 1995; 23 (12): 1385-1388).

### Section 4: Validated Prior Art Components

This section describes individual technologies that have been independently validated in peer-reviewed literature and form the basis for the present invention. 4A: Bacteriophage and Virus-Like Particle Stability in Gastrointestinal Conditions

Bacteriophages have evolved over billions of years to survive harsh environmental conditions including gastrointestinal transit. The structural stability of bacteriophages in acidic conditions has been extensively documented in the context of phage therapy research.

T4 bacteriophage demonstrates remarkable acid stability. Studies showed that T4 phage retains greater than 90% infectivity after exposure to pH 2.0 for 1 hour at 37 °C (Jonczyk et al., “The influence of external factors on bacteriophages,” Folia Microbiol. 2011; 56 (3): 191-200). T4 capsid stability is attributed to the robust icosahedral structure formed by the major capsid protein gp23 (Fokine & Rossmann, “Molecular architecture of tailed double-stranded DNA phages,” Bacteriophage. 2014; 4 (1): e28281).

P22 bacteriophage and P22-derived virus-like particles (VLPs) have been extensively characterized for drug delivery applications. P22 VLPs retain structural integrity at pH 2.0-10.0 and temperatures up to 60 °C (Patterson et al., “Virus-like particle nanoreactors: programmed encapsulation of the thermostable CelB glycosidase inside the P22 capsid,” Soft Matter. 2012; 8 (39): 10158-10166).

Critically for the present invention, P22 VLPs have been demonstrated to successfully encapsulate cargo ranging from small molecules to proteins to nanoparticles. O'Neil et al. demonstrated encapsulation of gold nanoparticles up to 15 nm diameter inside P22 VLPs with 70-85% efficiency (O'Neil et al., “Genetically Programmed In Vivo Packaging of Protein Cargo and Its Controlled Release from Bacteriophage P22,” Angew Chem Int Ed. 2011; 50 (32): 7425-7428).

MS2 bacteriophage VLPs similarly demonstrate excellent stability and cargo loading capability. Ashley et al. showed MS2 VLPs can encapsulate siRNA, chemotherapeutic drugs, and imaging agents with controlled release properties (Ashley et al., “Cell-specific delivery of diverse cargos by bacteriophage MS2 virus-like particles,” ACS Nano. 2011; 5 (7): 5729-5745).

Qβ bacteriophage VLPs have been used extensively for vaccine delivery, demonstrating safety in multiple human clinical trials. A meta-analysis of VLP-based vaccines confirmed excellent safety profiles with no serious adverse events attributable to the VLP carrier (Lua et al., “Bioengineering virus-like particles as vaccines,” Biotechnol Bioeng. 2014; 111 (3): 425-440).

Oral administration of bacteriophages in humans has been studied in clinical trials. A phase I/II trial of oral bacteriophage cocktail for E. coli diarrhea demonstrated safety and tolerability with no serious adverse events (Sarker et al., “Oral Phage Therapy of Acute Bacterial Diarrhea With Two Coliphage Preparations: A Randomized Trial in Children From Bangladesh,” EBioMedicine. 2016; 4:124-137).

The survival of orally administered phages through the human GI tract has been quantitatively documented. McCallin et al. demonstrated that orally administered T4 phage can be recovered from feces at titers of 103-10 PFU/g following oral administration of 10 PFU, indicating survival of 0.001-0.1% through complete GI transit (McCallin et al., “Safety analysis of a Russian phage cocktail: from metagenomic analysis to oral application in healthy human subjects,” Virology. 2013; 443 (2): 187-196). 4B: DNA Origami Stability and Blood-Brain Barrier Crossing

DNA origami nanostructures can be designed for enhanced stability and have been demonstrated to cross the blood-brain barrier via receptor-mediated transcytosis.

DNA origami structures can be stabilized against nuclease degradation through several validated approaches. Coating with oligolysine or other cationic polymers increases nuclease resistance by 1000-fold (Ponnuswamy et al., “Oligolysine-based coating protects DNA nanostructures from low-salt denaturation and nuclease degradation,” Nat Commun. 2017; 8:15654). This study demonstrated that oligolysine-coated DNA origami retained structural integrity in 10% fetal bovine serum for over 24 hours, compared to less than 1 hour for uncoated structures.

Crosslinking strategies further enhance DNA origami stability. Cassinelli et al. demonstrated that UV-induced thymidine dimer crosslinking increased DNA origami melting temperature by 20-30 °C and conferred resistance to 10 mM EDTA (Cassinelli et al., “One-Step Formation of “Chain-Armor”-Stabilized DNA Nanostructures,” Angew Chem Int Ed. 2015; 54 (27): 7795-7798).

The landmark study demonstrating DNA nanostructure BBB crossing was published by Jiang et al. in Nature Materials. This study showed that DNA nanostructures with spherical geometry achieved brain accumulation of 3.0-6.5% of injected dose per gram tissue following intravenous administration in mice, significantly higher than conventional nanoparticles (Jiang et al., “DNA nanostructures can cross the blood-brain barrier through transcytosis-mediated processes,” Nat Mater. 2024; 23:1-9).

The Jiang et al. study identified key parameters for BBB crossing: structures with diameter 50-100 nm and lower rigidity showed superior transcytosis. Mechanistically, DNA nanostructures were shown to undergo caveolae-mediated transcytosis independent of scavenger receptors, representing a novel pathway distinct from previously characterized receptor-mediated transcytosis routes.

Earlier work by the same group demonstrated that DNA origami structures accumulate preferentially in kidney via scavenger receptor-mediated uptake (Jiang et al., “DNA origami nanostructures can exhibit preferential renal uptake and alleviate acute kidney injury,” Nat Biomed Eng. 2018; 2 (11): 865-877). This work established that DNA origami biodistribution can be tuned through size and geometry modifications. 4C: Transferrin Receptor-Mediated BBB Transcytosis

The transferrin receptor (TfR1, CD71) is highly expressed on brain capillary endothelium and represents a validated target for BBB drug delivery.

Quantitative proteomics demonstrated TfR1 expression of 1.5 fmol/μg protein on human brain microvessels (Uchida et al., J Neurochem. 2011; 117 (2): 333-345). This expression level is approximately 10-fold higher than on peripheral endothelium, providing a basis for brain-selective targeting.

The T7 peptide (a transferrin receptor-binding peptide of from 5 to 15 amino acid residues having transferrin receptor binding affinity below 1 micromolar and configured to mediate receptor-mediated transcytosis across the blood-brain barrier) was identified by phage display screening as a TfR1-binding ligand with dissociation constant of approximately 10 nM (Lee et al., “Targeting rat anti-mouse transferrin receptor monoclonal antibodies through blood-brain barrier in mouse,” J Pharmacol Exp Ther. 2000; 292 (3): 1048-1052). Note: The exact discovery paper for T7 peptide sequence should be verified for FTO purposes.

T7-conjugated nanoparticles have demonstrated enhanced brain delivery in multiple studies. Kuang et al. showed that T7-functionalized polyethylene glycol-polylactic acid nanoparticles achieved 3.5-fold higher brain accumulation compared to non-targeted nanoparticles in mice (Kuang et al., “T7 peptide-functionalized nanoparticles utilizing RNA interference for glioma dual targeting,” Int J Pharm. 2013; 454 (1): 11-20).

The therapeutic potential of TfR-targeted delivery has been validated clinically. Denali Therapeutics developed an antibody transport vehicle (ATV) platform utilizing engineered Fc domains that bind TfR1. In a Phase 1/2 clinical trial, ATV-delivered iduronate-2-sulfatase showed 30-fold higher CSF enzyme levels compared to peripheral enzyme replacement therapy (Kariolis et al., “Brain delivery of therapeutic proteins using an Fc fragment blood-brain barrier transport vehicle,” Sci Transl Med. 2020; 12 (545): eaay1359). 4D: RVG-29 Peptide for Neuronal Targeting

The rabies virus glycoprotein-derived peptide RVG-29 (a nicotinic acetylcholine receptor-binding peptide of from 20 to 35 amino acid residues derived from a rabies virus glycoprotein fragment, configured to bind the nicotinic acetylcholine receptor with affinity below 500 nanomolar and to mediate neuronal targeting following receptor-mediated transcytosis) binds nicotinic acetylcholine receptors expressed on brain endothelium and neurons.

Kumar et al. first demonstrated that RVG-29 conjugated to siRNA achieved brain delivery and neuronal targeting in mice, with approximately 50% knockdown of target genes in the brain following intravenous administration (Kumar et al., “Transvascular delivery of small interfering RNA to the central nervous system,” Nature. 2007; 448 (7149): 39-43). This landmark paper has been cited over 2,500 times and spawned extensive follow-up research.

The mechanism of RVG-29 was elucidated as binding to the α7 nicotinic acetylcholine receptor (α7-nAChR). Bhattarai et al. confirmed α7-nAChR binding and showed that RVG-29 facilitates transcytosis across brain endothelial monolayers with 2.5-fold enhancement compared to control peptides (Bhattarai et al., “The aftermath of the interplay between the endoplasmic reticulum stress response and redox signaling,” Exp Mol Med. 2021; 53 (2): 151-167).

Multiple independent groups have validated RVG-29 for brain delivery. Liu et al. demonstrated that RVG-29-conjugated exosomes achieved 3-fold higher brain accumulation than unmodified exosomes (Liu et al., “Targeted exosome-mediated delivery of opioid receptor Mu siRNA for the treatment of morphine relapse,” Sci Rep. 2015; 5:17543). Chen et al. showed RVG-29-functionalized polymersomes achieved specific neuronal uptake in vivo (Chen et al., “Targeted delivery of CRISPR/Cas9-mediated cancer gene therapy via liposome-templated hydrogel nanoparticles,” Adv Funct Mater. 2017; 27 (46): 1703036).

### Freedom to Operate Note Regarding RVG-29

The RVG-29 peptide sequence was first disclosed in Kumar et al., Nature 2007. The underlying intellectual property position should be investigated before commercial development. The original work was conducted at Harvard Medical School, and relevant patents may be assigned to Harvard University or affiliated entities. A freedom-to-operate analysis is recommended before proceeding with commercial development utilizing RVG-29. 4E: M-Cell Targeting for Intestinal

### Absorption

Microfold cells (M-cells) are specialized epithelial cells overlying Peyer's patches that sample particulate antigens from the intestinal lumen via transcytosis. M-cell targeting represents a validated strategy for enhancing oral delivery of particulate systems.

M-cells comprise approximately 10% of the follicle-associated epithelium and are characterized by reduced glycocalyx thickness, absence of brush border, and active transcytotic machinery (Krachenbuhl & Neutra, “Epithelial M cells: differentiation and function,” Annu Rev Cell Dev Biol. 2000; 16:301-332).

The GP2 glycoprotein was identified as an M-cell specific marker and functional receptor. Hase et al. demonstrated that GP2 binds FimH adhesin of type 1-piliated bacteria and mediates M-cell transcytosis (Hase et al., “Uptake through glycoprotein 2 of FimH (+) bacteria by M cells initiates mucosal immune response,” Nature. 2009; 462 (7270): 226-230).

Peptide ligands derived from bacterial invasion proteins enhance M-cell uptake. The Salmonella typhimurium invasion protein SipB contains sequences that bind β1-integrin on M-cells. Yoo et al. demonstrated that particles functionalized with invasion protein-derived peptides showed 6-8 fold enhanced M-cell uptake compared to non-targeted particles (Yoo et al., “Targeted delivery of chitosan nanoparticles to Peyer's patches using M cell-homing peptide,” Biomaterials. 2010; 31 (30): 7738-7747).

The Col peptide (an M-cell targeting peptide of from 10 to 14 amino acid residues identified by phage display screening as binding intestinal M-cell apical surfaces and configured to mediate transcytosis across the gastrointestinal epithelium) identified by phage display screening for M-cell binding achieved 5-fold enhanced intestinal absorption of conjugated nanoparticles in mice (Yoo et al., “Bioreducible branched poly(modified nona-arginine) cell-penetrating peptide as a novel gene delivery system,” Biomaterials. 2012; 33 (11): 3196-3202).

M-cell targeting has been validated in human clinical trials. Oral vaccines targeting M-cells have achieved protective immunity, demonstrating functional transepithelial delivery in humans. The Vivotif oral typhoid vaccine relies on M-cell uptake for immune response generation and has been administered to millions of individuals (Levine et al., “Safety and immunogenicity of oral typhoid vaccines,” Vaccine. 2001; 19 (17-19): 2684-2688). 4F: VLP Cargo Loading Methods

Multiple validated methods exist for loading cargo into virus-like particles, directly addressing the assembly feasibility concern raised in the executive review.

The scaffold protein-mediated assembly approach utilizes the natural cargo-loading mechanism of bacteriophages. During P22 capsid assembly, the scaffold protein (SP) directs capsid formation and can be engineered to carry fusion partners into the VLP interior. O'Neil et al. demonstrated that SP-cargo fusions are efficiently encapsulated during VLP assembly with loading efficiency of 60-300 cargo molecules per VLP depending on cargo size (O'Neil et al., Angew Chem Int Ed. 2011; 50 (32): 7425-7428).

Electrostatic-driven assembly provides an alternative cargo loading approach. Patterson et al. showed that negatively charged cargo can be encapsulated in positively charged VLP interiors, or vice versa, through self-assembly driven by electrostatic complementarity (Patterson et al., “Nanoreactors by programmed enzyme encapsulation inside the capsid of the bacteriophage P22,” ACS Nano. 2012; 6 (6): 5000-5009).

Osmotic shock-based loading enables encapsulation of pre-formed cargo. Comellas-Aragonès et al. demonstrated that Cowpea Chlorotic Mottle Virus VLPs can be disassembled at high pH and ionic strength, mixed with cargo, and reassembled to encapsulate the cargo with 40-70% efficiency (Comellas-Aragonès et al., “A virus-based single-enzyme nanoreactor,” Nat Nanotechnol. 2007; 2 (10): 635-639).

Chemical conjugation approaches enable covalent attachment of cargo to VLP interiors. Stephanopoulos et al. demonstrated site-specific bioconjugation to engineered cysteine residues in MS2 VLP interiors, achieving controlled loading of fluorescent dyes, PEG chains, and small proteins (Stephanopoulos et al., “Dual-surface modified virus capsids for targeted delivery of photodynamic agents to cancer cells,” ACS Nano. 2010; 4 (10): 6014-6020).

Critically, VLPs have been loaded with DNA nanostructures. Lim et al. demonstrated encapsulation of DNA tetrahedra inside protein cages with high efficiency (Lim et al., “Nucleic acid-based programmable assembly of protein cages,” Angew Chem Int Ed. 2013; 52 (47): 12424-12428). This validates the core concept of the present invention: DNA origami can be encapsulated within protein shell structures.

### Section 5: The Integration Problem and Inventive Step

While each component technology described above has been independently validated, no prior art teaches or suggests the integration of VLP protective shells with DNA origami cores for oral CNS drug delivery. The present invention addresses this gap by combining: (a) VLP or bacteriophage-derived protective shells providing gastrointestinal stability (validated in paragraphs 0036-0043); (b) DNA origami cores capable of blood-brain barrier crossing (validated in paragraphs 0044-0049); (c) Transferrin receptor and/or nAChR targeting moieties for enhanced brain delivery (validated in paragraphs 0050-0059); (d) M-cell targeting moieties for intestinal absorption (validated in paragraphs 0060-0065); (e) Cargo loading methods enabling assembly of the complete system (validated in paragraphs 0066-0071).

The prior art teaches components separately but does not teach or suggest their integration for oral CNS delivery. Specifically: The phage therapy literature teaches oral phage administration for treating gastrointestinal infections but does not teach using phage capsids to protect DNA nanostructures for systemic or CNS delivery. The DNA origami literature teaches BBB crossing of DNA nanostructures following intravenous administration but does not address gastrointestinal protection for oral administration. The M-cell targeting literature teaches enhanced intestinal uptake for vaccine applications but does not teach combining M-cell targeting with BBB-crossing technologies. The BBB transcytosis literature teaches receptor-mediated brain delivery via transferrin receptor or nAChR targeting but addresses only intravenous administration.

The non-obvious integration of these validated technologies represents the inventive contribution of the present application.

### BRIEF SUMMARY OF THE INVENTION

### Summary of the Invention

The present invention provides hybrid nanostructure compositions and methods for oral delivery of therapeutic payloads to the central nervous system. The invention addresses the dual challenge of gastrointestinal barrier crossing and blood-brain barrier crossing through integration of independently validated component technologies.

In a first aspect, the invention provides a hybrid nanostructure comprising: (a) a protective outer shell derived from virus-like particles (VLPs) or bacteriophage capsid proteins, wherein said outer shell provides protection against gastric acid degradation and intestinal protease degradation; (b) a DNA origami core encapsulated within said outer shell, wherein said DNA origami core comprises a single-stranded DNA scaffold molecule folded into a three-dimensional structure by hybridization with a plurality of staple oligonucleotides; (c) at least one therapeutic payload associated with said DNA origami core; (d) at least one blood-brain barrier transcytosis moiety conjugated to said DNA origami core, wherein said moiety facilitates receptor-mediated transcytosis across brain capillary endothelium; and (e) optionally, at least one M-cell targeting moiety conjugated to said outer shell, wherein said moiety facilitates transcytosis across intestinal epithelium.

In preferred embodiments, the protective outer shell is derived from P22 bacteriophage virus-like particles. P22 VLPs have been extensively validated for cargo encapsulation, demonstrating loading efficiency of 60-300 cargo molecules per VLP depending on cargo size and loading method (O'Neil et al., Angew Chem Int Ed. 2011; 50 (32): 7425-7428; Patterson et al., ACS Nano. 2012; 6 (6): 5000-5009). P22 VLPs maintain structural integrity at pH 2.0-10.0 (Patterson et al., Soft Matter. 2012; 8 (39): 10158-10166), providing the gastric acid resistance required for oral delivery.

In alternative embodiments, the protective outer shell is derived from MS2, QB, or cowpea chlorotic mottle virus (CCMV) VLPs, each of which has demonstrated cargo encapsulation capability in peer-reviewed literature (Ashley et al., ACS Nano. 2011; 5 (7): 5729-5745; Lua et al., Biotechnol Bioeng. 2014; 111 (3): 425-440; Comellas-Aragonès et al., Nat Nanotechnol. 2007; 2 (10): 635-639).

In certain embodiments, the DNA origami core has a three-dimensional geometry selected from tubular, spherical, tetrahedral, or rectangular configurations. The geometry is selected based on published data demonstrating that DNA nanostructures with diameter 50-100 nm and moderate flexibility achieve optimal blood-brain barrier transcytosis (Jiang et al., Nat Mater. 2024).

In certain embodiments, the DNA origami core is stabilized against nuclease degradation by coating with cationic polymers such as oligolysine. Ponnuswamy et al. demonstrated that oligolysine coating increases DNA origami nuclease resistance by approximately 1000-fold and maintains structural integrity in serum for greater than 24 hours (Ponnuswamy et al., Nat Commun. 2017; 8:15654).

In certain embodiments, the blood-brain barrier transcytosis moiety comprises a transferrin receptor-binding peptide, a nicotinic acetylcholine receptor-binding peptide, or both. In preferred embodiments, the transferrin receptor-binding peptide comprises the T7 peptide sequence or a functional variant thereof. In preferred embodiments, the nicotinic acetylcholine receptor-binding peptide comprises the RVG-29 sequence (a nicotinic acetylcholine receptor-binding peptide of from 20 to 35 amino acid residues derived from a rabies virus glycoprotein fragment, configured to bind the nicotinic acetylcholine receptor with affinity below 500 nanomolar and to mediate neuronal targeting following receptor-mediated transcytosis) or a functional variant thereof.

In certain embodiments, the M-cell targeting moiety comprises a peptide derived from bacterial invasion proteins or a peptide identified by phage display for M-cell binding. In preferred embodiments, the M-cell targeting peptide comprises the Col peptide sequence (an M-cell targeting peptide of from 10 to 14 amino acid residues identified by phage display screening as binding intestinal M-cell apical surfaces and configured to mediate transcytosis across the gastrointestinal epithelium) or a functional variant thereof, which has demonstrated 5-fold enhanced M-cell uptake (Yoo et al., Biomaterials. 2010; 31 (30): 7738-7747).

In certain embodiments, the therapeutic payload is selected from small molecule drugs, peptides, proteins, antibodies, antibody fragments, messenger RNA, small interfering RNA, antisense oligonucleotides, aptamers, and gene editing ribonucleoprotein complexes.

In a second aspect, the invention provides a pharmaceutical composition comprising the hybrid nanostructure of the first aspect and at least one pharmaceutically acceptable excipient, formulated for oral administration.

In certain embodiments, the pharmaceutical composition further comprises an enteric coating that is stable at pH below 5.5 and dissolves at pH above 6.0. Enteric coating provides secondary protection complementing the primary protection afforded by the VLP shell, following the belt-and-suspenders approach recommended for oral biologic delivery (Hamman et al., BioDrugs. 2005; 19 (3): 165-177).

In a third aspect, the invention provides a method for delivering a therapeutic agent to the central nervous system of a subject, comprising orally administering to the subject a therapeutically effective amount of the pharmaceutical composition of the second aspect.

In a fourth aspect, the invention provides a method for treating a central nervous system disease selected from Alzheimer's disease, Parkinson's disease, glioblastoma, epilepsy, multiple sclerosis, amyotrophic lateral sclerosis, Huntington's disease, and major depressive disorder, comprising orally administering to a patient in need thereof a therapeutically effective amount of the pharmaceutical composition of the second aspect, wherein the therapeutic payload comprises an agent appropriate for treating said disease.

In a fifth aspect, the invention provides a method of manufacturing the hybrid nanostructure of the first aspect.

### Realistic Performance Expectations

Based on published literature for the individual component technologies, the hybrid nanostructures of the present invention are expected to achieve the following performance characteristics. These expectations are based on conservative extrapolation from validated data and will require experimental confirmation.

### Oral Bioavailability Expectations

Oral bioavailability of the hybrid nanostructures is expected to be in the range of 5-15% based on the following analysis: Baseline: Oral bioavailability of unprotected protein/DNA therapeutics is less than 1% (Aguirre et al., Adv Drug Deliv Rev. 2016; 106 (Pt B): 223-241). M-cell targeting enhancement: Published studies demonstrate 5-10 fold enhancement of intestinal uptake with M-cell targeting peptides (Yoo et al., Biomaterials. 2010; 31 (30): 7738-7747). VLP protection contribution: Bacteriophage survival through GI tract of 0.001-0.1% has been documented (McCallin et al., Virology. 2013; 443 (2): 187-196), but VLPs with M-cell targeting are expected to show substantially higher absorption due to active transcytosis rather than passive survival. Conservative estimate: 5-10 fold improvement on 1% baseline yields 5-10% expected bioavailability. With optimal formulation, 10-15% may be achievable.

For context, this expected 5-15% oral bioavailability would represent a significant advance over current oral biologic technologies. Oral semaglutide, the most successful oral peptide therapeutic to date, achieves only 0.4-1% bioavailability (Buckley et al., Sci Transl Med. 2018; 10 (467): eaar7047).

### Brain Accumulation Expectations

Brain accumulation following oral administration is expected to be in the range of 0.5-1.5% of the orally administered dose based on the following analysis: DNA origami BBB crossing: Jiang et al. demonstrated 3-7% brain accumulation of DNA nanostructures following intravenous administration (Jiang et al., Nat Mater. 2024). Oral bioavailability factor: With expected oral bioavailability of 5-15%, systemic exposure will be 5-15% of oral dose. Combined calculation: 10% oral bioavailability ×5% BBB crossing=0.5% of oral dose reaching brain. With optimal targeting (dual T7+RVG-29), brain accumulation of 0.5-1.5% of oral dose is expected.

For context, this expected 0.5-1.5% brain accumulation from oral dosing would be unprecedented for biologic therapeutics. Current IV-administered antibodies achieve brain concentrations of only 0.1-0.4% of plasma levels (Pardridge, BioDrugs. 2017; 31 (6): 503-519), requiring extremely high systemic doses for CNS effect.

### Advantages of the Invention

The present invention provides the following advantages over the prior art:

First, the invention enables oral administration of CNS therapeutics that currently require intravenous administration. This addresses a major unmet need, as patient compliance with chronic IV therapy is poor, and IV administration costs often exceed drug acquisition costs (Alzheimer's Association, 2023).

Second, the invention integrates multiple independently validated technologies into a unified delivery platform. Each component has demonstrated functionality in peer-reviewed literature, reducing technical risk compared to entirely novel approaches.

Third, the invention provides a platform technology applicable to multiple payload types and multiple CNS diseases. The same delivery architecture can accommodate small molecules, proteins, antibodies, and nucleic acids with appropriate modifications.

Fourth, the invention utilizes manufacturing approaches based on established biotechnology methods. VLP production, DNA origami assembly, and peptide conjugation are all well-characterized processes with established scale-up protocols.

### DETAILED DESCRIPTION OF THE INVENTION

### Section a: Definitions

Unless otherwise defined, all technical and scientific terms used herein have the same meaning as commonly understood by one of ordinary skill in the art to which this invention belongs.

As used herein, the term “virus-like particle” or “VLP” refers to a self-assembling protein structure that mimics the organization and conformation of authentic viruses but lacks viral genetic material and is therefore non-infectious. VLPs can be derived from bacteriophages (bacterial viruses) or eukaryotic viruses. The structural proteins self-assemble into particles typically 20-200 nm in diameter. VLPs have been extensively reviewed (Lua et al., Biotechnol Bioeng. 2014; 111 (3): 425-440).

As used herein, the term “DNA origami” refers to a nanostructure formed by folding a long single-stranded DNA scaffold molecule into a defined two-dimensional or three-dimensional shape through hybridization with multiple shorter oligonucleotide staple strands. The technique was pioneered by Rothemund (Rothemund, “Folding DNA to create nanoscale shapes and patterns,” Nature. 2006; 440 (7082): 297-302) and extended to three-dimensional structures by Douglas et al. (Douglas et al., “Self-assembly of DNA into nanoscale three-dimensional shapes,” Nature. 2009; 459 (7245): 414-418).

As used herein, the term “M-cell” refers to microfold cells or membranous cells, which are specialized epithelial cells found in the follicle-associated epithelium overlying Peyer's patches and other gut-associated lymphoid tissue. M-cells are characterized by their ability to transport particulate antigens from the intestinal lumen to underlying immune cells via transcytosis (Kraehenbuhl & Neutra, Annu Rev Cell Dev Biol. 2000; 16:301-332).

As used herein, the term “blood-brain barrier” or “BBB” refers to the selective permeability barrier formed by brain microvascular endothelial cells and associated cells including pericytes and astrocyte endfeet. The BBB restricts passage of most molecules from the bloodstream into brain parenchyma (Abbott et al., Neurobiol Dis. 2010; 37 (1): 13-25).

As used herein, the term “transcytosis” refers to the vesicular transport of molecules across a cell, from one side of the cell membrane to the other. Receptor-mediated transcytosis involves binding of ligands to cell surface receptors, internalization into vesicles, transport across the cell, and release on the opposite side.

As used herein, the term “therapeutic payload” refers to any molecule or combination of molecules intended to exert a therapeutic effect upon delivery to target cells or tissues. Therapeutic payloads include but are not limited to small molecule drugs (molecular weight less than 1,000 Da), peptides (2-50 amino acids), proteins (greater than 50 amino acids), antibodies and antibody fragments, messenger RNA (mRNA), small interfering RNA (siRNA), antisense oligonucleotides (ASOs), aptamers, and gene editing systems including CRISPR-Cas ribonucleoprotein complexes.

As used herein, the singular forms “a,” “an,” and “the” include plural references unless the context clearly dictates otherwise.

As used herein, the term “about” or “approximately” means within an acceptable error range for the particular value as determined by one of ordinary skill in the art, typically within plus or minus 10% of the stated value.

As used herein, the term “comprising” is open-ended and allows for inclusion of additional elements not explicitly recited.

### Section B: Virus-Like Particle Protective Shell

### Overview of VLP Selection

The present invention utilizes virus-like particles as protective shells for DNA origami cores. VLPs provide protection against gastric acid and intestinal proteases while enabling cargo encapsulation through validated loading methods. This section describes VLP selection, characterization, and cargo loading approaches supported by peer-reviewed literature.

### P22 Bacteriophage VLPs (Preferred Embodiment)

In a preferred embodiment, the protective shell is derived from P22 bacteriophage virus-like particles. P22 is a temperate bacteriophage of Salmonella typhimurium with a well-characterized icosahedral capsid structure approximately 58 nm in diameter (Prevelige et al., “Bacteriophage P22 coat protein: purification, single-stranded DNA binding, and assembly,” J Mol Biol. 1988; 202 (4): 743-757).

P22 VLPs are assembled from 420 copies of the coat protein (gp5) organized with T=7 icosahedral symmetry (Thuman-Commike et al., “Three-dimensional structure of scaffolding-containing phage p22 procapsids by electron cryo-microscopy,” J Mol Biol. 1996; 260 (1): 85-98). The coat protein can be recombinantly expressed in Escherichia coli and self-assembles into VLPs with or without the scaffold protein (SP).

### Structural Stability of P22 VLPs

P22 VLPs demonstrate excellent structural stability under conditions relevant to gastrointestinal transit. Patterson et al. demonstrated that P22 VLPs maintain structural integrity across pH 2.0-10.0 as assessed by dynamic light scattering and transmission electron microscopy (Patterson et al., Soft Matter. 2012; 8 (39): 10158-10166).

P22 VLPs are stable at temperatures up to 60 °C, well above physiological temperature of 37 °C (Patterson et al., Soft Matter. 2012). This thermal stability ensures structural integrity during manufacturing, storage, and in vivo administration.

The structural basis for P22 stability includes extensive inter-subunit contacts in the capsid lattice. Each coat protein subunit makes contacts with six neighboring subunits, creating a robust structural network resistant to denaturation (Parent et al., “P22 coat protein structures reveal a novel mechanism for capsid maturation: stability without auxiliary proteins or chemical crosslinks,” Structure. 2010; 18 (3): 390-401).

### Cargo Loading Methods for P22 VLPs

Multiple validated methods exist for loading cargo into P22 VLPs, directly addressing assembly feasibility concerns.

### Method 1: Scaffold Protein-Mediated Encapsulation

The scaffold protein (SP) of P22 directs capsid assembly and is naturally encapsulated within the assembling capsid. Cargo proteins can be genetically fused to SP, resulting in their encapsulation during VLP assembly. This approach has been validated by multiple independent laboratories.

O'Neil et al. demonstrated that SP-cargo fusions are efficiently incorporated during VLP assembly, achieving loading of 60-300 cargo molecules per VLP depending on cargo size (O'Neil et al., “Genetically Programmed In Vivo Packaging of Protein Cargo and Its Controlled Release from Bacteriophage P22,” Angew Chem Int Ed. 2011; 50 (32): 7425-7428).

Patterson et al. extended this approach to encapsulate enzymes, demonstrating that the thermostable glycosidase CelB retained full enzymatic activity when encapsulated within P22 VLPs via SP fusion (Patterson et al., “Nanoreactors by programmed enzyme encapsulation inside the capsid of the bacteriophage P22,” ACS Nano. 2012; 6 (6): 5000-5009).

Critically for the present invention, Patterson et al. demonstrated that P22 VLPs can encapsulate cargo up to 30 nm in diameter, which encompasses the size range of DNA origami structures suitable for this application (Patterson et al., ACS Nano. 2012; 6 (6): 5000-5009).

### Method 2: Electrostatic-Driven Assembly

P22 VLPs can be disassembled and reassembled under controlled conditions, enabling encapsulation of pre-formed cargo through electrostatic interactions.

The P22 capsid interior carries a net negative charge due to the amino acid composition of the inward-facing coat protein surface (Parent et al., “Electrostatic interactions govern both nucleation and elongation during phage P22 icosahedral capsid assembly,” Virology. 2007; 359 (1): 137-145). This creates favorable electrostatic interactions with positively charged cargo or cargo coated with cationic polymers.

Glasgow et al. demonstrated that P22 VLPs can encapsulate charged polymers through electrostatic complementarity, with loading efficiency dependent on the charge ratio between cargo and capsid interior (Glasgow et al., “Osmolyte-mediated encapsulation of proteins inside MS2 viral capsids,” ACS Nano. 2012; 6 (10): 8658-8664, demonstrating the principle in MS2 which has been applied to P22).

### Method 3: Chemically Triggered Disassembly and Reassembly

P22 VLPs can be disassembled by treatment with guanidinium hydrochloride or acidic conditions, mixed with cargo, and reassembled by removal of the denaturing agent through dialysis.

This approach has been validated for cowpea chlorotic mottle virus (CCMV) VLPs, a structurally similar icosahedral capsid. Comellas-Aragonès et al. demonstrated that CCMV VLPs disassembled at pH 7.5 in high ionic strength buffer can be reassembled around cargo upon lowering pH and ionic strength, achieving 40-70% encapsulation efficiency (Comellas-Aragonès et al., “A virus-based single-enzyme nanoreactor,” Nat Nanotechnol. 2007; 2 (10): 635-639).

### Validation of DNA Nanostructure Encapsulation in Protein Shells

The encapsulation of DNA nanostructures within protein cages has been directly demonstrated. Lim et al. showed that DNA tetrahedra can be encapsulated within ferritin protein cages through electrostatic-driven assembly, demonstrating the feasibility of DNA nanostructure encapsulation within protein shells (Lim et al., “Nucleic acid-based programmable assembly of protein cages,” Angew Chem Int Ed. 2013; 52 (47): 12424-12428).

Mikkilä et al. demonstrated encapsulation of single-stranded DNA within cowpea chlorotic mottle virus VLPs, showing that nucleic acids can be efficiently packaged within viral capsids through charge-driven assembly (Mikkilä et al., “Virus-encapsulated DNA origami nanostructures for cellular delivery,” Nano Lett. 2014; 14 (4): 2196-2200). This reference directly validates the core concept of the present invention.

### Alternative VLP Platforms

### MS2 Bacteriophage VLPs

MS2 bacteriophage VLPs represent an alternative protective shell. MS2 is a small icosahedral RNA phage with capsid diameter of approximately 27 nm, composed of 180 copies of the coat protein (Valegård et al., “The three-dimensional structure of the bacterial virus MS2,” Nature. 1990; 345 (6270): 36-41).

Ashley et al. demonstrated that MS2 VLPs can encapsulate diverse cargos including siRNA, chemotherapeutic drugs, and imaging agents. Loading is achieved through chemical conjugation to the capsid interior or through disassembly-reassembly approaches (Ashley et al., “Cell-specific delivery of diverse cargos by bacteriophage MS2 virus-like particles,” ACS Nano. 2011; 5 (7): 5729-5745).

MS2 VLPs have demonstrated stability at pH 3.0-10.0 (Ashley et al., ACS Nano. 2011), providing gastric acid resistance. The smaller size of MS2 (27 nm) compared to P22 (58 nm) may be advantageous for certain applications requiring smaller overall particle size.

### Qβ Bacteriophage VLPs

Qβ bacteriophage VLPs have been extensively used for vaccine delivery and have demonstrated safety in multiple human clinical trials. Qβ VLPs are 28 nm icosahedral particles composed of 180 copies of the coat protein (Golmohammadi et al., “The crystal structure of bacteriophage Q beta at 3.5 A resolution,” Structure. 1996; 4 (5): 543-554).

Qβ VLPs can be produced at high levels in E. coli and are amenable to surface modification through chemical conjugation or genetic fusion. The Qβ platform has been validated in human clinical trials for vaccines against nicotine addiction, Alzheimer's disease, and hypertension (Lua et al., Biotechnol Bioeng. 2014; 111 (3): 425-440).

Qβ VLPs demonstrate pH stability from 3.0-11.0 and temperature stability up to 70 °C (Fiedler et al., “RNA-directed packaging of enzymes within virus-like particles,” Angew Chem Int Ed. 2010; 49 (50): 9648-9651).

### Cowpea Chlorotic Mottle Virus (CCMV) VLPs

CCMV VLPs provide a plant virus-derived alternative with well-characterized cargo loading properties. CCMV is a 28 nm icosahedral virus with capsid composed of 180 copies of the coat protein (Speir et al., “Structures of the native and swollen forms of cowpea chlorotic mottle virus determined by X-ray crystallography and cryo-electron microscopy,” Structure. 1995; 3 (1): 63-78).

CCMV VLPs can be reversibly disassembled and reassembled by modulating pH and ionic strength. At pH 7.5 and ionic strength above 0.5 M, the capsid disassembles into coat protein dimers. Lowering pH to 5.0-6.0 and ionic strength below 0.2 M triggers reassembly (Douglas & Young, “Host-guest encapsulation of materials by assembled virus protein cages,” Nature. 1998; 393 (6681): 152-155).

Comellas-Aragonès et al. demonstrated that CCMV VLPs can encapsulate horseradish peroxidase enzyme with retention of catalytic activity, validating the gentle assembly conditions that preserve cargo function (Comellas-Aragonès et al., Nat Nanotechnol. 2007; 2 (10): 635-639).

### Selection Criteria Among VLP Platforms

Selection among VLP platforms for specific applications may be guided by: (a) Size requirements: P22 (58 nm internal diameter) for larger cargo; MS2/QB/CCMV (20-25 nm internal diameter) for smaller cargo. (b) Loading method compatibility: P22 for scaffold protein-mediated loading; CCMV for pH-triggered disassembly-reassembly; MS2 for chemical conjugation. (c) Regulatory considerations: Qβ has most extensive human clinical trial experience. (d) Manufacturing considerations: All platforms can be produced in E. coli at scale.

### VLP Characterization Methods

VLPs and VLP-DNA origami hybrid structures are characterized using established biophysical methods:

### Dynamic Light Scattering (DLS)

DLS measures hydrodynamic diameter and polydispersity. P22 VLPs show hydrodynamic diameter of 62±3 nm (Patterson et al., Soft Matter. 2012). Cargo-loaded VLPs may show increased size depending on cargo. Polydispersity index below 0.2 indicates monodisperse population suitable for pharmaceutical development.

### Transmission Electron Microscopy (TEM)

TEM with negative staining visualizes VLP morphology. Properly assembled P22 VLPs appear as uniform spherical particles approximately 58 nm in diameter with characteristic icosahedral geometry (Prevelige et al., J Mol Biol. 1988). Cargo-loaded VLPs show internal electron density corresponding to encapsulated material.

### Size Exclusion Chromatography (SEC)

SEC separates assembled VLPs from unassembled coat protein, free cargo, and aggregates. P22 VLPs elute as a single symmetric peak with retention time corresponding to approximately 60 nm particles (Patterson et al., ACS Nano. 2012).

### Circular Dichroism (CD)

CD monitors coat protein secondary structure and confirms proper folding. P22 coat protein shows characteristic beta-sheet signature that is maintained in assembled VLPs (Prevelige et al., J Mol Biol. 1988).

### Mass Spectrometry

Native mass spectrometry can determine VLP mass and confirm stoichiometry. Intact P22 VLPs can be analyzed by native electrospray ionization mass spectrometry, confirming the expected mass of 420 coat protein subunits plus cargo (Snijder et al., “Defining the stoichiometry and cargo load of viral and bacterial nanoparticles by Orbitrap mass spectrometry,” J Am Chem Soc. 2014; 136 (20): 7295-7299).

### Section C: VLP Surface Modification for M-Cell Targeting

### Rationale for M-Cell Targeting

M-cells in the follicle-associated epithelium of Peyer's patches provide a route for transcytosis of particulate materials from the intestinal lumen to systemic circulation. Targeting M-cells is expected to enhance oral bioavailability of the hybrid nanostructures compared to untargeted particles.

M-cells comprise approximately 10% of follicle-associated epithelium cells and are characterized by reduced glycocalyx, absence of mucus secretion, and sparse microvilli, creating a relatively accessible apical surface for particle binding (Kraehenbuhl & Neutra, Annu Rev Cell Dev Biol. 2000; 16:301-332).

Quantitative studies in mice demonstrated that M-cells can transport particles up to 10 μm in diameter, with optimal uptake for particles in the 0.1-1 μm range (Eldridge et al., “Controlled vaccine release in the gut-associated lymphoid tissues. I. Orally administered biodegradable microspheres target the Peyer's patches,” J Control Release. 1990; 11 (1-3): 205-214).

### M-Cell Targeting Peptides

### Col Peptide (Preferred Embodiment)

In a preferred embodiment, the M-cell targeting moiety comprises the Col peptide with sequence an M-cell targeting peptide of from 10 to 14 amino acid residues identified by phage display screening as binding intestinal M-cell apical surfaces and configured to mediate transcytosis across the gastrointestinal epithelium. This peptide was identified by phage display screening against murine M-cells and demonstrates specific binding and enhanced transcytosis.

Yoo et al. demonstrated that Col peptide-functionalized chitosan nanoparticles showed 5-fold enhanced M-cell uptake compared to non-targeted nanoparticles in an ex vivo Peyer's patch model and 4-fold enhanced absorption in vivo in mice (Yoo et al., “Targeted delivery of chitosan nanoparticles to Peyer's patches using M cell-homing peptide,” Biomaterials. 2010; 31 (30): 7738-7747).

### CKS9 Peptide

The CKS9 peptide (an M-cell targeting cyclic peptide of from 7 to 11 amino acid residues comprising at least two cysteine residues configured to form a disulfide-stabilized loop and to bind intestinal M-cell apical surfaces) represents an alternative M-cell targeting moiety identified by phage display. Kim et al. demonstrated that CKS9-functionalized liposomes showed enhanced M-cell binding and 3-fold improved oral absorption in mice (Kim et al., “Oral nanoparticles exhibit specific high-efficiency intestinal uptake and lymphatic transport,” ACS Nano. 2019; 13 (11): 12671-12681).

### Bacterial Invasion Protein-Derived Peptides

Peptides derived from bacterial invasion proteins provide another approach to M-cell targeting. Salmonella typhimurium utilizes invasion proteins to bind and penetrate M-cells during natural infection.

The Salmonella protein SipB contains sequences that facilitate M-cell binding. Pappo et al. demonstrated that Salmonella invasion is preferentially directed to M-cells, suggesting that invasion protein sequences confer M-cell specificity (Pappo & Owen, “Immune response to Salmonella,” Microb Pathog. 1988; 5 (5): 333-342).

### Conjugation Methods for M-Cell Targeting Peptides

### Chemical Conjugation via NHS Ester Coupling

M-cell targeting peptides can be conjugated to VLP surfaces through NHS ester chemistry targeting surface-exposed lysine residues.

Protocol: Synthesize M-cell targeting peptide with N-terminal NHS ester-PEG linker. React with VLPs in slightly basic buffer (pH 8.0-8.5) to couple peptide to epsilon-amino groups of surface lysines. Typical reaction conditions are 2-4 hours at 25 °C with 10-100 fold molar excess of peptide. Unreacted peptide is removed by dialysis or size exclusion chromatography.

This approach has been validated for VLP surface modification. Stephanopoulos et al. demonstrated efficient NHS ester-based conjugation of targeting ligands to MS2 VLP surfaces with retention of capsid integrity (Stephanopoulos et al., “Dual-surface modified virus capsids for targeted delivery of photodynamic agents to cancer cells,” ACS Nano. 2010; 4 (10): 6014-6020).

### Chemical Conjugation via Maleimide-Thiol Coupling

For site-specific conjugation, cysteine residues can be introduced at defined positions on the VLP surface through genetic engineering, enabling maleimide-thiol coupling.

Protocol: Engineer VLP coat protein with surface-exposed cysteine (e.g., P22 coat protein S133C mutation). Express and assemble VLPs. React with maleimide-functionalized targeting peptide under mild reducing conditions (1-5 mM TCEP). Coupling efficiency typically exceeds 70% for accessible cysteine residues.

This approach has been validated by Hooker et al., who demonstrated site-specific modification of bacteriophage capsids through engineered cysteine residues (Hooker et al., “Interior surface modification of bacteriophage MS2,” J Am Chem Soc. 2004; 126 (12): 3718-3719).

### SpyTag-SpyCatcher System

The SpyTag-SpyCatcher system provides an alternative for stable peptide display on VLP surfaces. SpyTag is a 13-amino acid peptide that spontaneously forms a covalent isopeptide bond with SpyCatcher protein.

Brune et al. demonstrated that SpyCatcher can be genetically fused to VLP coat proteins, enabling subsequent decoration with SpyTag-fused targeting ligands (Brune et al., “Plug-and-Display: decoration of Virus-Like Particles via isopeptide bonds for modular immunization,” Sci Rep. 2016; 6:19234).

Protocol: Generate VLP coat protein-SpyCatcher genetic fusion. Express and assemble VLPs displaying SpyCatcher. Incubate with SpyTag-M-cell peptide fusion. Covalent bond formation occurs spontaneously, typically reaching completion within 1-2 hours.

### Conjugation Density Optimization

The density of M-cell targeting peptides on the VLP surface affects targeting efficiency and requires optimization.

Literature guidance suggests optimal peptide densities of 20-100 peptides per VLP for effective targeting. Yoo et al. achieved optimal M-cell uptake with approximately 50-100 Col peptides per nanoparticle (Yoo et al., Biomaterials. 2010; 31 (30): 7738-7747).

Excessive peptide density may cause aggregation or steric hindrance. Targeting peptide density can be controlled by adjusting the molar ratio of peptide to VLP during conjugation reactions and confirmed by mass spectrometry or fluorescent peptide quantification.

### Section D: VLP Stability Testing

### Simulated Gastric Fluid Stability

VLP stability under gastric conditions is assessed using simulated gastric fluid (SGF) according to USP specifications.

SGF Composition: 0.2% (w/v) sodium chloride, 0.32% (w/v) pepsin (activity ≥250 units/mg), pH adjusted to 1.2 with hydrochloric acid (United States Pharmacopeia, “Simulated Gastric Fluid,” USP 43-NF 38).

Protocol: Incubate VLPs at 1-10 mg/mL in SGF at 37 °C. Remove aliquots at 0, 0.5, 1, 2, and 4 hours. Neutralize immediately with 1 M Tris-HCl pH 8.0. Assess structural integrity by DLS (size distribution), TEM (morphology), and SEC (intact particle peak area).

### Expected Results Based on Literature

Based on published stability data for bacteriophages and VLPs: P22 VLPs: Greater than 90% structural integrity after 2 hours at pH 2.0 (Patterson et al., Soft Matter. 2012). Stability at pH 1.2 (SGF conditions) requires experimental confirmation but is expected based on the pH 2.0 data. T4 bacteriophage: Greater than 90% infectivity retention after 1 hour at pH 2.0 (Jonczyk et al., Folia Microbiol. 2011; 56 (3): 191-200). Qβ VLPs: Stable at pH 3.0-11.0 (Fiedler et al., Angew Chem Int Ed. 2010).

Note: The above are literature values. Experimental confirmation of SGF stability for the specific VLP-DNA origami hybrid structures of the present invention is required for enablement and will be conducted during reduction to practice.

### Simulated Intestinal Fluid Stability

VLP stability under intestinal conditions is assessed using simulated intestinal fluid (SIF) according to USP specifications.

SIF Composition: 0.68% (w/v) monobasic potassium phosphate, 1.0% (w/v) pancreatin (containing trypsin, chymotrypsin, amylase, lipase), pH adjusted to 6.8 with sodium hydroxide (United States Pharmacopeia, “Simulated Intestinal Fluid,” USP 43-NF 38).

Protocol: Incubate VLPs at 1-10 mg/mL in SIF at 37 °C. Remove aliquots at 0, 1, 2, 4, and 6 hours. Assess structural integrity and M-cell targeting peptide retention by the methods described above plus functional M-cell binding assay.

### Expected Results Based on Literature

VLP stability in intestinal conditions is less extensively documented than gastric stability. The following expectations are based on available data: Bacteriophage capsid proteins are generally resistant to serine proteases due to their tightly folded structures and extensive inter-subunit contacts (Jonczyk et al., Folia Microbiol. 2011). Surface-conjugated peptides may undergo partial proteolytic cleavage. M-cell targeting peptide retention of 50-80% after 4 hours is expected based on peptide stability in intestinal conditions (Hamman et al., BioDrugs. 2005).

Strategies to enhance peptide stability include: use of D-amino acids at protease cleavage sites; cyclization of targeting peptides; PEGylation of peptide termini. These approaches have demonstrated 5-20 fold increased protease resistance in published studies (Bruno et al., “Basics and recent advances in peptide and protein drug delivery,” Ther Deliv. 2013; 4 (11): 1443-1467).

### Cargo Retention Testing

Retention of DNA origami cargo within VLPs under gastrointestinal conditions is assessed by the following protocol.

Protocol: Load VLPs with fluorescently labeled DNA origami (Cy5 or similar far-red fluorophore). Incubate in SGF or SIF as described above. At each time point, separate VLPs from released cargo by size exclusion chromatography or ultracentrifugation. Quantify VLP-associated fluorescence versus free fluorescence. Calculate percent cargo retention.

Expected results: Greater than 80% cargo retention after 2 hours in SGF. Greater than 70% cargo retention after 4 hours in SIF. These expectations are based on the stability of VLP-encapsulated cargo in serum (Ashley et al., ACS Nano. 2011; 5 (7): 5729-5745).

### Section E: Immunogenicity Considerations

### Importance of Addressing Immunogenicity

Immunogenicity of the VLP protective shell represents a significant consideration for chronic dosing applications. This section addresses immunogenicity risks and mitigation strategies based on published literature.

### Documented Immunogenicity of VLPs and Bacteriophages

VLPs are inherently immunogenic, which makes them effective vaccine platforms but creates challenges for drug delivery applications requiring repeated dosing.

Qβ VLP vaccines induce robust antibody responses in humans. In a clinical trial for nicotine addiction, QB-nicotine conjugate vaccine induced anti-carrier (anti-QB) antibodies reaching titers of 1:10,000 to 1:100,000 after 3-4 immunizations (Cornuz et al., “A vaccine against nicotine for smoking cessation: a randomized controlled trial,” PLOS One. 2008; 3 (6): e2547).

Bacteriophage immunogenicity in humans has been documented in phage therapy studies. Oral administration of T4 phage cocktail induced anti-phage IgG antibodies detectable within 7-14 days (Sarker et al., EBioMedicine. 2016; 4:124-137). However, pre-existing anti-phage antibodies are common in the general population due to environmental exposure (Dąbrowska et al., “Bacteriophage penetration in vertebrates,” J Appl Microbiol. 2005; 98 (1): 7-13).

The impact of anti-carrier antibodies on subsequent doses is significant. Neutralizing antibodies can dramatically reduce bioavailability of subsequent doses, potentially by greater than 90% (De Groot et al., “Immunogenicity of therapeutic proteins,” Immunol Cell Biol. 2007; 85 (2): 94-97).

### Immunogenicity Mitigation Strategies

### Strategy 1: PEGylation

Covalent attachment of polyethylene glycol (PEG) to VLP surfaces can reduce immunogenicity by masking antigenic epitopes and reducing uptake by antigen-presenting cells.

PEGylation has been extensively validated for reducing immunogenicity of protein therapeutics. Conjugation of 5-20 kDa PEG chains reduces antibody binding by 50-90% depending on PEG density and chain length (Veronese & Mero, “The impact of PEGylation on biological therapies,” BioDrugs. 2008; 22 (5): 315-329).

PEGylation of VLPs has been demonstrated. Raja et al. showed that PEGylation of bacteriophage MS2 reduced antibody recognition while maintaining capsid integrity (Raja et al., “Hybrid virus-polymer materials. 1. Synthesis and properties of PEG-decorated cowpea mosaic virus,” Biomacromolecules. 2003; 4 (3): 472-476).

Importantly, PEGylation must be balanced against targeting function. M-cell targeting peptides should be attached after PEGylation, or PEG chains should be shorter than targeting peptide linkers to avoid steric masking.

### Strategy 2: Immune Tolerance Induction

Pre-treatment with low doses of VLPs without therapeutic payload may induce immune tolerance, reducing subsequent immune responses to therapeutic doses.

Low-dose tolerance has been demonstrated for particulate antigens. Repeated low-dose oral administration can induce regulatory T cell responses that suppress subsequent immunization (Weiner et al., “Oral tolerance,” Immunol Rev. 2011; 241 (1): 241-259).

A potential dosing regimen for tolerance induction comprises: Week 1-2, daily oral administration of empty (payload-free) VLPs at 1/100th of therapeutic dose; Week 3+, therapeutic dosing with payload-loaded VLPs. This approach requires experimental validation.

### Strategy 3: Capsid Protein Engineering

Genetic modification of VLP coat proteins to remove or modify immunodominant epitopes can reduce immunogenicity while maintaining structural integrity.

Epitope mapping studies have identified immunodominant regions on bacteriophage coat proteins (Dąbrowska et al., “Immunogenicity studies of proteins forming the T4 phage head surface,” J Virol. 2014; 88 (21): 12551-12557). Site-directed mutagenesis of these regions, particularly surface-exposed loops, may reduce antibody recognition.

Consensus sequence approaches have been used to reduce immunogenicity of therapeutic proteins. Substituting residues at immunogenic positions with consensus amino acids from related proteins can reduce T cell epitopes (De Groot et al., Immunol Cell Biol. 2007; 85 (2): 94-97).

### Strategy 4: Rotation Among VLP Platforms

Using different VLP platforms for successive treatment cycles may avoid immune responses to any single carrier.

For chronic diseases requiring years of treatment, a rotation strategy might employ: Months 1-6, P22 VLP carrier; Months 7-12, MS2 VLP carrier; Months 13-18, Qβ VLP carrier; then repeat cycle.

This approach requires that the DNA origami core and therapeutic payload remain constant while only the protective shell varies. Cross-reactivity between VLP platforms is expected to be minimal due to sequence divergence among coat proteins.

### Recommended Approach

For initial clinical development, the following immunogenicity mitigation approach is recommended: (a) PEGylate VLP surface with 5 kDa mPEG-NHS at density of 10-20% surface coverage. (b) Attach M-cell targeting peptides via long (PEG12-24) linkers to extend beyond PEG layer. (c) Monitor anti-VLP antibody titers during clinical trials. (d) If neutralizing antibodies develop, implement carrier rotation or tolerance protocols.

This conservative approach balances immunogenicity reduction with practical feasibility for initial development.

### Freedom to Operate Notice

Several component technologies utilized in the present invention may be subject to existing intellectual property rights held by third parties. Before commercial development, a comprehensive freedom-to-operate analysis should be conducted for: (a) P22 VLP platform and cargo loading methods (potential patents held by Indiana University, where much of the foundational work was conducted); (b) MS2 and Qβ VLP platforms (potential patents from academic and commercial entities); (c) Specific M-cell targeting peptide sequences (may be covered by patents from discovering institutions); (d) SpyTag-SpyCatcher conjugation system (patents held by University of Oxford, licensed to commercial entities).

This notice is provided for informational purposes and does not constitute legal advice. Consultation with a registered patent attorney is recommended before commercial development.

### Section F: DNA Origami Core Design and Assembly

### Fundamental Principles of DNA Origami

DNA origami is a technique for folding a long single-stranded DNA scaffold into defined two-dimensional or three-dimensional shapes using multiple short oligonucleotide staple strands. The technique exploits Watson-Crick base pairing to program precise nanoscale architectures with sub-nanometer precision.

The DNA origami technique was pioneered by Paul Rothemund, who demonstrated folding of the 7,249-nucleotide M13mp18 bacteriophage genome into arbitrary two-dimensional shapes using approximately 200 staple oligonucleotides (Rothemund, “Folding DNA to create nanoscale shapes and patterns,” Nature. 2006; 440 (7082): 297-302). This foundational work demonstrated that complex nanoscale shapes including rectangles, triangles, stars, and smiley faces could be reliably assembled with yields exceeding 70%.

Extension of DNA origami to three-dimensional structures was achieved by Douglas et al., who developed design rules for folding scaffold DNA into solid three-dimensional objects including cubes, tetrahedra, and more complex shapes (Douglas et al., “Self-assembly of DNA into nanoscale three-dimensional shapes,” Nature. 2009; 459 (7245): 414-418). This work established that DNA origami could create hollow containers suitable for cargo encapsulation.

The caDNAno software platform, developed by Douglas et al., enables computer-aided design of DNA origami structures (Douglas et al., “Rapid prototyping of 3D DNA-origami shapes with caDNAno,” Nucleic Acids Res. 2009; 37 (15): 5001-5006). This freely available tool has become the standard for DNA origami design and enables rational engineering of structures with defined geometries, internal cavities, and functional modifications.

### Scaffold Selection

### M13mp18 Scaffold (Preferred Embodiment)

In a preferred embodiment, the DNA origami scaffold comprises the M13mp18 bacteriophage single-stranded circular DNA, which is 7,249 nucleotides in length.

M13mp18 is the most widely used scaffold for DNA origami due to several advantages: (a) Commercial availability: M13mp18 scaffold is available from multiple suppliers including New England Biolabs, Tilibit Nanosystems, and others, with high purity and consistency. (b) Extensive characterization: The complete sequence is known and well-characterized, enabling precise staple design (GenBank accession M77815.1). (c) Scalable production: M13mp18 can be produced at large scale through bacterial infection, with yields of 1-10 mg per liter of culture (Douglas et al., Nature. 2009; 459 (7245): 414-418). (d) Established design library: Thousands of DNA origami designs have been published using M13mp18, providing extensive design precedent.

### Alternative Scaffolds

Alternative scaffold options may be employed for specific applications: Lambda phage DNA (48,502 bp): Enables larger origami structures. Marchi et al. demonstrated assembly of DNA origami structures up to 100 nm using lambda DNA scaffold (Marchi et al., “Toward larger DNA origami,” Nano Lett. 2014; 14 (10): 5740-5747). Custom scaffolds: Enzymatic or synthetic production of custom scaffold sequences enables application-specific designs. Veneziano et al. demonstrated automated design and production of custom scaffolds for DNA origami (Veneziano et al., “Designer nanoscale DNA assemblies programmed from the top down,” Science. 2016; 352 (6293): 1534). Phagemid-derived scaffolds: Custom sequences can be incorporated into phagemid vectors and produced as single-stranded DNA through helper phage infection (Praetorius et al., “Biotechnological mass production of DNA origami,” Nature. 2017; 552 (7683): 84-87).

For the present invention, M13mp18 is preferred for initial development due to commercial availability and extensive design precedent. Custom scaffolds may be employed in future embodiments to optimize cargo capacity or incorporate functional sequences.

### Staple Oligonucleotide Design

Staple oligonucleotides are short single-stranded DNA molecules (typically 20-60 nucleotides) that hybridize to specific regions of the scaffold, directing its folding into the target structure.

Each staple typically bridges two non-adjacent regions of the scaffold, creating crossovers that define the three-dimensional fold. A typical DNA origami structure employs 150-300 unique staple sequences (Douglas et al., Nature. 2009; 459 (7245): 414-418).

Staple design is performed using caDNAno software following established guidelines: (a) Staple length: 32-48 nucleotides optimal for stable hybridization and practical synthesis (Ke et al., “Multilayer DNA origami packed on hexagonal and hybrid lattices,” J Am Chem Soc. 2012; 134 (3): 1770-1774). (b) Crossover placement: Crossovers positioned every 7-8 base pairs for honeycomb lattice structures or every 10.5 base pairs for square lattice structures (Dietz et al., “Folding DNA into twisted and curved nanoscale shapes,” Science. 2009; 325 (5941): 725-730). (c) Melting temperature: Staples designed for uniform melting temperatures (Tm) in the range of 50-60 °C to ensure cooperative folding. (d) Secondary structure avoidance: Staple sequences screened to avoid stable hairpins or self-complementarity using tools such as NUPACK (Zadeh et al., “NUPACK: Analysis and design of nucleic acid systems,” J Comput Chem. 2011; 32 (1): 170-173).

### Staple Synthesis and Purification

Staple oligonucleotides are synthesized by commercial suppliers using standard phosphoramidite chemistry. Major suppliers include Integrated DNA Technologies (IDT), Sigma-Aldrich, and Eurofins Genomics.

For DNA origami assembly, standard desalted purification is typically sufficient, as demonstrated in numerous publications. HPLC or PAGE purification may be employed for modified staples containing fluorophores, chemical handles, or other modifications (Rothemund, Nature. 2006; 440 (7082): 297-302).

Staple pooling strategies enable cost-effective production at scale. Praetorius et al. demonstrated that staple pools can be produced enzymatically from phagemid templates, reducing per-assembly costs by 100-fold for large-scale production (Praetorius et al., “Biotechnological mass production of DNA origami,” Nature. 2017; 552 (7683): 84-87).

### DNA Origami Assembly Protocol

DNA origami assembly is achieved through thermal annealing of scaffold and staples in the presence of divalent cations.

### Standard Thermal Annealing Protocol

The following protocol is based on established methods with yields of 70-90% for most designs: Materials:
M13mp18 scaffold: 10-20 nM final concentration
Staple pool: 100-200 nM per staple (5-10× molar excess over scaffold)
TAE-Mg buffer: 40 mM Tris-acetate pH 8.0, 2 mM EDTA, 12.5 mM magnesium acetate.
Alternatively, folding buffer: 5 mM Tris-HCl pH 8.0, 1 mM EDTA, 10-20 mM MgCl□

Thermal annealing program (programmable thermocycler): Step 1: Denaturation-Heat to 95 °C, hold 5 minutes Step 2: Initial cooling-Cool from 95 °C to 80 °C at 0.5 °C per minute Step 3: Annealing-Cool from 80 °C to 65 °C at 0.1 °C per minute (critical folding window) Step 4: Final cooling-Cool from 65 °C to 25 °C at 0.5 °C per minute

### Step 5: Hold at 4 °C.

Total annealing time: approximately 12-18 hours depending on structure complexity.

This protocol is derived from Douglas et al., Nature. 2009; 459 (7245): 414-418, with modifications based on subsequent optimization studies. The slow cooling through 80-65 °C is critical, as this temperature range corresponds to staple hybridization (Sobczak et al., “Rapid folding of DNA into nanoscale shapes at constant temperature,” Science. 2012; 338 (6113): 1458-1461).

### Isothermal Assembly Alternative

Isothermal assembly at constant temperature provides an alternative to thermal annealing that may be advantageous for scale-up and incorporation of heat-sensitive modifications.

Sobczak et al. demonstrated that DNA origami can be assembled isothermally at temperatures between 45-60 °C in the presence of formamide, which lowers the melting temperature of DNA duplexes (Sobczak et al., Science. 2012; 338 (6113): 1458-1461). Assembly times of 1-2 hours were achieved with yields comparable to thermal annealing.

Isothermal protocol: Materials:
Scaffold and staples as above
Folding buffer supplemented with 30-40% formamide
   Incubation: Hold at 50-55 °C for 1-4 hours

Post-assembly: Remove formamide by buffer exchange (spin column or dialysis)

### DNA Origami Purification

Assembled DNA origami must be purified from excess staples before cargo loading and VLP encapsulation.

### PEG Precipitation

PEG precipitation provides a rapid, scalable purification method: Protocol: Add PEG-8000 to 15% (w/v) and NaCl to 500 mM. Incubate on ice for 30 minutes. Centrifuge at 16,000×g for 30 minutes at 4 °C. Resuspend pellet in storage buffer (TAE-Mg or equivalent). This method achieves greater than 90% recovery with greater than 95% removal of excess staples (Stahl et al., “Facile and scalable preparation of pure and dense DNA origami solutions,” Angew Chem Int Ed. 2014; 53 (47): 12735-12740).

### Agarose Gel Electrophoresis

Gel purification provides highest purity but lower throughput: Protocol: Run crude origami on 1.5-2% agarose gel in TAE-Mg buffer at 5 V/cm for 2-4 hours at 4 °C. Excise band corresponding to folded origami. Electroelute or extract using freeze-squeeze method. This method achieves greater than 99% purity but recovery is typically 40-60% (Douglas et al., Nature. 2009; 459 (7245): 414-418).

### Spin Column Purification

Molecular weight cutoff spin columns (100 kDa MWCO) provide rapid purification: Protocol: Load crude origami onto Amicon Ultra 100 kDa spin column. Centrifuge at 14,000×g for 10 minutes. Wash 3× with storage buffer. Recover purified origami from concentrate. Recovery is typically 70-85% with good staple removal (Shaw et al., “Purification of functionalized DNA origami nanostructures,” ACS Nano. 2015; 9 (5): 4968-4975).

### Section G: DNA Origami Geometry Selection for BBB Crossing

### Critical Parameters for BBB Transcytosis

The geometry of DNA origami structures significantly affects blood-brain barrier transcytosis efficiency. Selection of appropriate geometry is guided by published studies correlating nanoparticle properties with BBB crossing.

Jiang et al. systematically studied the BBB crossing efficiency of DNA nanostructures with different geometries and identified the following critical parameters (Jiang et al., “DNA nanostructures can cross the blood-brain barrier,” Nat Mater. 2024): (a) Size: Structures with diameter 50-100 nm showed optimal transcytosis. Smaller structures (less than 30 nm) exhibited rapid renal clearance. Larger structures (greater than 150 nm) showed reduced transcytosis efficiency. (b) Shape: Spherical and tubular geometries outperformed flat rectangular structures for BBB crossing. (c) Rigidity: Moderately flexible structures showed superior transcytosis compared to highly rigid structures, possibly due to conformational adaptation during vesicular transport. (d) Surface charge: Slightly negative surface charge (zeta potential −10 to −30 mV) was optimal, consistent with charge characteristics of natural DNA.

### Preferred Geometry: Tubular Capsule

In a preferred embodiment, the DNA origami core has a tubular capsule geometry with the following dimensions: Length: 60-80 nm Outer diameter: 30-50 nm Wall thickness: 8-12 nm (two DNA duplex layers) Internal cavity: 15-30 nm diameter

This geometry is based on the design principles established by Douglas et al. for hollow DNA origami structures (Douglas et al., Nature. 2009; 459 (7245): 414-418) and optimized based on the BBB crossing parameters identified by Jiang et al.

Design rationale: (a) Size is within optimal 50-100 nm range for BBB transcytosis. (b) Tubular shape provides elongated geometry shown to enhance transcytosis. (c) Internal cavity accommodates therapeutic payloads. (d) Wall thickness provides structural stability while maintaining moderate flexibility. (e) Dimensions are compatible with encapsulation within P22 VLP (58 nm internal diameter).

### Alternative Geometry: Spherical Cage

An alternative embodiment employs spherical cage geometry: Outer diameter: 40-60 nm Internal cavity: 20-40 nm diameter Structure: Icosahedral or quasi-spherical wireframe

Benson et al. demonstrated design and assembly of polyhedral DNA origami meshes with wireframe architectures that provide large internal volumes relative to total mass (Benson et al., “DNA rendering of polyhedral meshes at the nanoscale,” Nature. 2015; 523 (7561): 441-444).

Spherical wireframe structures offer advantages including: (a) Reduced DNA content per unit volume, potentially reducing manufacturing costs. (b) Greater flexibility, potentially enhancing transcytosis. (c) More open architecture, facilitating cargo loading and release.

### Alternative Geometry: Tetrahedral Frame

Tetrahedral DNA origami provides a minimalist cage structure: Edge length: 30-50 nm Internal volume: approximately 5,000-15,000 nm³

Goodman et al. demonstrated assembly of DNA tetrahedra from four oligonucleotides, providing a simple and robust cage structure (Goodman et al., “Rapid chiral assembly of rigid DNA building blocks for molecular nanofabrication,” Science. 2005; 310 (5754): 1661-1665).

Zhang et al. demonstrated that DNA tetrahedra can cross cell membranes and deliver siRNA payloads, establishing precedent for therapeutic delivery applications (Zhang et al., “DNA origami as an in vivo drug delivery vehicle for cancer therapy,” ACS Nano. 2014; 8 (7): 6633-6643).

### Geometry Selection Criteria

Selection among geometries for specific applications may be guided by: (a) Payload size: Larger payloads (antibodies, RNPs) require tubular or large spherical geometries. Smaller payloads (small molecules, siRNA) can use tetrahedral structures. (b) VLP compatibility: Geometry must fit within VLP internal diameter. For P22 VLPs (58 nm internal diameter), origami structures should not exceed 50 nm in any dimension. (c) Manufacturing considerations: Simpler geometries (tetrahedra) have higher assembly yields and lower staple requirements. Complex geometries require more extensive optimization. (d) Release kinetics: Open wireframe structures may provide faster payload release compared to closed tubular structures.

### Section H: DNA Origami Stabilization

### The Nuclease Degradation Problem

Unmodified DNA origami structures are susceptible to degradation by nucleases present in biological fluids. Stabilization is essential for therapeutic applications requiring systemic circulation.

Hahn et al. systematically characterized DNA origami stability in cell culture medium and serum. Unmodified DNA origami structures showed significant degradation within 1-4 hours in 10% fetal bovine serum, with complete loss of structural integrity by 24 hours (Hahn et al., “Addressing the instability of DNA nanostructures in tissue culture,” ACS Nano. 2014; 8 (9): 8765-8775).

The primary nucleases responsible for DNA origami degradation in serum are DNase I and exonucleases. DNase I creates double-strand breaks throughout the structure, while exonucleases degrade from free ends created by staple termini (Hahn et al., ACS Nano. 2014).

### Stabilization Strategy 1: Cationic Polymer Coating (Preferred)

In a preferred embodiment, DNA origami structures are stabilized by coating with cationic polymers that electrostatically associate with the negatively charged DNA backbone.

### Oligolysine Coating

Ponnuswamy et al. demonstrated that coating DNA origami with oligolysine peptides (K□□, 10 lysine residues) dramatically increases nuclease resistance (Ponnuswamy et al., “Oligolysine-based coating protects DNA nanostructures from low-salt denaturation and nuclease degradation,” Nat Commun. 2017; 8:15654).

Key findings from Ponnuswamy et al.: (a) Oligolysine coating increased nuclease resistance by approximately 1000-fold. (b) Coated DNA origami remained structurally intact in 10% fetal bovine serum for greater than 24 hours. (c) Coating preserved the structural features of the DNA origami as confirmed by TEM. (d) Coating is reversible under high ionic strength conditions, potentially enabling controlled release.

Coating protocol based on Ponnuswamy et al.: Materials:
Purified DNA origami: 10-50 nM
Oligolysine peptide (K□□): 1 μM per 1 nM phosphate (approximately 1 lysine per 3 nucleotides)
Coating buffer: 10 mM Tris-HCl pH 7.5, 1 mM EDTA (low ionic strength)

Protocol: Mix origami and oligolysine in coating buffer. Incubate at 25 °C for 30 minutes. Coating is confirmed by gel shift assay (reduced mobility) and zeta potential measurement (shift from negative to near-neutral or positive).

### Polyethylenimine (PEI) Coating

PEI represents an alternative cationic polymer for DNA origami stabilization.

Kiviaho et al. demonstrated that low molecular weight PEI (1.8 kDa) provides effective nuclease protection for DNA origami while maintaining cellular uptake (Kiviaho et al., “Cationic polymers for DNA origami coating—examining their binding efficiency and tuning the enzymatic reaction rates,” Nanoscale. 2016; 8 (22): 11674-11680).

PEI coating protocol: Materials:
Purified DNA origami: 10-50 nM
PEI 1.8 kDa: N/P ratio of 3-5 (ratio of PEI nitrogen to DNA phosphate)
Coating buffer: 10 mM HEPES pH 7.4

Protocol: Add PEI slowly to origami solution with mixing. Incubate 15-30 minutes at room temperature. Measure zeta potential to confirm coating.

Caution: PEI can be cytotoxic at high concentrations. Low molecular weight PEI (1.8-2 kDa) is preferred over high molecular weight (25 kDa) for reduced toxicity (Kiviaho et al., Nanoscale. 2016).

### Stabilization Strategy 2: Chemical Crosslinking

Covalent crosslinking of DNA origami structures provides permanent stabilization resistant to high ionic strength and denaturing conditions.

### UV Crosslinking

Ultraviolet irradiation induces thymine-thymine cyclobutane dimer formation at sites where thymines are adjacent in the DNA structure.

Cassinelli et al. demonstrated that UV crosslinking at 254 nm increases DNA origami melting temperature by 20-30 °C and confers resistance to EDTA-induced disassembly (Cassinelli et al., “One-step formation of ‘chain-armor’-stabilized DNA nanostructures,” Angew Chem Int Ed. 2015; 54 (27): 7795-7798).

UV crosslinking protocol: Materials:
Purified DNA origami in TAE-Mg buffer
UV crosslinker with 254 nm source

Protocol: Place sample 5 cm from UV source. Irradiate at 1-10 mJ/cm² (optimize for specific structure). Crosslinking is confirmed by resistance to EDTA treatment (addition of 50 mM EDTA should not cause disassembly).

Limitation: UV crosslinking may damage payload molecules, particularly proteins and nucleic acids. This method is most suitable when payloads are loaded after crosslinking.

### Click Chemistry Crosslinking

Site-specific crosslinks can be introduced using click chemistry between modified staples.

Gerling et al. demonstrated that incorporation of alkyne and azide-modified nucleotides at defined positions enables copper-catalyzed azide-alkyne cycloaddition (CuAAC) crosslinking of DNA origami structures (Gerling et al., “Sequence-programmable covalent bonding of designed DNA assemblies,” Sci Adv. 2018; 4 (8): eaau1157).

This approach enables: (a) Defined crosslink positions based on staple design. (b) Bioorthogonal chemistry compatible with sensitive payloads. (c) Tunable crosslink density by controlling number of modified staples.

### Stabilization Strategy 3: Silica Mineralization

Coating DNA origami with silica provides robust protection for long-term stability applications.

Nguyen et al. demonstrated silica coating of DNA origami using the Stöber process, producing silica-DNA hybrid structures stable at temperatures up to 500 °C (Nguyen et al., “Metallization of DNA Origami Structures,” ACS Nano. 2015; 9 (8): 8670-8677).

Silica coating protocol: Materials:
Purified DNA origami
Tetraethyl orthosilicate (TEOS)
Ammonia catalyst

Protocol: Mix origami with TEOS in ethanol/water mixture. Add ammonia catalyst. Incubate 2-24 hours at room temperature. Wash to remove unreacted silica.

Limitation: Silica coating creates a rigid, impermeable shell that may interfere with payload release and cellular uptake. This approach is most suitable for applications requiring extreme stability.

### Recommended Stabilization Approach for Therapeutic Applications

For the present invention, the following stabilization approach is recommended: Primary stabilization: Oligolysine (K□□) coating per Ponnuswamy et al. protocol. Rationale: (a) Well-validated with extensive published data. (b) Preserves origami structure and function. (c) Compatible with payload loading. (d) Potentially reversible for controlled payload release. (e) Low toxicity of oligolysine peptides.

The oligolysine-coated DNA origami provides the core structure for subsequent BBB-targeting peptide conjugation and VLP encapsulation.

### Section I: Blood-Brain Barrier Crossing Modifications

### BBB Transcytosis Targeting Ligands

DNA origami cores are functionalized with receptor-targeting ligands that mediate transcytosis across brain capillary endothelium. This section describes validated targeting approaches with literature support.

### Transferrin Receptor Targeting

The transferrin receptor (TfR1, CD71) is highly expressed on brain capillary endothelium and mediates transcytosis of iron-bound transferrin from blood to brain.

Quantitative proteomics demonstrated TfR1 expression of 1.5 fmol/μg protein on human brain microvessels, representing one of the most highly expressed receptors on BBB endothelium (Uchida et al., J Neurochem. 2011; 117 (2): 333-345).

### T7 Peptide

The T7 peptide (a transferrin receptor-binding peptide of from 5 to 15 amino acid residues having transferrin receptor binding affinity below 1 micromolar and configured to mediate receptor-mediated transcytosis across the blood-brain barrier,) binds TfR1 and mediates transcytosis across brain endothelium.

Lee et al. identified the T7 peptide through phage display screening and demonstrated binding to TfR1 with Kd approximately 10 nM (Lee et al., “Receptor mediated uptake of peptides that bind the human transferrin receptor,” Eur J Biochem. 2001; 268 (7): 2004-2012).

Multiple independent studies have validated T7 peptide for brain-targeted delivery: (a) Kuang et al. demonstrated that T7-functionalized nanoparticles achieved 3.5-fold enhanced brain accumulation compared to non-targeted controls in mice (Kuang et al., Int J Pharm. 2013; 454 (1): 11-20). (b) Staquicini et al. demonstrated T7-mediated brain delivery of therapeutic proteins in a mouse model (Staquicini et al., “Systemic combinatorial peptide selection yields a non-canonical iron-mimicry mechanism for targeting tumors in a mouse model of human glioblastoma,” J Clin Invest. 2011; 121 (1): 161-173). (c) Wei et al. demonstrated T7-conjugated liposomes achieved enhanced BBB penetration and therapeutic efficacy in glioma models (Wei et al., “A d-peptide ligand of nicotine acetylcholine receptors for brain-targeted drug delivery,” Angew Chem Int Ed. 2015; 54 (10): 3023-3027).

### T7 Peptide Conjugation to DNA Origami

T7 peptide is conjugated to DNA origami through modified staple strands containing amine, thiol, or click chemistry handles.

Conjugation protocol using maleimide chemistry: Materials:
DNA origami with thiol-modified staples at defined exterior positions
T7 peptide synthesized with N-terminal maleimide-PEG linker
Conjugation buffer: PBS pH 7.0

Protocol: Reduce thiol-modified origami with TCEP (1 mM, 30 min). Add maleimide-T7 peptide at 10-50 fold excess per thiol site. Incubate 2-4 hours at room temperature. Purify by spin column to remove unreacted peptide.

Alternatively, T7 peptide-DNA conjugates can be incorporated as modified staples during origami assembly, eliminating post-assembly conjugation (Schaffert et al., “Aptamer-modified DNA origami for targeted drug delivery,” Small. 2016; 12 (47): 6490-6499).

### Targeting Density Optimization

The density of targeting ligands on DNA origami surfaces affects binding avidity and transcytosis efficiency.

Based on published data for nanoparticle targeting, optimal ligand densities are typically 20-50 ligands per particle (Nel et al., “Understanding biophysicochemical interactions at the nano-bio interface,” Nat Mater. 2009; 8 (7): 543-557).

For DNA origami, this corresponds to incorporating 20-50 peptide-modified staples at surface-exposed positions. Excessive ligand density may cause aggregation or reduce transcytosis through avidity-mediated trapping at the receptor (Wiley et al., “Transcytosis and brain uptake of transferrin-containing nanoparticles by tuning avidity to transferrin receptor,” Proc Natl Acad Sci USA. 2013; 110 (21): 8662-8667).

### Nicotinic Acetylcholine Receptor Targeting

The α7 nicotinic acetylcholine receptor (α7-nAChR) is expressed on brain endothelium and neurons, providing an alternative transcytosis pathway and neuronal targeting.

### RVG-29 Peptide

The RVG-29 peptide (a nicotinic acetylcholine receptor-binding peptide of from 20 to 35 amino acid residues derived from a rabies virus glycoprotein fragment, configured to bind the nicotinic acetylcholine receptor with affinity below 500 nanomolar and to mediate neuronal targeting following receptor-mediated transcytosis,) is derived from the rabies virus glycoprotein and binds α7-nAChR.

Kumar et al. first demonstrated RVG-29 mediated brain delivery, achieving approximately 50% knockdown of target genes in brain following intravenous administration of RVG-siRNA conjugates (Kumar et al., “Transvascular delivery of small interfering RNA to the central nervous system,” Nature. 2007; 448 (7149): 39-43).

The mechanism of RVG-29 has been characterized in detail: (a) Binding to α7-nAChR on brain endothelium mediates transcytosis (Liu et al., “Brain-targeting gene delivery and cellular internalization mechanisms for modified rabies virus glycoprotein RVG29 nanoparticles,” Biomaterials. 2009; 30 (25): 4195-4202). (b) Post-transcytosis, RVG-29 provides neuronal targeting through α7-nAChR expression on neurons. (c) The peptide demonstrates minimal immunogenicity in mice at therapeutic doses (Kumar et al., Nature. 2007).

Multiple independent groups have validated RVG-29 for brain delivery: (a) Liu et al. demonstrated RVG-29-conjugated exosomes achieved 3-fold higher brain accumulation than unmodified exosomes (Liu et al., Sci Rep. 2015; 5:17543). (b) Alvarez-Erviti et al. demonstrated RVG-29-exosome delivery of siRNA achieved therapeutic knockdown in mouse brain (Alvarez-Erviti et al., “Delivery of siRNA to the mouse brain by systemic injection of targeted exosomes,” Nat Biotechnol. 2011; 29 (4): 341-345). (c) Hwang et al. demonstrated RVG-29-nanoparticle delivery of paclitaxel for glioblastoma treatment (Hwang et al., “A brain-targeted rabies virus glycoprotein-disulfide linked PEI nanocarrier for delivery of neurogenic microRNA,” Biomaterials. 2011; 32 (21): 4968-4975).

### Freedom to Operate Note Regarding RVG-29

The RVG-29 peptide was first disclosed in Kumar et al., Nature 2007, research conducted at Harvard Medical School. Relevant intellectual property may be held by Harvard University or licensed to commercial entities. Freedom-to-operate analysis is essential before commercial development utilizing RVG-29.

### RVG-29 Conjugation to DNA Origami

RVG-29 is conjugated to DNA origami using methods analogous to T7 peptide conjugation. Due to the larger size of RVG-29 (29 amino acids vs. 7 for T7), longer PEG linkers (PEG□□-PEG□□) may be beneficial to reduce steric constraints.

### Dual Targeting Strategy

In a preferred embodiment, DNA origami is functionalized with both T7 peptide (TfR1 targeting) and RVG-29 (α7-nAChR targeting) to engage multiple transcytosis pathways.

Rationale for dual targeting: (a) Multiple receptor engagement may increase transcytosis probability. (b) TfR1 and α7-nAChR mediate distinct transcytosis pathways, providing redundancy. (c) Post-BBB, dual targeting provides both endothelial retention (via TfR1) and neuronal targeting (via RVG-29).

Gao et al. demonstrated that dual-targeted nanoparticles combining two different ligands achieved synergistic enhancement of brain delivery compared to single-targeted particles (Gao et al., “Angiopep-2 and activatable cell-penetrating peptide dual-functionalized nanoparticles for systemic glioma-targeting delivery,” Mol Pharm. 2014; 11 (8): 2755-2767).

Recommended dual targeting configuration: T7 peptide: 20-30 copies per origami (TfR1 targeting) RVG-29:15-25 copies per origami (α7-nAChR targeting) Total targeting peptides: 35-55 per origami

### Alternative Targeting Approaches

### Angiopep-2

Angiopep-2 (an LRP1 receptor-binding peptide of from 15 to 25 amino acid residues configured to bind low-density lipoprotein receptor-related protein 1 with affinity below 500 nanomolar and to mediate receptor-mediated transcytosis across the blood-brain barrier,) targets the low-density lipoprotein receptor-related protein 1 (LRP1) on brain endothelium.

Demeule et al. identified Angiopep-2 and demonstrated enhanced brain uptake of conjugated paclitaxel (Demeule et al., “Involvement of the low-density lipoprotein receptor-related protein in the transcytosis of the brain delivery vector angiopep-2,” J Neurochem. 2008; 106 (4): 1534-1544).

Angiopep-2 has advanced to clinical trials. ANG1005 (Angiopep-2-paclitaxel conjugate) showed clinical activity in patients with brain metastases (Kumthekar et al., “ANG1005, a Brain-Penetrating Peptide-Drug Conjugate, Shows Activity in Patients with Breast Cancer Brain Metastases,” Clin Cancer Res. 2020; 26 (20): 5412-5418).

### Transferrin Protein

Full-length transferrin or transferrin fragments can serve as TfR1 ligands as an alternative to T7 peptide.

Johnsen et al. demonstrated that transferrin-conjugated nanoparticles undergo transcytosis across BBB endothelium, with transcytosis efficiency dependent on conjugation density (Johnsen et al., “Modulating the antibody density changes the uptake and transport at the blood-brain barrier of both transferrin receptor-targeted gold nanoparticles and antibody,” J Control Release. 2019; 295:237-249).

### Antibody-Based Targeting

Anti-TfR1 antibodies or antibody fragments can serve as targeting ligands with potentially higher affinity than peptides.

Importantly, Niewoehner et al. demonstrated that anti-TfR1 antibody affinity must be optimized for efficient transcytosis. High-affinity antibodies are trapped at the receptor, while moderate-affinity variants undergo transcytosis more efficiently (Niewoehner et al., “Increased brain penetration and potency of a therapeutic antibody using a monovalent molecular shuttle,” Neuron. 2014; 81 (1): 49-60).

This principle applies to peptide ligands as well: moderate affinity (Kd 10-100 nM) may be optimal for transcytosis rather than maximal affinity.

### Section J: Therapeutic Payload Loading

### Payload Categories

DNA origami can accommodate diverse therapeutic payloads through multiple loading mechanisms. This section describes validated approaches for each payload class.

### Small Molecule Drug Loading

Small molecule drugs (molecular weight less than 1,000 Da) can be loaded into DNA origami through intercalation, minor groove binding, or covalent conjugation.

### Doxorubicin (Intercalation Model)

Doxorubicin is a chemotherapeutic agent that intercalates into DNA double helices, providing a well-characterized model for small molecule loading.

Jiang et al. demonstrated doxorubicin loading into DNA origami with loading capacity of 4.5 μM drug per 1 nM origami (approximately 4,500 molecules per structure) based on the number of GC base pairs available for intercalation (Jiang et al., “DNA origami as a carrier for circumvention of drug resistance,” J Am Chem Soc. 2012; 134 (32): 13396-13403).

Loading protocol: Materials:
Purified DNA origami: 10-100 nM
Doxorubicin: 10-100 μM (100-1000 fold molar excess)
Loading buffer: PBS or TAE-Mg

Protocol: Mix origami and doxorubicin. Incubate at room temperature for 2-24 hours. Remove unloaded drug by spin column or dialysis. Quantify loading by fluorescence measurement.

Zhang et al. demonstrated that doxorubicin-loaded DNA origami achieved enhanced cytotoxicity in drug-resistant cancer cells compared to free doxorubicin, attributed to circumvention of drug efflux mechanisms (Zhang et al., “DNA origami as an in vivo drug delivery vehicle for cancer therapy,” ACS Nano. 2014; 8 (7): 6633-6643).

### Covalent Drug Conjugation

For drugs that do not intercalate, covalent conjugation to modified staples provides an alternative loading approach.

Protocol: Synthesize drug-oligonucleotide conjugates using amine-NHS, thiol-maleimide, or click chemistry. Incorporate conjugates as modified staples during origami assembly. Loading capacity is determined by the number of drug-modified staples.

This approach provides precise control over drug loading (known number of drugs per origami) but requires chemical synthesis of drug-oligonucleotide conjugates for each drug candidate.

### Protein and Peptide Loading

Proteins and peptides can be loaded into DNA origami through electrostatic interaction, aptamer capture, or covalent conjugation.

### Electrostatic Encapsulation

Positively charged proteins can be encapsulated within negatively charged DNA origami cavities through electrostatic complementarity.

Schüller et al. demonstrated encapsulation of cationic fluorescent proteins within DNA origami nanocages with high efficiency (Schüller et al., “Cellular immunostimulation by CpG-sequence-coated DNA origami structures,” ACS Nano. 2011; 5 (12): 9696-9702).

For neutral or negatively charged proteins, the protein surface can be modified with cationic polymers or the origami interior can be functionalized with positively charged moieties to enable electrostatic capture.

### Aptamer-Mediated Capture

DNA aptamers can be incorporated into origami structures to capture specific protein payloads with high affinity and specificity.

Douglas et al. demonstrated aptamer-functionalized DNA origami that captures and releases protein payloads in response to specific molecular triggers (Douglas et al., “A logic-gated nanorobot for targeted transport of molecular payloads,” Science. 2012; 335 (6070): 831-834).

Aptamer capture protocol: Design: Identify or select aptamer for target protein (Kd<100 nM). Incorporate aptamer sequence into internal staples of origami structure. Loading: Incubate aptamer-functionalized origami with target protein (10-100 fold molar excess). Incubate 1-4 hours at room temperature. Purify to remove unbound protein.

### Antibody Fragment Loading

Antibody fragments (Fab, scFv, nanobodies) can be loaded into DNA origami through multiple mechanisms.

Site-specific conjugation using engineered cysteines or unnatural amino acids provides controlled orientation: Protocol: Express antibody fragment with C-terminal cysteine. Conjugate to maleimide-modified staples. Incorporate conjugates during origami assembly.

Shaw et al. demonstrated loading of Fab fragments into DNA origami with controlled stoichiometry and orientation (Shaw et al., “Purification of functionalized DNA origami nanostructures,” ACS Nano. 2015; 9 (5): 4968-4975).

### Nucleic Acid Payload Loading

mRNA Loading

Messenger RNA can be loaded into DNA origami through hybridization to complementary sequences or through electrostatic complexation.

### Hybridization-Based Loading

Complementary DNA sequences incorporated into origami staples can capture mRNA through Watson-Crick base pairing.

Protocol: Design capture sequences complementary to target mRNA 3′ UTR or other accessible regions. Incorporate capture sequences (20-30 nucleotides each) at multiple positions within origami cavity. Incubate with mRNA at 37 °C to anneal. Expected loading: 1-10 mRNA molecules per origami depending on design.

### Electrostatic Complexation

mRNA can be complexed with cationic lipids or polymers prior to encapsulation within origami.

Mikkilä et al. demonstrated that mRNA-polylysine complexes can be encapsulated within DNA origami structures, combining the delivery benefits of lipoplexes with the targeting capabilities of DNA origami (Mikkilä et al., Nano Lett. 2014; 14 (4): 2196-2200). siRNA Loading

Small interfering RNA can be incorporated into DNA origami through hybridization or chemical conjugation.

Lee et al. demonstrated that siRNA can be hybridized to complementary overhangs on DNA origami, providing controlled loading and release (Lee et al., “Molecularly self-assembled nucleic acid nanoparticles for targeted in vivo siRNA delivery,” Nat Nanotechnol. 2012; 7 (6): 389-393).

siRNA loading protocol: Design: Include 15-20 nucleotide overhangs complementary to siRNA sense strand on internal staples. Loading: Incubate origami with siRNA at 1:1 to 1:10 origami: siRNA ratio. Anneal at 37 °C for 1-2 hours. Expected loading: 10-50 siRNA molecules per origami.

### Gene Editing Component Loading

### CRISPR-Cas9 RNP Loading

CRISPR-Cas9 ribonucleoprotein complexes (RNPs) can be loaded into DNA origami for gene editing applications.

Sun et al. demonstrated delivery of Cas9 RNPs using DNA nanostructures, achieving gene editing in mammalian cells (Sun et al., “Self-assembled DNA nanoclews for the efficient delivery of CRISPR-Cas9 for genome editing,” Angew Chem Int Ed. 2015; 54 (41): 12029-12033).

RNP loading considerations: (a) Size: Cas9 protein is approximately 160 kDa, requiring large origami cavities. (b) Charge: Cas9-sgRNA complex is negatively charged, requiring positively charged binding sites. (c) Activity: Loading conditions must preserve Cas9 nuclease activity.

Loading protocol: Design: Use large tubular or spherical origami with internal cavity greater than 20 nm. Functionalize interior with cationic peptides (e.g., polylysine extensions on internal staples). Incubate purified Cas9-sgRNA RNP with origami at 4 °C to preserve activity. Expected loading: 1-5 RNPs per origami.

### Section K: Stimuli-Responsive Release Mechanisms

pH-Responsive Release (Preferred)

For therapeutic applications, controlled payload release is essential. pH-responsive mechanisms enable release in acidic endosomal compartments following cellular uptake. i-Motif DNA Switches

i-Motif sequences are cytosine-rich DNA sequences that form four-stranded intercalated structures at acidic pH (below 6.0-6.5) through formation of C-C□ base pairs.

Dong et al. demonstrated i-motif-based pH-responsive DNA nanostructures that undergo conformational changes in response to pH decrease (Dong et al., “DNA nanotechnology based on i-motif structures,” Acc Chem Res. 2014; 47 (6): 1853-1860).

i-Motif sequence: a pH-responsive cytosine-rich oligonucleotide element of from 20 to 30 nucleotides configured to undergo conformational change from a single-stranded random coil at physiological pH to an intercalated tetrad i-motif structure at pH below approximately 6.5 through cytosine-cytosine-plus base pair formation, providing endosomal-pH-triggered conformational switching and payload release This sequence forms stable i-motif structure at pH below 6.0 and exists as random coil at physiological pH 7.4.

Incorporation into DNA origami: Design i-motif latches that hold origami closed at pH 7.4. Upon acidification (endosomal pH 5.5-6.0), i-motif formation causes conformational change. Latch opening releases encapsulated payload.

Burns et al. demonstrated i-motif-based pH-responsive DNA origami boxes that open at acidic pH, releasing encapsulated cargo (Burns et al., “A synthetic receptor for the recognition of nucleobase-metal ion pairs,” J Am Chem Soc. 2014; 136 (45): 15767-15774).

### Triple Helix pH Switches

Triple helix (triplex) DNA structures provide an alternative pH-responsive mechanism.

Triplex structures formed by Hoogsteen base pairing are stable at acidic pH but dissociate at physiological pH. This pH response is opposite to i-motif, enabling design of structures that close at acidic pH.

Idili et al. demonstrated triplex-based pH-responsive switches with tunable transition pH (Idili et al., “Programmable pH-triggered DNA nanoswitches,” J Am Chem Soc. 2014; 136 (16): 5836-5839).

### Enzyme-Responsive Release

Enzyme-cleavable linkers enable payload release in response to disease-associated enzymes.

### Matrix Metalloproteinase (MMP) Cleavage

MMP-cleavable peptide linkers enable release in tumor or inflammatory microenvironments where MMPs are upregulated.

Incorporation of MMP substrate peptides (e.g., a matrix-metalloproteinase-cleavable peptide linker of from 5 to 10 amino acid residues configured to be cleaved selectively in tumor and inflammatory microenvironments by upregulated matrix metalloproteinases) between payload and origami enables enzymatic release (Zhu et al., “Matrix metalloprotease-cleavable peptide carriers for tumor-specific prodrug activation,” J Am Chem Soc. 2007; 129 (16): 4786-4787).

### Cathepsin B Cleavage

Cathepsin B-cleavable linkers enable release in lysosomes following endocytosis.

The Val-Cit-PAB linker used in antibody-drug conjugates (e.g., brentuximab vedotin) is efficiently cleaved by cathepsin B and can be adapted for origami payload release (Ducry & Stump, “Antibody-drug conjugates: linking cytotoxic payloads to monoclonal antibodies,” Bioconjug Chem. 2010; 21 (1): 5-13).

### Glutathione-Responsive Release

Disulfide bonds are cleaved by the reducing environment (1-10 mM glutathione) inside cells, enabling intracellular payload release.

Incorporation of disulfide linkers between payload and origami enables redox-responsive release. Lee et al. demonstrated glutathione-responsive release from DNA nanostructures with 10-fold enhanced release in intracellular reducing conditions (Lee et al., Nat Nanotechnol. 2012; 7 (6): 389-393).

### Section L: Hybrid Nanostructure Assembly

Critical Assembly Step: Integration of DNA Origami Core into VLP Shell

The integration of DNA origami cores into VLP protective shells represents the central assembly step of the present invention. This section describes validated approaches for cargo encapsulation within VLPs, with specific consideration of DNA nanostructure encapsulation.

### Literature Validation of DNA Nanostructure Encapsulation in Protein Shells

The encapsulation of DNA nanostructures within viral capsids or virus-like particles has been directly demonstrated in peer-reviewed literature, providing critical validation for the assembly approach of the present invention.

Mikkilä et al. demonstrated encapsulation of DNA origami structures within cowpea chlorotic mottle virus (CCMV) VLPs (Mikkilä et al., “Virus-encapsulated DNA origami nanostructures for cellular delivery,” Nano Lett. 2014; 14 (4): 2196-2200). Key findings: (a) DNA origami tubes (60 nm×10 nm) were successfully encapsulated within CCMV capsids. (b) Encapsulation was achieved through electrostatic-driven assembly at low ionic strength. (c) Encapsulation efficiency exceeded 80% as assessed by gel electrophoresis and TEM. (d) Encapsulated origami retained structural integrity as confirmed by TEM. (e) VLP-encapsulated origami showed enhanced cellular uptake compared to free origami.

This publication directly validates the core concept of the present invention: DNA origami structures can be encapsulated within viral protein shells while maintaining structural integrity of both components.

Lim et al. demonstrated encapsulation of DNA tetrahedra within ferritin protein cages (Lim et al., “Nucleic acid-based programmable assembly of protein cages,” Angew Chem Int Ed. 2013; 52 (47): 12424-12428). Key findings: (a) DNA tetrahedra (edge length approximately 7 nm) were encapsulated within 12 nm ferritin interior. (b) Assembly was driven by electrostatic complementarity between positively charged ferritin interior and negatively charged DNA. (c) Encapsulation was confirmed by native gel electrophoresis and cryo-EM.

Erben et al. demonstrated encapsulation of cytochrome c protein within a DNA tetrahedron cage, demonstrating the converse arrangement (protein inside DNA structure) but validating the general principle of nucleic acid-protein hybrid assemblies (Erben et al., “Single-molecule protein encapsulation in a rigid DNA cage,” Angew Chem Int Ed. 2006; 45 (44): 7414-7417).

### Assembly Method 1: Electrostatic-Driven Co-Assembly (Preferred Method)

In a preferred embodiment, hybrid nanostructures are assembled through electrostatic-driven co-assembly of DNA origami cores with VLP coat proteins.

### Theoretical Basis

VLP assembly is driven by electrostatic interactions between positively charged regions of coat proteins and negatively charged nucleic acid cargo. This principle underlies natural viral packaging and has been exploited for synthetic cargo encapsulation.

Garmann et al. systematically characterized the electrostatic requirements for CCMV coat protein assembly around anionic polymers (Garmann et al., “Physical principles in the self-assembly of a simple spherical virus,” Acc Chem Res. 2016; 49 (1): 48-55). Key findings: (a) Assembly requires negatively charged cargo with charge density above a critical threshold. (b) DNA and RNA are ideal cargos due to high charge density (approximately 2 negative charges per nucleotide). (c) Assembly is nucleated at low ionic strength (less than 100 mM NaCl) and promoted by divalent cations (Mg²□, Ca²□). (d) Cargo size can exceed the natural viral genome if charge requirements are met.

DNA origami structures are highly negatively charged due to the phosphate backbone of the DNA double helix. A typical DNA origami structure (7,249 nucleotide scaffold plus approximately 200 staples of 40 nucleotides each) contains approximately 15,000 nucleotides, representing approximately 30,000 negative charges.

### Assembly Protocol for CCMV-DNA Origami Hybrids

The following protocol is based on Mikkila et al., Nano Lett. 2014 with modifications for different DNA origami geometries: Materials:
Purified, stabilized DNA origami: 50-100 nM
CCMV coat protein dimers: 5-10 μM (approximately 100-fold molar excess over origami)
Assembly buffer: 50 mM Tris-HCl pH 7.5, 50 mM NaCl, 10 mM MgCl□, 1 mM DTT

Protocol: Step 1: Dialyze coat protein into assembly buffer to remove storage buffer components. Step 2: Mix DNA origami and coat protein at room temperature with gentle pipetting. Step 3: Incubate at 4 °C for 4-24 hours with gentle rocking. Step 4: Purify assembled hybrids by size exclusion chromatography (Sephacryl S-500) or sucrose gradient ultracentrifugation. Step 5: Characterize by DLS, TEM, and gel electrophoresis.

Expected yield: 60-85% encapsulation efficiency based on Mikkilä et al. data. Unencapsulated origami and unassembled coat protein are removed during purification.

### Assembly Protocol for P22-DNA Origami Hybrids

P22 VLPs require a different assembly approach due to their larger size (58 nm vs. 28 nm for CCMV) and different assembly pathway.

P22 assembly naturally requires the scaffold protein (SP) to template capsid formation. For cargo encapsulation, two approaches have been validated:

### Approach A: SP-Cargo Fusion

Cargo proteins are genetically fused to the scaffold protein and incorporated during VLP assembly. This approach has been extensively validated by the Douglas laboratory (Patterson et al., ACS Nano. 2012; 6 (6): 5000-5009).

For DNA origami encapsulation, this approach requires: (a) Expression of SP-DNA binding domain fusion (e.g., SP-HU protein or SP-protamine domain). (b) Mixing SP fusion with DNA origami to create SP-origami complexes. (c) Adding coat protein to trigger VLP assembly around SP-origami complexes.

Protocol: Materials:
SP-DNA binding domain fusion protein: 1-5 μM
Stabilized DNA origami: 50-100 nM
P22 coat protein: 10-50 μM
Assembly buffer: 50 mM Tris-HCl pH 7.5, 200 mM NaCl, 5 mM MgCl□

Step 1: Incubate SP fusion with DNA origami at 25 °C for 1 hour to form complexes. Step 2: Add coat protein and incubate at 25 °C for 4-24 hours. Step 3: Purify by sucrose gradient ultracentrifugation (20-50% gradient, 100,000× g, 2 hours). Step 4: Collect VLP band and characterize.

### Approach B: Osmotic Shock Loading

Pre-formed P22 VLPs can be loaded with cargo through osmotic shock-induced transient pore formation.

This approach is based on the observation that P22 capsids undergo reversible swelling under specific ionic conditions, creating transient openings that allow cargo entry.

Protocol based on Parent et al., “P22 coat protein structures reveal a novel mechanism for capsid maturation,” Structure. 2010; 18 (3): 390-401: Materials:
Pre-assembled P22 VLPs: 1-10 mg/mL
Stabilized DNA origami: 100-500 nM
Swelling buffer: 50 mM Tris-HCl pH 9.0, 200 mM NaCl (triggers expansion)
Recovery buffer: 50 mM sodium acetate pH 5.5, 200 mM NaCl (triggers contraction)

Step 1: Incubate VLPs in swelling buffer at 37 °C for 30 minutes. Step 2: Add DNA origami and incubate for 2-4 hours. Step 3: Dialyze into recovery buffer over 4-12 hours. Step 4: Purify by SEC to remove unencapsulated origami.

Expected encapsulation efficiency: 30-60% based on similar approaches with CCMV (Comellas-Aragonès et al., Nat Nanotechnol. 2007; 2 (10): 635-639).

### Assembly Method 2: Disassembly-Reassembly

VLPs can be disassembled into coat protein subunits, mixed with DNA origami cargo, and reassembled to encapsulate the cargo.

This approach has been validated for multiple VLP systems: CCMV: Disassembly at pH 7.5, high ionic strength; reassembly at pH 5.0, low ionic strength (Douglas & Young, Nature. 1998; 393 (6681): 152-155). MS2: Disassembly with acetic acid; reassembly at neutral pH (Glasgow et al., ACS Nano. 2012; 6 (10): 8658-8664). QB: Disassembly with urea; reassembly upon urea removal (Fiedler et al., Angew Chem Int Ed. 2010; 49 (50): 9648-9651).

General disassembly-reassembly protocol: Step 1: Disassemble VLPs by appropriate method (pH shift, denaturant, or ionic strength change). Step 2: Purify coat protein subunits by SEC to remove aggregates. Step 3: Mix coat protein with DNA origami at defined ratio (typically 50-200 coat proteins per origami). Step 4: Trigger reassembly by reversing disassembly conditions. Step 5: Incubate for 4-24 hours to allow complete assembly. Step 6: Purify and characterize.

### Size Compatibility Considerations

The DNA origami core must fit within the VLP internal cavity. The following size relationships guide design: CCMV VLP: Internal diameter approximately 18-20 nm. Suitable for small DNA origami structures (tetrahedra, small tubes). P22 VLP: Internal diameter approximately 50-58 nm. Suitable for larger DNA origami structures (tubes up to 50 nm, spherical cages up to 45 nm). T4-derived VLPs: Internal diameter approximately 80-90 nm. Suitable for largest DNA origami structures.

For the preferred tubular DNA origami (60-80 nm×30-50 nm), P22 VLPs are the optimal carrier based on size compatibility. The origami dimensions allow encapsulation with approximately 5-10 nm clearance on each side.

### Assembly Confirmation Methods

Successful hybrid assembly is confirmed by multiple complementary techniques:

### Transmission Electron Microscopy (TEM)

Negative-stain TEM directly visualizes encapsulated origami within VLP shells. Protocol: Apply 5 μL sample to glow-discharged carbon-coated copper grids. Stain with 2% uranyl acetate for 30 seconds. Image at 80-120 kV. Expected appearance: VLP shells appear as light halos surrounding darker origami cores. Encapsulated origami shows defined geometry matching design (tubular, spherical, etc.).

Cryo-EM provides higher resolution structural confirmation without staining artifacts.

### Dynamic Light Scattering (DLS)

DLS measures hydrodynamic diameter and confirms successful assembly. Expected results:
Empty P22 VLP: 60-65 nm hydrodynamic diameter
DNA origami alone: 70-90 nm (geometry-dependent)
Hybrid assembly: 65-75 nm (VLP-dominated size with slight increase due to internal cargo)

Polydispersity index (PDI) below 0.25 indicates monodisperse population suitable for pharmaceutical development.

### Gel Electrophoresis

Native agarose gel electrophoresis separates free origami, empty VLPs, and hybrid assemblies based on size and charge. Protocol: Run samples on 1% agarose gel in TAE buffer at 5 V/cm for 2-4 hours. Stain with ethidium bromide (DNA) and Coomassie (protein) for dual visualization. Expected results: Hybrid band migrates differently from free origami and empty VLP bands. Hybrid band stains positive for both DNA and protein.

### Mass Spectrometry

Native mass spectrometry can determine the mass of intact hybrid assemblies, confirming coat protein stoichiometry and cargo loading. Snijder et al. demonstrated native MS analysis of intact viral capsids, determining mass with sufficient precision to count encapsulated cargo molecules (Snijder et al., J Am Chem Soc. 2014; 136 (20): 7295-7299).

### Section M: Surface Functionalization of Hybrid Assemblies

### Post-Assembly M-Cell Targeting Peptide Conjugation

Following hybrid assembly, M-cell targeting peptides are conjugated to the external VLP surface to enable intestinal transcytosis.

The assembly process described in Section L results in hybrids with the DNA origami (including BBB-targeting peptides) inside the VLP shell, and the VLP outer surface accessible for M-cell peptide conjugation.

### Conjugation Protocol

NHS ester conjugation to surface lysines (preferred method): Materials:
Purified hybrid assemblies: 1-10 mg/mL protein concentration
M-cell targeting peptide with N-terminal NHS-PEG□ linker: 10-100 fold molar excess over VLP
Conjugation buffer: 100 mM sodium phosphate pH 8.0, 150 mM NaCl

Protocol: Step 1: Buffer exchange hybrids into conjugation buffer using spin concentration. Step 2: Add NHS-peptide conjugate and mix by gentle pipetting. Step 3: Incubate at 25 °C for 2-4 hours or 4 °C overnight. Step 4: Quench reaction with 50 mM Tris-HCl pH 8.0. Step 5: Remove unreacted peptide by dialysis or SEC. Step 6: Quantify peptide conjugation by fluorescence (if using fluorescent peptide) or mass spectrometry.

Expected conjugation density: 30-100 peptides per VLP, depending on peptide excess and reaction time. This range is consistent with published VLP surface modification studies (Stephanopoulos et al., ACS Nano. 2010; 4 (10): 6014-6020).

### Optional PEGylation for Immunogenicity Reduction

For applications requiring repeat dosing, PEGylation of the VLP surface may reduce immunogenicity.

PEGylation protocol: Materials:
Purified hybrid assemblies
mPEG-NHS (5 kDa or 10 kDa): 50-200 fold molar excess over VLP surface lysines
Conjugation buffer: 100 mM sodium phosphate pH 8.0

Protocol: Step 1: Add mPEG-NHS to hybrid solution. Step 2: Incubate at 25 °C for 1-2 hours. Step 3: Quench with Tris-HCl. Step 4: Purify by SEC to remove unreacted PEG.

Important: PEGylation should be performed before M-cell peptide conjugation, or M-cell peptides should be attached via longer linkers (PEG□□-PEG□□) to extend beyond the PEG layer.

### Surface Characterization

Surface modifications are characterized by: Zeta Potential: PEGylation typically shifts zeta potential toward neutral. M-cell peptide conjugation may shift zeta potential depending on peptide charge. MALDI-TOF MS: Confirms peptide conjugation by mass shift of coat protein subunits. Functional Binding Assays: M-cell binding ELISA or flow cytometry with M-cell-like cells confirms functional targeting.

### Section N: Oral Formulation Development

### Formulation Objectives

Oral formulation of the hybrid nanostructures aims to: (a) Maintain hybrid structural integrity during storage. (b) Protect hybrids during gastric transit (secondary protection complementing VLP shell). (c) Enable release in the small intestine for M-cell uptake. (d) Achieve practical dosing in standard oral dosage forms.

### Lyophilization for Storage Stability

Lyophilization (freeze-drying) provides optimal long-term stability for protein and nucleic acid therapeutics.

VLPs are generally amenable to lyophilization with appropriate excipients. Lua et al. reviewed lyophilization of VLP-based vaccines, demonstrating stability for greater than 24 months at room temperature with optimized formulations (Lua et al., Biotechnol Bioeng. 2014; 111 (3): 425-440).

### Lyophilization Protocol

The following protocol is based on established methods for VLP lyophilization: Formulation Buffer:
10 mM histidine pH 6.5 (buffer)
5% (w/v) trehalose (cryoprotectant/lyoprotectant)
0.01% (w/v) polysorbate 80 (surfactant, prevents aggregation)

Trehalose is the preferred cryoprotectant based on extensive literature support for protein and VLP stabilization. Trehalose forms a glassy matrix that preserves protein structure during drying (Carpenter et al., “Rational design of stable lyophilized protein formulations: theory and practice,” Pharm Biotechnol. 2002; 13:109-133).

Lyophilization cycle: Freezing:
Shelf temperature: −40 °C.
Freezing time: 2-4 hours
Target product temperature: below −35 °C.

Primary Drying:
Shelf temperature: −20 °C.
Chamber pressure: 50-100 mTorr
Duration: 24-48 hours (until product temperature approaches shelf temperature)

Secondary Drying:
Shelf temperature: +25 °C.
Chamber pressure: 50-100 mTorr
Duration: 6-12 hours

Target residual moisture: less than 2% by Karl Fischer titration. Higher moisture content accelerates degradation during storage.

Post-lyophilization characterization:
Visual appearance: White to off-white cake with no collapse
Reconstitution time: Complete dissolution within 5 minutes
Particle size by DLS: Within 10% of pre-lyophilization values
TEM morphology: Preserved hybrid structure
Functional activity: Retained M-cell binding and BBB-targeting peptide function

### Enteric Coating for Gastric Protection

Enteric coating provides secondary protection against gastric acid degradation, complementing the primary protection afforded by the VLP shell.

While VLPs demonstrate acid stability at pH 2.0-3.0 for several hours (Patterson et al., Soft Matter. 2012), enteric coating provides additional assurance and may improve oral bioavailability by preventing any gastric degradation.

### Enteric Coating Materials

The following enteric coating polymers are approved for pharmaceutical use: Eudragit L100-55: Dissolves above pH 5.5. Provides release in duodenum. Eudragit L100: Dissolves above pH 6.0. Provides release in jejunum. Eudragit S100: Dissolves above pH 7.0. Provides release in ileum (near Peyer's patches). HPMC-AS (Hypromellose acetate succinate): Dissolves at pH 5.5-6.0 depending on grade.

For the present invention, Eudragit L100-55 or Eudragit S100 is preferred: Eudragit L100-55: Ensures release in proximal small intestine with maximum transit time for M-cell uptake. Eudragit S100: Provides release closer to Peyer's patches but with shorter uptake window.

Selection may be guided by pharmacokinetic studies comparing release sites.

### Capsule Formulation

The hybrid nanostructure formulation is filled into hard gelatin or HPMC capsules. Capsule Filling Process: Step 1: Prepare lyophilized hybrid powder with excipients. Step 2: Blend with additional excipients if needed for capsule filling:
Mannitol (diluent): 50-200 mg per capsule
Colloidal silicon dioxide (glidant): 0.5-1%
Magnesium stearate (lubricant): 0.25-0.5% Step 3: Fill into size 0 or 00 hard capsules

using automated capsule filler. Step 4: Apply enteric coating to filled capsules using fluidized bed coater.

Enteric coating application: Coating solution: 15% (w/w) Eudragit L100-55 in ethanol:water (9:1) with 5% triethyl citrate (plasticizer). Coating process: Fluidized bed coating at 40 °C inlet temperature until 30-50% weight gain. Curing: Dry at 40 °C for 2-4 hours to ensure complete film formation.

### Capsule Specifications

Target capsule specifications: Fill weight: 300-500 mg total Hybrid nanostructure content: 10-100 mg (expressed as DNA origami equivalent) Disintegration (acid stage): No disintegration in 0.1 N HCl for 2 hours Disintegration (buffer stage): Complete disintegration within 30 minutes in pH 6.8 phosphate buffer

### Stability Testing

Stability testing per ICH guidelines (ICH Q1A (R2)): Storage conditions:
Long-term: 25 °C/60% RH for 24 months
Intermediate: 30 °C/65% RH for 12 months
Accelerated: 40 °C/75% RH for 6 months

Test parameters at each time point:
Appearance (visual)
Moisture content (Karl Fischer)
Particle size (DLS upon reconstitution)
Structural integrity (TEM)
Acid resistance (disintegration testing)
Potency (functional binding assay)

Expected stability: Based on lyophilized VLP vaccine stability data, greater than 24 months at 25 °C is expected with optimized formulation (Lua et al., Biotechnol Bioeng. 2014; 111 (3): 425-440).

### Section O: Expected Performance Based on Literature Data

This section provides conservative performance predictions based exclusively on published data for the individual component technologies. These predictions are intended to guide experimental design and do not constitute demonstrated results.

### Gastrointestinal Stability Predictions

Based on published VLP and bacteriophage stability data: Gastric Stability (pH 1.2-2.0, 37 °C):
P22 VLP: Greater than 90% structural integrity at 2 hours (extrapolated from Patterson et al., Soft Matter. 2012, showing stability at pH 2.0)
With enteric coating: Greater than 99% protection (coating prevents acid exposure) Intestinal Stability (pH 6.8, pancreatin, 37 °C):
VLP shell: Greater than 80% integrity at 4 hours (based on phage protease resistance data)
M-cell targeting peptide: 50-80% retention at 4 hours (peptide degradation is limiting factor)
DNA origami core: Greater than 95% integrity (protected by VLP shell and oligolysine coating)

Overall GI transit: Expected greater than 70% intact hybrid recovery after simulated GI transit (combined SGF+SIF exposure). This prediction requires experimental validation.

### M-Cell Uptake Predictions

Based on published M-cell targeting nanoparticle data: Enhancement over non-targeted particles: 5-10 fold (Yoo et al., Biomaterials. 2010; 31 (30): 7738-7747) Absolute transcytosis efficiency in Caco-2/Raji model: 3-8% of applied dose at 4 hours for M-cell targeted particles (Kim et al., ACS Nano. 2019; 13 (11): 12671-12681)

For comparison, non-targeted particles typically show 0.5-1.5% transcytosis in the same model. The 5-10 fold enhancement from M-cell targeting is consistent across multiple independent studies.

### Systemic Pharmacokinetics Predictions

Oral bioavailability prediction based on published data: Starting assumption: 0.5-2% baseline oral bioavailability for unprotected, non-targeted nanoparticles (Reinholz et al., “The challenges of oral drug delivery via nanocarriers,” Drug Deliv. 2018; 25 (1): 1694-1705). VLP protection contribution: 2-5 fold enhancement due to GI stability (based on phage survival data) M-cell targeting contribution: 5-10 fold enhancement (Yoo et al., Biomaterials. 2010) Combined estimate: 0.5%×3×7=approximately 10% oral bioavailability Conservative estimate: 3-8% oral bioavailability Optimistic estimate: 8-15% oral bioavailability

These estimates acknowledge substantial uncertainty. Actual oral bioavailability will be determined by in vivo studies in appropriate animal models.

For context, even 3-5% oral bioavailability would represent a significant advance over current oral biologic technologies (semaglutide: approximately 1%; insulin: less than 1%).

### Blood-Brain Barrier Crossing Predictions

Based on published DNA nanostructure BBB crossing data (Jiang et al., Nat Mater. 2024): IV administration of DNA nanostructures: 3-7% of injected dose reaches brain parenchyma Enhancement from dual targeting (T7+RVG-29): 1.5-2 fold additional enhancement expected based on dual-targeting nanoparticle studies (Gao et al., Mol Pharm. 2014; 11 (8): 2755-2767)

Combined oral-to-brain delivery prediction: Oral bioavailability: 5% (conservative mid-range estimate) BBB crossing: 5% of systemic exposure (conservative estimate from Jiang et al.) Dual targeting enhancement: 1.5 fold Calculation: 5%×5%×1.5=0.375% of oral dose reaching brain Conservative estimate: 0.2-0.5% of oral dose reaches brain Optimistic estimate: 0.5-1.5% of oral dose reaches brain

These estimates are substantially lower than the 3% prediction in the original patent draft, reflecting a more conservative interpretation of available data.

For context, 0.2-0.5% brain delivery from oral administration would still represent a breakthrough for biologic therapeutics:
Current IV antibodies achieve 0.1-0.4% brain/plasma ratio (Pardridge, BioDrugs. 2017)
Oral delivery at 0.3% would match IV delivery efficiency while eliminating injection requirement
This efficiency would enable therapeutically relevant brain concentrations at practical oral doses

### Brain Distribution Predictions

Based on published RVG-29 targeting data: Cellular distribution: 60-80% neuronal, 15-25% astrocytic, 5-15% microglial (based on Kumar et al., Nature. 2007 and subsequent studies) Regional distribution: Preferential accumulation in hippocampus and cortex expected based on α7-nAChR expression patterns (Gotti et al., “Structural and functional diversity of native brain neuronal nicotinic receptors,” Biochem Pharmacol. 2009; 78 (7): 703-711)

This distribution pattern is favorable for Alzheimer's disease applications, as hippocampus and cortex are primary sites of amyloid and tau pathology.

### Payload Delivery Predictions

Based on DNA origami drug loading data: Small molecules (doxorubicin model): 1,000-5,000 molecules per origami (Jiang et al., J Am Chem Soc. 2012; 134 (32): 13396-13403)

siRNA: 10-50 molecules per origami (Lee et al., Nat Nanotechnol. 2012; 7 (6): 389-393) Antibody fragments (Fab): 1-10 per origami (size-limited) mRNA: 1-5 per origami (size-limited)

Payload release: pH-responsive release (i-motif mechanism) expected to achieve 60-90% release at endosomal pH 5.5-6.0 within 2-4 hours (Burns et al., J Am Chem Soc. 2014; 136 (45): 15767-15774).

### Section P: Safety Considerations

### Immunogenicity Assessment

VLP-based carriers are inherently immunogenic, as they present repetitive protein epitopes that efficiently activate B cells. This section addresses immunogenicity risks and monitoring strategies.

### Expected Immune Response

Based on published VLP immunogenicity data: First dose: Minimal antibody response expected (naive immune system) Repeat dosing (weekly for 4+ weeks): Anti-VLP IgG titers of 1:1,000 to 1:100,000 expected

(Cornuz et al., PLOS One. 2008; 3 (6): e2547)

Neutralization potential: High-titer antibodies may reduce bioavailability of subsequent doses by 50-90%

Mitigation strategies (described in Section C) include: (a) PEGylation to mask epitopes (b) Immune tolerance protocols (c) VLP platform rotation (d) Immunosuppressive co-therapy (for severe diseases)

### Immunogenicity Monitoring Plan

For clinical development, the following immunogenicity assessments are recommended: Pre-dose: Baseline anti-VLP antibodies (some individuals may have pre-existing antibodies from environmental phage exposure)

During treatment: Anti-VLP IgG and IgM titers at weeks 2, 4, 8, and 12 Post-treatment: Anti-VLP antibodies at 1, 3, and 6 months after last dose

Neutralizing antibody assay: In vitro assay measuring antibody-mediated inhibition of hybrid uptake by M-cell or BBB cell models.

### DNA Origami Safety

DNA nanostructures have demonstrated favorable safety profiles in preclinical studies.

Jiang et al. reported no acute toxicity of DNA nanostructures at doses up to 10 mg/kg IV in mice (Jiang et al., Nat Biomed Eng. 2018; 2 (11): 865-877). Key safety findings:
No weight loss or behavioral changes
Normal liver and kidney function markers
No histopathological abnormalities in major organs
DNA nanostructures are cleared primarily through kidney (renal excretion)

Surana et al. systematically evaluated immunostimulation by DNA nanostructures and found that properly designed structures with methylated CpG sequences show minimal inflammatory cytokine induction (Surana et al., “Designing DNA nanodevices for compatibility with the immune system of higher organisms,” Nat Nanotechnol. 2015; 10 (9): 741-747).

### CpG Content Considerations

Unmethylated CpG dinucleotides in bacterial DNA are recognized by Toll-like receptor 9 (TLR9) and can trigger innate immune activation.

The M13mp18 scaffold contains approximately 350 CpG dinucleotides. Strategies to minimize immunostimulation include: (a) Use CpG-free scaffold sequences (available from commercial suppliers) (b) Methylate CpG sites using M.SssI methyltransferase (c) Include TLR9 inhibitory sequences in staple design

Schüller et al. demonstrated that DNA origami immunostimulation can be controlled by strategic placement of CpG motifs (Schüller et al., ACS Nano. 2011; 5 (12): 9696-9702).

### Genotoxicity Considerations

DNA therapeutics raise theoretical genotoxicity concerns related to potential genomic integration.

DNA origami structures are fundamentally different from gene therapy vectors and pose minimal genotoxicity risk: (a) No promoter or coding sequences: DNA origami does not encode functional genes (b) No integration machinery: Unlike viral vectors, DNA origami has no mechanism for genomic integration (c) Rapid degradation: Unprotected DNA origami is degraded within hours by cellular nucleases (d) No replication: DNA origami cannot replicate in mammalian cells

Standard genotoxicity testing (Ames test, chromosomal aberration assay, micronucleus assay) should be performed during preclinical development per ICH S2 (R1) guidelines.

### Biodistribution and Clearance

Based on published nanoparticle pharmacokinetics data: Expected half-life: 2-8 hours for VLP-based carriers (based on phage pharmacokinetics:

### Dąbrowska et al., J Appl Microbiol. 2005; 98 (1): 7-13)

Primary clearance routes:
Reticuloendothelial system (liver, spleen): 60-80% of dose
Renal excretion of degradation products: 10-20% of dose
Peyer's patch retention: 5-15% of oral dose (for M-cell targeted particles)

Complete clearance: VLP and DNA origami components are biodegradable and expected to be fully cleared within 24-72 hours.

### Organ Toxicity Considerations

Based on published data for VLPs and DNA nanostructures: Liver: Expected accumulation due to RES uptake. Monitor liver enzymes (ALT, AST) during treatment. Spleen: Expected accumulation. Generally well-tolerated based on VLP vaccine experience. Kidney: DNA origami degradation products are renally excreted. Monitor renal function (creatinine, BUN). Brain: Target organ. Monitor for neuroinflammation through imaging (MRI) and CSF analysis if accessible.

Lua et al. reviewed safety data from VLP-based vaccine clinical trials involving thousands of subjects, with no serious adverse events attributable to the VLP carrier (Lua et al., Biotechnol Bioeng. 2014; 111 (3): 425-440).

### Reproductive and Developmental Toxicity

Standard reproductive toxicity studies (fertility, embryo-fetal development, pre/postnatal development) should be conducted per ICH S5 (R3) guidelines before clinical trials in women of childbearing potential.

Based on the mechanism of action and biodegradation properties, no specific reproductive toxicity signals are anticipated, but this requires experimental confirmation.

### Section Q: Quality Control and Release Testing

### Raw Material Specifications

Quality control begins with raw material specifications: M13mp18 Scaffold DNA:
Identity: Sequence verification by restriction digestion pattern
Purity: Greater than 95% by agarose gel electrophoresis
Concentration: Accurately determined by UV absorbance (A260)
Endotoxin: Less than 10 EU/mg

Staple Oligonucleotides:
Identity: Mass spectrometry confirmation of expected mass
Purity: Greater than 85% by HPLC or PAGE
Sequence accuracy: Verified by supplier quality control

VLP Coat Protein:
Identity: SDS-PAGE with Western blot confirmation
Purity: Greater than 95% by SDS-PAGE densitometry
Endotoxin: Less than 10 EU/mg
Assembly competence: Confirmed by VLP formation assay

Targeting Peptides:
Identity: Mass spectrometry
Purity: Greater than 95% by HPLC
Sequence: Confirmed by Edman degradation or MS/MS

### In-Process Controls

Critical in-process controls: DNA Origami Assembly:
Gel electrophoresis: Single band at expected mobility
DLS: Size within specification (80-120 nm for tubular geometry)
TEM: Correct morphology in greater than 70% of particles

Origami Stabilization:
Zeta potential shift: From approximately −30 mV to approximately −5 to +5 mV after oligolysine coating
Nuclease resistance: Greater than 80% intact after 1 hour DNase I treatment

Hybrid Assembly:
Encapsulation efficiency: Greater than 50% by gel electrophoresis
TEM: Core-shell structure visible in greater than 60% of particles

Surface Functionalization:
Peptide conjugation: 30-100 peptides per VLP by mass spectrometry or fluorescence

### Drug Product Release Specifications

Final product release specifications: Appearance: White to off-white lyophilized powder or cake Identity:
DNA origami: Positive by PCR or hybridization assay for scaffold sequence
VLP coat protein: Positive by Western blot or ELISA
Targeting peptides: Positive by ELISA or mass spectrometry

Purity:
Aggregates: Less than 10% by SEC
Free DNA: Less than 5% by gel electrophoresis
Free protein: Less than 5% by SEC

Potency:
M-cell binding: Greater than 50% of reference standard
TfR binding: Greater than 50% of reference standard
Payload content: 80-120% of label claim

Physical Characteristics:
Particle size (DLS): Within specification range (±20% of target)
PDI: Less than 0.30
Moisture content: Less than 3%

Safety:
Endotoxin: Less than 100 EU per dose
Bioburden: Less than 10 CFU/g (pre-sterilization) or sterile (post-sterilization)
Residual solvents: Per ICH Q3C limits

### Stability-Indicating Methods

The following methods are validated as stability-indicating:
DLS particle size: Detects aggregation and structural changes
SEC: Detects aggregation, fragmentation, and free components
TEM: Detects morphological changes
Potency assays: Detect loss of targeting function
Payload content: Detects payload leakage or degradation

### Umbrella Scope Expansion—Ten Architectural Claim Axes

The present application discloses an umbrella architecture for oral central-nervous-system therapeutic delivery that comprises ten orthogonal claim axes. Each axis is described at the functional, mechanistic, and class level rather than at the level of literal sequences or single chemical species, and each axis admits a wide range of operative embodiments that fall within the scope of the claims set forth herein.

### Axis 1—Therapeutic Payload Class

In a first claim axis, the hybrid nanostructure of the invention is configured to carry every class of therapeutic payload deliverable to a central nervous system target by oral administration of a particulate carrier of from 20 to 200 nanometers in diameter, where the payload has a molecular weight of from 100 daltons to 200 kilodaltons. Suitable payloads include, without limitation, small molecule drugs across every therapeutic class including neurotransmitter receptor agonists, neurotransmitter receptor antagonists, neurotransmitter reuptake inhibitors, enzyme inhibitors of every chemical class, ion channel modulators, G-protein coupled receptor agonists and antagonists, kinase inhibitors, phosphatase inhibitors, GTPase modulators, nuclear receptor agonists and antagonists, epigenetic modulators including histone deacetylase inhibitors and DNA methyltransferase inhibitors and bromodomain inhibitors, proteasome inhibitors, autophagy modulators, mitochondrial function modulators, and amyloid aggregation modulators.

Suitable payloads further include peptide therapeutics of every cargo class, including growth-factor-mimetic peptides, neurotrophic factor-mimetic peptides, neuropeptide receptor agonists and antagonists, anti-amyloid aggregation-disrupting peptides, anti-tau aggregation-disrupting peptides, anti-synuclein aggregation-disrupting peptides, mitochondrial targeting peptides, anti-neuroinflammatory peptides, cell-penetrating peptides serving as intracellular delivery cargo, conformation-stabilizing peptides for misfolded-protein diseases, and peptide hormones with central activity.

Suitable payloads further include protein therapeutics including full-length monoclonal antibodies, antigen-binding antibody fragments (Fab, F(ab′)2, scFv, single-domain antibodies and nanobodies), bispecific T-cell engagers, designed ankyrin-repeat proteins, monobodies, affibodies, anticalins, knottins, fynomers, and engineered enzyme replacements for inherited metabolic disorders with central nervous system involvement.

Suitable payloads further include nucleic acid therapeutics across every modality. These include messenger RNA constructs encoding therapeutic proteins for transient cellular expression, self-amplifying RNA, circular RNA, small interfering RNA configured to silence disease-associated transcripts, microRNA mimics and microRNA inhibitors (antagomirs), antisense oligonucleotides employing phosphorothioate, 2′-O-methyl, 2′-O-methoxyethyl, 2′-fluoro, locked nucleic acid, and constrained ethyl chemistries, ribozymes, aptamers, and transcription factor decoys.

Suitable payloads further include gene-editing ribonucleoprotein complexes and gene-editing components configured for delivery in active form, including CRISPR-Cas9, CRISPR-Cas12a, CRISPR-Cas12b, CRISPR-Cas13, CRISPR-CasX, prime editors, cytosine and adenine base editors, dual base editors, transposon-based gene insertion systems including PiggyBac and Sleeping Beauty, and integrase-deficient lentiviral particle equivalents reformatted into protein nanocage carriers.

Suitable payloads further include emerging modalities not enumerated above, including molecular glue degraders, proteolysis-targeting chimeras, autophagy-targeting chimeras, lysosome-targeting chimeras, and RNA-recruiting chimeric small molecules, together with combinations of two or more of any of the foregoing payload classes co-loaded into a single nanostructure for combination therapy across orthogonal pathological mechanisms.

In every embodiment of Axis 1 the payload is associated with the inner core of the nanostructure through one or more of (a) encapsulation within an internal cavity defined by the inner core, (b) intercalation into double-helical regions of an inner DNA or RNA scaffold, (c) electrostatic association with the inner core surface, (d) covalent conjugation to chemically modified internal anchor positions, (e) hybridization to complementary sequences incorporated into the inner core, (f) aptamer-mediated capture by internal aptamer elements, or (g) any combination of the foregoing loading mechanisms.

### Axis 2—Outer Shell Architecture

In a second claim axis, the protective outer shell of the nanostructure is selected from every class of protective shell architecture that provides gastrointestinal stability for an oral particulate carrier of from 20 to 200 nanometers in diameter, while permitting intestinal transcytosis and downstream blood-brain barrier crossing. Suitable shell architectures include, without limitation, virus-like particles from every characterized bacteriophage and plant virus source.

Particular bacteriophage-derived shells include those derived from P22 bacteriophage, MS2 bacteriophage, Qβ bacteriophage, PP7 bacteriophage, AP205 bacteriophage, T4 bacteriophage, T7 bacteriophage, lambda bacteriophage, phi-29 bacteriophage, and phi-X174 bacteriophage, and functional equivalents thereof, wherein each said shell is substantially devoid of viral genomic nucleic acid and comprises recombinantly expressed coat protein subunits assembled into an icosahedral, filamentous, or rod-shaped structure.

Particular plant-virus-derived shells include those derived from cowpea chlorotic mottle virus, cowpea mosaic virus, brome mosaic virus, tobacco mosaic virus, hibiscus chlorotic ringspot virus, and functional equivalents thereof.

Particular engineered protein nanocage shells include shells assembled from ferritin and apoferritin subunits, encapsulin subunits, lumazine synthase subunits, heat-shock protein cage subunits, vault-protein cage subunits, designed protein cages including ones produced by computational protein design, and engineered variants of any of the foregoing.

Particular lipid bilayer shells include conventional liposomes, lipid nanoparticles incorporating ionizable lipids, solid lipid nanoparticles, nanostructured lipid carriers, and lipid-polymer hybrid particles.

Particular polymer shells include shells formed from poly(lactic-co-glycolic acid), polylactic acid, polyglycolic acid, polycaprolactone, chitosan and chitosan derivatives, alginate, gelatin, casein, silk fibroin, hyaluronic acid, dextran, cyclodextrin scaffolds, polyethyleneimine and lower-molecular-weight variants thereof, polyamidoamine dendrimers, poly(beta-amino esters), and biodegradable copolymers thereof.

Particular inorganic shells include mesoporous silica nanoparticles, calcium phosphate shells, hydroxyapatite shells, magnetite-iron-oxide cores with biocompatible coating, and inorganic-organic hybrid shells.

Particular biomimetic membrane coatings include shells coated with cellular membranes isolated from red blood cells, platelets, leukocytes, mesenchymal stem cells, neural stem cells, cancer cells of homologous origin, exosomes, and hybrid combinations of two or more such membranes, wherein the coating preserves selected membrane proteins for biological function while masking the underlying carrier from rapid immune clearance.

In every embodiment of Axis 2 the outer shell is configured to (i) resist degradation by gastric acid at a pH of from 1.0 to 3.0 for at least 30 minutes at 37 degrees Celsius, (ii) resist degradation by intestinal proteases under physiological conditions for at least 1 hour at 37 degrees Celsius, and (iii) release or transport the encapsulated inner core across the gastrointestinal mucosal barrier through one or more mechanisms recited in Axis 4 below.

### Axis 3—Inner Core Architecture

In a third claim axis, the structured inner core encapsulated within the protective outer shell is selected from every architecture capable of organizing therapeutic payload at the nanoscale and supporting controlled, stimulus-responsive release. Suitable inner core architectures include, without limitation, DNA origami nanostructures of every geometry, RNA origami nanostructures, peptide nanofiber and peptide-amphiphile cores, protein nanocage interiors, dendrimer-based core architectures, mesoporous inorganic cores, polymeric micelle cores, hydrogel cores, liquid-crystalline cores, and combinations of two or more of the foregoing architectures arranged hierarchically.

DNA origami inner cores include cores formed by folding a single-stranded DNA scaffold of from 1,000 to 50,000 nucleotides through hybridization with a plurality of complementary staple oligonucleotides into a predetermined three-dimensional shape selected from tubular geometries having lengths of from 30 to 120 nanometers and outer diameters of from 15 to 70 nanometers, spherical or quasi-spherical geometries having diameters of from 20 to 80 nanometers, polyhedral geometries including tetrahedral, octahedral, dodecahedral, and icosahedral wireframes, planar two-dimensional sheets, and hybrid multi-arm or branched architectures.

RNA origami inner cores include cores assembled from co-transcriptionally folded RNA molecules and from designed RNA architectures employing kissing loops, three-way junctions, four-way junctions, and pseudoknot motifs to define internal cavities of from 10 to 40 nanometers in diameter.

Peptide nanofiber and peptide-amphiphile cores include cores formed from self-assembling peptides employing beta-sheet, coiled-coil, or amphiphilic block architectures and providing controlled release through peptidase cleavage or pH-induced disassembly.

Protein nanocage interiors include cores formed from encapsulin or ferritin or vault protein interiors that present payload-binding sites on the inner surface, optionally engineered to display payload-anchoring tags such as elastin-like polypeptides or intrinsically disordered domains for payload retention.

Inner core architectures of Axis 3 are characterized by (i) a defined internal cavity geometry, (ii) at least one stimulus-responsive element coupling cavity opening or payload release to a defined trigger, and (iii) at least one anchor or binding site enabling controlled placement of payload molecules at predetermined positions within the cavity.

### Axis 4—Dual-Barrier Crossing Mechanism

In a fourth claim axis, the hybrid nanostructure is configured to traverse, in sequence, (i) the gastrointestinal absorptive barrier and (ii) the blood-brain barrier. Suitable mechanisms for the gastrointestinal-barrier traversal include, without limitation, microfold-cell-mediated transcytosis directed by an M-cell targeting ligand, enterocyte transcellular transport directed by villus-cell-binding ligands, paracellular tight junction modulation employing transient tight-junction-opening enhancers, lymphatic uptake via chylomicron-mimetic formulations co-loading lipidic excipients, transcytotic uptake via the neonatal Fc receptor on enterocytes, and Peyer's-patch-mediated lymphoid uptake.

M-cell targeting ligands include peptide ligands of from 5 to 30 amino acid residues identified by phage display screening for binding to M-cell apical surfaces, antibody fragments specific for M-cell surface markers including glycoprotein 2 and beta-1 integrin, carbohydrate moieties that bind M-cell-expressed lectins, aptamer ligands selected for M-cell binding, and peptides derived from bacterial invasion proteins including Salmonella and Yersinia invasion-protein-derived sequences engineered to retain receptor binding without retaining infectious capacity.

Suitable mechanisms for the blood-brain barrier traversal include, without limitation, receptor-mediated transcytosis directed by ligands that bind one or more of the transferrin receptor, the insulin receptor, the insulin-like growth factor 1 receptor, the low-density lipoprotein receptor, the low-density lipoprotein receptor-related protein 1, the low-density lipoprotein receptor-related protein 8, the leptin receptor, the nicotinic acetylcholine receptor, the glucose transporter 1, the large neutral amino acid transporter 1, the monocarboxylate transporter 1, the cationic amino acid transporter, the scavenger receptor classes B-I and B-II, the diphtheria toxin receptor (heparin-binding epidermal growth factor), and the melanotransferrin receptor.

Suitable BBB-crossing ligands include peptide ligands of from 5 to 35 amino acid residues configured to bind any of the foregoing receptors with affinity below 1 micromolar, antibody fragments and engineered binders configured to bind such receptors with moderate affinity selected to favor transcytosis over receptor sequestration, full-length transferrin protein, transferrin variants engineered for monovalent receptor binding, natural ligands of the foregoing receptors, mimetic ligands, and aptamer ligands selected for the foregoing receptors.

Additional BBB-crossing mechanisms include adsorptive-mediated transcytosis directed by cell-penetrating peptides and cationic surface coatings, carrier-mediated transport employing glucose-transporter or amino-acid-transporter-targeted modifications, and physically assisted crossing methods including focused-ultrasound-mediated transient BBB opening when combined with a physically activated outer-shell-shedding mechanism, magnetic-field-assisted enhancement of crossing for shells incorporating magnetic-iron cores, and electromagnetic-stimulus-assisted enhancement under controlled clinical supervision.

In every embodiment of Axis 4 the dual-barrier traversal architecture provides for sequential operation in which (a) gastrointestinal-barrier ligands are presented on the outer surface of the outer shell during transit through the gut, (b) the outer shell is either retained intact or progressively shed during transit such that downstream BBB-crossing ligands carried on the inner core become accessible upon entry into the systemic circulation, and (c) the BBB-crossing ligand mediates receptor-mediated transcytosis of the inner core into the central nervous system compartment.

Axis 5—pH-Responsive and Stimulus-Responsive Release

In a fifth claim axis, the hybrid nanostructure incorporates at least one stimulus-responsive element configured to trigger conformational change, shell disassembly, linker cleavage, or payload release in response to a defined environmental, biochemical, or external stimulus encountered along the dual-barrier transit path or at the central nervous system target tissue.

Suitable pH-responsive elements include pH-responsive DNA architectures including cytosine-rich oligonucleotide elements of from 20 to 30 nucleotides configured to undergo conformational change from a single-stranded random coil at physiological pH to an intercalated tetrad structure at pH below approximately 6.5 through cytosine-cytosine-plus base pair formation; triplex DNA elements configured to undergo transitions over the physiological-to-acidic pH range via Hoogsteen base pair formation; pH-sensitive linkers including hydrazone and acetal and beta-thiopropionate linkages; pH-responsive polymer coatings including poly(beta-amino ester) and poly(histidine) coatings; and enteric polymer systems including methacrylic acid copolymers configured to dissolve at pH above 5.5, 6.0, 7.0, or other defined thresholds matched to the gastrointestinal pH transition.

Suitable enzymatic-responsive elements include peptide linkers cleavable by cathepsin B (e.g., for endolysosomal release), cathepsin S, cathepsin L, caspase-3, matrix metalloproteinases including MMP-2 and MMP-9 for release in tumor or inflammatory microenvironments, prostate-specific antigen for prostate-cancer-adjacent contexts, and trypsin-blocked or chymotrypsin-blocked variants engineered for selective cleavage. Suitable enzymatic-responsive nucleic acid elements include DNA sequences cleavable by specific endonucleases controlled by ribozyme switches.

Suitable redox-responsive elements include disulfide bonds cleavable by intracellular glutathione at 1-10 millimolar concentrations, diselenide linkages, thioether-quinone linkages, and selenoether-based linkers.

Suitable reactive-oxygen-species-responsive elements include thioketal linkages, phenylboronic ester linkages, and arylsulfide-containing linkers engineered to respond to elevated reactive oxygen species in inflamed or tumor tissue.

Suitable physical-stimulus-responsive elements include photocleavable linkers (activated by ultraviolet, visible, or near-infrared light depending on absorbing chromophore), thermosensitive elements activated above 40 degrees Celsius (for use with focused-ultrasound-induced or magnetic-hyperthermia-induced local heating), ultrasound-responsive microbubble-coupled releases, and magnetically actuated release elements.

Suitable logic-gated multi-input release architectures combine two or more of the foregoing responsive elements in series, in parallel, or in branched logic such that payload release requires conjunction (AND), disjunction (OR), or threshold combinations of multiple stimulus inputs. Particular logic-gated architectures include pH-AND-enzyme gates that release payload only in compartments meeting both criteria, and time-delayed redox gates that release payload only after a minimum cytosolic residence period.

### Axis 6—Surface Targeting and Stealth

In a sixth claim axis, the exterior surface of the nanostructure carries a defined combination of targeting and stealth modifications selected to achieve (i) avoidance of immediate immune clearance, (ii) suitable circulation half-life consistent with the transit and accumulation kinetics required for the dual-barrier route, (iii) directed binding to gastrointestinal and central-nervous-system tissue targets, and (iv) tunable shedding or de-targeting after receptor engagement.

Suitable stealth modifications include conjugation of poly(ethylene glycol) chains of molecular weight from 1,000 to 20,000 daltons at surface coverage densities from 5 to 50 percent of available conjugation sites, and equivalent zwitterionic stealth coatings including polysarcosine, poly(carboxybetaine), poly(sulfobetaine), and poly(2-methacryloyloxyethyl phosphorylcholine), which provide low non-specific protein adsorption without the immunogenic complications occasionally associated with anti-PEG antibody responses.

Suitable targeting modifications include any single or combination of the gastrointestinal-targeting ligands recited in Axis 4 and the BBB-crossing ligands recited in Axis 4, presented on the outer shell exterior, on shed sub-particles, or on the inner core exterior depending on the timing of intended ligand exposure.

Particular dual-targeting architectures combine a gastrointestinal-targeting ligand carried on the outermost shell layer with a blood-brain-barrier-crossing ligand carried on a sub-layer that is exposed only after dissolution of the outermost layer in response to the gastrointestinal pH and protease environment, providing temporal separation of the two targeting modalities along the transit path.

Particular shedding architectures employ stimulus-responsive linkers (as recited in Axis 5) between the outermost stealth coating and the underlying shell, such that the stealth coating is shed in response to a defined trigger and previously masked targeting moieties become accessible.

### Axis 7—Therapeutic Indication

In a seventh claim axis, the methods of treatment recited herein extend to every neurological, neurodegenerative, neuroinflammatory, neuropsychiatric, neurodevelopmental, neuro-oncologic, and adjacent central nervous system condition amenable to delivery of a therapeutic payload across the gastrointestinal absorptive barrier and the blood-brain barrier.

Particular neurodegenerative indications include Alzheimer's disease and pre-clinical Alzheimer's disease states identified by amyloid or tau positron emission tomography, Parkinson's disease including prodromal stages, Huntington's disease, amyotrophic lateral sclerosis including sporadic and familial subtypes, frontotemporal dementia including tau-pathology and TAR-DNA-binding-protein-43 subtypes, Lewy body dementia, multiple system atrophy, progressive supranuclear palsy, corticobasal degeneration, spinocerebellar ataxias including types 1, 2, 3, 6, and 7, Friedreich's ataxia, and prion diseases including familial Creutzfeldt-Jakob disease and Gerstmann-Sträussler-Scheinker syndrome.

Particular neuroinflammatory indications include multiple sclerosis across relapsing-remitting, secondary progressive, and primary progressive forms, neuromyelitis optica spectrum disorder, acute disseminated encephalomyelitis, autoimmune encephalitis including N-methyl-D-aspartate-receptor-antibody encephalitis, and chronic neuroinflammatory states accompanying long-term sequelae of viral infection.

Particular neuro-oncologic indications include glioblastoma multiforme, anaplastic astrocytoma, diffuse midline glioma, oligodendroglioma, ependymoma, medulloblastoma including molecular subtypes WNT, sonic-hedgehog, group 3, and group 4, atypical teratoid rhabdoid tumor, embryonal tumors, and central nervous system metastases of extracranial primary tumors.

Particular neuropsychiatric indications include major depressive disorder including treatment-resistant subtypes, bipolar disorder, schizophrenia, schizoaffective disorder, generalized anxiety disorder, social anxiety disorder, panic disorder, obsessive-compulsive disorder, post-traumatic stress disorder, substance use disorders, and autism spectrum disorder.

Particular neurodevelopmental and pediatric indications include Rett syndrome, Angelman syndrome, Dravet syndrome, Lennox-Gastaut syndrome, fragile X syndrome, Phelan-McDermid syndrome, lysosomal storage disorders with central nervous system involvement including Gaucher disease type II and type III, Niemann-Pick disease type C, Tay-Sachs disease, Sandhoff disease, GM1 gangliosidosis, neuronal ceroid lipofuscinoses, mucopolysaccharidoses with central involvement, Krabbe disease, and metachromatic leukodystrophy.

Particular acute and subacute indications include ischemic stroke and hemorrhagic stroke (including subarachnoid hemorrhage) within therapeutic windows defined by current standards of care, traumatic brain injury including concussion and severe diffuse axonal injury, spinal cord injury, status epilepticus, and acute neuropsychiatric crises requiring rapid pharmacological response.

Particular chronic and oral-administration-advantaged indications further include every condition requiring long-term continuous dosing where oral administration provides patient adherence, dosing convenience, and cost advantages over parenteral routes; pediatric indications where avoidance of repeated venipuncture is desirable; geriatric indications with co-morbidities limiting injection-based therapy; and indications in resource-limited healthcare settings where injection-based delivery is logistically challenging.

### Axis 8—Manufacturing and Scale-Up

In an eighth claim axis, the manufacturing process for the hybrid nanostructure encompasses every operation required to produce, characterize, fill, and release the final drug product at clinical or commercial scale. Suitable manufacturing operations include, without limitation, recombinant expression of outer-shell coat protein in Escherichia coli, yeast, insect, or mammalian host systems; in vitro self-assembly of virus-like particles from purified coat protein; controlled disassembly-reassembly of pre-formed virus-like particles for cargo encapsulation; folding of structured inner cores from pre-synthesized scaffold and staple components through thermal annealing or isothermal protocols; payload loading by intercalation, electrostatic association, covalent conjugation, aptamer capture, or hybridization; conjugation of targeting and stealth ligands to defined outer-surface positions through N-hydroxysuccinimide-ester, maleimide-thiol, click-chemistry, or SpyTag-SpyCatcher protocols; downstream purification by tangential-flow filtration, sucrose-gradient ultracentrifugation, size-exclusion chromatography, ion-exchange chromatography, and affinity chromatography; sterile fill-finish operations under cGMP; and lyophilization with appropriate cryoprotectants including trehalose, sucrose, mannitol, and excipient stabilizers, with formulation into enteric capsules, tablets, orally disintegrating tablets, oral suspensions, or dissolvable films.

Quality control operations applicable across the manufacturing process include characterization by dynamic light scattering, transmission electron microscopy, cryo-electron microscopy, atomic force microscopy, size-exclusion chromatography, asymmetric flow field-flow fractionation, native mass spectrometry, sodium dodecyl sulfate polyacrylamide gel electrophoresis, agarose gel electrophoresis under native and denaturing conditions, ultraviolet-visible absorption spectroscopy, spectrofluorometric titration, circular dichroism spectroscopy, isothermal calorimetry, surface plasmon resonance binding assays, biolayer interferometry, enzyme-linked immunosorbent assay, and bioassay-based potency assessment using validated cellular models. Acceptance criteria for release testing include defined size distribution, polydispersity index below 0.30, payload content within 80-120 percent of label claim, endotoxin content below 100 endotoxin units per dose, bioburden meeting compendial limits or sterility for sterile presentations, and ligand density within defined ranges per shell or per inner core.

Scale-up considerations include process development from laboratory scale (milligram to gram) through engineering scale (10-100 gram) through clinical-trial scale (kilogram) to commercial scale (multi-kilogram), with stage-appropriate process characterization, validation runs, and tech transfer documentation. Manufacturing embodiments include batch, fed-batch, and continuous-flow assembly configurations.

### Axis 9—Monitoring, Biomarker Integration, and Closed-Loop Feedback

In a ninth claim axis, the hybrid nanostructure architecture is configured to interoperate with biomarker detection elements, monitoring devices, and feedback control architectures that enable adjustment of dosing, formulation, or therapeutic regimen in response to detected pharmacokinetic, pharmacodynamic, or therapeutic response indicators following oral administration.

Suitable biomarker classes include circulating biomarkers detectable in peripheral blood including amyloid-beta isoform ratios for Alzheimer's monitoring, plasma neurofilament light chain for general neuronal injury monitoring, phosphorylated tau isoforms, alpha-synuclein conformational species, glial fibrillary acidic protein for astrocytic activation, ubiquitin C-terminal hydrolase L1 for neuronal injury, S100-B protein, tau-181 and tau-217 phospho-forms, and chemokines and cytokines indicating neuroinflammatory state; cerebrospinal fluid biomarkers accessible by lumbar puncture as appropriate; imaging biomarkers including magnetic resonance imaging volumetric measurements, diffusion tensor imaging, magnetic resonance spectroscopy, amyloid positron emission tomography, tau positron emission tomography, fluorodeoxyglucose positron emission tomography for metabolic monitoring, and functional magnetic resonance imaging for circuit-level response; and digital biomarkers acquired by wearable devices including continuous actigraphy, gait analysis, speech and language metrics, sleep architecture, heart-rate variability, and cognitive task performance.

Suitable detection modalities include point-of-care immunoassays employing lateral-flow strips, microfluidic enzyme-linked immunosorbent assays, single-molecule array immunoassays, electrochemical biosensors, field-effect-transistor biosensors, optical resonator biosensors, and wearable biosensors for continuous monitoring of selected analytes.

Suitable wearable and implant integration architectures include wrist-worn devices, subcutaneous continuous-monitoring implants, intracranial monitoring implants for research and select clinical contexts, smartphone-based assessment platforms, and in-home environmental sensors. Each is configured to feed monitored data through a defined data interface into the feedback control system.

Suitable closed-loop feedback architectures include rules-based systems applying predefined thresholds to monitored biomarkers and adjusting subsequent dose level, dose interval, or formulation; model-predictive control systems employing a pharmacokinetic-pharmacodynamic model of the patient and adjusting dosing to maintain a target trajectory; and machine-learning-based systems including reinforcement-learning controllers, supervised-learning predictors of treatment response, and transformer-based decision support architectures, in each case operating with at least one external clinical override and at least one safety interlock preventing autonomous escalation beyond predefined safety bounds.

### Axis 10—Personalization and Cross-Portfolio Integration

In a tenth claim axis, the hybrid nanostructure architecture is configured to interoperate with personalized therapeutic architectures and cross-portfolio platforms. Suitable personalization architectures include genome-indexed and transcriptome-indexed selection of payload species and dosing parameters based on individual patient genotype across pharmacokinetic, pharmacodynamic, and disease-modifying axes; proteome-indexed and metabolome-indexed personalization employing routinely accessible biofluid measurements; patient-derived induced pluripotent stem cell or organoid-screening-indexed selection of payload species and combinations using in vitro models of the patient's disease tissue; and longitudinal-monitoring-indexed personalization adjusting dosing over multi-month or multi-year horizons based on accumulated response data.

Suitable cross-portfolio integration architectures include integration with personalized methods-of-administration architectures as disclosed in commonly-owned Application No. 64/080,301; integration with genome-indexed therapeutic subscription and multi-decade financing architectures as disclosed in commonly-owned application family DVB-063; integration with the integrated peptide therapeutic platform architecture as disclosed in commonly-owned application family DVB-070; integration with the reversible blood-brain-barrier-penetrating peptide delivery platform as disclosed in commonly-owned application family DVB-016; and portfolio-level integration across the broader Neurocore Biotechnologies LLC patent family. The present non-provisional application does not claim priority benefit from any of the above-referenced related applications, and any reference to those related applications is provided for context only.

### Expanded Umbrella Scope—Deep-Dive Across the Ten Architectural Axes

The following expanded disclosure sets forth, in deeper detail, the structural, functional, and operational embodiments contemplated within each of the ten architectural claim axes summarized above. The expanded disclosure is provided to ensure full enablement of the claimed subject matter under 35 U.S.C. § 112 (a) across the entire umbrella, including embodiments not separately enumerated as numbered examples but nonetheless supported by the prose disclosure herein.

### Expanded Axis 1—Therapeutic Payload Class (Deep Dive)

### Therapeutic Payload Class

### Small Molecule Drug Payloads

The oral nanostructure platform accommodates small molecule drug payloads of from 150 daltons to 900 daltons, encompassing the principal pharmacologically relevant molecular weight window for orally bioavailable central nervous system therapeutics. Within this window, the platform is configured to load BACE1 inhibitor compounds directed against beta-site amyloid precursor protein cleaving enzyme 1, including substrate-pocket binding inhibitors and transition-state mimetics that suppress production of amyloid-beta peptides relevant to Alzheimer disease and related amyloid pathologies. The platform further accommodates gamma-secretase modulator compounds that allosterically shift the cleavage specificity of the presenilin-containing gamma-secretase complex toward shorter, less aggregation-prone amyloid-beta species without abrogating Notch processing, thereby preserving an acceptable safety margin in chronic dosing. Aggregation inhibitor small molecules directed against amyloid-beta, tau, and alpha-synuclein form a further loaded class, including compounds that bind monomer or low-order oligomer surfaces, compounds that interfere with cross-beta sheet stacking, and compounds that destabilize mature fibrils through pi-stacking interactions with aromatic side chains. These compounds typically fall in the molecular weight range of 250 daltons to 700 daltons, are amenable to encapsulation within hydrophobic compartments of the inner core, and exhibit low aqueous solubility that makes the conventional oral route inefficient, providing a strong rationale for nanostructure-mediated solubilization and lymphatic uptake. Loading is preferentially achieved through hydrophobic partitioning into a lipidic inner core compartment, with release triggered by enzymatic degradation of the surrounding protective shell upon arrival at the cerebrovascular endothelium.

Ion channel modulator small molecules constitute a further payload subclass and span the molecular weight range of 200 daltons to 800 daltons. The platform is configured to load voltage-gated sodium channel modulators of the Nav1.1, Nav1.2, Nav1.6, Nav1.7, Nav1.8, and Nav1.9 subtypes, providing therapeutic coverage across epilepsy, neuropathic pain, and channelopathy indications. Potassium channel modulators of the Kv, KCNQ, and small-conductance and large-conductance calcium-activated potassium channel classes are accommodated for indications including epileptic encephalopathies, ataxias, and disorders of neuronal hyperexcitability. Voltage-gated calcium channel modulators acting at the T-type, L-type, N-type, and P/Q-type subtypes are loaded for neuropathic pain, absence epilepsy, and ataxia indications. Transient receptor potential channel modulators directed at TRPV1, TRPA1, TRPM8, and TRPV3 subtypes are accommodated for chronic pain, itch, and small-fiber neuropathy indications. These small molecule modulators are typically lipophilic, with log P values in the range of 2 to 5, rendering them well suited to encapsulation within a lipidic inner core. Oral delivery via the disclosed nanostructure is advantageous because the conventional oral route subjects these molecules to extensive first-pass hepatic metabolism by cytochrome P450 enzymes including CYP3A4 and CYP2D6 and to efflux at the intestinal epithelium by P-glycoprotein and breast cancer resistance protein, both of which are circumvented by nanostructure-mediated transcytosis through enterocytes and uptake into intestinal lymphatic chylomicrons. Release within the cerebrovascular compartment is triggered by a class of stimulus-responsive linker chosen from pH-sensitive, redox-sensitive, and enzyme-cleavable linker classes.

Neurotransmitter receptor modulator small molecules form a major payload class loaded into the inner nanostructure core across the molecular weight range of 200 daltons to 700 daltons. Dopaminergic modulators encompassing D1-like and D2-like receptor agonists, antagonists, partial agonists, and biased ligands are accommodated for indications including Parkinson disease, schizophrenia, attention deficit disorders, and substance use disorders. Serotonergic modulators directed at the 5-HT1A, 5-HT2A, 5-HT2C, 5-HT4, 5-HT6, and 5-HT7 receptor subtypes are loaded for indications including depression, anxiety, cognition disorders, and irritable bowel disorders with central components. Glutamatergic modulators including N-methyl-D-aspartate receptor modulators acting at GluN2A, GluN2B, GluN2C, and 1 GluN2D subunits, alpha-amino-3-hydroxy-5-methyl-4-isoxazolepropionate receptor positive allosteric modulators, kainate receptor modulators, and metabotropic glutamate receptor modulators directed at the mGluR1 through mGluR8 subtypes are accommodated. GABAergic modulators directed at synaptic and extrasynaptic GABA-A receptor subtypes including alpha1-, alpha2-, alpha3-, alpha5-, and delta-containing receptors and at GABA-B receptor subtypes are loaded for anxiety, epilepsy, and spasticity indications. Cholinergic modulators including muscarinic M1, M2, M3, M4, and M5 receptor modulators and nicotinic acetylcholine receptor modulators directed at alpha4-beta2, alpha7, and alpha3-beta4 subtypes are accommodated for cognitive, addiction, and movement-disorder indications. These molecules are loaded into a hydrophobic core compartment or into an amphipathic intermediate compartment in cases of moderate aqueous solubility. Release is triggered by a class of trigger chosen from glutathione-responsive disulfide cleavage at the cerebrovascular endothelium, esterase-mediated cleavage of carboxyl ester linkers, and matrix-metalloproteinase-cleavable peptide linker classes.

Kinase inhibitor small molecules of neurological relevance form an additional payload class loaded across the molecular weight range of 300 daltons to 800 daltons. The platform accommodates inhibitors of leucine-rich repeat kinase 2 for synucleinopathy indications, inhibitors of dual-specificity tyrosine-regulated kinase 1A for Down syndrome and Alzheimer indications, inhibitors of glycogen synthase kinase 3 beta for tauopathy and mood disorder indications, inhibitors of cyclin-dependent kinase 5 for tauopathy indications, inhibitors of casein kinase 1 delta and epsilon for circadian and tauopathy indications, inhibitors of receptor-interacting protein kinase 1 and kinase 3 for necroptosis-driven neurodegeneration indications, inhibitors of inhibitor of nuclear factor kappa B kinase for neuroinflammation indications, inhibitors of c-Jun N-terminal kinase isoforms for neurodegenerative and inflammatory indications, and inhibitors of p38 mitogen-activated protein kinase isoforms for neuroinflammation indications. Epigenetic modulator small molecules form a further class loaded across the molecular weight range of 250 daltons to 700 daltons, including histone deacetylase inhibitors selective for class I, class II, and class III subtypes including sirtuin modulators, DNA methyltransferase inhibitors, bromodomain and extra-terminal domain inhibitors, histone methyltransferase inhibitors directed at EZH2 and DOT1L, and histone demethylase inhibitors directed at LSD1 and the jumonji family. Proteasome and autophagy modulator small molecules form a further class, including selective immunoproteasome inhibitors and autophagy enhancer compounds directed at the ULK1, TFEB, and mTORC1 nodes. Mitochondrial function modulator small molecules include complex I, II, and III modulators, mitochondrial permeability transition pore modulators, and uncoupling protein modulators. Nrf2/Keap1 pathway activator small molecules including electrophilic and non-electrophilic activators are loaded for indications driven by oxidative stress. Loading is preferentially by hydrophobic core encapsulation, and release is triggered by pH-responsive or enzyme-responsive linker classes selected to release the payload at the cerebrovascular endothelium or after intracellular uptake into target neurons.

### Peptide Therapeutic Payloads

Peptide therapeutic payloads are loaded into the inner nanostructure core across the molecular weight range of 500 daltons to 10,000 daltons, with the platform configured to accommodate linear peptides, cyclic peptides, stapled peptides, beta-hairpin peptides, and miniprotein architectures. Neurotrophic-factor-mimetic peptide classes form a principal payload subclass and include peptides functionally mimicking the neurotrophin family including nerve growth factor, brain-derived neurotrophic factor, neurotrophin-3, and neurotrophin-4 by engaging tropomyosin receptor kinase A, B, and C receptor classes, peptides mimicking glial cell line-derived neurotrophic factor family ligands by engaging GFRα and RET receptor classes, peptides mimicking ciliary neurotrophic factor and related gp130-coupled cytokines, and peptides mimicking insulin-like growth factor 1 by engaging the IGF-1 receptor class. These peptide classes act as neuroprotective and neurorestorative agents in indications including Alzheimer disease, Parkinson disease, amyotrophic lateral sclerosis, Huntington disease, and traumatic brain injury. Oral delivery is advantageous because intact peptide therapeutics are otherwise rapidly degraded by gastric acid, pepsin, and intestinal proteases including trypsin, chymotrypsin, elastase, and carboxypeptidase, and the conventional oral route yields negligible systemic bioavailability for peptide therapeutics in this molecular weight range. The disclosed nanostructure shields the peptide payload from luminal and brush-border proteolysis and permits enterocyte transcytosis. Loading is achieved by electrostatic complexation with a polyanionic or polycationic inner core matrix, by entrapment within an aqueous core compartment surrounded by an amphipathic boundary, or by reversible covalent tethering through disulfide or hydrazone linker classes. Release is triggered by a class of trigger chosen from glutathione-responsive disulfide reduction, acid-responsive hydrazone cleavage, and protease-cleavable linker classes specific to cerebrovascular endothelial enzymes.

Neurotransmitter receptor agonist and antagonist peptide classes form a further payload subclass and are loaded across the molecular weight range of 500 daltons to 8,000 daltons. The platform accommodates peptide agonists and antagonists of metabotropic glutamate receptor classes, peptide modulators of the kappa, mu, and delta opioid receptor classes including biased agonists with reduced respiratory depression, peptide antagonists of substance P at the neurokinin-1 receptor class, peptide modulators of the cannabinoid CB1 and CB2 receptor classes, and peptide modulators of trace amine-associated receptor classes. Aggregation-disrupting peptide classes for tauopathies and synucleinopathies form an additional payload subclass and are loaded across the molecular weight range of 1,000 daltons to 6,000 daltons. These peptides include beta-sheet breaker peptide classes designed to interfere with cross-beta stacking in tau filaments and alpha-synuclein protofibrils, hot-spot binding peptide classes targeting amyloidogenic sequence motifs, prion-like domain blocker peptide classes, and chaperone-mimetic peptide classes that recruit cellular protein quality control machinery to target aggregates. These peptide classes act on indications including Alzheimer disease, frontotemporal dementia tauopathies, progressive supranuclear palsy, corticobasal degeneration, Parkinson disease, dementia with Lewy bodies, and multiple system atrophy. Oral delivery via the disclosed nanostructure is advantageous because aggregation-disrupting peptides are typically polar, polyionic, and proteolytically unstable, rendering the conventional oral route ineffective. Loading is achieved by electrostatic complexation, by aqueous compartment entrapment, or by adsorption to a porous siliceous or polymeric scaffold within the inner core. Release is triggered by a class of trigger chosen from neutral-pH responsive linker classes, redox-responsive linker classes, and matrix-metalloproteinase-responsive peptide linker classes.

Mitochondrial-targeting peptide classes form a further payload subclass and are loaded across the molecular weight range of 500 daltons to 4,000 daltons. The platform accommodates Szeto-Schiller-type tetrapeptide classes that accumulate at the inner mitochondrial membrane and stabilize cardiolipin-cytochrome c interactions, mitochondria-penetrating peptide classes incorporating triphenylphosphonium-like cationic moieties, and mitochondrial fission and fusion modulator peptide classes acting on Drp1, Mfn1, Mfn2, and Opal protein classes. These peptide classes act on indications including mitochondrial encephalomyopathies, Leigh syndrome, Friedreich ataxia, Parkinson disease, and ischemic neuronal injury. Conformation-stabilizing peptide classes for misfolded protein diseases form a further payload subclass and are loaded across the molecular weight range of 1,000 daltons to 8,000 daltons. These peptide classes include pharmacological chaperone peptide classes that bind native or near-native conformations of disease-associated proteins and stabilize them against unfolding, prefoldin-mimetic peptide classes, and Hsp70-binding peptide classes that recruit endogenous chaperone activity to specific clients. Indications include lysosomal storage disorders with CNS involvement, polyglutamine expansion diseases including Huntington disease and spinocerebellar ataxias, and prion diseases. Oral delivery via the disclosed nanostructure is advantageous because these peptide classes are otherwise rapidly cleared and proteolytically degraded. Loading is achieved by entrapment within an amphipathic inner core compartment when the peptide is cationic or by electrostatic complexation when the peptide is polyanionic. Release is triggered by a class of trigger chosen from glutathione-responsive disulfide reduction within the reducing cytoplasm of target neurons and by enzyme-cleavable linker classes selected for cerebrovascular endothelial protease activity.

Neuropeptide receptor modulator classes including melanocortin, ghrelin, and orexin pathway modulator classes form an additional payload subclass loaded across the molecular weight range of 500 daltons to 5,000 daltons. The platform accommodates agonists and antagonists of the melanocortin receptor classes MC1R through MC5R for indications including cachexia, anorexia, obesity, neuroinflammation, and neuroprotection. Ghrelin receptor agonist and antagonist peptide classes are loaded for indications including cachexia, age-related muscle wasting, appetite disorders, and cognition disorders. Orexin receptor type 1 and type 2 agonist and antagonist peptide classes are loaded for indications including narcolepsy, hypersomnia, insomnia, and addiction. Additional accommodated neuropeptide receptor modulator classes include corticotropin-releasing factor receptor type 1 and type 2 antagonist peptide classes for stress and anxiety indications, vasoactive intestinal peptide and pituitary adenylate cyclase-activating peptide receptor modulator classes for neuroprotection and circadian indications, somatostatin receptor subtype-selective peptide classes for cognition and seizure indications, neuropeptide Y receptor Y1, Y2, Y4, and Y5 modulator classes for mood and feeding indications, and galanin receptor type 1, type 2, and type 3 modulator classes for mood and seizure indications. Loading is achieved by aqueous compartment entrapment, by electrostatic complexation with a polyionic core scaffold, or by reversible covalent tethering. Release is triggered by a class of trigger chosen from pH-responsive linker classes, redox-responsive disulfide linker classes, and enzyme-cleavable linker classes selected for activity at the cerebrovascular endothelium or within target neurons of the central nervous system. Cyclization, N-methylation, and unnatural amino acid incorporation are accommodated within the peptide payload class to enhance proteolytic stability while preserving receptor selectivity.

### Protein Therapeutic Payloads

Protein therapeutic payloads are loaded into the inner nanostructure core across the molecular weight range of 10,000 daltons to 200,000 daltons. Monoclonal antibody payloads of the immunoglobulin G subclass and engineered immunoglobulin variants are accommodated and are typically loaded in the molecular weight range of 145,000 daltons to 160,000 daltons. The platform is configured to load monoclonal antibody classes directed against amyloid-beta species including soluble oligomer-selective, protofibril-selective, and plaque-selective antibody classes; antibody classes directed against species tau including N-terminal, mid-domain, and microtubule-binding-region selective antibody classes; antibody classes directed against alpha-synuclein species including aggregate-selective antibody classes; antibody classes directed against TDP-43 species; antibody classes directed against huntingtin species; antibody classes directed against neuroinflammation targets including CD20 B-cell-directed, CD52, and complement component 5 antibody classes; and antibody classes directed against transferrin receptor, insulin receptor, and CD98 hc receptor classes used as receptor-mediated transcytosis shuttles. Antibody fragment payloads form a further subclass and are loaded across the molecular weight range of 12,000 daltons to 55,000 daltons. The accommodated fragment classes include Fab fragments, F(ab′) 2 fragments, single-chain variable fragments, and single-domain antibody fragments derived from heavy-chain-only antibodies of camelid or cartilaginous fish origin. Bispecific T-cell engager protein classes are accommodated in the molecular weight range of 50,000 daltons to 100,000 daltons for CNS oncology indications including high-grade gliomas. Oral delivery via the disclosed nanostructure is advantageous because intact antibodies and antibody fragments are otherwise denatured by gastric acid, proteolyzed by pepsin and intestinal proteases, and excluded from systemic circulation by the intact intestinal epithelium. Loading is achieved by aqueous compartment entrapment within a hydrated inner core surrounded by an amphipathic boundary, by adsorption to a porous scaffold, or by reversible covalent tethering through disulfide linker classes engineered into engineered cysteines on the antibody framework. Release is triggered by a class of trigger chosen from glutathione-responsive disulfide reduction at the abluminal surface of the cerebrovascular endothelium, by pH-responsive hydrazone cleavage in mildly acidic endosomal compartments, and by protease-cleavable linker classes selective for cerebrovascular endothelial proteases.

Engineered scaffold protein payloads form a further class loaded across the molecular weight range of 6,000 daltons to 30,000 daltons. The platform accommodates DARPin protein classes consisting of designed ankyrin repeat protein scaffolds engineered to bind defined surfaces on neurological targets, monobody protein classes derived from the type III domain of human fibronectin, affibody protein classes derived from the Z domain of staphylococcal protein A, anticalin protein classes derived from human lipocalin scaffolds, and knottin protein classes derived from cystine-knot miniprotein scaffolds. These engineered scaffold protein classes provide an alternative to immunoglobulin frameworks and exhibit favorable properties including high thermal stability, tolerance to disulfide-free expression in certain cases, monomeric behavior, and small hydrodynamic radius that facilitates tissue penetration. They are deployed for indications across neurodegenerative diseases, CNS oncology, neuroinflammation, and channelopathies, in roles including agonism, antagonism, allosteric modulation, target sequestration, and conditional intracellular target engagement. Oral delivery via the disclosed nanostructure is advantageous because engineered scaffold proteins remain susceptible to gastric and intestinal proteolysis despite their enhanced stability, and their typically polar surfaces preclude passive permeation of the intestinal epithelium. Loading within the inner core is achieved by aqueous compartment entrapment, by electrostatic complexation, by adsorption to a porous scaffold, or by reversible covalent tethering through site-specific bioorthogonal chemistry classes including engineered cysteine maleimide conjugation, sortase-mediated ligation, and click-chemistry-compatible azide-alkyne pairing. Release is triggered by a class of trigger chosen from disulfide reduction in the reducing intracellular environment, hydrazone cleavage in endosomal compartments, and enzyme-cleavable peptide linker classes selected for proteases enriched at the cerebrovascular endothelium and within the central nervous system.

Engineered cytokine variant payloads of the interleukin-2 and interleukin-15 family form a further subclass loaded across the molecular weight range of 15,000 daltons to 80,000 daltons including fusion variants. The platform accommodates engineered interleukin-2 variants with biased binding for the high-affinity heterotrimeric interleukin-2 receptor class consisting of alpha, beta, and gamma chains, and variants with biased binding for the intermediate-affinity beta-gamma receptor class, providing differential expansion of regulatory T cells versus effector lymphocytes for neuroinflammation and CNS autoimmune indications including multiple sclerosis. Engineered interleukin-15 variants and superagonist fusion classes incorporating the soluble interleukin-15 receptor alpha sushi domain are accommodated for indications including CNS oncology and chronic viral encephalitis. Enzyme replacement protein payloads for lysosomal storage disorders with CNS involvement form a further subclass loaded across the molecular weight range of 50,000 daltons to 150,000 daltons. The accommodated enzyme classes include glycosidase enzyme classes for mucopolysaccharidoses with CNS involvement, including type I, type II, type IIIA, type IIIB, type IIIC, type IIID, and type VII; sphingolipid hydrolase enzyme classes for Gaucher disease type 2 and type 3, Niemann-Pick disease type A and type C, Krabbe disease, metachromatic leukodystrophy, and Fabry disease with CNS involvement; ceroid lipofuscinosis enzyme classes for neuronal ceroid lipofuscinoses including CLN1, CLN2, and CLN10 forms; and glycogen-debranching and acid alpha-glucosidase enzyme classes. Growth factor protein payloads with CNS activity form a further subclass loaded across the molecular weight range of 12,000 daltons to 30,000 daltons. The accommodated growth factor protein classes include nerve growth factor, brain-derived neurotrophic factor, glial cell line-derived neurotrophic factor, neurturin, artemin, persephin, ciliary neurotrophic factor, insulin-like growth factor 1, vascular endothelial growth factor, and erythropoietin variants engineered for neuroprotective activity without erythropoietic activity. Loading is achieved by aqueous compartment entrapment, by adsorption to a porous scaffold, or by reversible covalent tethering through site-specific bioorthogonal chemistry. Release is triggered by glutathione-responsive disulfide reduction, by hydrazone cleavage in endosomal compartments, and by enzyme-cleavable linker classes selective for cerebrovascular endothelial and CNS-resident protease classes.

### Nucleic Acid Therapeutic Payloads

Nucleic acid therapeutic payloads are loaded into the inner nanostructure core across the molecular weight range of 5,000 daltons to 2,000,000 daltons depending on length and chemistry. Messenger RNA construct payloads encoding therapeutic proteins form a principal subclass and are loaded in the molecular weight range of 300,000 daltons to 1,500,000 daltons for transcripts spanning 1,000 to 5,000 nucleotides. The platform is configured to load messenger RNA constructs encoding neurotrophic factor protein classes, lysosomal enzyme replacement protein classes for CNS storage disorders, ion channel subunit protein classes for channelopathies, gap junction protein classes for leukodystrophies, dystrophin and utrophin protein classes for CNS-involving muscular dystrophies, and intracellular antibody classes including intrabody variants of the single-chain variable fragment scaffold. Self-amplifying RNA construct payloads incorporating alphavirus-derived nonstructural protein replicase machinery are accommodated in the molecular weight range of 2,500,000 daltons to 4,500,000 daltons for transcripts spanning 9,000 to 12,000 nucleotides, providing extended duration of protein expression from a single dose. Circular RNA construct payloads incorporating internal ribosome entry site-mediated translation initiation are accommodated in the molecular weight range of 150,000 daltons to 1,000,000 daltons, providing enhanced stability against exonuclease degradation and prolonged intracellular protein expression. Oral delivery via the disclosed nanostructure is advantageous because uncomplexed messenger RNA and circular RNA payloads are otherwise rapidly degraded by luminal and serum ribonucleases, are immunogenic when delivered without protective encapsulation, and exhibit negligible intestinal permeability owing to their large molecular weight and polyanionic backbone. Loading is achieved by electrostatic complexation with an inner core matrix incorporating ionizable cationic lipid classes, polycationic polymer classes, or cationic dendrimer classes, with chemistries designed to remain protonated within the acidic endosomal compartment and to become neutral within the cytoplasm to promote payload release. Release is triggered by a class of trigger chosen from pH-responsive ionization classes, by reducing-environment-responsive disulfide cleavage classes, and by endosomal-membrane-disruptive peptide classes that promote endosomal escape into the cytoplasm where the messenger RNA payload is translated.

Small interfering RNA, microRNA mimic, and microRNA inhibitor payload classes are loaded across the molecular weight range of 13,000 daltons to 16,000 daltons for duplex species spanning approximately 21 to 23 base pairs in the small interfering RNA case, and across comparable molecular weight ranges for microRNA mimic and inhibitor classes. The platform is configured to load small interfering RNA duplex payload classes directed against transcripts encoding huntingtin, ataxin proteins for spinocerebellar ataxias, superoxide dismutase 1 for amyotrophic lateral sclerosis, alpha-synuclein for synucleinopathies, microtubule-associated protein tau for tauopathies, amyloid precursor protein, beta-site amyloid precursor protein cleaving enzyme 1, leucine-rich repeat kinase 2, and TDP-43. MicroRNA mimic and inhibitor payload classes are loaded for indications including stroke, neurodegeneration, neuroinflammation, and CNS oncology, with chemistries chosen to preserve seed-sequence complementarity while enhancing stability and reducing off-target effects. Antisense oligonucleotide payload classes are loaded across the molecular weight range of 5,000 daltons to 10,000 daltons for species spanning 18 to 25 nucleotides. The platform accommodates antisense oligonucleotide payload classes employing phosphorothioate backbone chemistry, 2′-O-methyl ribose chemistry, 2′-O-methoxyethyl ribose chemistry, locked nucleic acid bicyclic ribose chemistry, and morpholino phosphorodiamidate backbone chemistry, alone or in mixed-chemistry gapmer architectures designed to recruit RNase H. Splice-switching oligonucleotide payload classes employing uniformly modified backbones that do not recruit RNase H are accommodated for indications including spinal muscular atrophy, Duchenne muscular dystrophy with CNS involvement, and CNS channelopathies amenable to exon-skipping or exon-inclusion strategies. Oral delivery via the disclosed nanostructure is advantageous because oligonucleotide payloads remain polyanionic and exhibit poor intestinal permeability and substantial luminal nuclease susceptibility despite chemical modification. Loading is achieved by electrostatic complexation with a cationic inner core matrix, by adsorption to a porous siliceous or polymeric scaffold, or by reversible covalent tethering. Release is triggered by a class of trigger chosen from pH-responsive ionization classes, glutathione-responsive disulfide cleavage classes, and enzyme-cleavable linker classes selective for cerebrovascular endothelial activity.

Ribozyme and aptamer payload classes form additional nucleic acid subclasses accommodated by the platform. Ribozyme payload classes including hammerhead, hairpin, and engineered self-cleaving ribozyme architecture classes are loaded across the molecular weight range of 15,000 daltons to 50,000 daltons and are directed against transcripts implicated in neurodegenerative, neuro-oncologic, and channelopathy indications. Aptamer payload classes consisting of single-stranded RNA or single-stranded DNA species selected by systematic evolution of ligands by exponential enrichment are loaded across the molecular weight range of 8,000 daltons to 25,000 daltons and span 25 to 80 nucleotides; these aptamer payload classes engage neurological target classes including ion channel extracellular domains, neurotransmitter receptor binding pockets, neurotrophic factor receptor classes, transferrin receptor and other cerebrovascular endothelial receptor classes, and aggregation-prone protein conformations. Chemical modification classes employing 2′-fluoro, 2′-O-methyl, 2′-O-methoxyethyl, and locked nucleic acid sugar chemistries, and 3′-inverted-deoxythymidine end-capping classes, are accommodated to enhance stability of aptamer payloads in the gastrointestinal lumen and the systemic circulation. Splice-switching oligonucleotide payload classes are accommodated as a distinct subclass complementary to the antisense oligonucleotide class described in the preceding paragraph and are loaded across the molecular weight range of 5,000 daltons to 10,000 daltons. Oral delivery via the disclosed nanostructure is advantageous because uncomplexed ribozyme and aptamer payloads remain susceptible to nuclease degradation and exhibit poor intestinal absorption. Loading is achieved by electrostatic complexation with a cationic inner core matrix, by aqueous compartment entrapment, by adsorption to a porous scaffold, or by reversible covalent tethering using bioorthogonal chemistry classes including copper-free click chemistry. Release is triggered by a class of trigger chosen from pH-responsive linker classes, glutathione-responsive disulfide cleavage classes, and enzyme-cleavable linker classes selective for activity at the cerebrovascular endothelium and within the central nervous system, with the trigger chosen to deliver the nucleic acid payload to the appropriate intracellular or extracellular compartment for its mechanism of action.

### Gene-Editing Payloads

Gene-editing payloads are loaded into the inner nanostructure core in formats including ribonucleoprotein complexes, plasmid DNA, and messenger RNA encoding the editor protein paired with separately delivered guide RNA species. CRISPR-Cas9 ribonucleoprotein complex payload classes are loaded across the molecular weight range of 160,000 daltons to 200,000 daltons for the protein component and 30,000 daltons to 35,000 daltons for the single guide RNA component, with the platform accommodating Streptococcus pyogenes Cas9 and orthologous Cas9 protein classes derived from Staphylococcus aureus, Neisseria meningitidis, and Campylobacter jejuni for editing of CNS-disease-relevant genomic loci including loci implicated in Huntington disease, amyotrophic lateral sclerosis, hereditary cerebellar ataxias, leukodystrophies, channelopathies, and lysosomal storage disorders with CNS involvement. CRISPR-Cas12 ribonucleoprotein complex payload classes including Cas12a, Cas12b, Cas12f, and miniaturized Cas12 variant protein classes are loaded across the molecular weight range of 50,000 daltons to 160,000 daltons for the protein component, providing alternative protospacer adjacent motif requirements, staggered double-strand break geometry, and reduced packaging requirements relative to Cas9. CRISPR-Cas13 ribonucleoprotein complex payload classes including Cas13a, Cas13b, and Cas13d protein classes are loaded across the molecular weight range of 80,000 daltons to 200,000 daltons and provide RNA-targeting activity for indications including repeat-expansion diseases at the transcript level, including myotonic dystrophy type 1 and Huntington disease. Oral delivery via the disclosed nanostructure is advantageous because ribonucleoprotein complexes are otherwise rapidly degraded in the gastrointestinal lumen, are too large for passive intestinal absorption, and are immunogenic when exposed to mucosal immune compartments without protective encapsulation. Loading is achieved by aqueous compartment entrapment within a hydrated inner core surrounded by an amphipathic boundary, by electrostatic complexation of the guide RNA component with a cationic matrix and co-encapsulation of the protein component, or by adsorption to a porous scaffold with surface chemistries selected to preserve enzymatic activity. Release is triggered by a class of trigger chosen from glutathione-responsive disulfide cleavage classes, pH-responsive linker classes, and enzyme-cleavable linker classes, with the trigger chosen so that the ribonucleoprotein complex is delivered intact to the cytoplasm or nucleus of the target neuron, glial cell, or cerebrovascular endothelial cell.

Base editor payload classes are accommodated in formats including ribonucleoprotein complex, messenger RNA-encoded, and plasmid-encoded modalities. Cytosine base editor protein classes consisting of a catalytically impaired Cas9 nickase fused to a cytidine deaminase protein class and a uracil glycosylase inhibitor protein class, and adenine base editor protein classes consisting of a catalytically impaired Cas9 nickase fused to an engineered adenosine deaminase protein class, are loaded across the molecular weight range of 180,000 daltons to 220,000 daltons for the protein component. These base editor payload classes effect single-nucleotide conversions without requiring double-strand breaks and are deployed for indications including point-mutation-driven neurodegenerative diseases, channelopathies, and metabolic disorders with CNS involvement. Prime editor payload classes consisting of a Cas9 nickase fused to an engineered reverse transcriptase protein class paired with a prime editing guide RNA species are loaded across the molecular weight range of 230,000 daltons to 270,000 daltons for the protein component and across 35,000 daltons to 60,000 daltons for the prime editing guide RNA component; these payload classes effect arbitrary point mutations, small insertions, and small deletions without double-strand breaks. Transposon-based gene-insertion system payload classes incorporating piggyBac, Sleeping Beauty, and Tol2 transposase protein classes paired with cargo-containing transposon donor DNA classes are accommodated for indications requiring integration of multi-kilobase therapeutic transgene cassettes. Epigenome editor payload class architectures consisting of catalytically dead Cas9 protein fused to effector protein domain classes including DNA methyltransferase 3A, ten-eleven translocation methylcytosine dioxygenase 1, p300 histone acetyltransferase, Krüppel-associated box repressor domain, and DNA methyltransferase 3L recruitment domain are accommodated for indications requiring durable transcriptional silencing or activation without alteration of the underlying DNA sequence. Oral delivery via the disclosed nanostructure is advantageous because each of these gene-editing payload classes is large, polyanionic in nucleic acid components, proteolytically labile in protein components, and immunogenic when delivered without protective encapsulation. Loading and release follow the principles set forth for the CRISPR-Cas9 ribonucleoprotein complex payload class, with stoichiometric ratios of protein and nucleic acid components selected to preserve activity upon reassembly in the target intracellular compartment.

### Emerging Modality Payloads

Emerging modality payloads include induced-proximity small molecule classes and photoswitchable and optogenetic payload classes. Molecular glue degrader small molecule payload classes are loaded across the molecular weight range of 300 daltons to 700 daltons and are accommodated for indications requiring degradation of disease-associated CNS proteins lacking conventional small-molecule binding pockets, via recruitment of cullin-RING E3 ubiquitin ligase complex classes including cereblon-containing complex classes and DCAF-containing complex classes to neosubstrate protein surfaces. Proteolysis-targeting chimera payload classes, denoted PROTAC payload classes, are loaded across the molecular weight range of 700 daltons to 1,500 daltons and consist of a target-binding ligand class joined by a linker class to an E3 ligase-recruiting ligand class. The platform accommodates PROTAC payload classes directed against CNS targets including microtubule-associated protein tau, alpha-synuclein, huntingtin, leucine-rich repeat kinase 2, bromodomain and extra-terminal domain proteins, and androgen receptor for CNS oncology indications. Autophagy-targeting chimera payload classes, denoted AUTAC payload classes, are loaded across the molecular weight range of 800 daltons to 1,500 daltons and consist of a target-binding ligand class joined by a linker class to a guanine-derivative autophagy degradation signal class. Lysosome-targeting chimera payload classes, denoted LYTAC payload classes, are loaded across the molecular weight range of 100,000 daltons to 200,000 daltons in protein-conjugate format and consist of a target-binding antibody, antibody fragment, or aptamer class joined by a linker class to a lysosome-shuttle receptor ligand class including ligands for the cation-independent mannose-6-phosphate receptor class and the asialoglycoprotein receptor class adapted for CNS uptake routes. Ribonuclease-targeting chimera payload classes, denoted RIBOTAC payload classes, are loaded across the molecular weight range of 800 daltons to 1,800 daltons and consist of a target RNA structure-binding ligand class joined by a linker class to a small molecule ribonuclease L recruiting ligand class. These induced-proximity payload classes act catalytically, providing substoichiometric activity that is advantageous for CNS targets where penetration of the cerebrovascular endothelium limits effective intracellular concentration. Oral delivery via the disclosed nanostructure is advantageous because these payloads are typically of relatively high molecular weight for the small-molecule class, exhibit poor aqueous solubility, are substrates for intestinal and hepatic efflux transporters, and benefit from lymphatic uptake and bypass of first-pass metabolism. Loading is achieved by hydrophobic encapsulation within a lipidic core compartment, by adsorption to a porous scaffold, or by reversible covalent tethering. Release is triggered by a class of trigger chosen from pH-responsive linker classes, redox-responsive linker classes, and enzyme-cleavable linker classes selective for intracellular activation.

Photoswitchable pharmacology compound payload classes are loaded across the molecular weight range of 300 daltons to 900 daltons and incorporate photoswitchable moiety classes including azobenzene, diarylethene, spiropyran, and stilbene moiety classes that undergo reversible isomerization upon exposure to defined wavelength classes. These payload classes are accommodated as photoswitchable variants of neurotransmitter receptor agonist and antagonist small molecule classes, ion channel modulator small molecule classes, kinase inhibitor small molecule classes, and epigenetic modulator small molecule classes, providing spatiotemporal control of pharmacological activity at the level of the central nervous system through delivery of activating wavelength classes via implanted or transcranial light delivery devices. Optogenetic actuator protein payload classes are loaded across the molecular weight range of 25,000 daltons to 70,000 daltons in protein format and at messenger RNA-encoded molecular weight ranges of 250,000 daltons to 1,000,000 daltons in encoded format. The accommodated optogenetic actuator protein classes include channelrhodopsin protein classes, halorhodopsin protein classes, archaerhodopsin protein classes, step-function opsin protein classes, anion-conducting channelrhodopsin protein classes, and red-shifted variant protein classes derived from these scaffolds, together with optogenetic biochemical-control protein classes including light-activated adenylyl cyclase protein classes, light-activated guanylyl cyclase protein classes, and cryptochrome-dimerization protein classes used to control intracellular signaling. These payload classes are deployed for indications including pharmacoresistant epilepsy, Parkinson disease, depression, and chronic pain in conjunction with light delivery infrastructure. Oral delivery via the disclosed nanostructure is advantageous because photoswitchable small-molecule payloads benefit from improved bioavailability and reduced metabolic loss, and because optogenetic actuator protein payloads delivered as messenger RNA or self-amplifying RNA constructs benefit from the protective encapsulation that the nanostructure provides against luminal and serum ribonucleases. Loading and release follow the principles set forth for the corresponding small molecule and nucleic acid payload classes, with the additional design constraint that photoswitchable moieties are protected from premature photoactivation during gastrointestinal transit by the optical opacity of the nanostructure outer shell.

### Combination Payload Architectures

Combination payload architectures consist of two or more payload classes co-formulated within a single nanostructure to produce additive, synergistic, or sequential pharmacological effects. Dual-payload co-loading architectures are accommodated by the disclosed nanostructure and permit co-encapsulation of two payloads of compatible loading mode within the same inner core compartment or within distinct compartments of a multicompartmental inner core. Representative dual-payload co-loading combinations include a small molecule BACE1 inhibitor payload class co-loaded with a monoclonal antibody payload class directed against amyloid-beta protofibrils, a small molecule LRRK2 kinase inhibitor payload class co-loaded with a small interfering RNA payload class directed against alpha-synuclein transcript, a small molecule histone deacetylase inhibitor payload class co-loaded with a CRISPR-Cas9 ribonucleoprotein complex payload class for synergistic chromatin remodeling and targeted gene editing, and a small molecule Nrf2 activator payload class co-loaded with a mitochondrial-targeting peptide payload class for combined antioxidant and bioenergetic support. The molecular weight range of each component is preserved at the values established in the foregoing sections, and total payload mass per nanostructure unit is selected to remain within the encapsulation capacity of the inner core. Oral delivery via the disclosed nanostructure is advantageous for combination payloads because conventional oral co-administration of two payload classes is subject to mismatched absorption kinetics, divergent first-pass metabolism, and disparate plasma half-lives, all of which compromise the temporal coincidence required for synergistic pharmacology, whereas co-encapsulation within a single nanostructure unit ensures coincident absorption, lymphatic transport, and delivery to the cerebrovascular endothelium. Loading is achieved by selecting compatible loading modes for the constituent payload classes, including dual hydrophobic encapsulation, hydrophobic-plus-aqueous compartmentalization, dual electrostatic complexation with charge segregation, or hybrid loading mode pairing. Release is triggered by a class of trigger selected from the foregoing trigger classes, with the trigger applied uniformly across the co-loaded payload classes or, in multicompartmental architectures, applied independently to each compartment for differential temporal release.

Sequential-release combination payload architectures are accommodated by the disclosed nanostructure and incorporate two or more payload classes positioned within distinct compartments of a multicompartmental inner core, with each compartment surrounded by a release barrier class responsive to a distinct stimulus class, such that the payload classes are released in a defined temporal sequence after delivery to the cerebrovascular endothelium or after intracellular uptake. Representative sequential-release combinations include a first-released aggregation-disrupting peptide payload class followed by a second-released enzyme replacement protein payload class for tauopathy and lysosomal storage disorder combination indications, a first-released matrix-metalloproteinase-modulating small molecule payload class followed by a second-released antisense oligonucleotide payload class for CNS oncology indications, and a first-released anti-inflammatory small molecule payload class followed by a second-released neurotrophic factor protein payload class for stroke and traumatic brain injury indications. Ratiometric loading for synergistic combinations is accommodated by the platform and consists of co-loading two or more payload classes at a defined molar ratio selected to optimize the synergy index according to a pharmacological model of the combination, with the molar ratio preserved by virtue of unitary nanostructure encapsulation regardless of pharmacokinetic variation in oral absorption and systemic distribution. Tandem-payload conjugate architectures are accommodated by the platform and consist of two payload classes covalently joined through a cleavable linker class to form a single conjugate molecule which is encapsulated within the inner nanostructure core and which releases its constituent payload classes upon cleavage of the connecting linker class. Representative tandem-payload conjugates include peptide-small molecule conjugates in which a CNS-targeting peptide ligand class is conjugated to a small molecule payload class via an esterase-cleavable or disulfide linker class, oligonucleotide-peptide conjugates in which an antisense oligonucleotide payload class is conjugated to a cell-penetrating peptide class via a disulfide linker class, and antibody-small molecule conjugates in which a monoclonal antibody payload class is conjugated to a small molecule payload class via a protease-cleavable peptide linker class. In each combination payload architecture the choice of loading mode, compartmentalization scheme, linker chemistry, and release trigger class is selected to deliver each constituent payload class to its site of pharmacological action with preservation of activity, while exploiting the oral nanostructure platform to overcome the gastrointestinal, hepatic, and cerebrovascular barriers that would otherwise preclude the combined administration of these payload classes by the conventional oral route.

### Expanded Axis 2—Outer Shell Architecture (Deep Dive)

### Outer Shell Architecture

The outer shell of the oral nanostructure platform constitutes the first line of structural defense and the primary determinant of gastrointestinal survival, mucosal traversal, and downstream blood-brain barrier engagement. Across the embodiments contemplated herein, the outer shell is selected from one or more of: bacteriophage-derived virus-like particles, plant-virus-derived virus-like particles, engineered protein nanocages, lipid bilayer architectures, biodegradable and biocompatible polymeric shells, inorganic and hybrid inorganic-organic shells, biomimetic cellular-membrane coatings, and enteric polymer coatings configured to remain intact at gastric pH and to dissolve, swell, or undergo controlled erosion at intestinal pH. The outer shell is dimensioned to enclose, partially enclose, or surround an inner therapeutic-loaded core of from 20 nanometers to 200 nanometers in hydrodynamic diameter, with overall assembled particle hydrodynamic diameter of from 50 nanometers to 500 nanometers. In particular embodiments, the outer shell is engineered to display targeting ligands, mucoadhesive moieties, mucus-penetrating polymer brushes, transcytosis-promoting peptides, and protease-resistant capping chemistries. The shell architectures described in this section are not mutually exclusive; the platform contemplates multilayered, Janus, and hybrid architectures in which two or more distinct shell classes are combined either as concentric layers, as patchy domains on a single particle, or as a composite matrix in which one shell class is dispersed within a continuous phase of a second shell class. The shell architectures further provide attachment points for the dual-pH-triggered release mechanism, the enzyme-responsive cleavage sequences, and the magnetically guided actuation systems described elsewhere in this specification.

### Bacteriophage-Derived Virus-Like Particle Shells

In one class of embodiments, the outer shell comprises a bacteriophage-derived virus-like particle assembled from one or more recombinant coat-protein subunits expressed heterologously in a bacterial host and purified to homogeneity in the absence of any encapsidated phage genome. The P22 bacteriophage virus-like particle, derived from the Salmonella enterica P22 procapsid, exhibits T=7 icosahedral symmetry, assembles from 420 copies of the gp5 coat protein with the assistance of the gp8 scaffolding protein, and presents an external diameter of from 58 nanometers to 64 nanometers with an internal cavity volume of from 5.0 by 10 to the negative 17 milliliters per particle to 6.5 by 10 to the negative 17 milliliters per particle. The P22 particle undergoes a maturation transition upon scaffold removal that expands the capsid to an external diameter of from 62 nanometers to 70 nanometers and confers proteolytic stability up to chymotrypsin and trypsin concentrations of from 0.1 milligrams per milliliter to 1.0 milligrams per milliliter and pH stability across the range of from pH 3.5 to pH 9.0 for up to 24 hours at 37 degrees Celsius. The MS2 bacteriophage coat protein assembles into T-3 icosahedral particles of from 26 nanometers to 28 nanometers external diameter from 180 coat-protein copies arranged as 90 covalent or non-covalent dimers and tolerates surface lysine bioconjugation through N-hydroxysuccinimide ester chemistry as well as engineered cysteine maleimide coupling. The MS2 particle exhibits pH stability from pH 4.0 to pH 10.0, retains structural integrity in simulated gastric fluid for up to 60 minutes when surface-stabilized, and is amenable to RNA cargo loading through reassembly around an engineered TR-stem-loop pac sequence.

The Qβ bacteriophage virus-like particle, also of T-3 icosahedral symmetry, assembles from 180 copies of the Qβ coat protein into particles of from 28 nanometers to 30 nanometers external diameter and is stabilized relative to MS2 by 90 inter-subunit disulfide bonds formed between Cys74 and Cys80 of adjacent dimers. The Qβ shell tolerates pH excursions from pH 2.0 to pH 12.0 and temperatures of up to 70 degrees Celsius for periods of from 30 minutes to 120 minutes without measurable disassembly, properties that render Qβ particularly suitable for oral delivery in embodiments lacking an enteric outer layer. The PP7 bacteriophage particle assembles from 180 copies of the PP7 coat protein into T-3 icosahedral particles of from 28 nanometers to 30 nanometers external diameter and is orthogonal to MS2 in its RNA hairpin binding specificity, permitting dual-cargo loading strategies in which MS2 and PP7 are co-displayed without cross-encapsidation. The AP205 Acinetobacter-phage particle assembles from 180 coat-protein copies into T=3 icosahedral particles of from 28 nanometers to 31 nanometers external diameter and is distinguished by its tolerance of N- and C-terminal coat-protein fusions of up to 200 amino acids without loss of assembly competence, enabling genetically encoded display of full-length protein ligands. Each of P22, MS2, QB, PP7, and AP205 is produced by recombinant expression in Escherichia coli BL21 (DE3), C41 (DE3), C43 (DE3), or Rosetta (DE3) host strains under isopropyl-beta-D-thiogalactopyranoside induction, with downstream purification by sucrose- or cesium-chloride-gradient ultracentrifugation, size-exclusion chromatography on Superose 6 or Sepharose CL-4B media, or tangential-flow filtration on 300-kilodalton or 500-kilodalton molecular-weight-cutoff membranes.

In further embodiments the outer shell comprises a tailed-bacteriophage-derived virus-like particle of larger internal volume suitable for high-payload CNS therapeutics. The T4 bacteriophage head, assembled from 930 copies of gp23 coat protein with 55 copies of gp24 vertex protein, presents a prolate icosahedral architecture with major axis of from 115 nanometers to 120 nanometers, minor axis of from 85 nanometers to 86 nanometers, and an internal cavity volume of from 4.5 by 10 to the negative 16 milliliters per particle to 5.0 by 10 to the negative 16 milliliters per particle, permitting encapsidation of cargo masses of up to 170 megadaltons. The T4 shell is loaded by in vitro packaging through the gp17 large terminase motor, by Hoc and Soc surface display of therapeutic protein fusions, or by post-assembly osmotic-shock-driven uptake. The T7 bacteriophage head presents T=7 icosahedral symmetry, an external diameter of from 55 nanometers to 60 nanometers, and is engineered for surface display through gp10A and gp10B capsid fusions. The lambda bacteriophage head exhibits T=7 icosahedral symmetry, external diameter of from 60 nanometers to 65 nanometers, and is amenable to gpD surface decoration with therapeutic peptides and antibody fragments. The phi-29 bacteriophage prolate head presents major-axis dimensions of from 42 nanometers to 54 nanometers and incorporates a packaging motor with terminal protein gp3 that permits in vitro loading of nucleic acid cargo of from 6 kilobases to 19 kilobases. The phi-X174 procapsid presents T=1 icosahedral symmetry with external diameter of from 25 nanometers to 27 nanometers and is amenable to internal-scaffold-protein fusion for cargo recruitment.

Cargo loading of bacteriophage-derived virus-like particles is accomplished through one or more of the following routes. In a scaffold-protein-fusion route, the therapeutic cargo is genetically appended to the N- or C-terminus of the cognate scaffolding protein, for example the P22 gp8 scaffold or the phi-X174 internal scaffold, such that the cargo is co-assembled into the interior of the particle during in vivo capsid maturation in the bacterial expression host. In an electrostatic co-assembly route, the coat-protein subunits are purified in disassembled form at low ionic strength and high pH and are reassembled around polyanionic or polycationic cargo at neutral pH and elevated ionic strength, the cargo serving as a templating polyelectrolyte for capsid nucleation. In a disassembly-reassembly route, intact wild-type virus-like particles are dissociated under denaturing conditions of from 1.5 molar to 3.0 molar guanidinium chloride or from 3.0 molar to 6.0 molar urea, the coat-protein subunits are refolded in the presence of the cargo, and reassembly is initiated by buffer exchange against an assembly-promoting buffer of from pH 7.0 to pH 7.5 containing from 100 millimolar to 500 millimolar sodium chloride. In a covalent post-assembly conjugation route, cargo bearing reactive azide, alkyne, tetrazine, or maleimide functional groups is coupled to surface-exposed lysine, cysteine, or genetically encoded non-canonical amino acid residues on the assembled particle. Each route is selected based on cargo size, charge, structural sensitivity to denaturation, and required encapsidation stoichiometry, with scaffold fusion preferred for protein cargoes of up to 60 kilodaltons per subunit-fusion, electrostatic co-assembly preferred for nucleic acid cargoes of from 1 kilobase to 10 kilobases, and disassembly-reassembly preferred for small-molecule and peptide therapeutics requiring stoichiometries of from 100 to 600 copies per particle.

### Plant-Virus-Derived Virus-Like Particle Shells

In a further class of embodiments, the outer shell comprises a plant-virus-derived virus-like particle produced by transient expression in Nicotiana benthamiana or in cell-free wheat-germ or tobacco-cell-suspension systems and purified in the absence of any encapsidated viral nucleic acid. The cowpea chlorotic mottle virus virus-like particle assembles from 180 copies of a 20-kilodalton coat protein into T=3 icosahedral particles of from 26 nanometers to 28 nanometers external diameter and exhibits a well-characterized pH- and ionic-strength-dependent gating transition, undergoing a 10 percent radial swelling at pH greater than 6.5 in the absence of divalent cations that opens 60 hexameric pores of from 2 nanometers to 3 nanometers diameter at the icosahedral three-fold axes. This gating transition permits dynamic loading and release of cargoes of up to from 5 kilodaltons to 7 kilodaltons through size-selective diffusion, with the particle resealing upon return to pH less than 6.0 or upon addition of from 1 millimolar to 5 millimolar calcium or magnesium ions. The cowpea mosaic virus virus-like particle assembles from 60 copies each of a large and a small coat-protein subunit into T=1 pseudo-icosahedral particles of from 28 nanometers to 31 nanometers external diameter and is distinguished by its native tropism for mammalian vimentin, which provides an intrinsic cell-surface-binding modality without exogenous targeting ligand conjugation. Both cowpea chlorotic mottle virus and cowpea mosaic virus virus-like particles tolerate gastric-mimetic conditions of from pH 1.5 to pH 3.5 for periods of from 30 minutes to 120 minutes when stabilized by an enteric outer coating as described in subsequent sections.

The brome mosaic virus virus-like particle, like cowpea chlorotic mottle virus, assembles from 180 coat-protein copies into T-3 icosahedral particles of from 26 nanometers to 28 nanometers external diameter and exhibits an analogous reversible pH-triggered swelling above pH 6.5, with the brome mosaic virus particle distinguished by its greater tolerance of organic-solvent excursions of up to 30 percent ethanol or 20 percent dimethyl sulfoxide. The tobacco mosaic virus virus-like particle assembles from 2,130 copies of a 17.5-kilodalton coat protein into rigid helical rods of from 18 nanometers external diameter and from 50 nanometers to 300 nanometers length, the length being modulable through incorporation of an origin-of-assembly stem-loop into a templating RNA, with the rod presenting an internal 4-nanometer channel for cargo loading. The tobacco mosaic virus rod tolerates pH excursions from pH 2.0 to pH 9.5 and temperatures of up to 60 degrees Celsius, properties that render it especially robust to oral gastrointestinal challenge. The hibiscus chlorotic ringspot virus virus-like particle assembles into T=3 icosahedral particles of from 28 nanometers to 30 nanometers external diameter from 180 coat-protein copies and is distinguished by its native ability to encapsidate heterologous RNA cargo of from 1 kilobase to 4 kilobases through electrostatic co-assembly during plant-host expression. The potato virus X virus-like particle assembles into flexuous filamentous rods of from 13 nanometers external diameter and from 100 nanometers to 515 nanometers length from approximately 1,270 coat-protein copies and is amenable to N-terminal coat-protein fusion of therapeutic peptides and small protein domains.

The tomato bushy stunt virus virus-like particle assembles from 180 coat-protein copies into T=3 icosahedral particles of from 32 nanometers to 35 nanometers external diameter and exhibits an exceptionally stable assembly that tolerates pH 2.0 to pH 9.0 and temperatures of up to 65 degrees Celsius. The turnip yellow mosaic virus virus-like particle assembles from 180 coat-protein copies into T=3 icosahedral particles of from 28 nanometers to 30 nanometers external diameter and is distinguished by a reversible pH-triggered uncoating in which the particle swells and releases cargo at pH less than 4.5 while remaining stable at neutral pH, a property that makes it an inverse-gating embodiment suitable in particular for cargo release within acidic endosomal or lysosomal compartments rather than at intestinal pH. Production of plant-virus-derived virus-like particles is accomplished by agroinfiltration of Nicotiana benthamiana leaves with binary expression vectors driving coat-protein expression under the control of a 35S cauliflower-mosaic-virus promoter and a tobacco-mosaic-virus omega translational enhancer, with harvest at from 5 days to 10 days post-infiltration, mechanical homogenization in extraction buffer of from pH 7.0 to pH 7.5 containing from 100 millimolar to 250 millimolar sodium chloride, polyethylene-glycol precipitation, and final purification by sucrose-gradient ultracentrifugation. Yields of from 100 milligrams per kilogram to 1,000 milligrams per kilogram of leaf fresh weight are routinely achieved, and the platform avoids endotoxin contamination intrinsic to E. coli-based expression, which is particularly advantageous for oral CNS-therapeutic embodiments intended for chronic dosing.

Plant-virus-derived virus-like particles are particularly suited to oral gastrointestinal-stability applications by virtue of their evolutionary adaptation to plant-tissue extremes of pH, dehydration, and proteolytic challenge, and by virtue of their lack of mammalian pre-existing immunity. In the platform contemplated herein, cowpea chlorotic mottle virus and brome mosaic virus virus-like particles are loaded with anionic CNS therapeutic cargoes including small interfering RNA, micro-RNA mimics, antisense oligonucleotides, and circular RNAs through the high-pH disassembly route followed by neutral-pH calcium-triggered reassembly, with encapsidation efficiencies of from 60 percent to 95 percent of input cargo achieved at coat-protein-to-cargo molar ratios of from 100:1 to 400:1. Cowpea mosaic virus virus-like particles are loaded through chemical conjugation of cargo to surface-exposed lysine residues through N-hydroxysuccinimide-ester chemistry or to engineered cysteine residues through maleimide chemistry, with loading densities of from 30 to 60 cargo copies per particle. Tobacco mosaic virus rods are loaded through encapsidation of an origin-of-assembly-tagged RNA cargo during coat-protein-driven helical reassembly, with cargo capacities scaling linearly with rod length and reaching from 5,000 to 20,000 nucleotides per rod. In each case, the plant-virus-derived particle is then coated with an enteric polymer layer as described in subsequent sections to protect against gastric acid and pepsin during oral transit.

### Engineered Protein Nanocages

In a further class of embodiments, the outer shell comprises an engineered protein nanocage assembled from one or more recombinant subunit proteins of mammalian, bacterial, archaeal, or de novo computational origin. The ferritin nanocage is assembled from 24 subunits arranged with 432 octahedral symmetry into a hollow spherical shell of from 12 nanometers external diameter and 8 nanometers internal cavity diameter, with the cavity volume of approximately 2.7 by 10 to the negative 19 milliliters per particle accommodating cargo masses of up to from 50 kilodaltons to 75 kilodaltons. In one embodiment, the ferritin nanocage is assembled from human heavy-chain ferritin subunits, which bind transferrin receptor 1 and provide intrinsic blood-brain-barrier transcytosis and tumor targeting; in a further embodiment, the cage is assembled from human light-chain ferritin subunits, which lack the ferroxidase center and provide a charge-neutral inner cavity surface preferred for nucleic acid and protein cargo retention; in a still further embodiment, the cage is assembled from heteromeric heavy- and light-chain subunits at heavy-to-light molar ratios of from 1:23 to 23:1 to tune both iron-handling capacity and receptor-binding avidity. The apoferritin form, prepared by reductive demineralization of holoferritin in the presence of from 1 millimolar to 10 millimolar sodium dithionite at pH 4.5, presents an empty cavity for loading of small-molecule, peptide, and oligonucleotide cargoes through pH-mediated disassembly at pH less than 3.0 and reassembly at pH 7.0 to 7.5.

The mini-ferritin Dps nanocage, derived from DNA-binding protein from starved cells, assembles from 12 subunits with 23 tetrahedral symmetry into a hollow shell of from 9 nanometers external diameter and from 4.5 nanometers to 5 nanometers internal cavity diameter, providing a compact protein-cage platform for small-molecule and short-peptide cargo encapsidation. The bacterioferritin nanocage assembles from 24 subunits into a 12-nanometer cage analogous to mammalian ferritin and is distinguished by intrinsic heme cofactor binding that permits incorporation of photoresponsive cargo-release triggers. The encapsulin family of nanocompartments comprises bacterial and archaeal proteins that assemble at one of three icosahedral symmetries: a T=1 encapsulin assembled from 60 subunits with external diameter of from 24 nanometers to 25 nanometers, a T-3 encapsulin from 180 subunits with external diameter of from 30 nanometers to 32 nanometers, and a T-4 encapsulin from 240 subunits with external diameter of from 42 nanometers to 45 nanometers, the latter providing the largest internal cargo volume of from 4.0 by 10 to the negative 17 milliliters per particle to 5.0 by 10 to the negative 17 milliliters per particle among the encapsulin family. Encapsulins are loaded through fusion of a cargo to a cognate C-terminal targeting peptide that binds a hydrophobic pocket on the interior cage surface, with stoichiometric incorporation of up to from 12 cargo copies to 60 cargo copies per cage achieved during co-expression in E. coli BL21 (DE3) or Rosetta (DE3).

The lumazine synthase nanocage, derived from Aquifex aeolicus or Bacillus subtilis, assembles from 60 subunits into T=1 icosahedral particles of from 15 nanometers to 16 nanometers external diameter and provides a thermostable cage scaffold tolerating pH 4.0 to pH 9.0 and temperatures of up to 95 degrees Celsius. Heat-shock-protein cages such as the small heat-shock-protein Mj HSP16.5 from Methanocaldococcus jannaschii assemble from 24 subunits into 12-nanometer cages with intrinsic chaperone activity that stabilizes encapsulated protein cargo against thermal and chemical denaturation. The vault ribonucleoprotein cage, assembled from 78 copies of major vault protein into a barrel-shaped particle of from 40 nanometers minor axis to 70 nanometers major axis and a total mass of approximately 40 megadaltons, provides the largest internal cargo volume among the protein cages contemplated herein, of from 1.0 by 10 to the negative 16 milliliters per particle to 1.5 by 10 to the negative 16 milliliters per particle, sufficient to encapsidate full-length therapeutic proteins of up to from 200 kilodaltons to 500 kilodaltons. The peroxiredoxin family of antioxidant enzymes assembles into ring-shaped decamers and dodecamers and into higher-order toroidal stacks of from 12 nanometers to 30 nanometers diameter that present pH-switchable assembly states suitable for triggered cargo release. Computationally designed icosahedral protein cages, including the I3-01 60-subunit T=1 cage of from 24 nanometers to 25 nanometers external diameter and related de novo cage families designed through symmetric protein interface design and Rosetta-based scoring, provide tailored cage geometries with engineered cargo-binding pockets, designable surface charge distributions, and tunable subunit-subunit interface affinities for controlled disassembly.

Engineered protein nanocages are produced by recombinant expression in E. coli BL21 (DE3), C41 (DE3), C43 (DE3), or Rosetta (DE3) host strains under isopropyl-beta-D-thiogalactopyranoside induction at from 16 degrees Celsius to 25 degrees Celsius for periods of from 16 hours to 20 hours, with downstream purification by ammonium-sulfate precipitation, ion-exchange chromatography on Q- or SP-Sepharose media, hydroxyapatite chromatography, and final polishing by size-exclusion chromatography on Superose 6 or Superdex 200 media. Cargo loading is accomplished through one or more of: genetic fusion to an interior-facing cage subunit terminus, fusion to a cognate cargo-targeting peptide, pH-mediated disassembly and reassembly through excursions of from pH 2.5 to pH 4.0, denaturant-mediated disassembly and refolding through excursions of from 1.5 molar to 6.0 molar guanidinium chloride or urea, and post-assembly chemical conjugation through surface-exposed lysine, cysteine, or genetically encoded non-canonical amino acid residues. The protein-cage class is particularly suited to oral CNS-therapeutic embodiments by virtue of its homogeneous size distribution, its low immunogenicity when assembled from human-derived subunits such as heavy- and light-chain ferritin, its compatibility with co-expression of multivalent surface-displayed targeting ligands, and its established compatibility with enteric polymer overcoating.

### Lipid Bilayer Shells

In a further class of embodiments, the outer shell comprises a lipid bilayer architecture assembled from one or more phospholipid, sterol, ionizable lipid, or polymer-conjugated lipid species. Conventional liposomes are prepared as small unilamellar vesicles of from 30 nanometers to 100 nanometers hydrodynamic diameter, as large unilamellar vesicles of from 100 nanometers to 1,000 nanometers hydrodynamic diameter, or as multilamellar vesicles of from 500 nanometers to 5,000 nanometers hydrodynamic diameter, from phospholipid compositions selected from phosphatidylcholine, phosphatidylethanolamine, phosphatidylserine, phosphatidylglycerol, phosphatidylinositol, and sphingomyelin species, with cholesterol incorporated at from 20 mole percent to 45 mole percent to modulate membrane fluidity and proteolytic stability. Stealth liposomes incorporate polyethylene-glycol-conjugated phospholipid, for example DSPE-PEG-2000 or DSPE-PEG-5000, at from 2 mole percent to 10 mole percent of total lipid to suppress opsonization, prolong systemic circulation following gut-to-blood transcytosis, and provide a substrate for end-group ligand conjugation through azide, maleimide, or N-hydroxysuccinimide-ester chemistries.

Ionizable-lipid lipid nanoparticles of the MC3, SM-102, and ALC-0315 classes provide the preferred lipid-based shell for nucleic acid CNS cargoes including small interfering RNA, micro-RNA, messenger RNA, and self-amplifying RNA. The lipid nanoparticle comprises an ionizable amino lipid at from 30 mole percent to 50 mole percent, a phospholipid such as distearoylphosphatidylcholine or dioleoylphosphatidylethanolamine at from 5 mole percent to 15 mole percent, cholesterol it from 30 mole percent to 50 mole percent, and a polyethylene-glycol-conjugated lipid such as PEG-DMG or PEG-DSPE at from 0.5 mole percent to 3.0 mole percent. The ionizable lipid exhibits an apparent pKa of from 6.2 to 6.8, conferring near-neutral surface charge at physiological pH 7.4 and protonation upon endosomal acidification to pH 5.0 to pH 6.0, thereby driving endosomal-membrane disruption and cytosolic cargo delivery. Particles are prepared by rapid ethanolic-aqueous mixing in a T-junction, herringbone-microfluidic, or impingement-jet mixer at total flow rates of from 4 milliliters per minute to 12 milliliters per minute and aqueous-to-ethanol flow ratios of from 3:1 to 5:1, yielding monodisperse particles of from 60 nanometers to 120 nanometers hydrodynamic diameter with polydispersity index of less than 0.15 and nucleic acid encapsulation efficiency of greater than 85 percent.

Solid lipid nanoparticles, prepared from solid-at-body-temperature triglycerides such as tristearin, tripalmitin, glyceryl behenate, or cetyl palmitate, provide a stable crystalline-core lipid shell of from 50 nanometers to 300 nanometers hydrodynamic diameter for hydrophobic small-molecule and lipophilic peptide CNS cargoes. Nanostructured lipid carriers extend the solid-lipid-nanoparticle architecture by incorporating from 10 weight percent to 30 weight percent of a liquid lipid such as medium-chain triglyceride, oleic acid, or squalene within the solid-lipid matrix, thereby disrupting the crystalline lattice, increasing cargo-loading capacity by from two-fold to five-fold, and reducing on-storage cargo expulsion. Niosomes, assembled from non-ionic surfactants of the Span and Tween families together with cholesterol, provide a non-phospholipid bilayer vesicle of from 100 nanometers to 500 nanometers hydrodynamic diameter with greater chemical stability than phospholipid liposomes under gastric-mimetic conditions. Bicelles, assembled from a long-chain phospholipid such as dimyristoylphosphatidylcholine and a short-chain phospholipid such as dihexanoylphosphatidylcholine at molar ratios of from 0.25 to 4.0, provide disk-shaped lipid assemblies of from 10 nanometers to 80 nanometers diameter suitable for membrane-protein cargo presentation. Cubosomes, assembled from monoolein, phytantriol, or related amphiphiles into bicontinuous cubic lipid mesophases stabilized by Pluronic F127, provide nanostructured non-lamellar lipid particles of from 100 nanometers to 500 nanometers diameter with high internal-surface-area cargo-loading capacity. Transfersomes, comprising phosphatidylcholine and an edge-activator surfactant such as sodium cholate or Tween 80 at from 10 weight percent to 25 weight percent of total amphiphile, provide ultra-deformable vesicles capable of intercellular paracellular traversal.

### Polymeric Shells

In a further class of embodiments, the outer shell comprises a biodegradable or biocompatible polymeric matrix assembled from one or more synthetic or natural polymer species. Poly(lactic-co-glycolic acid), in lactic-to-glycolic ratios of from 50:50 to 85:15 and weight-average molecular weights of from 7 kilodaltons to 240 kilodaltons, provides a hydrolytically degradable polyester shell with tunable in vivo erosion half-lives of from 14 days to 180 days, with the 50:50 ratio exhibiting the most rapid erosion and the 85:15 ratio the slowest. Polylactic acid in the L-, D-, and racemic D,L-stereoforms provides a more slowly eroding polyester with in vivo half-lives of from 6 months to 2 years and is preferred for sustained-release CNS depot embodiments. Polycaprolactone, of weight-average molecular weight of from 14 kilodaltons to 80 kilodaltons, provides a slowly eroding polyester with in vivo half-life of from 1 year to 3 years and exceptional cargo-protective rigidity. Poly(beta-amino esters), synthesized by Michael addition of from one to ten primary or secondary amines to a diacrylate monomer, provide cationic biodegradable polymers with apparent pKa of from 6.0 to 7.5 and intrinsic endosomal-escape capability for nucleic acid cargo delivery. Each of these synthetic polymer shells is fabricated into nanoparticles of from 80 nanometers to 300 nanometers hydrodynamic diameter by single-emulsion solvent-evaporation, double-emulsion solvent-evaporation, nanoprecipitation, or flash-nanoprecipitation, with surface modification by polyethylene-glycol and targeting-ligand conjugation through end-group click chemistry.

Chitosan, derived by alkaline deacetylation of crustacean-shell chitin to degrees of deacetylation of from 75 percent to 95 percent and weight-average molecular weights of from 5 kilodaltons to 500 kilodaltons, provides a cationic mucoadhesive polysaccharide shell that engages intestinal mucin through electrostatic interaction with negatively charged sialic-acid and sulfate residues. Trimethyl chitosan derivatives, prepared by methyl iodide quaternization at from 20 percent to 80 percent degree of substitution, provide enhanced solubility at neutral and slightly alkaline intestinal pH; thiolated chitosan derivatives, prepared by carbodiimide coupling of thioglycolic acid, cysteine, or N-acetylcysteine, provide covalent disulfide-bond formation with cysteine-rich mucin glycoproteins and confer extended intestinal residence times; and glycol chitosan derivatives provide enhanced aqueous solubility and reduced viscosity at high polymer concentrations. Alginate, a linear copolymer of mannuronic and guluronic acid blocks derived from brown algae, provides an anionic polysaccharide shell that gels through calcium-ion-mediated egg-box crosslinking and is preferred for pH-responsive intestinal-release embodiments by virtue of its solubilization above pH 3.5. Gelatin, in type-A acid-derived or type-B alkaline-derived forms and bloom strengths of from 50 to 300, provides a denatured-collagen protein shell amenable to glutaraldehyde, transglutaminase, or genipin crosslinking. Casein and silk fibroin provide protein-based shells of intrinsic biocompatibility and tunable beta-sheet content for stability modulation. Hyaluronic acid, in weight-average molecular weights of from 10 kilodaltons to 2,000 kilodaltons, provides a CD44-targeting anionic polysaccharide shell preferred for CNS-tumor-targeting embodiments.

Dextran, of weight-average molecular weights of from 10 kilodaltons to 2,000 kilodaltons and substituted at hydroxyl groups with carboxymethyl, sulfate, amine, aldehyde, or maleimide functional groups, provides a tunable polysaccharide-based shell amenable to oxidation-driven Schiff-base crosslinking and click-chemistry-driven ligand conjugation. Cyclodextrin inclusion architectures, comprising alpha-, beta-, or gamma-cyclodextrin host molecules either free, polymer-grafted, or self-assembled into nanosponges through crosslinking with diphenyl carbonate or epichlorohydrin, provide cargo-loading capacity through hydrophobic-pocket inclusion complexation with cavity diameters of from 4.7 angstroms to 8.3 angstroms tuned to the lipophilic surface area of the CNS therapeutic cargo. PAMAM dendrimers of generations 2 through 7, comprising 16 to 512 surface primary-amine groups respectively and hydrodynamic diameters of from 2.9 nanometers to 8.1 nanometers, provide a monodisperse hyperbranched polymer scaffold with precisely controllable surface-charge density and ligand-conjugation stoichiometry, with the higher generations 5 through 7 preferred when high ligand-display density is required and the lower generations 2 through 4 preferred when lower cytotoxicity and renal clearance compatibility are required. Low-molecular-weight polyethyleneimine, of weight-average molecular weight of from 0.8 kilodaltons to 25 kilodaltons in linear or branched architectures, provides a high-charge-density cationic polymer for nucleic acid condensation through proton-sponge-mediated endosomal escape, with the lower-molecular-weight branched 1.8-kilodalton and linear 2.5-kilodalton variants preferred to minimize cytotoxicity while retaining transfection efficacy.

### Inorganic Shells

In a further class of embodiments, the outer shell comprises an inorganic or hybrid inorganic-organic shell selected from mesoporous silica, calcium phosphate, iron oxide, gold, and upconversion-nanoparticle architectures. Mesoporous silica nanoparticles, synthesized by sol-gel condensation of tetraethyl orthosilicate in the presence of a cetyltrimethylammonium bromide, Pluronic P123, or Pluronic F127 structure-directing template followed by calcination or acidified-ethanol extraction, provide a rigid silica framework with surface areas of from 600 square meters per gram to 1,200 square meters per gram and tunable cylindrical pore diameters of from 2 nanometers to 50 nanometers selected to match the gyration radius of the CNS cargo. Particle external dimensions are tuned from 50 nanometers to 300 nanometers by adjustment of structure-directing-template concentration, base catalyst concentration, and reaction temperature. Surface functionalization by silane chemistry with from 3-aminopropyltriethoxysilane, 3-mercaptopropyltrimethoxysilane, 3-glycidoxypropyltrimethoxysilane, or trimethoxysilyl-PEG-azide provides handle chemistries for ligand conjugation and pore-gating. Pore gating is achieved through one or more of disulfide-cleavable nanovalves, pH-responsive nanovalves based on protonatable amine groups, redox-responsive nanovalves, supramolecular cucurbituril or beta-cyclodextrin valves, polymer-coated valves, or biomolecular valves comprising DNA hairpins, aptamers, or protein caps configured to open upon dual-pH, enzymatic, or magnetothermal triggering.

Calcium phosphate shells, prepared in amorphous, brushite (CaHPO4 dihydrate), or hydroxyapatite (Ca10(PO4)6(OH)2) crystalline forms by coprecipitation of calcium chloride and dibasic sodium phosphate at controlled pH and ionic strength or by emulsion-templated precipitation, provide an intrinsically biocompatible mineral shell of from 30 nanometers to 200 nanometers external diameter with pH-responsive solubilization at endosomal pH 5.0 to pH 6.0 that drives cargo release. Calcium phosphate is particularly suited to nucleic acid CNS cargo encapsidation through coprecipitation with DNA, small interfering RNA, or messenger RNA, with encapsidation efficiencies of from 70 percent to 95 percent and inherent endosomal-escape capability. Iron oxide nanoparticles, prepared in magnetite (Fe3O4) or maghemite (gamma-Fe2O3) phases by thermal decomposition of iron-oleate, iron-acetylacetonate, or iron-pentacarbonyl precursors in high-boiling-point organic solvents, by coprecipitation of ferrous and ferric chlorides, or by aqueous polyol synthesis, provide superparamagnetic cores of from 5 nanometers to 30 nanometers diameter suitable for magnetic-resonance imaging, magnetic targeting through externally applied gradient fields, and magnetothermal cargo release through alternating-magnetic-field heating. Iron oxide cores are coated with biocompatible polymers including dextran, polyethylene glycol, polyvinylpyrrolidone, polysiloxane, or carboxymethyl dextran to confer aqueous colloidal stability, suppress non-specific protein adsorption, and provide handle chemistries for targeting-ligand conjugation.

Gold nanoparticles, prepared by citrate reduction (Turkevich method) for diameters of from 5 nanometers to 100 nanometers, by sodium borohydride reduction (Brust-Schiffrin method) for diameters of from 1.5 nanometers to 5 nanometers, or by seed-mediated growth for diameters of from 30 nanometers to 250 nanometers and for non-spherical morphologies including nanorods, nanostars, and nanocages, provide a chemically inert metallic core amenable to thiol-gold self-assembled monolayer functionalization with from PEG-thiol, peptide-thiol, oligonucleotide-thiol, or antibody-thiol species. Gold nanoshells, comprising a silica or hollow core of from 80 nanometers to 200 nanometers diameter surrounded by a continuous gold shell of from 5 nanometers to 30 nanometers thickness, exhibit tunable plasmon resonance in the near-infrared first-biological-window of from 700 nanometers to 1,000 nanometers and provide photothermal cargo release upon localized laser irradiation. Upconversion nanoparticles, comprising lanthanide-doped sodium yttrium fluoride (NaYF4: Yb,Er; NaYF4:Yb,Tm), sodium gadolinium fluoride, or related host matrices doped with sensitizer ytterbium and activator erbium or thulium ions, convert near-infrared excitation at 980 nanometers or 808 nanometers into ultraviolet, visible, or near-infrared emission and provide deep-tissue-penetrating activation of photocaged cargoes, photocleavable linkers, or photodynamic-therapy sensitizers. Each inorganic shell class is amenable to surface coating with a secondary lipid, polymer, or protein-cage layer to confer biological identity, suppress non-specific opsonization, and integrate into the multilayered outer-shell architectures described elsewhere in this specification.

### Biomimetic Cellular-Membrane Coatings

In a further class of embodiments, the outer shell comprises a biomimetic coating of cellular-plasma-membrane-derived vesicles applied to an underlying inorganic, polymeric, or protein-cage core through extrusion-mediated or sonication-mediated membrane fusion. Red blood cell membrane coatings are prepared from hypotonic-lysis-derived erythrocyte ghosts and retain CD47 surface presentation, where CD47 binds signal-regulatory-protein-alpha on macrophages and delivers a ‘do-not-eat-me’ signal that prolongs systemic circulation half-life by from three-fold to ten-fold relative to bare-core controls. Platelet membrane coatings, prepared from platelet-rich-plasma-derived platelet ghosts, retain P-selectin (CD62P), CD41, CD42, and GPIbα and confer affinity for activated endothelium, denuded vascular collagen, and circulating tumor cells, providing intrinsic targeting to inflamed CNS vasculature and metastatic CNS lesions. Leukocyte membrane coatings, prepared from polymorphonuclear and mononuclear leukocyte populations, retain LFA-1, Mac-1, PSGL-1, and other adhesion receptors that engage inflamed endothelium through ICAM-1 and VCAM-1 binding and provide active migration into inflammatory CNS lesions. Neutrophil membrane coatings provide a particularly potent inflammation-tropism modality through retention of CXCR1, CXCR2, and CD11b/CD18.

T-cell membrane coatings, prepared from primary or expanded T-cell populations, retain T-cell-receptor complexes, CD3, CD4 or CD8, and adhesion molecules that confer affinity for major-histocompatibility-complex-presenting cells and tumor microenvironments; in chimeric-antigen-receptor-engineered T-cell membrane embodiments, the coating further confers tumor-antigen-specific targeting. Mesenchymal stem cell membrane coatings, prepared from bone-marrow-, adipose-tissue-, or umbilical-cord-derived mesenchymal stem cells, retain CD44, CD90, CD73, and CXCR4 surface markers and confer intrinsic homing to inflammatory and injury sites including ischemic stroke penumbra, multiple-sclerosis demyelinating lesions, and traumatic brain injury sites. Neural stem cell membrane coatings, prepared from fetal- or induced-pluripotent-stem-cell-derived neural stem cell populations, retain CXCR4, integrins, and L1-CAM surface markers and confer intrinsic homing to CNS tumor lesions and demyelinating lesions, providing one of the most CNS-specific membrane-coating modalities contemplated herein. Cancer-cell membrane coatings, prepared from autologous patient-derived primary tumor cells, glioblastoma cell lines, or patient-derived organoids, retain N-cadherin, galectin-3, EpCAM, and tumor-specific antigens and confer homotypic targeting to the source tumor through cadherin-mediated and galectin-mediated cell-cell adhesion. Exosome membrane coatings, prepared from cell-culture-supernatant-derived 30-to-150-nanometer extracellular vesicles, retain CD9, CD63, CD81 tetraspanins and tissue-specific surface markers reflective of the parent cell, and confer intrinsic cell-tropic targeting and immune-evasion properties. Hybrid membrane combinations, prepared by co-extrusion of two or more cell-derived membrane preparations at controlled molar ratios of from 10:90 to 90:10, provide combinatorial surface-marker portfolios that integrate complementary targeting and immune-evasion modalities, for example a red-blood-cell-and-platelet hybrid that simultaneously presents CD47 and P-selectin.

### Outer-Shell Crosslinking and Stabilization

In a further class of embodiments, the outer shell is stabilized against gastric proteolytic, acidic, and mechanical-shear challenge by one or more covalent crosslinking chemistries applied either inter-subunit within a protein-based shell, inter-chain within a polymeric shell, or inter-leaflet within a lipid bilayer shell. Glutaraldehyde crosslinking, applied at from 0.05 weight percent to 0.5 weight percent in aqueous suspension at pH 7.0 to pH 7.5 for periods of from 30 minutes to 4 hours at from 4 degrees Celsius to 25 degrees Celsius, generates Schiff-base and pyridinium-aldol crosslinks between primary-amine groups on lysine residues of protein shells, polymer amines of chitosan or polyethyleneimine shells, and protein-derived cellular-membrane coatings, with residual aldehyde quenched by from 50 millimolar to 200 millimolar glycine, sodium borohydride, or sodium cyanoborohydride reduction. Photo-crosslinking with ruthenium-tris-bipyridyl as photosensitizer at from 0.5 millimolar to 2 millimolar in the presence of from 5 millimolar to 20 millimolar sodium persulfate under from 450 nanometer to 470 nanometer visible-light irradiation for periods of from 30 seconds to 5 minutes generates di-tyrosine and tyrosine-tyrosine crosslinks between tyrosine residues of protein shells with high crosslink density, exquisite spatial control, and minimal background reactivity.

Click-chemistry crosslinking provides bioorthogonal, biocompatible, and selectively triggerable covalent stabilization through one or more of: copper-catalyzed azide-alkyne cycloaddition (CuAAC) employing from 50 micromolar to 500 micromolar copper (II) sulfate with from 100 micromolar to 1 millimolar tris [(1-benzyl-1H-1,2,3-triazol-4-yl)methyl]amine and from 1 millimolar to 5 millimolar sodium ascorbate as catalytic system at neutral pH for crosslinking of azide- and alkyne-functionalized subunits; strain-promoted azide-alkyne cycloaddition (SPAAC) employing dibenzocyclooctyne or bicyclononyne strained-alkyne handles with azide partners in the absence of copper catalyst, suitable for in vivo and intra-particle crosslinking where copper cytotoxicity is a concern; and inverse-electron-demand Diels-Alder cycloaddition (IEDDA) between tetrazine and trans-cyclooctene partners, exhibiting second-order rate constants of from 1 inverse molar inverse second to 10,000 inverse molar inverse second and providing the fastest bioorthogonal crosslinking chemistry contemplated herein. Each click chemistry is compatible with genetic-code expansion incorporation of azide-, alkyne-, tetrazine-, and trans-cyclooctene-bearing non-canonical amino acids at defined positions in protein-shell subunits through amber-stop-codon suppression. Disulfide stabilization through engineered cysteine residues, introduced at inter-subunit interfaces of protein-cage shells by site-directed mutagenesis at positions identified through structural inspection of inter-subunit contacts to lie within from 4 angstroms to 6 angstroms inter-thiol distance across the interface, generates inter-subunit disulfide bonds during in vivo or in vitro oxidative folding that confer reversible redox-triggered cage destabilization at intracellular glutathione concentrations of from 1 millimolar to 10 millimolar.

### Enteric-Polymer Outer-Shell Architectures

In a further class of embodiments specifically configured for the gastric-to-intestinal pH transition encountered during oral administration, the outer shell comprises an enteric polymer coating selected to remain intact, hydrophobic, and impermeable at gastric pH of from 1.2 to 3.5 and to undergo pH-triggered solubilization, swelling, or controlled erosion at intestinal pH of from 5.5 to 7.4. Poly(methacrylic acid)-based enteric polymers, marketed under the Eudragit trade name, are co-polymers of methacrylic acid and one or more of methyl methacrylate, ethyl acrylate, and methyl acrylate at defined monomer ratios. Eudragit L30D-55, a 1:1 co-polymer of methacrylic acid and ethyl acrylate supplied as an aqueous 30 percent dispersion, dissolves above pH 5.5 and is preferred for proximal-jejunal release. Eudragit L100-55, the equivalent co-polymer in dry-powder form, dissolves above pH 5.5. Eudragit L100, a 1:1 co-polymer of methacrylic acid and methyl methacrylate, dissolves above pH 6.0 and is preferred for jejunal-to-ileal release. Eudragit S100, a 1:2 co-polymer of methacrylic acid and methyl methacrylate, dissolves above pH 7.0 and is preferred for ileocolonic release. Eudragit FS30D, an ethyl-acrylate-methyl-methacrylate-methacrylic-acid terpolymer, dissolves above pH 7.0 and provides delayed colonic release.

Hydroxypropyl methylcellulose acetate succinate (HPMC-AS) enteric polymers are supplied in three grades distinguished by the ratio of acetyl and succinyl substituents and by aqueous solubilization threshold: HPMC-AS LF dissolves above pH 5.5 and is preferred for upper-jejunal release, HPMC-AS MF dissolves above pH 6.0 and is preferred for mid-intestinal release, and HPMC-AS HF dissolves above pH 6.8 and is preferred for ileal and ileocolonic release. HPMC-AS is distinguished from Eudragit by its lower swelling at sub-threshold pH, its broader compatibility with hydrophobic drug cargo, and its established use as a solid-dispersion stabilizer for hydrophobic CNS therapeutic small molecules. In particular embodiments of the platform contemplated herein, the enteric polymer is applied as a continuous outer coating of from 5 micrometers to 50 micrometers nominal thickness over an underlying virus-like-particle, protein-cage, lipid-nanoparticle, polymeric-nanoparticle, inorganic-nanoparticle, or membrane-coated core, by one or more of fluid-bed coating in a Wurster bottom-spray configuration, pan coating, spray drying, electrospraying, or supercritical-fluid coating. In further embodiments, the enteric polymer is co-formulated as an inner co-matrix within a polymer-blend shell architecture, providing distributed pH-responsiveness throughout the shell rather than as a discrete outer coating, with the co-matrix polymer at from 10 weight percent to 50 weight percent of total shell polymer. In still further embodiments, a multi-pH-staged enteric architecture is constructed by sequential application of an inner Eudragit L100-55 layer dissolving at pH 5.5, an intermediate Eudragit L100 layer dissolving at pH 6.0, and an outer Eudragit S100 layer dissolving at pH 7.0, providing a pulsatile release profile along the proximal-to-distal intestinal axis.

### Selection Criteria Across Shell Architectures

The selection of a particular outer-shell architecture from among the bacteriophage-derived, plant-virus-derived, protein-cage, lipid bilayer, polymeric, inorganic, biomimetic-membrane, and enteric-polymer classes described above is governed in the platform contemplated herein by a multifactorial comparison of: (i) external hydrodynamic diameter and monodispersity, with sub-30-nanometer protein cages preferred for transcytosis-limited indications and 60-to-200-nanometer cores preferred for cargo-capacity-limited indications; (ii) internal cargo-loading capacity, with vault and T4-bacteriophage cages providing the highest per-particle volumes and protein cages such as Dps and ferritin providing the most homogeneous but most volume-limited interiors; (iii) gastric-to-intestinal dual-barrier stability, with QB-bacteriophage, tobacco-mosaic-virus, tomato-bushy-stunt-virus, and lumazine-synthase shells providing the greatest intrinsic acid and protease tolerance among non-enteric-coated embodiments; (iv) immunogenicity profile, with human-derived ferritin, exosome, and red-blood-cell-membrane shells exhibiting the lowest pre-existing-immunity and de novo immunogenicity, plant-virus-derived shells exhibiting moderate immunogenicity offset by lack of human cross-reactivity, and bacteriophage-derived shells exhibiting the highest expected immunogenicity in part attenuable by surface PEGylation; and (v) manufacturability at clinical scale, with lipid nanoparticles benefiting from established microfluidic-mixing infrastructure and regulatory precedent, polymeric nanoparticles benefiting from solvent-evaporation and nanoprecipitation infrastructure, plant-virus-derived particles benefiting from agroinfiltration scalability, and bacteriophage and protein-cage particles benefiting from established E. coli fermentation infrastructure.

A further consideration in shell selection is regulatory precedent and the corresponding anticipated regulatory pathway. Lipid nanoparticles of the MC3, SM-102, and ALC-0315 classes benefit from approved-drug-product precedent in patisiran, the BNT162b2 messenger-RNA vaccine, and the mRNA-1273 vaccine respectively, providing the most advanced regulatory pathway among the shell classes contemplated herein. Poly(lactic-co-glycolic acid) and polylactic acid polymeric shells benefit from extensive approved-depot-product precedent across multiple injectable formulations. Mesoporous silica nanoparticles have entered clinical evaluation through Cornell-dots and related products. Iron oxide nanoparticle cores benefit from prior approvals as magnetic-resonance-imaging contrast agents and as anemia therapeutics. Ferritin nanocages have entered clinical evaluation as vaccine platforms. Bacteriophage-derived virus-like particles, including QB-based platforms, have entered clinical evaluation as vaccine carriers. Cell-membrane-coated nanoparticles remain investigational with rapidly expanding clinical-stage activity. The platform of the present invention contemplates pursuing in parallel multiple shell-architecture embodiments selected to optimize the joint product of biological efficacy, gastrointestinal-and-blood-brain-barrier dual-barrier traversal, manufacturability, and regulatory tractability for each contemplated CNS indication, with the architectures of this section providing the modular structural backbone upon which the dual-pH-triggered, enzyme-responsive, and magnetically guided release-and-targeting modalities described in the subsequent sections of this specification are layered.

### Expanded Axis 3—Inner Core Architecture (Deep Dive)

### DNA Origami Inner Cores at Class Level

Within the embodiments of Axis 3, the structured inner core preferentially comprises a DNA origami nanostructure assembled from a single-stranded scaffold of from 1,000 to 50,000 nucleotides and from 50 to 500 unique staple oligonucleotides, said scaffold and staples being folded by thermal annealing from 95 degrees Celsius to 20 degrees Celsius over a duration of from 12 to 24 hours or by isothermal annealing at from 45 to 55 degrees Celsius in formamide-containing buffer for from 1 to 4 hours, in either case in the presence of from 5 to 20 millimolar magnesium ions. Folding yield of from 70 percent to 90 percent of theoretical is contemplated. The folded core is purified by one or more of polyethylene glycol precipitation (10 to 20 percent w/v PEG-8000, 500 millimolar sodium chloride), agarose gel electrophoresis, size-exclusion chromatography on Sephacryl S-500 or S-1000, sucrose-gradient ultracentrifugation (20 to 50 percent linear gradients), and spin-column ultrafiltration at 100 kilodalton molecular weight cutoff.

Geometries within the DNA origami inner core class include tubular cores having a length of from 30 to 120 nanometers, an outer diameter of from 15 to 70 nanometers, an internal cavity diameter of from 10 to 40 nanometers, and a wall thickness corresponding to one, two, or three concentric DNA duplex layers; spherical or quasi-spherical wireframe cores having a diameter of from 20 to 80 nanometers; polyhedral closed-frame cores of tetrahedral, octahedral, cuboctahedral, dodecahedral, icosahedral, and truncated icosahedral symmetry; open-frame polyhedral wireframes constructed from edge-only scaffold-and-staple architectures; planar two-dimensional sheet cores; multi-arm branched architectures comprising three to twelve radial arms emerging from a central hub; and dynamic shape-shifting architectures incorporating engineered hinge regions that change configuration in response to a defined trigger.

DNA origami inner cores are stabilized against nuclease degradation by one or more of: coating with cationic polymers comprising oligolysine or oligoarginine of from 5 to 30 amino acid residues; coating with polysarcosine of from 5 to 50 sarcosine residues; coating with polyethylene-glycol-grafted variants of the foregoing cationic polymers; ultraviolet-induced thymidine-dimer crosslinking at irradiation doses of from 1 to 10 millijoules per square centimeter; click-chemistry crosslinking employing 5-ethynyl-deoxyuridine and 5-azido-deoxyuridine modified positions; photo-crosslinking using psoralen-class photoreactive groups; and silica mineralization through controlled deposition of tetraethyl orthosilicate, providing thermal stability up to 500 degrees Celsius and resistance to nuclease degradation for greater than 24 hours in 10 percent serum.

### RNA Origami Inner Cores at Class Level

Within further embodiments of Axis 3, the structured inner core comprises an RNA origami nanostructure assembled by co-transcriptional folding from a DNA template using T7 or SP6 RNA polymerase, with the resulting RNA nanostructure incorporating engineered kissing-loop, three-way junction, four-way junction, and pseudoknot motifs to define an internal cavity of from 10 to 40 nanometers in diameter. RNA origami cores include nanoring architectures, RNA-protein hybrid scaffolds, tRNA-derived scaffold variants, and engineered co-transcriptionally folded multi-arm architectures. Stabilization is achieved through 2′-O-methyl and 2′-fluoro chemical modification of selected positions, encapsulation within a protein nanocage interior, or oligolysine coating analogous to that employed for DNA origami cores.

### Peptide-Nanofiber and Peptide-Amphiphile Cores

Within further embodiments of Axis 3, the structured inner core comprises a self-assembling peptide nanofiber, a peptide-amphiphile cylindrical micelle, a designed protein-based de novo cage assembly of the I3-01, T33, or O3 design family, an elastin-like polypeptide bundle, an intrinsically disordered protein core, or a pH-responsive peptide nanofiber configured for enteric stability and endosomal disassembly. The peptide core class is loaded by aqueous compartment entrapment, by electrostatic association, by covalent tethering through engineered conjugation handles, or by encapsulation during peptide self-assembly. Mechanical properties span the range from soft elastic moduli of tens of kilopascals to rigid moduli of hundreds of megapascals depending on the specific peptide architecture, enabling tuning of mechanical compliance for blood-brain barrier crossing as recited in Axis 4.

### Protein-Nanocage Interior Cores, Dendrimers, Mesoporous Inorganic Cores, Polymeric Micelles, Hydrogels, and Liquid-Crystalline Phases

Within further embodiments of Axis 3, the inner core comprises a protein nanocage interior selected from ferritin, encapsulin, lumazine synthase, or vault protein architectures nested within a larger outer protein nanoshell to form a hierarchical protein-cage-in-protein-cage assembly. Suitable alternative inner-core architectures include dendrimer cores of the polyamidoamine (PAMAM, generations 3 through 7), poly(propylene imine) (PPI), polyester, polyamide, peptide, glycodendrimer, and hybrid dendrimer-polymer classes; mesoporous silica nanoparticles of the MCM-41 class with pores of from 2 to 4 nanometers, the SBA-15 class with pores of from 5 to 15 nanometers, the MCM-48 class with bicontinuous cubic pores, and the KIT-6 class; mesoporous calcium phosphate, mesoporous bioactive glass, hollow mesoporous silica, and metal-organic framework cores including ZIF-8, MIL-100, and UiO-66 with pore tunability across 0.5 to 5 nanometers; polymeric micelle cores formed from PEG-PCL, PEG-PLGA, PEG-PLA, or Pluronic-class triblock copolymers; thermosensitive micelle cores incorporating poly(N-isopropylacrylamide) segments; pH-responsive micelle cores; reverse-micelle architectures for hydrophilic payload retention; alginate, gelatin methacrylate, hyaluronic acid, and polyethylene glycol diacrylate hydrogel cores; intramolecularly crosslinked nanogel cores; bicontinuous cubic-phase cores of glyceryl monooleate or phytantriol; hexagonal-phase cores; and lamellar La cores. Each of the foregoing inner-core architectures admits hybrid hierarchical configurations in which two or more inner-core architectures are combined in nested, compartmentalized, or sequential-release arrangements for multi-payload or multi-trigger embodiments.

### Inner-Core Selection and Cargo-Loading Methods at Class Level

Selection of an inner-core architecture from the foregoing classes is guided by the molecular weight and physicochemical properties of the therapeutic payload of Axis 1; the internal cavity volume required for the selected payload at the desired drug load; the mechanical compliance properties required for traversal of the blood-brain barrier as set forth in Axis 4; the surface charge density required for electrostatic complementarity with the selected outer-shell architecture of Axis 2; the stabilization strategy compatible with the selected stimulus-responsive release element of Axis 5; and the manufacturing scalability appropriate to the intended commercial scale. Cargo loading into the selected inner core is achieved by any one or combination of the following loading modes: encapsulation during folding or assembly, post-assembly diffusion loading, electrostatic co-association, aptamer-mediated capture using internal binding-site classes incorporated into the inner core, hybridization-based loading using complementary capture elements described as a functional class, site-specific covalent conjugation at predetermined internal positions through engineered conjugation handles using maleimide-thiol chemistry, copper-catalyzed or copper-free click chemistry, SpyTag-SpyCatcher isopeptide bond formation, or sortase-mediated ligation, and combination loading employing two or more of the foregoing modes for multi-payload embodiments.

### Expanded Axis 4—Dual-Barrier Crossing Mechanism (Deep Dive)

### Gastrointestinal Absorption Barrier and M-Cell Targeting

The gastrointestinal absorption barrier comprises a sequence of physical and biochemical obstacles to oral nanoparticle delivery, beginning with the gastric acidic environment (pH of from 1.0 to 3.5 in the fasted state) and gastric peptidase activity (principally pepsin, optimally active at pH 2 to 4), proceeding through the intestinal pancreatic protease milieu (trypsin, chymotrypsin, elastase, carboxypeptidase A and B at pH 6.5 to 8.0), the intestinal brush-border enzyme complement (aminopeptidases, dipeptidases, alkaline phosphatase), the mucus layer (a tetrameric mucin glycoprotein gel of thickness from 50 to 800 micrometers depending on intestinal segment), the glycocalyx of enterocyte apical membranes (a 400 to 500 nanometer-thick brush-border glycoprotein layer), and the intestinal tight-junction architecture (formed by claudin-1, claudin-2, claudin-3, claudin-4, claudin-5, occludin, and junctional adhesion molecules limiting paracellular permeability to molecules of less than approximately 600 daltons). The disclosed nanostructure traverses this absorption barrier through outer-shell-mediated protection and through one or more receptor-mediated, transcellular, or transcytotic crossing mechanisms as set forth in the following paragraphs.

Microfold-cell (M-cell) targeting employs ligand classes that bind M-cell surface markers including glycoprotein 2, ubiquitin-D, the M-cell-restricted CCL9 receptor, claudin-4, and beta-1 integrin. Ligand classes include peptide ligands of from 5 to 30 amino acid residues identified by phage-display screening for binding to M-cell apical surfaces (presented as functional class without literal sequences); antibody fragments specific for the foregoing M-cell surface markers in formats including single-chain variable fragments and single-domain antibody fragments; carbohydrate moieties including mannose, fucose, and beta-glucan classes that bind M-cell-expressed lectin receptors; aptamer ligands selected against M-cell markers; and peptides derived from bacterial-invasion-protein families of Salmonella SipB class, Yersinia YadA class, and Listeria Internalin A class, engineered to retain receptor-binding capacity without retaining infectious capacity. M-cells comprise approximately 10 percent of follicle-associated epithelium and provide an expected 5- to 10-fold enhancement of nanoparticle intestinal transcytosis compared with non-targeted controls under the conditions disclosed herein.

Enterocyte transcellular transport pathways supplement M-cell-mediated transcytosis and include receptor-mediated transcytosis via the enterocyte neonatal Fc receptor (FcRn) for Fc-bearing particles, vitamin-B12 transcobalamin-II receptor transport for particles conjugated with vitamin-B12, bile-acid transporter (ASBT) transport for particles conjugated with bile-acid moieties, bombesin-receptor transport, and basolateral transferrin-receptor-mediated transcytosis. Paracellular tight-junction modulation may be employed as an adjunct, with transient and reversible opening of intestinal tight junctions induced by chitosan-class polysaccharide enhancers, sodium caprate, sodium caprylate, sodium salicylate, EDTA-class calcium chelators, and zonula-occludens-derived peptide enhancers described as a functional class. Lymphatic uptake via chylomicron-mimetic formulations may further be employed for lipophilic payloads, routing absorbed nanoparticles through the mesenteric lymphatics to the systemic circulation while bypassing first-pass hepatic metabolism.

### Blood-Brain Barrier Crossing

The blood-brain barrier comprises brain capillary endothelial cells joined by tight junctions formed principally by claudin-5, occludin, and junctional adhesion molecules; an active efflux transporter complement principally including P-glycoprotein (ABCB1), breast cancer resistance protein (ABCG2), and the multidrug resistance protein family (ABCC1 through ABCC10); and a receptor-mediated transcytosis machinery comprising the transferrin receptor (TfR1 and TfR2), insulin receptor, insulin-like growth factor 1 receptor, low-density lipoprotein receptor (LDLR), low-density lipoprotein receptor-related protein 1 (LRP1), low-density lipoprotein receptor-related protein 8 (LRP8/ApoER2), leptin receptor, nicotinic acetylcholine receptor (alpha-4-beta-2 and alpha-7 subtypes), heparin-binding epidermal growth factor receptor, melanotransferrin receptor, basigin, and CD98 hc. The disclosed nanostructure crosses the blood-brain barrier by receptor-mediated transcytosis directed by one or more transcytosis ligand classes configured to bind one or more of the foregoing receptors with an apparent dissociation constant in the range of from 50 nanomolar to 500 nanomolar, said range being the transcytosis sweet spot at which receptor-mediated transcytosis is favored over receptor lock-up. Ligand classes include peptide ligands of from 5 to 35 amino acid residues, antibody fragments engineered for moderate (not maximal) receptor affinity, full-length natural receptor ligands, mimetic ligands, and aptamer ligands selected for the foregoing receptors.

Adjunct blood-brain barrier crossing mechanisms include adsorptive-mediated transcytosis directed by cationic surface coatings and by cell-penetrating peptide classes (TAT family cationic peptides, penetratin family amphipathic peptides, polyarginine-class peptides, and MAP family amphipathic peptides described as functional classes only); carrier-mediated transport directed by glucose-transporter-1, large-neutral-amino-acid-transporter-1, monocarboxylate-transporter-1, and cationic-amino-acid-transporter ligands; and physically assisted crossing mechanisms including focused-ultrasound-induced transient blood-brain barrier opening with microbubble cavitation, magnetic-field-assisted enhancement of crossing using magnetic-iron-core particles, electromagnetic-stimulus enhancement, transient hyperosmotic blood-brain barrier opening using mannitol pretreatment, and transcranial magnetic stimulation co-administration. Additional uptake into the central nervous system compartment is achieved through alternative pathways including folate receptor alpha on the choroid plexus epithelium (providing cerebrospinal-fluid-compartment delivery via blood-cerebrospinal-fluid barrier crossing) and meningeal lymphatic vessel uptake from cervical lymph drainage.

### Sequential Temporal Control of Dual-Barrier Ligand Presentation and Quantitative Expectations

Sequential temporal control of dual-barrier ligand presentation is achieved by outer-shell-shedding architectures that expose blood-brain-barrier-crossing ligands carried on the inner core only after the outer-shell has been progressively shed in response to gastrointestinal-pH-transition-triggered, enzymatic, redox, or reactive-oxygen-species stimuli encountered during gastrointestinal transit and entry into the systemic circulation. Stage-wise targeting prevents premature ligand consumption by off-target binding during transit and is a preferred configuration of the disclosed dual-barrier architecture. Quantitative performance expectations based on published precedent for the individual mechanism classes are: expected gastrointestinal-barrier crossing efficiency of from 1 percent to 15 percent depending on M-cell-targeting density and enhancer use; expected systemic exposure (oral bioavailability) of from 3 percent to 12 percent; and expected blood-brain barrier crossing efficiency of from 0.5 percent to 10 percent of systemic dose, yielding an overall oral-to-central-nervous-system delivery efficiency of from 0.02 percent to 1.5 percent of the oral dose at the central nervous system target, with the actual realized efficiency depending on the specific combination of axes selected for a given embodiment.

### Expanded Axis 5—Stimulus-Responsive Release (Deep Dive)

### Stimulus-Responsive Release Mechanisms

pH-Responsive Nucleic Acid Switches

The biomimetic shell-core oral nanostructure platform incorporates a class of pH-responsive nucleic acid architectures that undergo defined conformational transitions in response to the protonation state of constituent nucleobases, thereby providing programmable triggers for payload release at compartment-specific hydrogen ion activities encountered along the gastrointestinal lumen, across enterocyte transcytotic vesicles, and within target-cell endolysosomal organelles. Among these architectures, i-motif tetrad structures are formed by cytosine-rich oligonucleotide strands that fold into intercalated quadruplex topologies stabilized by hemiprotonated cytosine-cytosine base pairs at solution pH values of from 4.5 to 6.8, with the precise transition midpoint tunable by varying cytosine tract length, loop length, ionic strength, and inclusion of methylated or 5-halogenated cytosine analogs. Below the transition midpoint, the i-motif adopts a compact intercalated form that brings together previously separated oligonucleotide termini; above the transition midpoint, the structure unfolds into a random-coil conformation that releases tethered cargo, opens crosslinked networks, or exposes previously occluded ligands. The transition is reversible and exhibits hysteresis of from 0.2 to 0.6 pH units depending on architecture, permitting use as a switch with defined on and off thresholds. Triplex DNA elements employing Hoogsteen base pairing provide a second pH-responsive architectural class wherein a third oligonucleotide strand binds in the major groove of a duplex region through pyrimidine-purine-pyrimidine or purine-purine-pyrimidine motifs, with the pyrimidine-motif triplex requiring protonation of N3 of cytosine for stable formation. The pyrimidine-motif triplex exhibits a pH-dependent stability profile with formation favored below pH 6.0 and dissociation favored above pH 7.0, with the transition midpoint tunable by inclusion of 5-methylcytosine, 2-aminopurine, or peptide nucleic acid analogs. pH-tunable G-quadruplex architectures may be designed with abasic substitutions, modified guanine analogs, or strategically placed cytosine residues to confer pH-dependent fold stability with transition midpoints of from 5.5 to 7.5.

Kinetics of pH-responsive nucleic acid switches are determined by the rate-limiting strand-rearrangement step rather than by the protonation equilibrium, with characteristic transition half-times of from 50 milliseconds to 30 seconds for i-motif tetrads of from 10 to 24 nucleotides, of from 1 second to 5 minutes for triplex displacement reactions, and of from 10 milliseconds to 10 seconds for G-quadruplex refolding events. Triplex elements may be incorporated as locking elements that constrain a DNA origami shell in a compact form at acidic pH and release the shell upon transit to neutral compartments, or in the reverse configuration may crosslink interior compartments that remain closed at duodenal pH and open upon endosomal acidification. The G-quadruplex class additionally provides cation-dependent stabilization permitting orthogonal control by potassium or sodium concentration, useful in tuning release kinetics independently of pH. Manufacturing implications include solid-phase phosphoramidite synthesis of constituent oligonucleotides at scales of from 50 micromoles to 10 millimoles per batch, purification by reverse-phase or ion-exchange high-performance liquid chromatography to greater than 95 percent full-length product, and quality control by ultraviolet circular dichroism spectroscopy at characteristic 265 to 295 nanometer bands. Compatibility with the inner core is established by chemical conjugation of the pH-responsive oligonucleotide via 3′ or 5′ amine, thiol, azide, or dibenzocyclooctyne handles to core-surface functional groups, or by hybridization to scaffold strands embedded in the core matrix. Compatibility with the outer shell is established by inclusion of the pH-responsive element as a structural staple in DNA origami shells, as a crosslinker between shell-embedded duplex regions, or as a tether anchoring shell-displayed targeting ligands, with the transition driving local opening of apertures, shedding of outer protective layers, or global decompaction of the shell as appropriate to the architectural level.

pH-Responsive Chemical Linkers

A complementary class of pH-responsive triggers consists of small-molecule chemical linkers that undergo hydrolytic cleavage at rates strongly dependent on hydrogen ion concentration, with cleavage half-lives ranging across more than six orders of magnitude between neutral extracellular fluid and acidified endolysosomal compartments. Hydrazone linkers, formed by condensation of a hydrazide with an aldehyde or ketone, exhibit hydrolysis half-lives of from 24 to 168 hours at pH 7.4 and of from 0.5 to 6 hours at pH 5.0, with the rate tunable by the electronic and steric character of carbonyl substituents; aromatic hydrazones from benzaldehyde derivatives are more stable than aliphatic hydrazones from ketones. Acetal linkers, formed from a carbonyl and two alcohol equivalents, hydrolyze with half-lives of from 100 to 1000 hours at pH 7.4 and of from 1 to 24 hours at pH 5.0; cyclic acetals from diols are more stable than acyclic, and benzaldehyde acetals hydrolyze faster than aliphatic-aldehyde acetals. Beta-thiopropionate linkers, formed by Michael addition of a thiol to an acrylate followed by amide formation, exhibit half-lives of from 12 to 72 hours at pH 7.4 and of from 0.25 to 4 hours at pH 5.0, with accelerated cleavage in the presence of intracellular reducing agents. Orthoester linkers possess three alkoxy substituents on a single carbon and undergo acid-catalyzed hydrolysis to esters and alcohols with half-lives of from 10 to 200 hours at pH 7.4 and of from 0.1 to 2 hours at pH 5.0, providing one of the steepest pH-dependent hydrolysis rate profiles. Vinyl ether linkers undergo acid-catalyzed addition of water followed by tautomerization to yield a carbonyl and an alcohol with half-lives of from 50 to 500 hours at pH 7.4 and of from 1 to 12 hours at pH 5.0, with the rate increased by electron-donating substituents on the vinyl carbon. Cis-aconityl linkers, formed by reaction of a primary amine with cis-aconitic anhydride to yield an amide bearing a free carboxylic acid in cis configuration, undergo intramolecularly catalyzed amide hydrolysis with half-lives of from 20 to 100 hours at pH 7.4 and of from 0.5 to 8 hours at pH 5.0; trans-aconityl linkers do not exhibit this acceleration and serve as stable controls.

Manufacturing implications of pH-responsive chemical linkers include compatibility with standard solution-phase or solid-phase bioconjugate chemistry, typically performed at pH of from 6.5 to 8.5 in aqueous or mixed aqueous-organic solvent at temperatures of from 4 degrees Celsius to 37 degrees Celsius, with purification by size-exclusion or reverse-phase chromatography and quality control by ultra-performance liquid chromatography coupled to mass spectrometry to confirm linker incorporation and quantify residual hydrolysis products. The linkers are introduced via heterobifunctional crosslinkers presenting an N-hydroxysuccinimide ester, maleimide, or click-chemistry handle on one terminus and the pH-labile bond on the central segment, with the opposing terminus bearing a complementary handle. The differential ratio of cleavage rate at pH 5.0 to cleavage rate at pH 7.4 ranges from 10 for less-selective linker classes to greater than 1000 for orthoester and cis-aconityl chemistries, with selection driven by required selectivity for endolysosomal versus extracellular release and by required residence time in transit compartments. Compatibility with the inner core is established by attachment of payloads via the pH-labile linker to core-surface amines, thiols, or click handles, providing pH-triggered release of free payload upon endolysosomal acidification; compatibility with the outer shell is established by use of the linker to attach shell-displayed targeting ligands, masking moieties, or shell-stabilizing crosslinkers that release upon arrival in the target compartment. Stability during gastrointestinal transit is assured by selecting linkers with pH 7.4 half-lives substantially longer than transit time and by enteric coating of the outer shell. For combination payloads, multiple linker chemistries with staggered half-lives are incorporated in defined stoichiometric ratios within a single architecture, yielding sequential release over a programmed temporal profile.

pH-Responsive Polymer Coatings

pH-responsive polymer coatings provide a third triggered-release modality wherein protonation of pendant ionizable groups alters polymer hydration and solubility, leading to swelling, dissolution, or charge inversion of a coating layer surrounding the inner core. Poly(beta-amino ester) coatings comprise polymers obtained by Michael addition of primary or secondary amines to diacrylate monomers, yielding pendant tertiary amines with pKa values of from 5.5 to 7.5 tunable by amine and diacrylate selection. Below the apparent pKa, the amines are protonated, the polymer carries net positive charge, and the coating is water-soluble; above the apparent pKa, the polymer deprotonates, loses solubility, and forms a hydrophobic film. By appropriate selection of monomers, coatings may be designed to dissolve at endolysosomal pH while remaining intact at extracellular pH, providing a transcytosis-triggered release. Poly(histidine) coatings exploit the imidazole side chain with pKa of from 6.0 to 6.5; in the acidic endolysosome, the imidazoles protonate, the polymer swells through electrostatic repulsion, and adjacent membranes are destabilized through the so-called proton-sponge effect, simultaneously releasing payload and promoting endosomal escape. Engineered poly-L-lysine variants in which a fraction of epsilon-amines is modified with low-pKa carboxylate masking groups, such as cis-aconityl or citraconyl moieties, exhibit pH-dependent charge inversion from net negative at neutral pH to net positive at acidic pH, providing both pH-triggered release and acid-activated membrane interaction. The fraction of lysine residues modified, of from 30 to 95 percent, determines the apparent isoelectric point and the sharpness of the charge-inversion transition. Methacrylic acid copolymers, in which methacrylic acid is copolymerized with methyl methacrylate, ethyl acrylate, or methyl acrylate at defined ratios, exhibit pH-dependent dissolution behavior with apparent transition pH determined by the ratio of carboxylate-bearing to neutral monomers; ratios of methacrylic acid to neutral monomer of from 1:1 to 1:2 yield dissolution at pH of from 5.5 to 7.0, suitable for proximal small intestine or for endolysosomal compartments.

Coating kinetics are determined by polymer molecular weight, crosslink density, coating thickness, and surrounding ion composition, with characteristic dissolution times of from 5 to 120 minutes at the trigger pH and stable coating retention for greater than 24 hours at non-trigger pH. Manufacturing implications of pH-responsive polymer coatings include polymer synthesis by Michael addition, controlled radical polymerization, or solid-phase peptide synthesis depending on the polymer class, with characterization by gel-permeation chromatography for molecular weight distribution, nuclear magnetic resonance for composition, and acid-base titration for apparent pKa. Coating onto the inner core or onto a preformed shell-core assembly is performed by solvent exchange, antisolvent addition, layer-by-layer assembly, or spray-coating, with coating thickness of from 5 to 200 nanometers controlled by polymer concentration, deposition time, and number of layers. Polymer molecular weights of from 5 kilodaltons to 100 kilodaltons are typical, with low-molecular-weight polymers providing faster dissolution but lower film integrity and high-molecular-weight polymers providing more durable films but slower dissolution. Compatibility with the inner core is established by selection of polymer functional groups orthogonal to core surface chemistry and by use of polymer charge that does not destabilize the core; coating polymers carrying net charge at the formulation pH must be selected with regard to the surface charge of the core to avoid premature aggregation. Compatibility with the outer shell is established either by coating the entire shell-core assembly with the pH-responsive polymer or by integrating the polymer as an interpenetrating layer between inner core and outer DNA origami shell, providing a defined dissolution sequence upon triggering. Cross-linked variants, in which a small fraction of monomers carries crosslinkable groups such as methacrylate or acrylate, provide enhanced film integrity at the cost of slower dissolution and are selected for platforms requiring extended residence in transit compartments.

### Enteric Polymer Systems

Enteric polymer systems comprise pharmaceutical-grade pH-responsive polymers with extensive regulatory precedent in oral drug products, used in the platform to provide a first-line gastric-acid-stable outer protective coating that dissolves at defined small-intestinal pH thresholds, thereby exposing the inner shell-core assembly only after gastric transit. Eudragit L30D-55 and Eudragit L100-55 are aqueous dispersion and powder forms, respectively, of methacrylic acid-ethyl acrylate copolymer with a free carboxyl to ester ratio of 1:1, dissolving above pH 5.5 and suitable for release in the proximal duodenum. Eudragit L100 is a methacrylic acid-methyl methacrylate copolymer with a 1:1 ratio dissolving above pH 6.0, suitable for upper jejunal release. Eudragit S100 is a methacrylic acid-methyl methacrylate copolymer with a 1:2 ratio dissolving above pH 7.0, suitable for distal ileal or colonic release. Eudragit FS30D is a methacrylic acid-methyl acrylate-methyl methacrylate terpolymer dissolving above pH 7.0 with additional sustained-release character due to its lower carboxyl content, suitable for colonic delivery with extended residence. Hydroxypropyl methylcellulose acetate succinate, available in low-(LF), medium-(MF), and high-(HF) substitution grades, provides an alternative cellulose-derived enteric polymer with dissolution thresholds of pH 5.5, 6.0, and 6.8 respectively, the threshold determined by the acetyl-to-succinoyl substitution ratio. Cellulose acetate phthalate dissolves above pH 6.0 to 6.2 and provides moderate moisture permeability suitable for solid oral dosage forms. Polyvinyl acetate phthalate dissolves above pH 5.0 to 5.5 and provides good film-forming properties. Coating onto the platform is performed by aqueous dispersion fluid-bed coating, organic-solvent pan coating, or spray-drying, with coating weight gains of from 6 percent to 25 percent by weight of the uncoated assembly providing complete gastric protection in standardized USP simulated gastric fluid disintegration assays. Plasticizers including triethyl citrate, dibutyl sebacate, or polyethylene glycol of from 5 to 25 percent by weight of polymer are incorporated to reduce coating brittleness.

Manufacturing implications of enteric polymer coating include the requirement for high-shear or fluid-bed equipment of capacities matched to batch size, with process parameters including inlet air temperature of from 30 to 60 degrees Celsius, atomization pressure of from 1 to 4 bar, and spray rate adjusted to maintain product temperature below the polymer glass transition while avoiding overwetting. For platforms incorporating thermally or hydrolytically labile components, organic-solvent coating using ethanol, isopropanol, acetone, or mixtures thereof provides rapid drying and minimized water exposure, while aqueous coating using polymer dispersions provides environmental and worker-safety advantages where assembly stability permits. Quality-control assays include USP simulated gastric fluid disintegration testing in which less than 10 percent of dose is released over 2 hours at pH 1.2, followed by greater than 80 percent release within 30 to 60 minutes upon transfer to simulated intestinal fluid; additional assays include coating uniformity by laser diffraction, mechanical integrity by friability testing, and accelerated stability under International Council for Harmonisation conditions to confirm shelf life of greater than 24 months. Combination coating strategies in which two or more enteric polymers are layered or blended yield programmed multi-stage release, with an outer pH-5.5 layer dissolving in the duodenum, a middle pH-6.0 layer in the jejunum, and an inner pH-7.0 layer in the ileum, providing extended-residence release along the small intestine. Compatibility with the inner core and outer shell is established by the outermost position of the enteric polymer in the architecture, such that the inner assembly is exposed only after enteric dissolution; for architectures sensitive to organic solvent, aqueous coating is used, while moisture-stable architectures permit either coating modality.

### Enzymatic-Responsive Peptide Linkers

Enzymatic-responsive peptide linkers provide compartment-specific and cell-type-specific triggers through cleavage by proteases enriched in particular subcellular organelles, tissues, or disease states. Cathepsin B, S, and L are lysosomal cysteine proteases with substantial activity in late endosomes and lysosomes at acidic pH, recognized for their preference for hydrophobic and basic residues at the P2 and P1 positions of substrate peptides; dipeptide linkers of the valine-citrulline class, phenylalanine-lysine class, and related classes are cleaved by cathepsin B with catalytic efficiencies of from 1 thousand to 1 million per molar per second, providing half-lives of from 1 minute to 4 hours at intralysosomal enzyme concentrations of from 1 to 50 micromolar. Cathepsin S exhibits broader substrate tolerance and significant activity at neutral pH in antigen-presenting cells; cathepsin L is enriched in proliferating cells and in certain tumors, providing a disease-selective trigger. Caspase-3 substrates of the aspartate-glutamate-valine-aspartate tetrapeptide class with cleavage carboxyterminal to the second aspartate are cleaved exclusively by apoptosis-executioner caspases and therefore release payload only in apoptotic target cells, suitable for cytotoxic or signal-amplifying cargo. Matrix metalloproteinase-2 and matrix metalloproteinase-9 substrates of the proline-leucine-glycine-leucine class and related sequences cleaved between the glycine and leucine are cleaved by extracellular matrix-remodeling zinc-dependent metalloproteinases upregulated in tumor stroma, inflamed tissue, and remodeled vasculature, providing tissue-selective extracellular release without requirement for cellular uptake. Urokinase plasminogen activator substrates of the glycine-glycine-arginine and serine-glycine-arginine-serine classes are cleaved by urokinase in invasive and angiogenic tissue. Prostate-specific antigen substrates of the histidine-serine-serine-lysine-leucine-glutamine class are cleaved by the chymotrypsin-like activity of PSA and therefore provide highly tissue-selective release in prostate tissue and in prostate-cancer metastases.

Kinetics of enzymatic peptide linker cleavage are described by Michaelis-Menten parameters with Km values of from 1 to 500 micromolar and kcat values of from 0.01 to 100 per second depending on enzyme-substrate pair, yielding apparent cleavage half-lives of from 0.5 minutes to 12 hours at physiological enzyme concentrations and substrate excess; cleavage selectivity over off-target proteases ranges from 10 to greater than 10000 fold, with the higher selectivity achievable by extended-recognition substrates incorporating P3 and P4 positions in addition to the canonical P2-P1 cleavage motif. Manufacturing implications include solid-phase peptide synthesis of the linker, frequently as a heterobifunctional crosslinker with a maleimide, N-hydroxysuccinimide, click handle, or oligonucleotide phosphoramidite on the termini, with purification to greater than 95 percent purity by preparative reverse-phase HPLC and characterization by high-resolution mass spectrometry and analytical HPLC; in-process controls include amino acid analysis, residual reagent quantification, and orthogonal cleavage assay against the cognate enzyme to confirm functional integrity. Compatibility with the inner core is established by use of the peptide linker to tether payload molecules to core surface groups, with cleavage releasing free payload at the appropriate compartment; alternative architectures use the linker to tether multiple payload copies to a single core anchor in a branched or dendritic configuration, with sequential cleavage events releasing payload in a programmed dose. Compatibility with the outer shell is established by use of the linker as a cleavable tether for shell-displayed ligands, as a crosslinker between shell domains, or as a connector between the inner core and the outer shell, with the cleavage event triggering shell shedding, core decompaction, or payload release as appropriate to the architecture. For platforms requiring both extracellular and intracellular protease triggers, combinations of MMP-cleavable linkers in the outer shell and cathepsin-cleavable linkers in the inner core provide compartment-keyed sequential release.

### Enzymatic-Responsive Nucleic Acid Elements

Enzymatic-responsive nucleic acid elements provide a complementary enzymatic trigger class wherein cleavage of defined DNA or RNA segments by endogenous nucleases, or by co-delivered catalytic nucleic acids, drives release of payload, exposure of cryptic elements, or reconfiguration of nanostructure architecture. Endonuclease-cleavable sequences include single-stranded regions susceptible to endolysosomal DNase II, double-stranded regions susceptible to extracellular DNase I, and RNA segments susceptible to RNase A-family enzymes and compartment-specific RNases. By placement of single-stranded gaps, mismatched bulges, or modified internucleotide linkages with defined enzymatic susceptibility within the structural backbone of the outer shell or within tethers to the inner core, cleavage timing is programmed to the desired compartment. Ribozyme-controlled switches employ catalytic RNA segments such as the hammerhead, hairpin, hepatitis delta virus, and twister ribozyme classes engineered as ligand-gated allosteric switches that cleave a target RNA strand only when a small-molecule, metabolite, or oligonucleotide input is present; engineered aptazymes incorporating an aptamer recognition domain coupled to a ribozyme catalytic domain provide programmable input-output relationships with sensitivity tunable across multiple orders of magnitude of input concentration. DNAzyme-based switches employ catalytic DNA oligonucleotides such as the 10-23 and 8-17 deoxyribozyme classes that cleave RNA substrates in a sequence-specific manner with rate enhancement of from 1 thousand to 1 million fold over background in the presence of divalent metal cofactors including magnesium, zinc, manganese, or lead at concentrations of from 0.1 to 50 millimolar. DNAzyme switches may be engineered to function only at specific pH ranges, only with specific metal cofactors, or only when activated by a cognate input strand, providing logical control over cleavage. Endonuclease-cleavable sequences and DNAzyme switches are integrated into the outer DNA origami shell as structural staples or as linker strands, such that cleavage releases shell components and triggers decompaction; ribozyme switches are integrated as RNA segments embedded in chimeric RNA-DNA architectures.

Kinetics of enzymatic nucleic acid cleavage are described by single-turnover and multi-turnover catalytic constants depending on whether the catalytic species is consumed or regenerated, with cleavage half-times of from 10 milliseconds to 30 minutes at typical endogenous nuclease concentrations and from 1 second to 60 minutes for engineered ribozyme and DNAzyme switches operating intramolecularly or in trans. The differential between cleavage rate in the trigger compartment and in non-trigger compartments ranges from 100 to greater than 10000 fold, with the higher selectivity achievable by combination of compartment-specific cofactor requirements such as the elevated divalent metal concentration of endolysosomes and the elevated potassium concentration of cytosol. Manufacturing implications include solid-phase synthesis of oligonucleotide components with site-specific incorporation of cleavage-resistant modifications such as 2′-O-methyl, 2′-fluoro, phosphorothioate, or locked nucleic acid where stability is required, and incorporation of cleavage-susceptible native phosphodiester linkages at the intended cleavage site; quality control includes denaturing polyacrylamide gel electrophoresis to confirm full-length product, mass spectrometry to confirm modification placement, and orthogonal cleavage assays against the cognate nuclease or in trigger-mimicking buffers to confirm functional cleavage. Compatibility with the inner core is established by chemical conjugation of the nucleic acid element to the core surface or by hybridization to a complementary handle, with cleavage releasing the tethered payload; compatibility with the outer shell is established by integration of the element as a structural component of the DNA origami architecture, with cleavage triggering release events ranging from local opening of a single shell aperture to global decompaction of the entire outer shell. For platforms requiring orthogonal control by both protein nucleases and DNAzymes, distinct cleavage motifs are placed in defined positions, providing addressable release pathways.

### Redox-Responsive Elements

Redox-responsive elements exploit the substantial concentration gradient of intracellular reducing agents, principally glutathione at intracellular concentrations of from 1 to 10 millimolar versus extracellular concentrations of from 1 to 20 micromolar, a difference of three orders of magnitude providing one of the steepest natural triggers available for compartment-selective release. Disulfide bonds cleavable by intracellular glutathione exhibit cleavage half-times of from 10 minutes to 24 hours in extracellular fluid and of from 0.1 second to 30 minutes in cytosol or glutathione-rich organelles, with the rate strongly dependent on disulfide steric and electronic environment; aromatic disulfides cleave faster than aliphatic, and hindered tertiary disulfides cleave slower than unhindered primary or secondary disulfides. Diselenide linkages, formed by oxidation of two selenols to a Se—Se bond, exhibit lower bond-dissociation energies than disulfides and cleave under milder reducing conditions, with characteristic half-times of from 1 to 60 minutes in cytosolic glutathione; diselenides additionally respond to reactive oxygen species via oxidation to seleninic acid and selenonic acid intermediates that fragment, providing dual redox sensitivity. Thioether-quinone linkers comprise a quinone moiety conjugated through a thioether bond that undergoes reductive activation in cells expressing NAD(P)H quinone oxidoreductase 1, an enzyme upregulated in many tumors and in oxidatively stressed tissues; reduction of the quinone to the corresponding hydroquinone triggers intramolecular cyclization and payload liberation. Selenoether linkages, in which a selenium atom bridges two carbon substituents, undergo oxidative cleavage under elevated reactive oxygen species to yield selenoxide intermediates that undergo syn-elimination to liberate olefin and selenenic acid products. Combination of glutathione-cleavable disulfide and reactive-oxygen-species-cleavable selenoether or thioketal elements within a single architecture provides redundant cytosol-selective release with reduced sensitivity to single-pathway perturbation.

Manufacturing implications of redox-responsive elements include incorporation of disulfide bonds via thiol-disulfide exchange chemistry between two cysteine-equivalent thiol groups, via direct coupling of pyridyldithio activated species with target thiols, or via solid-phase oligonucleotide or peptide synthesis with disulfide-containing phosphoramidites or amino-acid derivatives. Diselenide and selenoether elements require specialized selenium-containing building blocks and additional precautions to prevent premature oxidation during synthesis and storage, including degassed solvents, inert atmosphere handling, and addition of low concentrations of reducing buffer such as tris(2-carboxyethyl)phosphine where compatibility permits. Quality control includes Ellman's assay or equivalent thiol quantification to confirm complete oxidation to disulfide, mass spectrometry to confirm correct linkage formation, and stability studies under simulated extracellular conditions to confirm absence of premature cleavage over the intended residence time. Storage stability of redox-responsive elements is preserved by exclusion of trace reducing agents and oxygen from formulations, with frozen storage at −20 degrees Celsius or below and shelf-life qualification typically yielding stable retention of greater than 90 percent intact linker over 24 months. Compatibility with the inner core is established by use of redox linkers to tether payloads, with cleavage in cytosol triggering payload release; for nucleic acid payloads sensitive to reducing conditions, the cleavage product is engineered to retain payload integrity. Compatibility with the outer shell is established by use of redox-cleavable crosslinkers within the shell or between shell and core, with cleavage triggering decompaction following endosomal escape into the reducing cytosol. The complementary combination of redox-responsive elements with pH-responsive and enzyme-responsive elements within a single architecture provides multi-compartment programmed release with high specificity.

### Reactive-Oxygen-Species-Responsive Elements

Reactive-oxygen-species-responsive elements respond to hydrogen peroxide, superoxide, hydroxyl radical, hypochlorite, and peroxynitrite, with intracellular reactive-oxygen-species concentrations elevated in inflamed tissue, solid tumors, ischemic-reperfusion regions, and mitochondrially stressed cells, providing disease- and organelle-selective triggering distinct from glutathione-driven reduction. Thioketal linkers comprise two thioether sulfurs bridged by a quaternary central carbon and undergo oxidative cleavage to ketones and thiols upon exposure to reactive oxygen species at concentrations of from 10 micromolar to 1 millimolar with half-times of from 1 to 60 minutes, with the rate tunable by central-carbon substitution; thioketal linkers offer the additional advantage of stability against intracellular glutathione, providing orthogonal control with respect to disulfide-based redox elements. Phenylboronic ester linkers comprise an aryl boronic acid esterified with a diol and undergo selective cleavage by hydrogen peroxide via oxidation of the carbon-boron bond to a phenol with release of the diol, with cleavage half-times of from 10 minutes to 8 hours at intracellular hydrogen peroxide concentrations and selectivity for hydrogen peroxide over other reactive species and common biological nucleophiles. Arylsulfide linkers undergo oxidation by reactive oxygen species to sulfoxides and sulfones, with the sulfoxide intermediate undergoing solvolysis at rates dependent on aryl substitution, providing a tunable release with half-times of from 30 minutes to 24 hours. Aryloxalate linkers, comprising a phenolic ester of oxalic acid, react with hydrogen peroxide to yield dioxetanedione intermediates that decompose with release of phenol and emission of chemiluminescence, providing simultaneous reactive-oxygen-species-triggered payload release and optical reporter signal useful for imaging-coupled therapeutic applications. Combination of multiple reactive-oxygen-species-responsive linker chemistries within a single architecture, each tuned to a different threshold, permits graded response to local oxidative status.

Manufacturing implications of reactive-oxygen-species-responsive elements include solution-phase synthesis of the linkers using standard organic transformations, with thioketal synthesis from a ketone and two thiol equivalents under acid catalysis, boronic ester synthesis by condensation of a boronic acid and a diol with azeotropic water removal, arylsulfide synthesis by thiol-aryl-halide coupling, and aryloxalate synthesis by acylation of a phenol with oxalyl chloride or oxalic acid derivatives. Stability under processing conditions is preserved by exclusion of oxygen during synthesis and storage and by use of antioxidant excipients during formulation, including ascorbate, tocopherol, butylated hydroxytoluene, and butylated hydroxyanisole at concentrations compatible with subsequent in vivo administration. Quality control includes nuclear magnetic resonance for structural confirmation, high-resolution mass spectrometry for molecular weight verification, and orthogonal oxidative cleavage assays under reactive-oxygen-species mimicking conditions including incubation with hydrogen peroxide at 1 millimolar concentration to confirm cleavage at expected rates. The differential between cleavage rate in reactive-oxygen-species-rich pathologic tissue and in healthy tissue ranges from 10 to greater than 1000 fold, with the higher selectivity achievable by linker chemistries specific to hydrogen peroxide over other reactive species. Compatibility with the inner core is established by use of these linkers as cleavable tethers for payloads sensitive to oxidative environments, with appropriate antioxidant excipient inclusion to protect both linker and payload during transit; compatibility with the outer shell is established by use as oxidation-cleavable crosslinkers within the shell or between architectural layers, with cleavage triggered upon entry into reactive-oxygen-species-rich compartments. Combination with redox-responsive disulfide elements provides redundant cytosol-selective release that proceeds via either reducing or oxidizing trigger, with the combination offering robustness against pathologic perturbation of either redox pathway.

### Photo-Responsive Elements

Photo-responsive elements provide an externally addressable trigger class enabling spatial and temporal control of release through delivery of light at defined wavelength, intensity, and duration to the platform after administration. Ortho-nitrobenzyl photolabile groups undergo Norrish-type photocleavage upon absorption of ultraviolet light at 320 to 365 nanometers with quantum yields of from 0.05 to 0.6 depending on substitution, releasing a carboxylic acid, alcohol, amine, or thiol leaving group on the millisecond to second timescale; substitution at the benzylic position with a methyl or carboxyl group, or on the aromatic ring with methoxy, dimethoxy, or veratryl moieties, shifts the absorption maximum and tunes the byproduct profile. Coumarin and 7-aminocoumarin photolabile groups absorb in the near-ultraviolet to visible range of from 350 to 470 nanometers depending on substitution, with quantum yields of from 0.01 to 0.3, and undergo heterolytic cleavage to release the protected leaving group with formation of a coumarinyl alcohol byproduct; the longer wavelength absorption of coumarin classes provides reduced phototoxicity and deeper tissue penetration. Azobenzene photoswitches undergo reversible trans-to-cis photoisomerization upon ultraviolet illumination at 320 to 365 nanometers and reverse cis-to-trans isomerization upon visible illumination at 420 to 470 nanometers or by thermal relaxation, with the resulting geometric change of from 0.9 to 0.5 nanometers in end-to-end distance driving conformational changes in attached structures that can open or close apertures, displace cargo, or modulate ligand presentation. The reversibility of azobenzene photoswitches permits use as a binary switch with bidirectional optical control, distinguishing this class from the irreversibly photocleavable ortho-nitrobenzyl and coumarin classes.

Near-infrared activation, required for in vivo applications in which ultraviolet penetration is limited to fractions of a millimeter, is achieved via upconversion nanoparticles comprising lanthanide-doped sodium yttrium fluoride or related host matrices that absorb 980 or 808 nanometer near-infrared photons and emit upconverted ultraviolet or visible light at 365, 450, or 540 nanometers that activates a colocalized photolabile group; the upconversion efficiency of from 0.1 to 5 percent under continuous wave excitation at 1 to 10 watts per square centimeter is sufficient for practical photoactivation in tissues of from 1 to 30 millimeter depth, with the precise penetration depth determined by tissue scattering and absorption at the excitation wavelength. Two-photon-activated chemistries, in which simultaneous absorption of two near-infrared photons drives a single electronic excitation equivalent to absorption of one photon at half the wavelength, provide an alternative deep-tissue photoactivation modality, with two-photon cross-sections of from 0.01 to 100 Goeppert-Mayer units characterizing the relative activatability of different photolabile groups under pulsed near-infrared illumination at 700 to 900 nanometers and at intensities of from 10 to 1000 megawatts per square centimeter in focal volumes of from 0.1 to 10 cubic micrometers. Manufacturing implications include synthesis of the photolabile group as a heterobifunctional crosslinker by solution-phase organic chemistry with protection of photolabile groups from ambient light at all stages, and quality control by ultraviolet-visible spectroscopy to confirm characteristic absorption maxima and by mass spectrometry to confirm structural identity. Upconversion nanoparticles are synthesized by thermal decomposition or hydrothermal methods with sizes of from 10 to 50 nanometers, with surface functionalization by silica overcoating, polymer coating, or ligand exchange. Compatibility with the inner core is established by integration of upconversion nanoparticles into the core matrix and by tethering of photolabile groups to payload molecules; compatibility with the outer shell is established by attachment of photolabile groups as cleavable shell crosslinkers or by display of azobenzene photoswitches that open and close shell apertures upon illumination.

### Thermosensitive Elements

Thermosensitive elements respond to temperature shifts arising from external focused heating, focal disease-associated hyperthermia, or physiologic temperature gradients, with response thresholds engineered into the range of from 37 to 45 degrees Celsius for in vivo applications. Poly(N-isopropylacrylamide) and related thermoresponsive polymers exhibit a lower critical solution temperature, classically near 32 degrees Celsius, that may be tuned to from 37 to 42 degrees Celsius by copolymerization with hydrophilic monomers including acrylamide, acrylic acid, or polyethylene glycol methacrylate; below the lower critical solution temperature the polymer is hydrated and extended, while above it the polymer dehydrates and collapses, driving structural transitions in attached or matrix-incorporated structures. Elastin-like polypeptides, comprising recombinant or synthetic repeats of pentapeptide units, exhibit an inverse transition temperature tunable by repeat composition, molecular weight, and added salt to fall in the range of from 37 to 42 degrees Celsius. Thermosensitive lipids, exemplified by phosphatidylcholine and phosphatidylglycerol formulations with defined acyl-chain saturation profiles and incorporation of lyso-lipids, exhibit gel-to-liquid-crystalline phase transitions tunable to the hyperthermia range, with membrane permeability increasing by orders of magnitude across the transition and providing rapid payload release upon thermal trigger. Shape-memory polymers store a deformed configuration that returns to a defined permanent shape upon heating above a programmed transition temperature, with the transition tunable by polymer composition and crosslink density; shape-memory elements may be used to drive geometric reconfiguration of the outer shell upon thermal triggering, opening apertures or shedding outer layers. Kinetics of thermal release are limited by heat transfer and polymer chain dynamics, with response times of from 0.1 second to 30 minutes following thermal equilibration achieved by focused ultrasound, magnetic hyperthermia, infrared illumination, or microwave hyperthermia. Manufacturing implications include controlled radical polymerization of N-isopropylacrylamide-class monomers, recombinant expression of elastin-like polypeptides, lipid film hydration for thermosensitive liposomes, and shape-memory polymer fabrication by solution casting. Compatibility with the inner core is established by incorporation of thermosensitive lipids as core membrane components or by conjugation of thermosensitive polymer chains to core surface, and compatibility with the outer shell is established by display of thermosensitive chains on the shell surface, by inclusion of thermosensitive crosslinkers, or by integration of shape-memory polymer struts that geometrically reconfigure the shell upon heating.

### Ultrasound-Responsive Elements

Ultrasound-responsive elements respond to mechanical and thermal effects of focused acoustic energy delivered at frequencies of from 0.5 to 10 megahertz and intensities of from 0.1 to 100 watts per square centimeter, providing externally addressable, deep-tissue triggering without ionizing radiation. Microbubble cavitation employs gas-cored microbubbles of from 1 to 5 micrometer diameter stabilized by lipid, polymer, or protein shells, which upon ultrasound exposure undergo stable oscillation at low pressure and inertial collapse at higher pressure with localized release of mechanical energy as shock waves, microjets, and transient elevated temperature; the cavitation threshold is determined by microbubble shell composition, gas core identity such as perfluoropropane or sulfur hexafluoride, and ambient acoustic pressure, with mechanical indices of from 0.1 to 2.0 separating stable from inertial cavitation regimes. Sonoporation, a consequence of cavitation adjacent to cell membranes, transiently permeabilizes the plasma membrane with pores of from 10 to 500 nanometers persisting for seconds to minutes, providing intracellular delivery of co-administered payload. Ultrasound-tunable polymer matrices comprise hydrogels, microspheres, or shell-stabilizing polymers in which acoustic energy disrupts crosslinks, induces local heating, or drives cavitation of trapped gas pockets, with payload release proportional to acoustic dose. Kinetics are determined by the acoustic dose, with release proportional to time-integrated intensity above the cavitation or thermal threshold, and characteristic release times of from 0.1 second to 30 minutes following onset of exposure. Manufacturing implications include preparation of stabilized microbubbles by mechanical agitation, microfluidic flow focusing, or coaxial electrohydrodynamic atomization with size sorting by flotation or filtration; co-formulation with the shell-core assembly is performed by physical mixing or by direct integration of gas-filled vesicles into the inner core. Quality control includes acoustic attenuation spectroscopy to characterize size and stability, and in vitro acoustic release assays to confirm dose-response and threshold consistency. Compatibility with the inner core is established by incorporation of gas-filled microvesicles within the core or inclusion of acoustic-sensitive polymer matrices, and compatibility with the outer shell is established by use of acoustic-cleavable crosslinkers within the shell, with cavitation events at the shell surface triggering disruption and release of inner core contents.

### Magnetic-Stimulus-Responsive Elements

Magnetic-stimulus-responsive elements respond to externally applied magnetic fields through mechanical actuation, hyperthermic heating, or guided localization, with iron oxide nanoparticles of from 5 to 50 nanometer diameter serving as the principal component owing to their biocompatibility, regulatory precedent in approved imaging agents, and tunable magnetic properties. Iron-oxide-mediated actuation employs static or low-frequency oscillating fields to apply piconewton-scale forces on nanoparticle clusters embedded in the platform, with the resulting strain disrupting weak crosslinks, opening tethered apertures, or driving directional motion under applied field gradients of from 10 to 1000 tesla per meter. Magnetic-hyperthermia release in alternating fields of from 100 to 700 kilohertz and amplitudes of from 5 to 50 kiloamperes per meter generates heat through Neel and Brown relaxation of single-domain iron oxide particles, with specific absorption rates of from 100 to 1500 watts per gram iron yielding local temperature elevations of from 2 to 15 degrees Celsius and triggering colocalized thermosensitive release elements without bulk tissue heating; spatial selectivity is determined by field-applicator geometry and by local iron oxide concentration, with focal-volume control achievable in single-cubic-centimeter regions. Kinetics of magnetic actuation occur on the millisecond timescale, while hyperthermia release follows the thermal response of colocalized thermosensitive elements with characteristic times of from 10 seconds to 30 minutes depending on iron loading, field amplitude, and thermal element transition. Manufacturing implications include synthesis of iron oxide nanoparticles by thermal decomposition or coprecipitation with size control to within 10 percent, surface functionalization by silica overcoating or polymer ligand exchange, and incorporation into the platform within the inner core, as a surface layer, or embedded within shell matrix. Quality control includes vibrating-sample magnetometry to characterize saturation magnetization and coercivity, dynamic light scattering for hydrodynamic size, and calorimetric measurement of specific absorption rate under alternating field exposure. Compatibility with the inner core is established by direct inclusion of iron oxide nanoparticles within the core matrix, and compatibility with the outer shell is established by tethering iron oxide particles to the shell exterior via cleavable or non-cleavable linkers.

### Mechanical-Stress-Responsive Elements

Mechanical-stress-responsive elements respond to forces arising from physiologic shear, focal pressure, or applied external mechanical perturbation, with response thresholds engineered into the range of pathologically elevated stress while remaining stable at physiologic baseline. Shear-stress linkers comprise polymer or peptide tethers with force-dependent bond rupture thresholds of from 10 to 200 piconewtons, cleaving preferentially in high-shear vascular regions such as stenotic arteries with wall shear stresses of from 100 to 1000 dynes per square centimeter or in mechanically perturbed tissues; the rupture threshold is tunable by polymer chemistry, tether length, and inclusion of sacrificial bonds. Mechanophore polymers incorporate force-sensitive chemical groups, including spiropyran ring-opening units, gem-dichlorocyclopropane fragmentation units, and Diels-Alder retro-cycloaddition units, that undergo defined chemical transformations under tensile or compressive force, releasing reporter or therapeutic payload proportional to mechanical loading; spiropyran-based mechanophores additionally undergo a colorimetric transition upon mechanoactivation, providing a self-reporting feature useful in formulation development. Pressure-responsive architectures comprise porous matrices, collapsible vesicles, or compressible polymer networks that release payload upon hydrostatic or external pressure exceeding a programmed threshold of from 0.1 to 10 megapascals. Kinetics of mechanical release follow a Bell-Evans exponential relationship, with characteristic release times decreasing by orders of magnitude across the design force window. Manufacturing implications include incorporation of mechanophore monomers by controlled radical or step-growth polymerization, peptide-tether attachment by solid-phase synthesis, and pressure-vesicle fabrication by extrusion or microfluidic templating; quality control includes single-molecule force spectroscopy or bulk rheological characterization. Compatibility with the inner core is established by use of mechanical linkers as cleavable tethers within the core matrix, and compatibility with the outer shell is established by inclusion of mechanophore polymer struts or shear-stress linkers as connectors between shell and core.

### Logic-Gated Multi-Input Release

Logic-gated multi-input release architectures combine two or more of the foregoing trigger classes into structures that release payload only upon coincident, sequential, or threshold-summed satisfaction of multiple input conditions, providing dramatically improved specificity over single-trigger release and enabling pathology-selective release at the intersection of multiple disease markers. pH-AND-enzyme gates require both acidic environment and enzymatic activity for release; one implementation incorporates an enteric pH-responsive outer coating concealing an enzyme-cleavable inner linker, such that release occurs only when pH-driven coating dissolution is followed by enzymatic cleavage in the appropriate compartment. pH-OR-enzyme gates release upon either acidic environment or enzymatic activity, providing fault-tolerant release suitable for heterogeneous target populations; one implementation incorporates parallel pH-labile and enzyme-cleavable linkers tethering the same payload, with cleavage of either yielding free payload. Time-delayed redox gates couple a slow first step, such as enteric coating dissolution and transcytotic uptake taking minutes to hours, with a fast second step, such as cytosolic disulfide cleavage taking seconds to minutes, ensuring that release occurs only in the appropriate intracellular compartment and only after the requisite delivery delay. Threshold-summation gates accumulate input signals from multiple triggers, with release occurring only when the cumulative input exceeds a defined threshold; implementation employs DNA strand-displacement circuits in which multiple input strands progressively displace a payload-bearing strand from a holding complex. Feedback-controlled DNA logic circuits employ catalytic strand-displacement networks, hybridization chain reactions, and DNAzyme cascades to amplify weak inputs, integrate multiple signals over time, and produce switch-like output responses; such circuits provide tunable thresholds and may incorporate negative-feedback elements that suppress release at low input levels and positive-feedback elements that drive rapid release upon threshold crossing.

Kinetics of logic-gated release are determined by the slowest input-processing step in the cascade and by the architecture of the integrating circuit, with characteristic gate-completion times of from 1 minute to 24 hours and signal-to-noise discrimination ratios of from 5 to 1000 for typical disease-marker concentration differentials. Manufacturing implications include integrated assembly of multiple linker chemistries within a single architecture, requiring orthogonal conjugation chemistries to permit sequential assembly without cross-reactivity; the orthogonality may be ensured by use of chemically distinct conjugation handles such as amine, thiol, azide, dibenzocyclooctyne, tetrazine, trans-cyclooctene, and oligonucleotide hybridization across the multiple coupling steps required for full gate assembly. Design of DNA circuits is performed with computer-aided sequence-design software to avoid spurious hybridization, with sequence selection optimized for the intended energetic landscape across all designed conformations and against unintended off-pathway interactions. Quality control includes truth-table verification under defined input combinations to confirm logical correctness, with each gate tested against the full combinatorial input space and the resulting release quantified to confirm gain and threshold parameters; quantitative release assays under physiologically relevant input conditions confirm gate performance in mimicking buffers. Compatibility with the inner core and outer shell is established by integration of the gate elements as structural components of the architecture, with the output of the gate driving the release event appropriate to the architectural level at which the gate is incorporated; gates may be placed at the outer shell to control shell shedding, at intermediate layers to control sequential decompaction, or at the inner core to control terminal payload release, with each gate keyed to the input combination appropriate to the corresponding compartment. The combination of gate logic with the inherent compartment-selectivity of the underlying triggers yields cumulative selectivity factors that may exceed 100000 fold between trigger and off-trigger compartments.

### Sequential-Release Architectures

Sequential-release architectures program release events in defined temporal and spatial order from oral administration to intracellular destination, providing matched protection and exposure at each step. Outer-shell shedding at gastrointestinal pH transition employs an enteric polymer layer that dissolves at proximal small-intestinal pH, exposing the underlying DNA origami shell or biomimetic shell for subsequent transcytosis without prior acid exposure. Inner-core decompaction at endosomal acidification employs pH-responsive nucleic acid switches, polymer coatings, or chemical linkers within the inner core that respond to the pH transition from 7.4 in the extracellular space to of from 6.0 to 4.5 in the endolysosomal pathway, triggering release within the endosome or following endosomal escape into cytosol. Payload release via dual pH-AND-enzyme trigger combines pH-driven decompaction with concurrent enzymatic cleavage of payload tethers by endolysosomal cathepsins, providing both intracellular-compartment selectivity and temporal sharpening. Ratiometric sequential release programs the release of multiple distinct payloads at different stages of the delivery cascade, with rapidly releasing payloads providing initial activity, intermediately releasing payloads providing sustained activity, and slowly releasing payloads providing prolonged tail; ratios are controlled by the relative number of fast, medium, and slow linkers between the architecture and each payload class, with manufacturing precision of within 10 percent of design ratio achievable by stoichiometric control. Sequential architectures additionally incorporate transcytosis-triggered release at the basolateral enterocyte membrane, blood-brain-barrier-crossing-triggered release at brain endothelial tight junctions, and target-cell-surface-receptor-triggered release at the destination cell, with each event keyed to a distinct biological signature. Kinetics of sequential release are described by a cascade of first-order or pseudo-first-order steps, with cumulative release at each compartment determined by the product of upstream survival fractions and local release fraction. Manufacturing implications include independent qualification of each step under simulated compartment conditions, with end-to-end qualification under serial-compartment in vitro assays approximating the in vivo cascade. Compatibility with the inner core and outer shell is established by the layered architecture, in which sequential triggers act on sequential structural layers with each event exposing the next layer; design ensures that no trigger acts prematurely on an inappropriate layer and that all required triggers are encountered along the intended delivery path.

### Quantitative Release-Kinetic Ranges

Quantitative release-kinetic ranges for the foregoing trigger classes characterize expected platform behavior under physiologically relevant conditions and provide the basis for in vitro qualification and for in vivo pharmacokinetic modeling. For pH-responsive payload-tether linkers based on hydrazone, acetal, beta-thiopropionate, orthoester, vinyl ether, and cis-aconityl chemistries, expected payload release at pH 7.4 over a 1-hour interval is of from 0.5 to 10 percent of conjugated dose, over a 6-hour interval is of from 2 to 30 percent, and over a 24-hour interval is of from 8 to 60 percent. At pH 5.5 simulating endolysosomal exposure, expected release over a 1-hour interval is of from 20 to 90 percent, over a 6-hour interval is of from 60 to 99 percent, and over a 24-hour interval is of from 85 to greater than 99 percent. The ratio of release rate at pH 5.5 to release rate at pH 7.4 ranges from 10 to greater than 1000 across linker classes, with orthoester and cis-aconityl chemistries providing the steepest differential. For pH-responsive nucleic acid switches and polymer coatings, expected payload exposure at pH 7.4 over a 24-hour interval is of from 0 to 5 percent, with structures remaining intact, while at pH 5.5 structures undergo transition with completion of from 80 to greater than 99 percent within of from 1 to 60 minutes. For enteric polymer outer coatings, expected release at simulated gastric pH 1.2 over a 2-hour interval is of from 0 to 10 percent of total dose, while at simulated intestinal pH of from 5.5 to 7.0 release occurs within of from 30 to 120 minutes with completion of greater than 80 percent. For enzymatic peptide linkers, expected cleavage half-lives at lysosomal cathepsin concentrations of from 1 to 50 micromolar are of from 1 minute to 4 hours for cathepsin B substrates and of from 0.5 minutes to 2 hours for caspase-3 substrates in apoptotic cells; at extracellular matrix-metalloproteinase concentrations of from 1 to 100 nanomolar in remodeling tissue, cleavage half-lives are of from 10 minutes to 24 hours.

For redox-responsive disulfide linkers, expected cleavage half-lives at extracellular glutathione concentrations of from 1 to 20 micromolar are of from 10 minutes to 24 hours, while at intracellular glutathione concentrations of from 1 to 10 millimolar are of from 0.1 second to 30 minutes, providing a release differential between extracellular and cytosolic compartments of from 100 to 10000 fold. For reactive-oxygen-species-responsive linkers, expected cleavage half-lives at normal physiologic levels are of from 12 to greater than 72 hours, while at pathologically elevated levels in inflamed or tumor tissue are of from 5 minutes to 8 hours. For photo-, ultrasound-, magnetic-, and mechanical-responsive elements, release kinetics are determined by applied stimulus dose and follow zero-order kinetics during continuous stimulation with characteristic release times of from 1 second to 30 minutes following stimulus onset; in the absence of applied stimulus, release is of from 0 to 2 percent over a 24-hour interval. For logic-gated and sequential-release architectures, expected release in any single compartment lacking the full required trigger combination is of from 0 to 5 percent of total dose, while expected release in the compartment satisfying the full trigger combination is of from 80 to greater than 99 percent within of from 5 minutes to 24 hours. These ranges define the design envelope for the platform and provide benchmarks against which manufactured lots are qualified in vitro prior to release for in vivo use. Lot-release acceptance criteria are established at the most clinically relevant pair of compartments for each configuration, with typical specifications requiring less than 10 percent off-target release over the relevant transit interval and greater than 70 percent on-target release within the clinically relevant exposure interval, providing a quantitative basis for batch acceptance and rejection.

### Expanded Axis 6—Surface Targeting and Stealth (Deep Dive)

### Stealth Modifications and Anti-PEG-Antibody Avoidance

Within the embodiments of Axis 6, the exterior surface of the protective outer shell is modified by conjugation of polyethylene glycol (PEG) chains of molecular weight of from 1,000 daltons to 20,000 daltons at a surface coverage density of from 5 percent to 50 percent of available conjugation sites, configured to provide stealth function through reduction of non-specific protein adsorption and reduction of mononuclear phagocyte system clearance. PEG architectures include linear methoxy-PEG, branched two-arm and four-arm PEG, and amine-functional PEG variants. Alternative or complementary stealth coatings include zwitterionic coatings selected from polysarcosine of from 5 to 100 sarcosine residues, poly(carboxybetaine), poly(sulfobetaine), and poly(2-methacryloyloxyethyl phosphorylcholine), in each case at a surface coverage comparable to that of PEG. Zwitterionic coatings provide an anti-PEG-antibody avoidance strategy for patient populations exhibiting pre-existing anti-PEG IgM or IgG responses observed in published clinical literature, and may be selected as primary stealth coating in such populations.

### Targeting Ligand Classes for Dual-Barrier Crossing

Surface targeting ligand classes for gastrointestinal-barrier crossing include M-cell-targeting peptide ligands of from 5 to 30 amino acid residues identified by phage display, antibody fragments specific for M-cell surface markers including glycoprotein 2 and beta-1 integrin, carbohydrate moieties including mannose, fucose, and beta-glucan classes binding M-cell lectins, aptamer ligands selected for M-cell binding, and bacterial-invasion-protein-derived peptide ligands of the Salmonella SipB family, Yersinia YadA family, and Listeria Internalin A family engineered to retain receptor binding without infectious capacity. Surface targeting ligand classes for blood-brain barrier crossing include peptide ligands of from 5 to 35 amino acid residues configured to bind one or more of the transferrin receptor, insulin receptor, insulin-like growth factor 1 receptor, LRP1, LRP8/ApoER2, leptin receptor, nicotinic acetylcholine receptor, CD98 hc, and basigin with an apparent dissociation constant in the range of from 50 nanomolar to 500 nanomolar; antibody fragments engineered for moderate-affinity binding to the foregoing receptors in single-chain variable fragment, single-domain antibody, and engineered low-affinity bispecific shuttle formats; and aptamer ligands selected for the foregoing receptors.

### Cell-Penetrating Peptide Classes and Dual-Targeting Architectures

Cell-penetrating peptide classes presented on the outer shell or on the inner core to facilitate adsorptive-mediated transcytosis include the TAT family of cationic peptides derived from the trans-activator of transcription protein, the penetratin family of amphipathic peptides derived from the antennapedia homeodomain, polyarginine class peptides of from 6 to 12 arginine residues, and the MAP family of amphipathic peptides (all described as functional classes without literal sequences). Dual-targeting architectures combine a gastrointestinal-targeting ligand carried on the outermost shell layer with a blood-brain-barrier-crossing ligand carried on a sub-layer that is exposed only after dissolution of the outermost layer in response to the gastrointestinal pH and protease environment, providing temporal separation of the two targeting modalities along the dual-barrier transit path. Stimulus-responsive ligand shedding architectures additionally employ stimulus-responsive linkers (as set forth in Axis 5) between the outermost stealth coating and the underlying shell, such that the stealth coating is shed in response to a defined trigger and previously masked targeting moieties become accessible.

### Surface Conjugation Chemistries, Density Optimization, and Biomimetic Membrane Coatings

Surface conjugation chemistries for attaching the foregoing ligand and stealth classes include N-hydroxysuccinimide-ester conjugation to surface-exposed lysine residues in phosphate buffer at pH of from 7.5 to 8.5; maleimide-thiol conjugation to engineered cysteine residues under mild reducing conditions; copper-catalyzed (CuAAC) and copper-free (SPAAC) azide-alkyne click chemistry employing engineered alkyne and azide handles; inverse electron-demand Diels-Alder (IEDDA) cycloaddition between engineered tetrazine and trans-cyclooctene handles; SpyTag-SpyCatcher covalent isopeptide bond formation between engineered SpyTag-containing ligand and SpyCatcher-containing shell subunit; and sortase A-mediated transpeptidation between engineered LPXTG-containing ligand and glycine-containing shell subunit. Ligand density is controlled by molar ratio during conjugation, with optimal density ranges of from 10 to 200 ligands per particle depending on ligand class, target receptor density on the relevant epithelium, and desired avidity. Biomimetic membrane coatings as a combined stealth-plus-targeting strategy include coatings derived from red blood cell membrane (retaining CD47 don't-eat-me signaling), platelet membrane (retaining P-selectin and integrin alpha-IIb-beta-3 for targeted adhesion at sites of vascular injury), leukocyte membrane (retaining selectin and integrin profiles for targeted adhesion at sites of inflammation), homologous tumor cell membrane (providing self-recognition for autologous tumor targeting), and exosome membrane (retaining tetraspanin and adhesion molecule complements). Brush-border-mimetic and mucoadhesive coatings of the chitosan class, thiolated polymer class, and lectin-mediated mucoadhesive class are further available for extended intestinal residence time. Zeta potential is tuned across the range of from minus 20 millivolts to plus 10 millivolts depending on the barrier-crossing stage targeted, with slightly negative to near-neutral zeta potentials preferred for blood-brain barrier crossing and cationic surfaces preferred for endosomal escape stages.

### Expanded Axis 7—Therapeutic Indication (Deep Dive)

### Therapeutic Indications

The therapeutic indications addressable by the disclosed oral nanostructure drug delivery platform encompass a comprehensive range of central nervous system disorders for which simultaneous traversal of the intestinal epithelial barrier and the blood-brain barrier provides a definite pharmacological advantage. The platform comprises a multi-compartment shell-core architecture configured to survive gastric pH ranging from 1.2 to 3.5 for 30 to 120 minutes, transit the small intestinal lumen at pH 5.5 to 7.4 for 90 to 240 minutes, and engage transcytosis-competent receptors at the intestinal apical membrane and at brain microvascular endothelial cells with apparent binding affinities in the range of 1 to 100 nanomolar. Across the indication classes described herein, the platform achieves an estimated brain-to-plasma partition coefficient of 0.15 to 0.65 measured by simultaneous cisternal cerebrospinal fluid and venous plasma sampling at 1, 4, 12, and 24 hours post-administration. The indications are organized by neuroanatomical and pathophysiological grouping and are individually addressable by selection of (i) targeting ligand class, (ii) payload class, (iii) release-trigger mechanism, (iv) administration schedule, and (v) clinical monitoring biomarker panel. The selections recited in the following paragraphs are non-limiting embodiments and may be combined as combinatorial therapeutic regimens within a single dosing protocol.

Alzheimer disease and pre-clinical Alzheimer states constitute a primary indication for the disclosed platform across the full disease continuum spanning amyloid-positive cognitively unimpaired individuals through severe dementia. Pathophysiology comprises the deposition of amyloid-beta peptide aggregates of 40 and 42 amino-acid lengths into extracellular plaques accompanied by intraneuronal accumulation of hyperphosphorylated tau protein in paired helical filaments, synaptic loss progressing at a rate of 1.5 to 4 percent per year as measured by cortical thickness on volumetric magnetic resonance imaging, microglial activation with elevated soluble TREM2 in cerebrospinal fluid in the range of 4,000 to 10,000 picograms per milliliter, and progressive neuronal loss in hippocampal CA1, entorhinal cortex, and basal forebrain cholinergic nuclei. Prior-art treatment with monoclonal antibodies including lecanemab and donanemab requires biweekly to monthly intravenous infusions of 10 milligrams per kilogram administered in clinical infusion suites, produces amyloid-related imaging abnormalities of edema and microhemorrhage in 12 to 35 percent of treated subjects, and addresses only the amyloid axis of pathophysiology. The oral platform disclosed herein delivers an estimated 0.5 to 5 percent of an orally administered antibody-equivalent dose to brain parenchyma without infusion-suite logistics, enables daily or three-times-weekly dosing in home settings, and accommodates payload combinations addressing amyloid, tau, and neuroinflammatory targets in a single dosage unit. Preferred payload classes for early and preclinical AD include single-chain antibody fragments and bispecific nanobodies of 12 to 30 kilodaltons targeting amyloid-beta oligomers and pyroglutamate-modified amyloid-beta species; preferred payloads for moderate to severe AD include tau-targeting antisense oligonucleotides of 15 to 25 nucleotides, anti-tau immunotherapeutics, and small-molecule O-GlcNAcase inhibitors of 250 to 600 daltons.

Release-trigger preference for Alzheimer-disease embodiments comprises a tandem trigger architecture in which the outer shell releases payload at the blood-brain barrier endothelial-cell luminal surface in response to receptor-mediated endocytosis through transferrin receptor or insulin receptor binding, followed by intracellular endosomal acidification at pH 5.0 to 6.0 triggering payload release into brain parenchyma within 10 to 60 minutes of internalization. For familial Alzheimer disease populations carrying PSEN1, PSEN2, or APP autosomal-dominant mutations, the platform is dosed beginning 10 to 15 years prior to predicted symptom onset based on parental age of onset, delivering gamma-secretase modulator small molecules or APP-targeting antisense oligonucleotides at a frequency of three times per week. For APOE4 homozygous and heterozygous individuals, the platform incorporates APOE-targeting antisense oligonucleotides or APOE-isoform-converting protein payloads. Biomarker monitoring across all AD subgroups comprises plasma phosphorylated-tau-217 measured every 3 months with target reduction of 30 to 60 percent from baseline, plasma glial fibrillary acidic protein measured every 6 months, cerebrospinal fluid amyloid-beta-42 to amyloid-beta-40 ratio measured at baseline, 12 months, and 24 months, and amyloid positron emission tomography centiloid value measured at baseline, 18 months, and 36 months with target reduction of 25 to 70 centiloids. Clinical-development considerations include a regulatory pathway following the 2018 FDA AD framework permitting biomarker-supported accelerated approval, requirement for cardiac safety monitoring given gastrointestinal absorption pharmacokinetics, and parallel development of companion diagnostic for amyloid PET and plasma p-tau-217.

Parkinson disease and atypical parkinsonian syndromes constitute a second indication class for which the oral platform provides advantage over current intravenous, subcutaneous, intrajejunal, and deep-brain-stimulation modalities. Pathophysiology of idiopathic Parkinson disease comprises the misfolding of alpha-synuclein protein into oligomers and amyloid fibrils that deposit as Lewy bodies and Lewy neurites with prion-like propagation from enteric nervous system to dorsal motor nucleus of vagus and subsequently to substantia nigra pars compacta dopaminergic neurons; nigral dopaminergic neuron loss progresses at 4 to 10 percent per year with motor symptoms emerging at 50 to 70 percent neuronal loss. Prodromal PD encompassing rapid-eye-movement sleep behavior disorder and hyposmia identifies subjects 5 to 15 years prior to motor manifestation with annual phenoconversion rate of 6 to 8 percent. Multiple system atrophy involves oligodendroglial alpha-synuclein deposition; progressive supranuclear palsy and corticobasal degeneration involve 4-repeat tauopathy with median survival of 6 to 9 years from diagnosis. Prior-art treatment limitations include the requirement for carbidopa-levodopa dosing every 2 to 4 hours throughout the day with on-off motor fluctuations, dyskinesia in 50 to 80 percent of subjects after 5 years, surgical placement of deep brain stimulators with attendant infection and hardware-revision rates of 5 to 15 percent at 5 years, and absence of disease-modifying agents addressing alpha-synuclein pathology. The oral platform disclosed herein bypasses the unreliable absorption of levodopa across competing dietary amino acids, delivers anti-alpha-synuclein antibodies and antisense oligonucleotides directly to enteric and central nervous tissue, and supports once-daily dosing schedules.

Preferred payload classes for the Parkinson disease indication group comprise anti-alpha-synuclein nanobodies of 12 to 15 kilodaltons binding pathological oligomeric and fibrillar conformations with selectivity ratios of 50 to 500 over monomeric alpha-synuclein, SNCA-targeting antisense oligonucleotides of 18 to 22 nucleotides achieving 40 to 70 percent transcript knockdown, GBA-modulating molecular chaperones for GBA-mutation-positive subjects, LRRK2 kinase inhibitors of 350 to 550 daltons for LRRK2-mutation-positive subjects, and neurotrophic factor proteins including glial-derived neurotrophic factor delivered as encapsulated recombinant protein or mRNA encoding GDNF. For multiple system atrophy embodiments, the platform delivers payloads targeting oligodendroglial alpha-synuclein with co-delivery of remyelination-promoting payloads. For progressive supranuclear palsy and corticobasal degeneration, the platform delivers 4-repeat-tau-selective antisense oligonucleotides and microtubule-stabilizing small molecules. Release-trigger preference comprises a dual gastrointestinal-cleavable enteric coating dissolving at pH 5.5 to 6.5 in the proximal small intestine combined with brain-microvascular-endothelial-cell-cleavable disulfide linkers releasing payload upon glutathione exposure at intracellular concentrations of 1 to 10 millimolar. Biomarker monitoring comprises Movement Disorder Society Unified Parkinson Disease Rating Scale Part III measured every 3 months with target slowing of progression by 30 to 50 percent over 24 months, dopamine transporter single-photon-emission computed tomography striatal binding ratio measured every 12 months, plasma neurofilament light chain measured every 6 months, and skin-biopsy phosphorylated alpha-synuclein quantification annually. Clinical-development considerations include adaptive enrichment design based on prodromal-PD risk score, parallel development for atypical parkinsonian syndromes under FDA orphan-drug designation, and stratification by GBA, LRRK2, SNCA, and PRKN genotype.

Huntington disease and CAG-repeat-expansion disorders constitute an indication class characterized by autosomal-dominant inheritance of expanded trinucleotide repeats with full penetrance above defined repeat thresholds. Pathophysiology of Huntington disease comprises expansion of the CAG trinucleotide repeat in exon 1 of the HTT gene above 36 repeats with age of onset inversely correlated with repeat length according to the Langbehn formula, accumulation of N-terminal mutant huntingtin protein fragments forming intranuclear and cytoplasmic inclusions, progressive striatal medium-spiny-neuron loss with 50 to 95 percent caudate volume reduction at clinical diagnosis, cortical neuronal loss layers III, V, and VI, and somatic CAG repeat expansion in vulnerable neuronal populations driven by mismatch repair pathway activity. Juvenile Huntington disease with onset before age 20 involves repeat lengths above 60 and exhibits more rapid progression with prominent rigidity and seizures. Premanifest Huntington disease subjects identified by predictive genetic testing experience subtle neuropsychiatric and motor changes 10 to 15 years prior to formal clinical diagnosis. Prior-art treatment is limited to symptomatic agents for chorea including tetrabenazine and deutetrabenazine with depression and parkinsonism side effects in 20 to 40 percent of treated subjects; intrathecally administered antisense oligonucleotides require quarterly lumbar puncture procedures in interventional radiology suites and have shown mixed results in phase 3 trials. The oral platform disclosed herein delivers HTT-lowering antisense oligonucleotides, allele-selective small-interfering RNAs, and CAG-repeat-targeting payloads via daily or three-times-weekly oral dosing, avoiding intrathecal procedures and enabling earlier intervention in premanifest cohorts.

Preferred payload classes for Huntington disease and related CAG-expansion disorders comprise HTT-allele-selective antisense oligonucleotides of 18 to 22 nucleotides exploiting single-nucleotide polymorphisms in linkage disequilibrium with the expanded allele, non-allele-selective HTT-lowering antisense oligonucleotides for subjects without linkage-tagged polymorphisms, CAG-repeat-directed small molecules of 400 to 700 daltons, MSH3-targeting payloads to reduce somatic expansion, and zinc-finger transcription repressors of 30 to 50 kilodaltons delivered as encoded mRNA. For Huntington-like syndromes including HDL1 prion-related, HDL2 junctophilin-3, HDL3, and C9orf72-associated phenocopies, the platform employs gene-specific antisense payloads. Release-trigger preference comprises enteric pH-triggered release in proximal small intestine combined with neuronal-uptake-triggered cytoplasmic release using cathepsin-cleavable linkers exposed in late endosomes. Biomarker monitoring comprises cerebrospinal fluid mutant huntingtin protein measured by single-molecule arrays every 3 months with target reduction of 40 to 75 percent, plasma neurofilament light chain measured every 3 months with target stabilization, Unified Huntington Disease Rating Scale Total Motor Score and Total Functional Capacity measured every 6 months, and volumetric magnetic resonance imaging caudate and putamen volumes measured every 12 months. Clinical-development considerations include enrollment of premanifest subjects with CAG repeat length 40 to 50 and CAP score above defined thresholds, requirement for genetic counseling protocols, parallel development of allele-selective and non-allele-selective payload variants, and adaptive trial design with futility analyses at 12 and 24 months.

Amyotrophic lateral sclerosis and motor neuron diseases constitute an indication class with rapid clinical progression and high unmet medical need. Pathophysiology of ALS comprises progressive degeneration of upper motor neurons in primary motor cortex and lower motor neurons in brainstem and spinal cord anterior horn, with median survival of 3 to 5 years from symptom onset; approximately 10 percent of cases are familial with C9orf72 hexanucleotide repeat expansion accounting for 40 percent of familial cases, SOD1 mutations for 20 percent, FUS for 5 percent, and TARDBP for 4 percent. Pathological hallmarks include cytoplasmic TDP-43 aggregates in 97 percent of cases, mislocalized nuclear-cytoplasmic transport machinery, and stress granule pathology. Spinal muscular atrophy types I through IV result from biallelic SMN1 mutations with disease severity inversely correlated with SMN2 copy number; type I presents within 6 months with median survival under 2 years without therapy. Primary lateral sclerosis involves selective upper motor neuron degeneration with slower progression; progressive muscular atrophy involves selective lower motor neuron degeneration; hereditary spastic paraplegia comprises over 80 genetic subtypes with corticospinal tract degeneration. Prior-art treatment limitations include modest survival benefit of 3 to 6 months with riluzole and edaravone, weekly or biweekly intravenous edaravone infusions, intrathecal nusinersen for spinal muscular atrophy requiring lumbar puncture every 4 months, single-dose intravenous onasemnogene abeparvovec at cost exceeding 2 million United States dollars per dose with hepatotoxicity risk, and tofersen for SOD1-ALS requiring monthly intrathecal injection.

Preferred payload classes for motor neuron disease indications comprise gene-specific antisense oligonucleotides for SOD1, FUS, TARDBP, ATXN2, and C9orf72 of 16 to 25 nucleotides, SMN2 splice-switching oligonucleotides for spinal muscular atrophy, dipeptide-repeat-protein-targeting nanobodies for C9orf72-ALS, TDP-43 aggregation inhibitors and chaperone-modulating small molecules, and neurotrophic factor mRNA payloads encoding IGF-1, BDNF, or GDNF. The oral platform delivers payloads to both brain and spinal cord parenchyma via systemic circulation with capillary-density-matched distribution. Release-trigger preference comprises acid-stable enteric coating, transferrin-receptor or LRP1-mediated transcytosis across blood-brain and blood-spinal-cord barriers, and motor-neuron-selective uptake using rabies-virus-glycoprotein-derived peptide ligands of 29 amino acids or analogous neuronal-tropic peptides. Biomarker monitoring comprises plasma neurofilament light chain measured every month with target reduction of 25 to 60 percent, cerebrospinal fluid SOD1 protein for SOD1-ALS embodiments, ALS Functional Rating Scale Revised measured every 4 weeks, slow vital capacity every 8 weeks, and electrical-impedance myography every 12 weeks. Clinical-development considerations include adaptive platform-trial designs such as the HEALEY ALS Platform with shared placebo arm, enrichment by genetic subtype, presymptomatic dosing in genetic-mutation-carrier cohorts identified through cascade testing, and bulbar-onset versus limb-onset stratification. For SMA embodiments, the platform supports newborn-screening-positive presymptomatic dosing replacing current intrathecal and intravenous gene therapy modalities.

Frontotemporal dementia spectrum disorders constitute an indication class with high prevalence in early-onset dementia and substantial caregiving burden. Pathophysiology comprises focal degeneration of frontal and temporal lobes with three principal molecular subtypes: tauopathy with 3-repeat or 4-repeat or mixed tau inclusions accounting for 40 to 45 percent of cases, TDP-43 proteinopathy accounting for 50 percent of cases with subtypes A through E defined by inclusion morphology, and FUS proteinopathy accounting for 5 to 10 percent of cases. Approximately 30 to 50 percent of FTD cases are familial with mutations in MAPT, GRN, and C9orf72 accounting for over 90 percent of genetic cases. Behavioral variant FTD presents with disinhibition, apathy, loss of empathy, perseverative behaviors, and dietary changes; semantic variant primary progressive aphasia presents with loss of word meaning and semantic memory; non-fluent variant PPA presents with agrammatism and apraxia of speech; FTD with motor neuron disease combines behavioral or language symptoms with ALS-spectrum motor findings; FTD with parkinsonism associated with MAPT mutations presents with parkinsonism and behavioral change. Prior-art treatment is restricted to off-label symptomatic management; no disease-modifying therapies are approved. Preferred payload classes comprise GRN-elevating small molecules and progranulin-replacement protein payloads for GRN-mutation carriers, MAPT-lowering antisense oligonucleotides for MAPT-mutation tauopathies, C9orf72-directed antisense oligonucleotides for hexanucleotide-repeat-expansion carriers, and TDP-43 nuclear-import-restoring payloads. Release-trigger preference comprises enteric pH release followed by sortilin-mediated or VPS10P-domain-receptor-mediated brain uptake to target progranulin trafficking. Biomarker monitoring comprises plasma progranulin for GRN-carriers with target normalization above 80 nanograms per milliliter, cerebrospinal fluid poly-GP dipeptide repeat protein for C9orf72 carriers, plasma neurofilament light chain, and Frontotemporal Dementia Clinical Dementia Rating sum-of-boxes every 6 months. Clinical-development considerations include presymptomatic genetic-carrier trial designs leveraging the GENFI and ALLFTD natural history cohorts.

Spinocerebellar ataxias and inherited ataxias constitute an indication class characterized by progressive cerebellar dysfunction with varying inheritance and molecular etiologies. Pathophysiology of polyglutamine-disease SCAs including types 1, 2, 3, 6, and 7 involves CAG-repeat expansion in respective ataxin genes producing toxic polyglutamine-expanded proteins; Friedreich ataxia involves GAA-trinucleotide-repeat expansion in intron 1 of FXN producing frataxin deficiency with mitochondrial iron-sulfur cluster biogenesis impairment; ataxia-telangiectasia involves biallelic ATM mutations with defective double-strand-break DNA damage response; ataxia with vitamin E deficiency involves TTPA mutations impairing alpha-tocopherol transport. Cerebellar Purkinje cell loss progresses at rates measurable by volumetric magnetic resonance imaging at 1 to 4 percent per year. Prior-art treatment is largely symptomatic; for Friedreich ataxia, omaveloxolone provides modest improvement of approximately 1.55 points on modified Friedreich Ataxia Rating Scale over 48 weeks. Preferred payload classes comprise ataxin-gene-allele-selective antisense oligonucleotides for SCA1, SCA2, SCA3, and SCA7, frataxin-restoring mRNA or protein payloads and frataxin-enhancing small molecules for Friedreich ataxia, vitamin E and tocopherol-transporter-protein payloads for ataxia with vitamin E deficiency, and DNA-damage-response-modulating payloads for ataxia-telangiectasia. Release-trigger preference comprises enteric release followed by transferrin-receptor-mediated brain uptake with secondary Purkinje-cell-selective targeting using calbindin-1-binding aptamers or BoNT-receptor-binding peptides modified for safety. Biomarker monitoring comprises Scale for the Assessment and Rating of Ataxia every 3 months, magnetic resonance imaging cerebellar volume every 12 months, plasma neurofilament light chain every 3 months, and disease-specific biomarkers including frataxin protein in buccal cells for Friedreich ataxia. Clinical-development considerations include orphan-drug designation, presymptomatic genetic-carrier cohorts, and natural-history-control trial designs given the inadequacy of placebo control in slowly progressive ataxias.

Multiple sclerosis and central nervous system demyelinating diseases constitute a heterogeneous indication class. Pathophysiology of multiple sclerosis comprises immune-mediated demyelination of central nervous system axons with focal plaques, oligodendrocyte loss, axonal transection accumulating at rates of 11,000 to 22,000 axons per cubic millimeter of plaque per year, and progressive neurodegeneration with brain atrophy rates of 0.4 to 1.35 percent per year in untreated subjects. Relapsing-remitting MS comprises 85 percent of initial presentations with clinical relapses every 1 to 3 years on average; secondary progressive MS evolves from relapsing-remitting disease in 50 percent of subjects within 15 years; primary progressive MS comprises 10 to 15 percent of cases with continuous progression from onset and male predominance. Neuromyelitis optica spectrum disorder involves aquaporin-4 IgG autoantibodies attacking astrocytes with severe optic neuritis and longitudinally extensive transverse myelitis. MOG-antibody-associated demyelination overlaps clinically with NMOSD but with distinct serology and frequent monophasic course. Acute disseminated encephalomyelitis presents as a monophasic post-infectious demyelinating event predominantly in children. Prior-art treatments include monoclonal antibodies ocrelizumab, ofatumumab, and natalizumab requiring intravenous or subcutaneous administration every 4 weeks to 6 months with progressive multifocal leukoencephalopathy risk for natalizumab in JCV-seropositive subjects, oral sphingosine-1-phosphate receptor modulators with first-dose cardiac monitoring requirements, and high-efficacy therapies requiring infusion-suite logistics. Preferred payload classes comprise CD20-targeting nanobodies or single-chain antibody fragments delivering B-cell depletion through gut-associated lymphoid tissue and central nervous system simultaneously, remyelination-promoting payloads including clemastine fumarate analogs, LINGO1-blocking antibodies, and oligodendrocyte-precursor-cell-stimulating mRNA payloads encoding olig2 and sox 10. Release-trigger preference comprises immune-cell-uptake-triggered release in Peyer patches and mesenteric lymph nodes combined with central nervous system delivery for direct remyelinating action. Biomarker monitoring comprises plasma neurofilament light chain every 3 months, serum glial fibrillary acidic protein every 6 months, brain MRI with gadolinium every 6 months, and Expanded Disability Status Scale every 6 months.

Autoimmune encephalitis spectrum disorders constitute a class of subacute neuroinflammatory conditions caused by autoantibodies against neuronal cell-surface antigens. Pathophysiology of NMDA-receptor encephalitis involves IgG autoantibodies against the GluN1 subunit of N-methyl-D-aspartate receptors causing receptor internalization, reduced synaptic NMDA-receptor density, and characteristic clinical syndrome of psychiatric symptoms followed by seizures, dyskinesias, autonomic dysregulation, and central hypoventilation. LGI1 encephalitis involves autoantibodies against leucine-rich glioma-inactivated 1 protein with faciobrachial dystonic seizures and limbic encephalitis. CASPR2, GABA-B receptor, and AMPA-receptor encephalitis target additional synaptic and cell-surface antigens with characteristic syndromes. Prior-art treatment comprises intravenous methylprednisolone, intravenous immunoglobulin, plasma exchange, rituximab, cyclophosphamide, and emerging anti-CD19 and anti-CD38 therapies; treatment delays and incomplete responses occur in 20 to 40 percent of subjects. The oral platform disclosed herein enables daily home dosing of B-cell-depleting and plasma-cell-depleting payloads, intrathecal-equivalent delivery without lumbar puncture, and combination immunomodulation. Preferred payload classes comprise CD19-targeting and CD20-targeting antibody fragments, plasma-cell-depleting anti-CD38 nanobodies, antigen-specific tolerogenic payloads presenting GluN1, LGI1, CASPR2, GABA-B receptor, or GluA1/GluA2 peptide epitopes with regulatory-T-cell-inducing adjuvants, and BAFF-neutralizing payloads. Release-trigger preference comprises immune-cell-uptake release in gut-associated lymphoid tissue with secondary blood-brain barrier traversal for direct intrathecal-compartment access. Biomarker monitoring comprises serum and cerebrospinal fluid autoantibody titers every 4 weeks, modified Rankin Scale every 4 weeks, and seizure frequency for subjects with seizures. Clinical-development considerations include rapid-response trial designs for acute presentations and antigen-specific tolerogenic-payload development for chronic relapsing subjects.

Central nervous system oncology constitutes an indication class with high mortality and limited therapeutic penetrance across the blood-brain barrier and the blood-tumor barrier. Pathophysiology of glioblastoma multiforme IDH-wildtype, WHO grade 4, comprises diffusely infiltrating astrocytic neoplasms with median survival of 14 to 16 months under standard chemoradiotherapy with temozolomide; IDH-mutant astrocytoma grades 2 through 4 exhibits longer median survival of 5 to 10 years and harbors 2-hydroxyglutarate accumulation amenable to mutant-IDH inhibition. Oligodendroglioma with 1p/19q co-deletion exhibits median survival of 10 to 15 years. Diffuse midline glioma with H3K27M mutation predominantly affects pediatric and young-adult populations with median survival of 9 to 12 months. Medulloblastoma comprises WNT, SHH, group 3, and group 4 molecular subgroups with subgroup-defined prognosis and therapy. Atypical teratoid rhabdoid tumor involves SMARCB1 or SMARCA4 loss with infant predominance. Central nervous system metastasis of extracranial primary cancers including breast, lung, melanoma, gastrointestinal, and renal cell occurs in 10 to 30 percent of advanced-stage cancers with median survival of 4 to 16 months by primary type. Prior-art treatment limitations include marginal blood-brain barrier penetration of systemic chemotherapy with brain-to-plasma ratios below 0.05 for most cytotoxics, requirement for craniotomy-based surgical resection with attendant 1 to 5 percent operative mortality, radiation-induced cognitive decline with full-scale IQ decrements of 5 to 15 points in pediatric subjects, and resistance emergence in 90 percent of treated glioblastoma within 6 to 12 months.

Preferred payload classes for central nervous system oncology indications comprise mutant-IDH1-targeted small-molecule inhibitors such as ivosidenib analogs at 50 to 500 daltons for IDH-mutant astrocytoma and oligodendroglioma, H3K27M-directed peptide vaccines and EZH2 modulators for diffuse midline glioma, BRAF-V600E inhibitors and MEK inhibitors for BRAF-mutant CNS tumors, antibody-drug conjugates against EGFR variant III for EGFRvIII-positive glioblastoma, chimeric-antigen-receptor T-cell-engaging bispecific T-cell engagers targeting B7-H3, GD2, or interleukin-13-receptor-alpha-2, immune-checkpoint inhibitors as nanobody payloads with avoidance of systemic immune-related adverse events, and tumor-treating-fields-sensitizing payloads. Smoothened inhibitors and Hedgehog-pathway antagonists for SHH-medulloblastoma; mutant-SMARCB1-restoring or EZH2-targeting payloads for atypical teratoid rhabdoid tumor; HER2-targeting and brain-metastasis-directed payloads for breast-cancer brain metastasis; ALK, EGFR, and KRAS-G12C inhibitors for lung-cancer brain metastasis. Release-trigger preference comprises tumor-microenvironment-cleavable linkers responsive to matrix metalloproteinase-2 and matrix metalloproteinase-9 at concentrations of 50 to 500 nanomolar combined with hypoxia-triggered release in regions with oxygen tension below 10 millimeters of mercury; alternative trigger comprises pH-responsive release at acidic tumor extracellular pH 6.0 to 6.8. Biomarker monitoring comprises volumetric magnetic resonance imaging every 8 to 12 weeks by Response Assessment in Neuro-Oncology criteria, plasma circulating tumor DNA for tumor-specific mutations every 4 weeks, cerebrospinal fluid circulating tumor DNA for leptomeningeal disease, and tumor-specific positron emission tomography tracers. Clinical-development considerations include adaptive platform trial designs for glioblastoma, age-stratified pediatric trial design, accelerated-approval pathways for diseases with no approved therapy, and parallel tumor-tissue and liquid biopsy biomarker development.

Neuropsychiatric disorders constitute a high-prevalence indication class with substantial inadequacy of current oral psychotropics for treatment-resistant subgroups. Pathophysiology comprises heterogeneous combinations of monoaminergic, glutamatergic, GABAergic, and neuroinflammatory mechanisms with structural and functional brain abnormalities demonstrable on magnetic resonance imaging. Major depressive disorder including treatment-resistant subtypes affects approximately 280 million individuals globally with 30 percent failing two or more adequate trials of selective serotonin reuptake inhibitors or serotonin-norepinephrine reuptake inhibitors. Bipolar disorders types I and II involve mood-state cycling with manic, hypomanic, and depressive episodes; schizophrenia involves positive, negative, and cognitive symptom domains with onset typically in late adolescence to early adulthood; schizoaffective disorder combines mood and psychotic features; generalized anxiety disorder, social anxiety disorder, panic disorder, obsessive-compulsive disorder, and post-traumatic stress disorder constitute the anxiety- and trauma-related-disorder spectrum. Prior-art treatment limitations include 2 to 6 week onset latency for conventional antidepressants, extrapyramidal and metabolic side effects of antipsychotics, abuse-liability and withdrawal risks of benzodiazepines, narrow therapeutic indices for lithium and clozapine with monitoring requirements, and treatment-resistance rates of 20 to 40 percent across indications. The oral platform disclosed herein enables delivery of glutamatergic and GABAergic neuromodulators with rapid onset, sustained-release formulations reducing dosing frequency, and combination delivery of psychotropic and neurotrophic payloads.

Preferred payload classes for neuropsychiatric indications comprise NMDA-receptor modulators including ketamine analogs and esketamine-related molecules of 200 to 350 daltons with controlled brain exposure, sigma-1-receptor agonists, brain-derived neurotrophic factor mRNA payloads with hippocampal and prefrontal-cortex enrichment, neurosteroid GABA-A receptor modulators of 300 to 450 daltons, serotonergic 2A-receptor agonist and biased-agonist payloads for treatment-resistant depression embodiments with controlled release reducing hallucinogenic onset for non-perceptual therapeutic use, vasopressin VIB-receptor antagonists for stress-related disorders, orexin-receptor antagonists for hyperarousal subtypes, and FK506-binding protein modulators for trauma-related disorders. For schizophrenia embodiments, payload classes comprise muscarinic M1/M4 receptor agonists, trace-amine-associated-receptor-1 agonists, and metabotropic-glutamate-receptor modulators. Release-trigger preference comprises pH-triggered enteric release combined with circadian-timed release using physiologically-mimetic clock-aligned coatings releasing payload in defined diurnal windows for diurnal symptom control, and stress-responsive release using cortisol-responsive linkers for stress-disorder embodiments. Biomarker monitoring comprises Patient Health Questionnaire-9 and Montgomery-Asberg Depression Rating Scale every 2 weeks for depression embodiments, Young Mania Rating Scale for bipolar embodiments, Positive and Negative Syndrome Scale for schizophrenia embodiments, Scale, Hamilton Anxiety Rating Yale-Brown Obsessive Compulsive Scale, and Clinician-Administered PTSD Scale for respective embodiments, plasma inflammatory markers C-reactive protein interleukin-6, and quantitative electroencephalography. Clinical-development considerations include adaptive enrichment by inflammatory biomarker subtype, careful abuse-liability assessment for serotonergic 2A-receptor agonist payloads, and pediatric exclusivity extensions where applicable.

Substance use disorders constitute an indication class for which oral CNS-targeted delivery offers advantages of patient acceptability, avoidance of injection sites in subjects with intravenous drug use history, and combination delivery of relapse-prevention and craving-reduction payloads. Pathophysiology comprises neuroadaptations in mesolimbic dopamine pathway, prefrontal cortex executive-function circuits, and habenular and extended-amygdala stress circuits, with substance-specific receptor and signaling perturbations. Opioid use disorder involves mu-opioid receptor neuroadaptation with tolerance, withdrawal, and craving cycles; alcohol use disorder involves GABA-A, NMDA-receptor, and opioid-receptor neuroadaptations; stimulant use disorder involves dopaminergic and trace-amine-associated-receptor-1 neuroadaptations with current absence of FDA-approved pharmacotherapies; cannabis use disorder and nicotine use disorder involve CB1-receptor and nicotinic-acetylcholine-receptor neuroadaptations respectively. Prior-art treatment limitations include long-acting injectable naltrexone and buprenorphine requiring monthly clinical-setting administration, daily methadone clinic visits in regulated treatment programs, acamprosate three-times-daily dosing with poor adherence, and absence of approved pharmacotherapies for stimulant and cannabis use disorders. Preferred payload classes comprise mu-opioid partial-agonist and antagonist payloads with abuse-deterrent formulation, GABA-B-receptor agonist payloads for alcohol use disorder, trace-amine-associated-receptor-1 agonists and metabotropic-glutamate-receptor modulators for stimulant use disorder, nicotinic-receptor partial agonists for nicotine use disorder, and cannabinoid-receptor inverse agonists with peripheral restriction balanced against central efficacy. Release-trigger preference comprises sustained release over 24 to 168 hours for relapse-prevention payloads with abuse-deterrent crush-resistant matrix. Biomarker monitoring comprises Timeline Followback substance-use assessment, urine toxicology, breath alcohol concentration, and substance-craving visual analog scales. Clinical-development considerations include abuse-liability assessment under FDA guidance, opioid-overdose-rescue co-administration considerations, and pragmatic trial designs in community treatment settings.

Neurodevelopmental disorders constitute an indication class with pediatric and lifelong manifestations, frequent oral-acceptability advantages over injection in pediatric subjects, and emerging molecular targets across genetic subtypes. Pathophysiology of autism spectrum disorder involves heterogeneous combinations of synaptic, glutamatergic, GABAergic, and connectivity perturbations with hundreds of identified risk genes; Rett syndrome involves MECP2 mutations causing X-linked dominant disease in females with regression at 6 to 18 months of age; Angelman syndrome involves UBE3A maternal-allele loss with characteristic gait, seizures, and laughter; fragile X syndrome involves FMRI CGG-repeat expansion above 200 with FMRP loss; Phelan-McDermid syndrome involves 22q13.3 deletion encompassing SHANK3; Cornelia de Lange syndrome involves cohesin-complex-gene mutations; Williams syndrome involves 7q11.23 deletion. Prior-art treatment limitations include absence of disease-modifying therapy for autism core symptoms, restricted symptomatic options for Rett syndrome with trofinetide as first approved therapy, and absence of approved therapies for Angelman, fragile X, Phelan-McDermid, Cornelia de Lange, and Williams syndromes. Preferred payload classes comprise MECP2-replacement mRNA or protein payloads for Rett syndrome, UBE3A-paternal-allele-activating antisense oligonucleotides targeting UBE3A-ATS for Angelman syndrome, FMR1-reactivating epigenetic-editing payloads for fragile X syndrome, SHANK3-elevating small molecules for Phelan-McDermid syndrome, and synaptic-function-modulating payloads addressing common downstream targets across autism-spectrum genetic subtypes. Release-trigger preference comprises pediatric-palatable enteric release with taste-masked outer coating and age-appropriate dosing volumes of 1 to 10 milliliters of liquid suspension or 5 to 50 milligrams of chewable tablet. Biomarker monitoring comprises age-appropriate behavioral and developmental scales, electroencephalography for seizure-prone embodiments, and genotype-specific biomarkers. Clinical-development considerations include pediatric study planning under FDA Pediatric Research Equity Act, rare-disease orphan-drug designations, family-burden and caregiver-reported-outcome measures, and adaptive seamless phase 2/3 designs for ultra-rare populations.

Pediatric epileptic encephalopathies constitute a high-mortality, high-morbidity indication class with current treatment limitations including pharmacoresistance, polypharmacy, and developmental sequelae. Pathophysiology comprises genetic, structural, metabolic, and infectious etiologies producing developmental and epileptic encephalopathy syndromes with cognitive regression and refractory seizures. Dravet syndrome involves SCN1A haploinsufficiency with febrile and afebrile seizures, sudden unexpected death in epilepsy risk of approximately 15 percent over 20 years, and progressive cognitive impairment; Lennox-Gastaut syndrome involves multiple seizure types including tonic, atonic, and atypical absence with characteristic slow spike-and-wave electroencephalography pattern; infantile spasms with West syndrome triad involves epileptic spasms, hypsarrhythmia, and developmental arrest with response to adrenocorticotropic hormone, vigabatrin, or prednisolone in 50 to 75 percent of subjects but with relapse risk; KCNQ2 encephalopathy involves potassium-channel dysfunction with neonatal-onset seizures; SCN8A encephalopathy involves sodium-channel gain-of-function with early-onset refractory seizures. Prior-art treatment limitations include polytherapy with three to six antiseizure medications with cumulative cognitive side effects, intrathecal nusinersen-equivalent products for SCN1A under development, and absence of mutation-specific therapies for most genotypes. Preferred payload classes comprise SCNIA-allele-specific antisense oligonucleotides enhancing healthy allele expression for Dravet syndrome, sodium-channel-isoform-selective small molecules for SCN8A encephalopathy, potassium-channel openers for KCNQ2 encephalopathy, and broad-spectrum neuronal-excitation-reducing payloads. Release-trigger preference comprises pediatric-acceptable enteric release with sustained release over 24 hours to reduce dosing frequency. Biomarker monitoring comprises 24-hour video-electroencephalography monthly, seizure diary, and developmental-milestone assessment.

Lysosomal storage disorders with central nervous system involvement constitute an indication class for which oral CNS-targeted delivery provides definite advantage over current intravenous and intrathecal enzyme replacement therapies and emerging gene therapies. Pathophysiology comprises biallelic mutations in lysosomal hydrolase or lysosomal-membrane-protein genes producing substrate accumulation in neuronal and glial lysosomes with secondary mitochondrial dysfunction, neuroinflammation, and progressive neurodegeneration. Gaucher disease types II and III involve GBA mutations with glucosylceramide accumulation and neuronopathic involvement; Niemann-Pick disease type C involves NPC1 or NPC2 mutations with cholesterol and sphingolipid trafficking impairment; Tay-Sachs and Sandhoff diseases involve HEXA or HEXB mutations with GM2 ganglioside accumulation; GM1 gangliosidosis involves GLB1 mutations with GM1 ganglioside accumulation; neuronal ceroid lipofuscinoses CLN1 through CLN14 involve diverse molecular etiologies with lipopigment accumulation and pediatric-to-adult onset; mucopolysaccharidoses with CNS involvement including MPS I Hurler, MPS II Hunter, MPS III Sanfilippo subtypes A through D, and MPS VII Sly involve glycosaminoglycan accumulation; Krabbe disease involves GALC mutations with galactosylceramide and psychosine accumulation; metachromatic leukodystrophy involves ARSA mutations with sulfatide accumulation and demyelination. Prior-art treatment limitations include intravenous enzyme replacement therapy without blood-brain barrier penetration for CNS manifestations, intrathecal enzyme replacement therapy requiring lumbar puncture every 2 to 4 weeks, gene therapy products with single-administration cost exceeding 2 to 3 million United States dollars, and substrate-reduction therapy with modest CNS efficacy.

Preferred payload classes for lysosomal storage disorder indications comprise enzyme-replacement-payload variants engineered for blood-brain barrier transcytosis through transferrin receptor or insulin receptor binding domains fused to the lysosomal enzyme, mRNA encoding the deficient enzyme with lysosomal targeting signal, substrate-reduction small molecules of 250 to 600 daltons, and gene-editing payloads for in vivo correction of common pathogenic variants. For Gaucher disease, glucocerebrosidase enzyme with blood-brain barrier shuttle and substrate-reduction inhibitor of glucosylceramide synthase; for Niemann-Pick C, 2-hydroxypropyl-beta-cyclodextrin analog or cholesterol-trafficking-restoring payloads; for MPS III Sanfilippo, heparan-N-sulfatase or similar subtype-specific enzyme with brain shuttle; for metachromatic leukodystrophy, arylsulfatase A with brain shuttle. Release-trigger preference comprises pH-triggered enteric release followed by transferrin-receptor-mediated brain transcytosis with lysosomal-trafficking peptide ensuring lysosomal delivery within neurons; lysosomal pH 4.5 to 5.0 triggers final payload release from endolysosomal compartment. Biomarker monitoring comprises cerebrospinal fluid enzyme activity, urine or cerebrospinal fluid substrate concentration including heparan sulfate for MPS III and sulfatide for metachromatic leukodystrophy, magnetic resonance spectroscopy for selected substrates, and disease-specific neurocognitive assessment scales. Clinical-development considerations include newborn-screening positive cohort enrollment for presymptomatic dosing, ultra-rare-disease natural-history-control designs, and accelerated-approval pathways using biomarker endpoints.

Acute neurological injury constitutes an indication class with narrow therapeutic time windows and current logistical barriers to widespread treatment. Pathophysiology of acute ischemic stroke comprises arterial occlusion producing core infarct and surrounding penumbra of metabolically compromised but salvageable tissue with progressive penumbral conversion over 6 to 24 hours; hemorrhagic stroke including subarachnoid hemorrhage comprises vascular rupture with primary hematoma expansion in the first 4 to 24 hours, secondary brain injury through inflammation and edema, and delayed cerebral ischemia in 20 to 30 percent of aneurysmal subarachnoid hemorrhage subjects; traumatic brain injury comprises primary mechanical injury and secondary biochemical cascades with diffuse axonal injury, blood-brain barrier disruption, and chronic post-traumatic neurodegeneration; spinal cord injury comprises primary mechanical injury and secondary inflammation, excitotoxicity, and ischemia with progressive demyelination and scar formation. Prior-art treatment limitations include 4.5-hour intravenous thrombolysis window for alteplase or tenecteplase with eligibility in only 5 to 15 percent of acute ischemic stroke subjects, 24-hour mechanical thrombectomy window for large-vessel occlusion in comprehensive stroke centers, absence of effective neuroprotective therapies despite over 1,000 failed clinical trials, and supportive-care-only treatment for hemorrhagic stroke beyond blood-pressure and intracranial-pressure management. Oral administration is feasible only in conscious subjects without dysphagia; for unconscious subjects, the platform may be administered through nasogastric or orogastric tube within minutes of presentation. Preferred payload classes comprise free-radical scavengers, edaravone analogs, NMDA-receptor partial antagonists, complement-pathway inhibitors, and neurotrophic-factor mRNA payloads for stroke and traumatic brain injury; for spinal cord injury, anti-inhibitory-molecule payloads including anti-Nogo and anti-myelin-associated-glycoprotein antibodies, scar-modulating and payloads, remyelination-promoting payloads. Release-trigger preference comprises rapid enteric release with absorption within 30 to 90 minutes and brain delivery within 60 to 180 minutes of administration. Biomarker monitoring comprises serial National Institutes of Health Stroke Scale or Glasgow Coma Scale, computed tomography or magnetic resonance imaging at 24 hours and 7 days, and plasma neurofilament light chain trajectory.

Drug-resistant epilepsies constitute an indication class affecting approximately one-third of subjects with epilepsy across focal and generalized syndromes with failure of two or more appropriately selected and dosed antiseizure medications. Pathophysiology comprises diverse etiologies including focal cortical dysplasia, mesial temporal sclerosis, post-traumatic epilepsy, autoimmune epilepsy, and genetic epilepsies with channelopathies, synaptopathies, and metabolic etiologies. Mesial temporal lobe epilepsy involves hippocampal sclerosis with neuronal loss in CA1 and CA3, granule cell dispersion, and mossy fiber sprouting; focal-onset drug-resistant epilepsy in extratemporal locations involves various cortical dysplasias and focal lesions; generalized drug-resistant epilepsy includes idiopathic generalized epilepsies and developmental and epileptic encephalopathies; status epilepticus comprises continuous or recurrent seizure activity exceeding 5 minutes with refractory status epilepticus failing two or more antiseizure medications and super-refractory status epilepticus failing 24 hours of anesthetic therapy. Prior-art treatment limitations include surgical resection as the only currently curative intervention with eligibility in only 10 to 30 percent of drug-resistant cases, vagus nerve stimulation and responsive neurostimulation with hardware implantation, and limited disease-modifying interventions. Preferred payload classes comprise sodium-channel-selective small molecules with state-dependent and use-dependent block, potassium-channel openers, GABA-A-receptor modulators with selectivity for non-sedating subunit compositions, mTOR inhibitors for focal cortical dysplasia and tuberous sclerosis complex epilepsies, glutamate-receptor antagonists, and epigenetic-editing payloads for status-epilepticus-induced epileptogenesis prevention. Release-trigger preference comprises sustained release over 24 hours for chronic embodiments and rapid release for status epilepticus rescue embodiments with absorption within 15 to 45 minutes. Biomarker monitoring comprises seizure diary with electronic capture, ambulatory electroencephalography for selected embodiments, and brain magnetic resonance imaging at baseline and 12 months.

Chronic pain and small-fiber neuropathies constitute a high-prevalence indication class with substantial unmet need for non-opioid analgesics and disease-modifying neuropathy therapies. Pathophysiology of neuropathic pain comprises peripheral and central sensitization with maladaptive plasticity at primary afferent nociceptors, dorsal horn neurons, thalamus, and cortex; diabetic peripheral neuropathy involves hyperglycemia-induced metabolic and microvascular nerve injury with predominantly small-fiber involvement in early disease and mixed fiber involvement in advanced disease; chemotherapy-induced peripheral neuropathy involves platinum-, taxane-, vinca-alkaloid-, and proteasome-inhibitor-induced sensory and autonomic nerve injury; post-herpetic neuralgia involves persistent sensitization after varicella-zoster reactivation; trigeminal neuralgia involves trigeminal-nerve-root demyelination commonly from neurovascular compression; cluster headache involves hypothalamic and trigeminal-autonomic activation; chronic migraine involves cortical spreading depression, trigeminal-cervical complex activation, and calcitonin-gene-related-peptide release. Prior-art treatment limitations include opioid analgesics with addiction and overdose risk, gabapentinoids with sedation and dependence concerns, tricyclic and serotonin-norepinephrine reuptake inhibitor antidepressants with anticholinergic and cardiac side effects, and parenteral calcitonin-gene-related-peptide-pathway monoclonal antibodies for migraine requiring monthly subcutaneous administration. Preferred payload classes comprise voltage-gated-sodium-channel-Nav1.7-selective small molecules and Nav1.8-selective payloads, voltage-gated-calcium-channel alpha-2-delta modulators, calcitonin-gene-related-peptide-receptor antagonists and gepant payloads, transient-receptor-potential-channel modulators, and neurotrophic-factor mRNA payloads for nerve regeneration. Release-trigger preference comprises sustained release over 12 to 24 hours with circadian-aligned release for migraine prophylaxis embodiments and rapid release for acute migraine and cluster headache rescue. Biomarker monitoring comprises Numeric Rating Scale and Brief Pain Inventory, validated neuropathic-pain questionnaires, quantitative sensory testing, intraepidermal nerve fiber density on skin biopsy, and headache diary.

Geriatric chronic indications for which oral administration is advantageous over parenteral administration constitute an indication class characterized by polypharmacy, frailty, reduced subcutaneous tissue volume for injection, and frequent administration burden. Indications include vascular cognitive impairment with small-vessel ischemic disease, lacunar infarction, and white-matter hyperintensities producing executive dysfunction and processing-speed decline; mixed dementia comprising co-existent Alzheimer pathology and vascular pathology with synergistic cognitive decline; age-related sleep disturbance including reduced slow-wave sleep, fragmented sleep architecture, and circadian-rhythm advancement; and sarcopenia with neurological components encompassing motor-unit loss, neuromuscular junction degradation, and central motor-cortical changes. Prior-art treatment limitations include parenteral monoclonal antibody therapies for Alzheimer pathology with infusion-suite requirements challenging in geriatric subjects with mobility limitations, hypnotic and sedative agents with fall and confusion risk in geriatric subjects, and absence of approved therapies for sarcopenia. Preferred payload classes comprise the previously described Alzheimer-disease payloads for mixed-dementia embodiments, endothelial-function-restoring payloads and small-vessel-disease-modifying payloads for vascular cognitive impairment, orexin-receptor antagonists with controlled pharmacokinetics avoiding morning sedation for sleep embodiments, and myostatin-pathway modulators and motor-neuron-trophic-factor payloads for sarcopenia embodiments. Release-trigger preference comprises sustained release over 24 hours with once-daily morning dosing to maximize adherence and circadian-aligned release for sleep embodiments releasing payload 30 to 60 minutes after bedtime administration. Biomarker monitoring comprises age-appropriate cognitive assessment including Montreal Cognitive Assessment, gait speed measurement, grip strength, polysomnography for sleep embodiments, and biomarkers as previously described. Clinical-development considerations include enrollment of geriatric subjects with planning for fall, fracture, and delirium safety signals; polypharmacy interaction screening; and adherence-friendly packaging design.

Pediatric chronic indications for which avoidance of injection is desirable constitute an indication class with particular advantages of oral administration including child acceptability, school-day-compatible dosing, and avoidance of injection-site reactions and procedural sedation. Indications include pediatric neurodegenerative diseases encompassing neuronal ceroid lipofuscinoses, mucopolysaccharidoses with CNS involvement, leukodystrophies, and rare-disease pediatric neurodegenerative syndromes; pediatric epilepsies as previously described; and pediatric neuro-oncology supportive care including chemotherapy-induced peripheral neuropathy, radiation-induced cognitive sequelae, post-treatment fatigue, and post-treatment depression and anxiety. Prior-art treatment limitations include intrathecal enzyme replacement therapy requiring lumbar puncture under sedation every 2 to 4 weeks in pediatric subjects, weekly or monthly intravenous enzyme replacement requiring central venous access in many subjects, intravenous chemotherapy and supportive-care infusions requiring port placement, and adult-oriented formulations requiring compounded liquid preparations. Preferred payload classes comprise the disease-specific payloads described in the lysosomal storage disorder and pediatric epileptic encephalopathy sections, with pediatric-formulation considerations including taste-masking, age-appropriate dosing volumes, and weight-band-adjusted unit doses. Release-trigger preference comprises pediatric-acceptable taste-masked enteric release with palatability optimization and dose-form selection based on developmental stage including liquid suspension below age 4, chewable tablet ages 4 to 8, and conventional tablet or capsule above age 8. Biomarker monitoring comprises age-appropriate developmental and cognitive assessments, disease-specific biomarkers, growth and pubertal-development tracking, and school-performance metrics with parental and teacher reporting. Clinical-development considerations include Pediatric Research Equity Act compliance with pediatric study plans, rare-pediatric-disease priority-review-voucher eligibility for selected indications, age-stratified dosing studies, and family-burden assessment.

Indications in low-resource healthcare settings constitute an indication class with particular advantages of oral administration including avoidance of injection-related infection risk, simplified cold-chain requirements, community-health-worker administration feasibility, and reduced cost of goods enabling access pricing. Indications include chronic central nervous system infections such as neurocysticercosis with Taenia solium larval cysts in brain parenchyma and ventricles; neurosyphilis with Treponema pallidum CNS invasion; HIV-associated neurocognitive disorder with HIV CNS reservoirs and neuroinflammation; cryptococcal meningitis with Cryptococcus neoformans CNS infection in immunocompromised hosts; and parasitic CNS disease including cerebral malaria, neurotoxoplasmosis, African trypanosomiasis with CNS involvement, schistosomiasis with CNS involvement, and toxocariasis. Prior-art treatment limitations include intravenous amphotericin B for cryptococcal meningitis with nephrotoxicity and infusion-site complications, intravenous melarsoprol for late-stage African trypanosomiasis with reactive encephalopathy in 5 to 10 percent of treated subjects, intramuscular benzathine penicillin requiring repeated injections for neurosyphilis, and barriers to long-term oral combination antiretroviral therapy adherence for HIV neurocognitive disorder. Preferred payload classes comprise antiparasitic albendazole and praziquantel analogs with enhanced CNS penetration, antifungal payloads with h cryptococcal capsule-targeting moieties, antitreponemal payloads with CNS-compartment delivery, antiretroviral payloads selected for CNS penetration effectiveness with controlled-release pharmacokinetics, and anti-inflammatory payloads for treatment-associated immune-reconstitution-inflammatory-syndrome management. Release-trigger preference comprises sustained release over 24 to 168 hours with thermostable formulation requiring no cold-chain storage and compatibility with community-health-worker directly-observed-therapy programs. Biomarker monitoring comprises cerebrospinal fluid pathogen-specific testing, plasma and cerebrospinal fluid pathogen-load assays, and clinical and cognitive assessment scales. Clinical-development considerations include World Health Organization prequalification pathway planning, access-pricing commitments, and partnership with global health organizations and ministries of health.

Emerging indications encompass long COVID neurological sequelae, chronic neuroinflammation, and microbiome-gut-brain axis disorders. Pathophysiology of long COVID neurological sequelae comprises persistent neuroinflammation following acute SARS-COV-2 infection with cognitive fog, fatigue, autonomic dysfunction, and small-fiber neuropathy in 10 to 30 percent of post-acute subjects; mechanisms include persistent viral reservoirs, autoimmune cross-reactivity, endothelial dysfunction, and persistent microglial activation. Chronic neuroinflammation constitutes a transdiagnostic mechanism contributing to Alzheimer disease, Parkinson disease, traumatic brain injury sequelae, treatment-resistant depression, and other indications with shared microglial and astroglial activation states identifiable by translocator-protein positron emission tomography and by emerging plasma biomarkers including soluble TREM2, glial fibrillary acidic protein, and interleukin-6. Microbiome-gut-brain axis disorders comprise emerging indications in which gut microbial composition, bile-acid metabolism, short-chain fatty acid production, tryptophan metabolism, and gut-derived inflammatory signals influence central nervous system function; relevant indications include autism spectrum disorder, Parkinson disease, depression, and anxiety. Prior-art treatment limitations include absence of approved therapies for long COVID neurological sequelae, off-label use of conventional anti-inflammatory and neuroactive agents, and absence of microbiome-targeted CNS therapeutics. The disclosed oral platform offers particular advantage for microbiome-gut-brain-axis embodiments through co-delivery of microbiome-modulating and CNS-active payloads in a single dosage unit. Preferred payload classes comprise interleukin-6-receptor antagonists, complement-pathway modulators, microglial-state-modulating payloads including colony-stimulating-factor-1-receptor modulators, vagal-afferent-stimulating peptides, postbiotic short-chain fatty acid derivatives, bile-acid-receptor modulators including farnesoid-X-receptor and TGR5 agonists, and microbiome-modulating payloads. Release-trigger preference comprises dual-compartment release with upper-small-intestinal release of microbiome-modulating payloads and systemic absorption and brain delivery of CNS-active payloads. Biomarker monitoring comprises plasma inflammatory marker panels, fecal microbiome metagenomic and metabolomic profiling, autonomic-function testing, and disease-specific symptom scales. Clinical-development considerations include adaptive enrichment designs based on inflammatory and microbiome biomarker stratification, novel endpoint development for long COVID, and parallel development of companion diagnostics for transdiagnostic neuroinflammation indications.

The therapeutic indications enumerated in the foregoing paragraphs are intended as non-limiting embodiments of the disclosed oral nanostructure drug delivery platform and may be combined as combinatorial therapeutic regimens within a single dosing protocol where pathophysiology is overlapping or where co-morbidity is present. For example, an embodiment for mixed dementia combines payloads from the Alzheimer disease and vascular cognitive impairment indication classes; an embodiment for Parkinson disease with depression combines payloads from the Parkinson disease and neuropsychiatric disorder indication classes; an embodiment for amyotrophic lateral sclerosis with frontotemporal dementia combines payloads from the motor neuron disease and frontotemporal dementia indication classes; an embodiment for autism spectrum disorder with epilepsy combines payloads from the neurodevelopmental disorder and drug-resistant epilepsy indication classes. The platform supports combinatorial payload loading of up to 4 distinct payload classes per dosage unit with independent release-trigger control of each payload class, enabling temporal sequencing of payload exposure within the brain compartment over windows of 1 to 48 hours after dosing. Across all indications described herein, the platform achieves an estimated 5- to 50-fold increase in brain payload exposure relative to free-drug administration of the same payload, with reduction of systemic exposure and associated peripheral side effects by 30 to 70 percent. The clinical-development strategy across the indication portfolio comprises a master-protocol platform design with shared safety database, biomarker-stratified enrichment, and indication-specific efficacy substudies, supporting parallel development across the 21 indication categories enumerated herein with shared regulatory submissions for chemistry, manufacturing, and controls and indication-specific submissions for efficacy and safety in each indication class.

### Expanded Axis 8—Manufacturing and Scale-Up (Deep Dive)

### Recombinant Expression of Outer-Shell Coat Proteins

Outer-shell coat proteins of the platform are produced by recombinant expression in Escherichia coli host strains selected to match the biochemical demands of the particular coat sequence. Standard high-yield expression is performed in E. coli BL21 (DE3), which carries a chromosomal copy of T7 RNA polymerase under control of the lacUV5 promoter and supports tightly regulated T7-driven transcription of pET-class expression plasmids. Coat sequences encoding hydrophobic domains, membrane-proximal segments, or proteins observed to be toxic upon overexpression are produced in C41 (DE3) and C43 (DE3) derivative strains, which carry mutations that reduce basal expression and tolerate proteins that perturb the inner membrane. Coat constructs that contain rare codons relative to the E. coli codon table, including AGG, AGA, CGG, CUA, AUA, CCC, and GGA, are expressed in Rosetta (DE3), which supplies the corresponding tRNAs on a chloramphenicol-resistant plasmid. Coats that benefit from titratable T7 polymerase levels for soluble accumulation are produced in Lemo21 (DE3), in which T7 polymerase activity is tuned by addition of rhamnose at concentrations of from 0 to 2000 micromolar. Expression vectors are pET-class plasmids selected for the desired fusion configuration, including pET-3, pET-11, pET-15b, pET-21, pET-22b, pET-24, pET-28, pET-30, and pET-32, providing N-terminal or C-terminal hexahistidine, SUMO, MBP, GST, thioredoxin, or NusA fusion partners and corresponding protease cleavage sites. Promoter induction is achieved by addition of isopropyl beta-D-1-thiogalactopyranoside (IPTG) to a final concentration of from 0.05 to 1.0 millimolar after the culture reaches an optical density at 600 nanometers (OD600) of from 0.6 to 1.0, with post-induction incubation of from 3 hours at 37 degrees Celsius for high-yield soluble expression to from 16 to 20 hours at 16 to 20 degrees Celsius for proteins that require slower folding kinetics. In-process controls during induction include OD600 measurements every 30 minutes, dissolved-oxygen monitoring above 30 percent saturation, and pH maintenance between 6.8 and 7.2 by automated addition of ammonium hydroxide or phosphoric acid.

Auto-induction is used as an alternative to IPTG induction for protocols that benefit from operator-free unattended culture progression and consistent expression timing across multiple bioreactors operating in parallel. Auto-induction medium contains glucose at from 0.05 to 0.10 percent weight per volume, lactose at from 0.2 to 0.5 percent weight per volume, and glycerol at from 0.5 to 0.6 percent weight per volume in a buffered base of 25 millimolar disodium hydrogen phosphate and 25 millimolar potassium dihydrogen phosphate, with 50 millimolar ammonium chloride and 5 millimolar sodium sulfate. Cells consume glucose preferentially while suppressing lac operon induction, then transition to lactose metabolism as glucose is depleted; intracellular conversion of lactose to allolactose by beta-galactosidase relieves LacI repression of the lacUV5 promoter, initiating T7 polymerase production and downstream coat-protein expression. Scale progression proceeds from shake-flask culture in 1-liter to 5-liter baffled Erlenmeyer flasks at 200 to 250 revolutions per minute for process development, through 10-liter and 20-liter benchtop stirred-tank bioreactors for engineering runs, to 100-liter, 500-liter, and 2000-liter cGMP fermentors for clinical and commercial supply. Fed-batch fermentation extends biomass accumulation beyond the limits of batch culture by controlled addition of a glucose- or glycerol-based feed solution at rates of from 5 to 25 grams per liter per hour, maintaining residual glucose below 1 gram per liter to suppress acetate accumulation and Crabtree-like overflow metabolism. Cell-density targets at induction are OD600 of from 60 to 150 in fed-batch operation, corresponding to wet cell weights of from 90 to 220 grams per liter. Process variables monitored continuously include temperature within plus or minus 0.5 degrees Celsius of setpoint, dissolved oxygen by polarographic or optical sensors, pH by gel-filled electrode, off-gas oxygen and carbon dioxide by mass spectrometry, and stir-rate and air-flow adjustments cascaded to maintain dissolved oxygen above 30 percent of saturation. Cells are harvested at predetermined post-induction times by continuous-flow centrifugation at from 8,000 to 12,000 times gravity and frozen at minus 80 degrees Celsius until lysis.

### Yeast and Insect Cell Production Alternatives

Coat proteins that require post-translational modifications absent in E. coli, that fold poorly in the bacterial cytoplasm, or that benefit from secretion into the extracellular medium for simplified downstream processing are produced in Pichia pastoris using methanol-induced expression vectors of the pPIC and pPICZ classes. The methanol-utilizing strain X-33 or the protease-deficient strain SMD1168H is transformed by electroporation with a linearized vector targeting integration into the AOX1 or GAP locus, and high-copy integrants are selected on zeocin gradients of from 100 to 2000 micrograms per milliliter. Fermentation proceeds in three phases: a glycerol batch phase to accumulate biomass to OD600 of from 100 to 200, a glycerol-limited fed-batch transition to derepress the AOX1 promoter, and a methanol-induction phase with controlled methanol addition at from 1 to 6 milliliters per liter per hour to maintain residual methanol between 0.5 and 1 gram per liter as measured by online sensor or offline gas chromatography. Insect-cell production employs Spodoptera frugiperda Sf9 and Sf21 lines infected with recombinant baculovirus produced by transposition into the bacmid in DH10Bac E. coli, with baculovirus titers of from 1 times 10 to the 8th to 1 times 10 to the 9th plaque-forming units per milliliter. Sf9 cultures grown in serum-free medium at 27 degrees Celsius to a density of from 1.5 to 2.5 times 10 to the 6th cells per milliliter are infected at a multiplicity of infection of from 1 to 5 and harvested between 48 and 96 hours post-infection. Mammalian production for glycosylation-dependent shells is performed in HEK293 derivatives, including HEK293-F, HEK293-6E, and Expi293F, and in CHO derivatives, including CHO-K1, CHO-S, and ExpiCHO, in chemically defined serum-free media in orbital-shaken or stirred-tank bioreactors of from 250 milliliters to 2000 liters working volume. Transient transfection with polyethyleneimine or cationic-lipid reagents at DNA-to-cell ratios of from 1 to 3 micrograms per million cells produces yields of from 50 to 500 milligrams of secreted glycoprotein per liter at 5 to 10 days post-transfection. Stable pools and clones selected with glutamine synthetase or dihydrofolate reductase amplification systems support sustained production at densities of from 10 to 30 times 10 to the 6th cells per milliliter in perfusion or fed-batch mode.

### Virus-Like Particle Assembly Methods

Virus-like particles forming the outer shell are assembled from purified coat-protein monomers or pentameric subunits by one of three methods selected according to coat-protein biophysics and cargo-loading requirements. In vitro self-assembly from purified coat protein is performed at coat-protein concentrations of from 0.5 to 5 milligrams per milliliter in assembly buffers containing from 50 to 500 millimolar sodium chloride, from 10 to 50 millimolar sodium phosphate or HEPES at pH of from 6.5 to 7.5, and where applicable from 1 to 10 millimolar magnesium chloride or calcium chloride to stabilize capsomer-capsomer interactions. Assembly is initiated by dialysis from disassembly buffer (typically 1.5 molar urea or 0.5 molar guanidinium chloride at alkaline pH) into assembly buffer over 4 to 24 hours at 4 degrees Celsius, with intermediate buffer exchanges to control the rate of denaturant removal. Scaffold-protein-mediated assembly employs an internal scaffold protein, typically a truncated phage scaffold or a recombinant scaffold bearing a charged or aromatic interior surface, present at a stoichiometry of from 60 to 240 scaffold monomers per particle. Scaffold-bound coat assembles into a procapsid intermediate that is converted to the mature shell by scaffold removal through proteolysis, pH-induced dissociation, or competing-ligand displacement. Disassembly-reassembly is used when cargo must be loaded after coat purification but before final encapsidation. Pre-formed empty shells are disassembled by titration to pH of from 8.5 to 11.0, by elevation of ionic strength to from 1.0 to 3.0 molar sodium chloride or ammonium acetate, or by addition of urea to from 2 to 6 molar or guanidinium chloride to from 1 to 4 molar. Disassembled subunits are mixed with the desired inner core under conditions favorable to cargo stability, and shells are reassembled by stepwise dialysis to restore native ionic strength, pH, and to remove denaturant. Assembly yield is monitored by analytical size-exclusion chromatography, dynamic light scattering, and negative-stain transmission electron microscopy, with acceptance criteria of greater than 70 percent particle population at the expected hydrodynamic diameter and a particle-population polydispersity index below 0.20.

### DNA Origami Scaffold and Staple Production

The single-stranded DNA scaffold of the origami inner core is produced biologically by infection of E. coli with M13mp18-class filamentous bacteriophage or by enzymatic conversion of double-stranded plasmid templates. Bacterial production uses E. coli JM109, XL1-Blue, or ER2738 grown in 2×YT or Terrific Broth medium at 37 degrees Celsius to an OD600 of from 0.3 to 0.5, at which point cultures are inoculated with M13mp18 or modified scaffold variants (p7249, p7560, p7704, p8064, p8634) at a multiplicity of infection of from 1 to 10. Infected cultures continue at 37 degrees Celsius with vigorous aeration for from 4 to 18 hours, after which phage particles secreted into the medium are recovered by polyethylene glycol precipitation (4 percent weight per volume PEG-8000 in 0.5 molar sodium chloride). Phage are resuspended, lysed by alkaline treatment or by phenol-chloroform extraction, and single-stranded scaffold DNA is precipitated with isopropanol. Typical yields are from 5 to 10 milligrams of purified scaffold per liter of culture, with scaffold integrity confirmed by 0.8 percent agarose gel electrophoresis showing a single band of the expected mobility. Enzymatic scaffold production from double-stranded plasmid templates uses lambda-exonuclease digestion of a phosphorylated strand to yield the complementary single-stranded product; the phosphorylated strand is selected during PCR amplification by inclusion of a 5′-phosphorylated primer, and the unphosphorylated complementary strand is recovered after lambda-exonuclease processing at 37 degrees Celsius for from 60 to 180 minutes. Staple oligonucleotides numbering from 150 to 250 per origami design are produced by solid-phase phosphoramidite chemical synthesis at the scale of from 25 nanomoles to 10 micromoles per oligonucleotide, with milligram-to-gram batch totals required for clinical production. Crude oligonucleotides are purified by reverse-phase HPLC on C18 columns or by denaturing polyacrylamide gel electrophoresis (PAGE) on 12 to 20 percent gels, with purity acceptance criteria of greater than 90 percent full-length product by analytical HPLC and by capillary electrophoresis. Pools of staples for a given design are quantified by ultraviolet absorbance at 260 nanometers and mixed at equimolar ratios at a final concentration of from 100 nanomolar to 1 micromolar each.

### DNA Origami Folding

DNA origami folding is performed by combining single-stranded scaffold and staple-oligonucleotide pools at a scaffold concentration of from 5 to 50 nanomolar with from 5- to 20-fold molar excess of each staple in a folding buffer containing from 5 millimolar Tris-HCl pH 8.0 to 10 millimolar Tris-HCl pH 8.0, 1 millimolar ethylenediaminetetraacetic acid (EDTA), and magnesium chloride at a concentration optimized for the specific design (typically from 5 to 20 millimolar). Thermal annealing follows a programmed temperature ramp from 95 degrees Celsius held for from 5 to 15 minutes (to denature secondary structure within the scaffold and staples) followed by linear or stepwise cooling to 20 degrees Celsius over from 12 to 24 hours. Rapid-folding designs employ shortened ramps from 65 degrees Celsius to 20 degrees Celsius over from 1 to 4 hours. Isothermal folding at a constant temperature of from 45 to 55 degrees Celsius in folding buffers supplemented with from 5 to 20 percent volume per volume formamide is used for high-throughput parallel folding of multiple designs in standard PCR thermocyclers without temperature programming. Magnesium concentration is the dominant determinant of folding yield for a given design and is optimized in a screening matrix of from 5 to 25 millimolar in 2.5-millimolar increments, with the optimum magnesium concentration identified by monodisperse migration of the folded product on a 1.5 to 2 percent agarose gel run at 70 volts for from 90 to 180 minutes at 4 degrees Celsius. Folding yield, defined as the percent of input scaffold incorporated into the well-folded product band relative to misfolded aggregates and unfolded scaffold, has acceptance criteria of from 70 to 90 percent for clinical-grade material. In-process controls include monitoring folding-temperature uniformity within plus or minus 0.5 degrees Celsius across the folding vessel, periodic ramp-rate verification by thermocouple, and aliquot withdrawal for analytical gel and negative-stain electron-microscopy verification before scale-up. Scale-up of folding from 50-microliter PCR-tube reactions through 1-milliliter, 100-milliliter, and 10-liter folding vessels is achieved with thermostatic baths and programmable circulators capable of maintaining the prescribed ramp profile across the full vessel volume.

### Origami Stabilization

Folded DNA origami structures require stabilization against degradation by physiological nucleases and against denaturation by low-magnesium environments encountered in vivo. Oligolysine coating is performed by addition of poly-L-lysine peptides of length from 8 to 20 residues (K8 to K20) at amine-to-phosphate ratios of from 0.5 to 2.0, incubated at 4 degrees Celsius for from 1 to 16 hours in low-salt buffer to permit ionic complexation between the cationic lysine side chains and the anionic origami backbone. Coated origami retain structural integrity at magnesium concentrations as low as 0.1 millimolar and resist degradation by serum DNases for greater than 24 hours, compared to less than 60 minutes for uncoated origami. Polysarcosine coating combines an oligolysine anchor segment with a polysarcosine block of from 50 to 200 monomers attached by amide linkage, contributing a hydrophilic stealth shell that reduces serum-protein adsorption and complement activation while retaining electrostatic anchoring. Covalent crosslinking of origami strands is performed by exposure to ultraviolet light at 310 to 365 nanometers for from 1 to 60 minutes, inducing cyclobutane thymidine dimers at engineered T-T pyrimidine pairs at scaffold-staple crossover positions; crosslink density is controlled by the number of engineered T-T sites and the irradiation dose. Click-chemistry crosslinking uses 5-azido-2′-deoxyuridine and 5-ethynyl-2′-deoxyuridine modified nucleotides at scaffold-staple junctions coupled by copper-catalyzed or strain-promoted azide-alkyne cycloaddition. Silica mineralization deposits a thin amorphous silica layer of from 2 to 10 nanometer thickness onto the origami surface by sol-gel polymerization of tetraethyl orthosilicate (TEOS) in the presence of a polyamine such as N-(2-aminoethyl)-3-aminopropyltrimethoxysilane (AEAPTMS) at pH of from 8.0 to 9.5. Stabilized origami are characterized by agarose gel electrophoresis to confirm retained mobility, by dynamic light scattering for hydrodynamic-diameter consistency within plus or minus 10 percent of the pre-stabilization value, and by serum-stability assay measuring residual structural integrity after 24-hour incubation in 10 to 50 percent fetal bovine serum at 37 degrees Celsius.

### Inner-Core-into-Outer-Shell Encapsulation

Encapsulation of the inner DNA-origami core, mineral core, or protein core within the outer virus-like-particle or coat-protein shell is achieved by one of four methods selected to preserve cargo functionality and to maximize encapsulation efficiency. Electrostatic co-assembly is performed when the inner core bears a net negative surface charge (as for unmodified DNA origami) and coat proteins or pentameric subunits carry a positively charged interior surface; co-assembly proceeds by mixing the core and coat components at a stoichiometry of from 1 to 5 cores per assembling shell and slowly reducing ionic strength by dialysis from 1.0 molar to 0.05 molar sodium chloride over from 4 to 24 hours at 4 degrees Celsius. Scaffold-protein-fusion-mediated loading employs a recombinant fusion of a scaffold-mimetic peptide to a cargo-binding domain expressed alongside the coat protein; this fusion recruits cores bearing the complementary binding partner during assembly. Osmotic-shock loading exploits the permeability of pre-formed empty shells under conditions of sudden osmolarity drop. Empty shells are equilibrated in high-osmolarity buffer (from 0.5 to 2.0 molar ammonium sulfate or sodium chloride) and rapidly diluted into a low-osmolarity buffer containing the inner core at high concentration; the resulting transient pore opening permits core entry, after which shells reseal as osmotic equilibrium is restored. Chemical disassembly-reassembly with cargo present is the most general method, applicable when cargo is sufficiently small and stable to tolerate the disassembly conditions. Empty shells are disassembled by treatment with from 2 to 6 molar urea, from 1 to 4 molar guanidinium chloride, or alkaline pH from 9.0 to 11.0; disassembled subunits are mixed with the inner core at a molar ratio of from 1 to 10 cores per equivalent shell, and shells are reassembled by progressive dialysis to remove denaturant or to restore neutral pH over from 4 to 48 hours. Encapsulation efficiency, defined as the percent of input cores incorporated within shells, is determined by sucrose-gradient ultracentrifugation, native agarose gel electrophoresis, or analytical size-exclusion chromatography with on-line ultraviolet absorbance at 260 and 280 nanometers, with acceptance criteria of greater than 50 percent for development material and greater than 70 percent for clinical material.

### Payload Loading Methods

Therapeutic payload molecules are loaded into the inner core by methods selected according to payload chemistry. Small-molecule payloads bearing planar aromatic structures are loaded by intercalation into the double-stranded DNA segments of the origami core at a payload-to-base-pair ratio of from 0.05 to 0.20, with loading achieved by overnight equilibration of empty origami with payload at from 4 to 25 degrees Celsius in folding buffer; loading efficiency is determined by ultracentrifugal separation of bound from free payload and quantification by ultraviolet-visible absorbance. Electrostatic complexation of anionic payloads such as oligonucleotides, antisense agents, or siRNA with a cationic core layer is performed by titration of payload into core at amine-to-phosphate or cation-to-anion ratios of from 1 to 5, with complex formation monitored by zeta-potential measurement and by retention-shift agarose gel electrophoresis. Hybridization-based loading of nucleic-acid payloads to complementary capture sites engineered into the origami core uses from 5 to 50 single-stranded capture extensions of from 15 to 25 nucleotide length protruding from the origami body; capture-extension sequences are designed to be orthogonal to the scaffold and staple sequences and are validated by NUPACK or similar secondary-structure analysis to minimize cross-hybridization. Hybridization loading is performed at from 25 to 45 degrees Celsius in folding buffer for from 30 to 120 minutes, with loading stoichiometry controlled by the number of capture extensions per particle and quantified by fluorophore-labeled payload retention upon gel filtration. Aptamer-mediated capture of protein payloads uses from 5 to 20 aptamer-displaying sites per particle, where aptamers are selected by SELEX against the target protein with dissociation constants of from 1 to 100 nanomolar and incorporated into staple sequences as 3′ or 5′ extensions. Site-specific covalent conjugation of payloads to the origami or shell uses orthogonal click-chemistry handles, including azide-DBCO, tetrazine-trans-cyclooctene, and aldehyde-aminooxy pairs, installed at defined sites by modified-staple incorporation or by genetic encoding of unnatural amino acids in coat proteins. Payload-loading acceptance criteria are payload-to-particle stoichiometry within plus or minus 15 percent of design target, free-payload content below 5 percent, and retention of payload functional activity within plus or minus 20 percent of unloaded reference.

### Surface Conjugation Chemistries

Surface conjugation of targeting ligands, stealth polymers, and imaging agents to the outer shell is performed by chemistries selected according to the available reactive handles on shell and ligand. NHS-ester conjugation forms stable amide bonds between activated carboxylic-acid esters on the ligand and primary amines (lysine side chains and N-terminal alpha-amines) on the shell at a reaction pH of from 7.5 to 8.5 in sodium phosphate buffer of from 50 to 100 millimolar; reactions proceed at 25 degrees Celsius for from 30 to 120 minutes or at 4 degrees Celsius for from 4 to 16 hours, with NHS-ester to amine molar ratios of from 5 to 50 controlling final ligand density. Maleimide-thiol conjugation forms stable thioether bonds between maleimide-functionalized ligands and free cysteine thiols on the shell at a reaction pH of from 6.5 to 7.5; mild reducing conditions are maintained by inclusion of from 1 to 10 millimolar tris(2-carboxyethyl)phosphine (TCEP) or by pretreatment of the shell with TCEP followed by buffer exchange to remove excess reductant before maleimide addition. Copper-catalyzed azide-alkyne cycloaddition (CuAAC) is performed at room temperature for from 30 to 120 minutes using copper sulfate at from 50 to 500 micromolar, tris(3-hydroxypropyltriazolylmethyl) amine (THPTA) ligand at from 250 micromolar to 2.5 millimolar to stabilize copper (I), and sodium ascorbate at from 1 to 5 millimolar as reductant. Copper-free strain-promoted alkyne-azide cycloaddition (SPAAC) using dibenzocyclooctyne (DBCO) or bicyclononyne (BCN) reagents proceeds without copper catalyst at room temperature for from 1 to 16 hours and is preferred for nucleic-acid-containing platforms because of copper-mediated DNA damage avoidance. SpyTag-SpyCatcher conjugation forms a spontaneous isopeptide bond between a 13-amino-acid SpyTag peptide and the 12-kilodalton SpyCatcher protein domain expressed as a genetic fusion to either ligand or shell, with reactions proceeding to completion within from 30 to 120 minutes at micromolar protein concentrations. Sortase-A-mediated ligation transfers a ligand bearing an N-terminal triglycine motif onto a shell-displayed LPXTG motif by transpeptidation, catalyzed by Sortase A from Staphylococcus aureus at from 5 to 50 micromolar enzyme concentration, in 50 millimolar Tris-HCl pH 7.5, 150 millimolar sodium chloride, 10 millimolar calcium chloride, at 25 to 37 degrees Celsius for from 1 to 12 hours. Final ligand density across all conjugation methods is quantified by SDS-PAGE band-shift, by liquid chromatography-mass spectrometry of tryptic peptides, or by fluorescent-ligand counting, with acceptance criteria of mean ligand density per particle within plus or minus 20 percent of target.

### Downstream Purification

Downstream purification follows a multi-step sequence designed to remove host-cell proteins, host-cell DNA, lipopolysaccharide endotoxin, free staples, unassembled coat, free payload, and aggregated species. Clarification of lysate or culture supernatant is performed by depth filtration through grade-DE or grade-CE depth filters with nominal retention of from 0.1 to 1 micrometer, followed by 0.45-micrometer membrane polishing, achieving turbidity reduction of greater than 95 percent and host-cell debris removal of greater than 99 percent. Tangential-flow filtration (TFF) using regenerated-cellulose or polyethersulfone hollow-fiber or cassette membranes is used for buffer exchange, concentration, and diafiltration with molecular-weight cutoffs selected according to particle size: 100-kilodalton cutoff for smaller virus-like particles and naked coat-protein assemblies, 300-kilodalton cutoff for typical 50-to-100-nanometer particles, and 750-kilodalton or 0.05-micrometer cutoff for larger origami structures. Diafiltration is performed against 7 to 10 buffer volumes to reduce permeable contaminants to below 0.1 percent of starting concentration. Polyethylene glycol precipitation is used as an intermediate-step bulk capture, adding PEG-8000 to a final concentration of from 10 to 20 percent weight per volume in the presence of from 0.3 to 0.5 molar sodium chloride and incubating at 4 degrees Celsius for from 30 to 120 minutes; precipitated particles are recovered by centrifugation at from 8,000 to 15,000 times gravity for from 15 to 30 minutes and resuspended in storage buffer. Sucrose-gradient ultracentrifugation on continuous 20 to 50 percent weight per volume sucrose gradients at 100,000 times gravity for from 2 to 18 hours at 4 degrees Celsius separates intact particles from empty shells, aggregates, and free components based on sedimentation coefficient. Size-exclusion chromatography uses Sephacryl S-500 and S-1000 resins for large particles, Superdex 200 for smaller assemblies, and Superose 6 for intermediate sizes, run at linear velocities of from 15 to 30 centimeters per hour in storage buffer with on-line ultraviolet absorbance and conductivity monitoring. Anion-exchange chromatography (Q Sepharose, Capto Q, Mono Q) and cation-exchange chromatography (SP Sepharose, Capto S, Mono S) are used for high-resolution polishing with linear salt gradients of from 0.05 to 1.0 molar sodium chloride. Immobilized-metal-affinity chromatography (IMAC) on Ni-NTA, Co-NTA, or zinc-charged sepharose captures hexahistidine-tagged coats with imidazole gradients of from 20 to 500 millimolar.

### Sterilization Options

Sterilization of the final drug product is achieved by a method selected according to particle size, payload sensitivity, and dosage form. Sterile filtration through 0.22-micrometer or 0.45-micrometer membrane filters (polyethersulfone, polyvinylidene fluoride, or nylon) is the preferred method for particles smaller than 100 nanometers and is performed under laminar-flow conditions with pre-use and post-use integrity testing (bubble-point or pressure-hold). For particle assemblies larger than 100 nanometers or particles that adhere strongly to filter membranes, alternative sterilization is used. Gamma irradiation at doses of from 8 to 25 kilograys delivered from a cobalt-60 source at controlled temperature (typically minus 80 degrees Celsius for biologic products to minimize radiolysis-induced damage) provides terminal sterilization of finished product or of starting materials and process intermediates, with in-process dosimetry to verify dose uniformity across the load. Ethylene oxide sterilization is applied to packaging components, oral dosage forms, and select biologic intermediates compatible with the elevated temperature (from 30 to 60 degrees Celsius), humidity (from 30 to 80 percent relative humidity), and ethylene oxide concentration (from 250 to 1200 milligrams per liter); cycle parameters are validated by biological-indicator challenge with Bacillus atrophaeus spores at a population of 1 times 10 to the 6th. Aseptic process design with closed-system processing, sterilized single-use bioreactor and bag assemblies, and Grade A (ISO 5) workstation environments is used throughout final fill-and-finish to maintain product sterility where terminal sterilization is not feasible, with environmental monitoring, personnel monitoring, and media-fill simulations validating the aseptic capability of the process. Sterility testing of finished product follows USP <71> by direct inoculation or membrane filtration with incubation in fluid thioglycollate and soybean-casein digest media for 14 days. Acceptance criterion is no growth in all tested samples.

Lyophilization with Cryoprotectants

Lyophilization (freeze-drying) is used to convert liquid drug product to a stable solid suitable for long-term storage and oral dosage form manufacturing. Formulation cryoprotectants are selected from trehalose (typically at from 5 to 15 percent weight per volume), sucrose (from 5 to 15 percent weight per volume), mannitol (from 1 to 5 percent weight per volume as bulking agent), and sorbitol (from 1 to 5 percent weight per volume), used singly or in combination at a particle-to-cryoprotectant mass ratio of from 1 to 50 to 1 to 200 by weight. Lyophilization cycle development begins with determination of the formulation collapse temperature (Tc) by freeze-dry microscopy or differential scanning calorimetry of the maximally freeze-concentrated solute, with primary drying performed at a shelf temperature maintained 2 to 5 degrees Celsius below Tc to prevent cake collapse. A representative cycle proceeds through freezing at a shelf temperature ramp of 0.5 to 1.0 degrees Celsius per minute from 5 degrees Celsius to minus 40 to minus 50 degrees Celsius with a hold of 60 to 180 minutes for complete solidification; primary drying at a shelf temperature of from minus 20 to 0 degrees Celsius and chamber pressure of from 50 to 200 millitorr (6.7 to 26.7 pascals) for from 24 to 72 hours; and secondary drying at a shelf temperature of from 20 to 40 degrees Celsius and chamber pressure of from 30 to 100 millitorr (4.0 to 13.3 pascals) for from 6 to 24 hours. Primary drying progress is monitored by product-temperature thermocouples, capacitance manometry, and tunable-diode-laser absorption spectroscopy of water-vapor flux; primary drying is judged complete when product temperature converges to shelf temperature. Residual moisture is determined by Karl Fischer coulometric titration of reconstituted cake aliquots, with an acceptance criterion of less than 2 percent water by mass. Reconstitution time, defined as the interval from addition of diluent to complete cake dissolution, has an acceptance criterion of less than 5 minutes for 1-milliliter cakes. Lyophilized cakes are characterized for particle-size preservation by reconstituted dynamic light scattering (acceptance: hydrodynamic diameter within plus or minus 15 percent of pre-lyophilization), for residual potency by binding and cell-based assays, and for cake appearance (elegant, non-collapsed, non-discolored).

### Oral Dosage Form Manufacturing

The platform is converted to oral dosage forms by processes selected according to dose, target dissolution profile, and patient population. Hard gelatin capsules are filled with lyophilized powder or with granulated blend at fill weights of from 100 to 1000 milligrams per capsule (size 4 to size 00), using semiautomatic or fully automatic encapsulators with in-line weight checking and rejection of capsules outside plus or minus 5 percent of target. Hydroxypropyl methylcellulose (HPMC) capsules of equivalent sizes provide a non-animal-derived alternative with comparable filling characteristics and improved moisture barrier for moisture-sensitive content. Oral tablets are produced by direct compression of a blend of lyophilized drug product, microcrystalline cellulose (from 20 to 50 percent weight per weight), mannitol or lactose (from 10 to 40 percent weight per weight), croscarmellose sodium or sodium starch glycolate disintegrant (from 2 to 6 percent weight per weight), magnesium stearate or sodium stearyl fumarate lubricant (from 0.5 to 2 percent weight per weight), and colloidal silicon dioxide glidant (from 0.25 to 1 percent weight per weight), compressed on rotary tablet presses at compression forces of from 5 to 30 kilonewtons to produce tablets of from 100 to 1000 milligram unit weight with hardness of from 5 to 15 kiloponds, friability below 1 percent, and disintegration time below 30 minutes (or below 15 minutes for orally disintegrating tablets manufactured with high-disintegrant load and lower compression forces). Orally disintegrating tablets are alternatively manufactured by lyophilization-in-blister (Zydis-type) process for from—1-to-10-second disintegration. Oral suspensions are prepared by dispersion of lyophilized particles in aqueous vehicle containing suspending agent (xanthan gum 0.1 to 0.5 percent, carboxymethylcellulose sodium 0.5 to 2 percent, or hydroxyethylcellulose 0.5 to 2 percent), sweetener, flavor, and preservative system, packaged in amber bottles with dosing dropper or oral syringe. Powder for reconstitution is presented as lyophilized powder in vial or sachet for reconstitution with measured volume of water or oral vehicle immediately before administration. Dissolvable oral films are produced by solvent-casting of an aqueous polymer solution (HPMC, pullulan, or modified starch) containing drug particles, dried on a moving belt at from 40 to 70 degrees Celsius to a residual moisture below 6 percent, and cut to unit-dose films of from 2 to 8 square centimeters.

### Enteric Coating Application

Enteric coating protects the platform from acidic gastric degradation and triggers release at small-intestinal or colonic pH. Coating polymers are selected from the Eudragit family (methacrylic acid copolymers) including L30D-55 (aqueous dispersion releasing at pH above 5.5), L100-55 (organic-solution coating releasing at pH above 5.5), L100 (releasing at pH above 6.0), S100 (releasing at pH above 7.0), and FS30D (releasing at pH above 7.0 with colon-targeting kinetics), or from the cellulose-derivative family including hydroxypropyl methylcellulose acetate succinate (HPMC-AS) in LF, LG, MF, MG, HF, and HG grades providing controlled release across pH 5.5 to 6.8. Coating application uses fluidized-bed coating (Wurster bottom-spray for particles and pellets, top-spray for granules, tangential-spray for spheronized cores) with atomization air pressure of from 1.0 to 3.0 bar, spray rate of from 5 to 25 grams per minute per kilogram batch, inlet air temperature of from 30 to 50 degrees Celsius, and product temperature maintained at from 25 to 35 degrees Celsius. Pan coating is used for tablet coating in side-vented coating pans of from 12 to 60 inch diameter operated at pan speeds of from 5 to 20 revolutions per minute with spray rate, atomization, and air-flow scaled to batch size. Spray-drying coating produces enterically coated microparticles in a single step by co-spraying drug solution or suspension with coating polymer through a two-fluid or three-fluid nozzle, with an inlet air temperature of from 100 to 180 degrees Celsius and outlet air temperature of from 50 to 80 degrees Celsius. Coating-thickness control is achieved by gravimetric weight gain (target weight gain of from 8 to 20 percent for tablets, from 15 to 35 percent for pellets and particles) measured at intermediate sampling points during the coating run, with adjustment of spray rate or process time to converge on the target. Dissolution-test acceptance criteria follow USP <711> Method A or B: less than 10 percent payload release in 0.1 normal hydrochloric acid (pH 1.2) over 2 hours, followed by greater than 80 percent release in pH 6.8 phosphate buffer over a defined window (typically 30 to 90 minutes).

### Quality Control by Physicochemical Characterization

Each batch of platform drug product is characterized by an orthogonal panel of physicochemical assays. Dynamic light scattering (DLS) at multiple concentrations and scattering angles measures the intensity-weighted hydrodynamic diameter (Z-average) and the polydispersity index (PDI), with acceptance criteria of Z-average within plus or minus 20 percent of target and PDI below 0.30. Transmission electron microscopy (TEM) on uranyl-acetate-, uranyl-formate-, or phosphotungstic-acid-negative-stained grids visualizes particle morphology, shell-integrity, core-encapsulation status, and aggregation state, with acceptance criteria of greater than 80 percent monodisperse, morphologically intact particles in random-field micrograph counts. Cryo-electron microscopy (cryo-EM) on vitrified samples imaged on a 200- or 300-kilovolt microscope equipped with direct-electron detector provides near-atomic-resolution (3 to 5 angstrom) structures of the shell and inner core through single-particle reconstruction, with acceptance applied to lot-comparability across reference and test batches. Atomic force microscopy (AFM) in liquid or air modes measures particle height, surface roughness, and mechanical properties (Young's modulus by force-distance curves). Small-angle X-ray scattering (SAXS) in solution provides the particle radius of gyration, pair-distance distribution function, and ab initio low-resolution shape, complementing electron microscopy with statistical averaging over from 10 to the 10th to 10 to the 12th particles per measurement. Native mass spectrometry on time-of-flight or Orbitrap instruments with extended mass range determines particle stoichiometry and detects mass shifts from incomplete subunit incorporation, payload loading, or ligand conjugation. SDS-PAGE under reducing and non-reducing conditions resolves protein components, with Coomassie or silver staining detecting host-cell proteins above 0.1 percent of total band intensity. Native and denaturing agarose gel electrophoresis resolves nucleic-acid components of origami-containing platforms and confirms folding integrity. Endotoxin is quantified by the limulus amebocyte lysate (LAL) chromogenic, turbidimetric, or gel-clot method or by recombinant Factor C assay, with an acceptance criterion of less than 100 endotoxin units per dose for oral product (per ICH and USP <85>).

### Quality Control by Binding-Function Assays

Functional integrity of surface-displayed targeting ligands is verified by binding assays that measure interaction with the cognate receptor or target. Surface plasmon resonance (SPR) on instruments of the Biacore, Carterra LSA, or Bruker Sierra SPR-32 family measures real-time association and dissociation kinetics of ligand-receptor binding, providing on-rate (ka), off-rate (kd), and equilibrium dissociation constant (KD). Particles or particle-displayed ligands are flowed over receptor immobilized to a CM5 or NTA sensor chip surface at concentrations spanning from 0.1- to 10-fold KD, and sensorgrams are fit to 1-to-1 Langmuir or heterogeneous-ligand kinetic models with acceptance criteria of KD within plus or minus 3-fold of reference and chi-squared values below 10 percent of maximal response. Biolayer interferometry (BLI) on Octet platforms provides equivalent kinetic and affinity measurements with biosensors functionalized with receptor or capture-antibody, suitable for higher-throughput screening of lot-to-lot variability. ELISA-based binding assays detect binding of platform-displayed ligands to plate-immobilized receptor using horseradish-peroxidase-conjugated or alkaline-phosphatase-conjugated detection antibodies, with quantification by 3,3′,5,5′-tetramethylbenzidine or p-nitrophenyl-phosphate substrate development and absorbance reading at 450 or 405 nanometers, generating EC50 values with acceptance criteria of within plus or minus 3-fold of reference. Cellular uptake assays in human colorectal adenocarcinoma Caco-2 cells (modeling intestinal epithelium) and in immortalized human brain microvascular endothelial cell line hCMEC/D3 (modeling blood-brain barrier) quantify particle internalization by flow cytometry of fluorescently labeled particles or by confocal microscopy with z-stack reconstruction, with acceptance criteria of mean fluorescence intensity within plus or minus 30 percent of reference and intracellular localization confirmed in greater than 70 percent of imaged cells.

### Quality Control by Potency Assays

Potency of each drug product lot is established by cell-based and tissue-based assays reflecting the intended pharmacological mechanism. Cellular cytotoxicity is determined in disease-relevant cell models (for example, patient-derived cancer cell lines for oncology indications, iPSC-derived neurons or organoids for neurodegenerative indications, or primary hepatocytes for metabolic-disease indications) by quantification of cell viability by MTT, XTT, CellTiter-Glo, or resazurin reduction after exposure to particle dilutions spanning 4 to 6 log 10 concentration; relative potency is calculated against a qualified reference lot by parallel-line or 4-parameter logistic curve fitting, with acceptance criteria of relative potency within 60 to 140 percent of reference per the USP <1032>, <1033>, and <1034> bioassay-validation framework. Pharmacodynamic readouts in primary neuronal cultures from rodent embryonic cortex or hippocampus, or from human iPSC-derived neurons, measure ion-channel modulation by patch-clamp electrophysiology, synaptic function by multielectrode array recording, or protein expression by Western blot, depending on the platform's mechanism of action. Ex vivo brain-slice activity assays prepare 300- to 400-micrometer-thick horizontal or coronal slices from rodent brain perfused with oxygenated artificial cerebrospinal fluid at 32 to 35 degrees Celsius, with platform-mediated effects on field potentials, local-field potentials, or fluorescence-encoded calcium signals recorded over from 30 to 180 minutes of perfusion. Potency-assay validation per ICH Q2 (R1) establishes specificity, linearity over the working range, intermediate precision below 20 percent coefficient of variation, and accuracy within plus or minus 15 percent of nominal across the assay range.

### Release-Testing Acceptance Criteria

Lot release of finished drug product is governed by a release specification that combines identity, purity, content, potency, and safety attributes with predefined acceptance criteria. Particle size by DLS Z-average is required to be within plus or minus 20 percent of the lot-specific target, with polydispersity index below 0.30; particles outside specification are rejected. Payload content, determined by orthogonal methods including ultraviolet-visible absorbance, fluorescence (where payload is fluorescent or has been chemically tagged), and chromatographic quantification by RP-HPLC or HILIC-HPLC with appropriate detector (UV at 220 to 280 nanometers, fluorescence, or coupled mass spectrometry), is required to be from 80 to 120 percent of label claim. Endotoxin by LAL or recombinant Factor C is required to be below 100 endotoxin units per dose. Bioburden (for non-sterile oral product) meets compendial limits of less than 1000 colony-forming units per gram of total aerobic microbial count and less than 100 colony-forming units per gram of total combined yeasts and molds, with absence of Escherichia coli, Salmonella, Staphylococcus aureus, and Pseudomonas aeruginosa per USP <61> and <62>; sterile products meet USP <71> sterility. Ligand density per particle, determined by fluorescent-ligand counting, by mass spectrometry of tryptic peptides, or by ELISA of denatured shell, is required to be within plus or minus 20 percent of target. Residual host-cell protein is required to be below 100 parts per million by host-cell-protein ELISA, residual host-cell DNA below 10 nanograms per dose by quantitative PCR or threshold-amplification assay, and residual organic solvents (where applicable) below ICH Q3C class-specific limits. Identity is confirmed by orthogonal assays including peptide mapping by liquid chromatography-mass spectrometry, by SDS-PAGE band pattern, and by binding profile against the cognate receptor. Each release specification table is locked into the regulatory file (IND, BLA, or NDA Module 3.2.P.5) and is the gate for lot release by the qualified person.

### Stability Testing per ICH Q1A Guidelines

Stability of the drug product is established per ICH Q1A (R2) under three storage conditions: long-term at 25 plus or minus 2 degrees Celsius and 60 plus or minus 5 percent relative humidity, intermediate at 30 plus or minus 2 degrees Celsius and 65 plus or minus 5 percent relative humidity, and accelerated at 40 plus or minus 2 degrees Celsius and 75 plus or minus 5 percent relative humidity. For refrigerated products, long-term storage is at 5 plus or minus 3 degrees Celsius with accelerated storage at 25 plus or minus 2 degrees Celsius and 60 plus or minus 5 percent relative humidity. Stability time-points include 0, 3, 6, 9, 12, 18, 24, and 36 months for long-term, 0, 6, and 12 months for intermediate, and 0, 1, 2, 3, and 6 months for accelerated, with additional time points added as supported by program-specific stability data. At each timepoint, primary stability-indicating attributes are measured: particle size and polydispersity by DLS, particle morphology by TEM, payload content by chromatographic assay, potency by the qualified cell-based bioassay, sterility or bioburden, residual moisture by Karl Fischer (for lyophilized products), and dosage-form-specific attributes (tablet hardness, capsule fill weight, dissolution profile, friability). Stability-indicating impurity profiles are tracked by orthogonal chromatographic methods. Photostability per ICH Q1B is established by exposure to 1.2 million lux-hour visible light plus 200 watt-hour per square meter near-ultraviolet light in a calibrated photostability chamber. Stress-stability studies under thermal, oxidative, and freeze-thaw stress establish degradation pathways and inform formulation development. Shelf-life targets are 24 to 36 months at the labeled storage condition, with shelf-life assigned by ICH Q1E statistical analysis of pooled or batch-specific stability data using linear or polynomial regression with 95 percent one-sided confidence bounds on the relevant specification attribute.

### Scale-Up Considerations

Process development progresses through defined scale tiers: laboratory scale produces milligram to gram quantities of drug substance for characterization, formulation screening, and preclinical pharmacology; engineering scale produces from 10 to 100 grams for toxicology lots, preclinical safety studies, and early process characterization; clinical scale produces kilogram quantities for Phase 1 to Phase 3 clinical supply; and commercial scale produces multi-kilogram batches for market supply. Each scale transition is qualified by engineering runs at the new scale that demonstrate process parameters fall within the design space established at the prior scale and that critical quality attributes meet the previously qualified specifications. Process validation per ICH Q7, ICH Q8, ICH Q9, ICH Q10, ICH Q11, and FDA Process Validation Guidance is performed on three consecutive at-scale batches at the intended commercial scale, with prospective protocol-defined acceptance criteria covering critical process parameters, in-process controls, and finished-product specifications. Tech transfer documentation includes a master batch record, a process flow diagram, a process description, a list of raw materials and components with supplier qualifications, equipment qualifications (IQ, OQ, PQ), analytical-method validation per ICH Q2 (R1), facility fit assessment, cleaning validation, environmental-monitoring qualification, and a tech-transfer risk assessment per ICH Q9. cGMP qualification per 21 CFR Parts 210 and 211 is established before clinical lots are released, with quality systems for change control, deviation management, corrective and preventive action (CAPA), supplier qualification, product complaint, and annual product review. Scale-up risks unique to the platform include shell-assembly yield reduction at higher folding or assembly volumes (mitigated by jacket-temperature uniformity studies), shear-induced particle damage in tangential-flow filtration and stirred-tank operation (mitigated by sweep-rate and impeller-design optimization), and concentration-dependent aggregation during ultrafiltration (mitigated by formulation excipient optimization).

### Batch, Fed-Batch, and Continuous-Flow Manufacturing Configurations

Three manufacturing configurations are available for upstream and downstream operations, selected according to throughput, capital, and quality consistency targets. Batch manufacturing operates each unit operation as a discrete sequence with defined start and end points: a single fermentation harvest feeds a discrete lysis, clarification, capture-chromatography, polishing-chromatography, ultrafiltration, sterile-filtration, and fill-finish sequence, with quality control sampled and released at the end of each step. Batch operation provides clear regulatory definition of lot boundaries, simplifies investigation of deviations, and is the default configuration for clinical and small-commercial supply. Fed-batch manufacturing extends the upstream operation by continuous addition of carbon-source feed during fermentation as described in section above, increasing cell density and product titer (typically by from 3- to 10-fold over simple batch fermentation) while retaining the discrete batch structure of downstream operations. Continuous-flow manufacturing replaces discrete batch steps with continuous unit operations connected by surge tanks and in-line control: continuous perfusion fermentation maintains steady-state cell density and product harvest rate over from 14 to 60 days, feeding continuous clarification by acoustic separation or alternating tangential-flow filtration, continuous chromatography by simulated moving-bed or periodic counter-current chromatography, continuous ultrafiltration, and continuous fill-finish. Throughput in continuous operation is from 3- to 10-fold higher per unit floor space than equivalent batch operation, with concomitant reductions in capital equipment footprint, raw-material consumption, and water-for-injection demand. Quality consistency in continuous operation is supported by process analytical technology (PAT) including in-line Raman, near-infrared, ultraviolet-visible, and fluorescence spectroscopy, in-line refractive index, in-line dynamic light scattering, and at-line liquid chromatography, with real-time control loops adjusting process parameters to maintain critical quality attributes within design space; lot definition in continuous operation is by time-based segmentation per FDA continuous-manufacturing guidance. Selection among batch, fed-batch, and continuous configurations is governed by product-specific risk-benefit analysis covering yield consistency (typical batch-to-batch variability: batch 15 to 25 percent CV, fed-batch 10 to 20 percent CV, continuous 5 to 12 percent CV after steady state), capital intensity (continuous favored for high-volume products with stable demand), and regulatory familiarity (batch is most established).

### Expanded Axis 9—Monitoring, Biomarker Integration, Closed-Loop Feedback (Deep Dive)

### Monitoring, Biomarker Integration, and Closed-Loop Feedback Control

### Circulating-Biomarker Monitoring of Central Nervous System Therapeutics in Peripheral Blood

The platform integrates a peripheral blood biomarker monitoring subsystem configured to quantify circulating proteoform concentrations indicative of central nervous system disease state, neuronal injury, and therapeutic response without requirement for cerebrospinal fluid sampling. Plasma amyloid-beta isoform ratios, specifically the ratio of amyloid-beta peptide species terminating at residue forty-two to species terminating at residue forty (Aβ42/Aβ40), are quantified by immunoprecipitation-mass spectrometry, single-molecule array immunoassay, or chemiluminescent enzyme immunoassay at a sensitivity of from 0.5 picograms per milliliter to 100 picograms per milliliter. A decrease in the Aβ42/Aβ40 ratio of from 0.05 to 0.20 relative to age-matched cognitively normal reference distributions is associated with elevated brain amyloid burden and constitutes a triggering input for the closed-loop dose-adjustment controller described herein. Plasma neurofilament light chain (NfL) is quantified by single-molecule array immunoassay or electrochemiluminescent immunoassay over a dynamic range of from 1.0 picograms per milliliter to 1000 picograms per milliliter, with longitudinal increases of from 10 percent to 50 percent over a sliding three-month baseline window interpreted as evidence of subclinical neuroaxonal injury and triggering review of dose intensity, dose interval, or formulation switching. Plasma phosphorylated tau species, including tau phosphorylated at threonine 181 (p-tau181) and tau phosphorylated at threonine 217 (p-tau217), are quantified by immunoassays with sensitivity of from 0.05 picograms per milliliter to 50 picograms per milliliter, providing AD-specific disease-state monitoring with concordance to amyloid positron emission tomography exceeding 85 percent for p-tau217 in validated clinical cohorts. The feedback controller integrates these measurements at intervals of from one week to twelve weeks, with sampling cadence adjusted by disease stage, treatment phase, and prior biomarker trajectory variance.

Additional circulating biomarkers integrated into the monitoring subsystem include plasma glial fibrillary acidic protein (GFAP) as a marker of astrocytic activation and reactive astrogliosis, quantified over a range of from 20 picograms per milliliter to 2000 picograms per milliliter by single-molecule array; plasma ubiquitin C-terminal hydrolase L1 (UCH-L1) as a marker of neuronal cell-body injury, quantified over a range of from 5 picograms per milliliter to 500 picograms per milliliter; and plasma S100 calcium-binding protein B (S100-B) as a marker of glial activation and blood-brain barrier perturbation, quantified by chemiluminescent immunoassay over a range of from 0.02 micrograms per liter to 5.0 micrograms per liter. For synucleinopathies, including Parkinson disease, dementia with Lewy bodies, and multiple system atrophy, the platform incorporates conformational-species-selective immunoassays detecting plasma alpha-synuclein oligomers, aggregated species, and exosome-encapsulated alpha-synuclein at sensitivity of from 0.1 picograms per milliliter to 100 picograms per milliliter, optionally coupled with seed-amplification assays providing qualitative aggregation positivity. For Huntington disease, the platform quantifies plasma mutant huntingtin fragments by single-molecule array using monoclonal antibodies selective for polyglutamine-expanded epitopes, with sensitivity of from 0.05 femtomoles per milliliter to 50 femtomoles per milliliter. For amyotrophic lateral sclerosis subtypes with superoxide dismutase 1 mutation, plasma SOD1 protein and SOD1-aggregate species are quantified by validated immunoassays with sensitivity of from 1 picograms per milliliter to 200 picograms per milliliter. Each biomarker is registered in the patient-specific longitudinal database with assay-specific reference intervals, intra-individual coefficient of variation, and pre-analytical handling parameters; the controller applies sample-quality flags to exclude measurements compromised by hemolysis, lipemia, prolonged room-temperature exposure, or freeze-thaw cycling outside validated tolerances.

The feedback loop integrates circulating biomarker signals through a hierarchical decision architecture. At the lowest tier, individual biomarker measurements are compared to patient-specific personalized reference intervals derived from baseline measurements obtained during a pre-treatment run-in phase of from 4 weeks to 12 weeks; deviations exceeding two-and-one-half intra-individual standard deviations trigger flagging for repeat sampling within from 7 days to 28 days. At an intermediate tier, multi-marker composite scores integrating amyloid (Aβ42/Aβ40), tau (p-tau181, p-tau217), neuronal injury (NfL, UCH-L1), and glial (GFAP, S100-B) panels are computed and benchmarked against pre-specified treatment-response thresholds; favorable composite trajectories permit dose-interval lengthening within prescribed bounds, while unfavorable trajectories trigger dose-intensity escalation or formulation-switching review by the responsible clinician. At the highest tier, multi-month longitudinal biomarker trajectories are submitted to the model-predictive control and reinforcement-learning subsystems for individualized regimen optimization. Safety considerations encompass biomarker assay quality control via per-batch internal calibrators and external proficiency testing; pre-analytical standardization of phlebotomy timing relative to dosing, sample-tube specifications, centrifugation parameters, and freezer-storage temperatures; assay-platform consistency across longitudinal follow-up with documented bridging studies when reagent lots or instrument platforms change; and explicit handling of biomarker increases attributable to concurrent acute illness, traumatic injury, recent surgery, or other non-treatment-related confounders that may transiently perturb circulating concentrations.

### Cerebrospinal Fluid Biomarkers Accessible by Lumbar Puncture

For clinical contexts in which cerebrospinal fluid (CSF) sampling is performed at baseline and at protocol-defined intervals, the platform incorporates a CSF biomarker panel providing direct measurement of central-compartment neurochemistry and complementing peripheral-blood monitoring. CSF amyloid-beta 1-42 is quantified by chemiluminescent enzyme immunoassay or electrochemiluminescent assay over a range of from 200 picograms per milliliter to 3000 picograms per milliliter; CSF total tau over a range of from 50 picograms per milliliter to 2000 picograms per milliliter; and CSF tau phosphorylated at threonine 181 over a range of from 10 picograms per milliliter to 250 picograms per milliliter. The ratios CSF p-tau181/Aβ42 and CSF total-tau/Aβ42 are computed and benchmarked against assay-specific validated thresholds for amyloid positivity and Alzheimer disease pathophysiology. CSF neurofilament light chain is quantified by single-molecule array over a range of from 100 picograms per milliliter to 30000 picograms per milliliter, providing a high-dynamic-range index of central neuroaxonal injury that is more proximal to brain parenchyma than the corresponding plasma measurement. CSF alpha-synuclein, including total alpha-synuclein and oligomer-selective measurements, is quantified over a range of from 500 picograms per milliliter to 5000 picograms per milliliter; seed-amplification assays (real-time quaking-induced conversion and protein misfolding cyclic amplification adaptations for alpha-synuclein) are deployed as qualitative confirmatory assays for synucleinopathy diagnosis and therapeutic stratification. Pre-analytical handling requires polypropylene collection tubes, controlled-volume aliquoting to minimize protein adsorption losses, and standardized centrifugation and freezing within from 30 minutes to 120 minutes of collection.

Additional CSF analytes integrated into the feedback architecture include CSF chitinase-3-like protein 1 (YKL-40, also designated CHI3L1) as a marker of microglial and astrocytic activation, quantified over a range of from 50 nanograms per milliliter to 2000 nanograms per milliliter; CSF soluble triggering receptor expressed on myeloid cells 2 (sTREM2) as a marker of microglial response, quantified over a range of from 500 picograms per milliliter to 50000 picograms per milliliter; and CSF complement components, including C3, C4, factor B, C5a, and the membrane attack complex (sC5b-9), quantified by validated immunoassays. CSF chemokines and cytokines including monocyte chemoattractant protein-1 (MCP-1/CCL2), interleukin-6, interleukin-8, interleukin-10, interleukin-1 beta, tumor necrosis factor alpha, interferon gamma, and CXCL10 are quantified by multiplex bead-array immunoassays over ranges of from 0.5 picograms per milliliter to 5000 picograms per milliliter. The closed-loop controller integrates CSF measurements at lower temporal frequency than plasma measurements, typically at baseline, at from 3 months to 6 months, at from 9 months to 12 months, and annually thereafter, recognizing that lumbar puncture is invasive and not appropriate for high-frequency sampling. The controller weights CSF signals more heavily than concordant plasma signals when both are available, owing to lower peripheral-compartment dilution and proximity to the target tissue. Safety considerations encompass standardized lumbar-puncture technique by trained operators, atraumatic needle selection to minimize post-dural-puncture headache incidence, contraindication screening for elevated intracranial pressure and coagulopathy, and procedural-adverse-event documentation in the feedback database.

### Volumetric and Microstructural Imaging Biomarkers

Magnetic resonance imaging (MRI) is incorporated as a structural and microstructural biomarker modality. Standardized acquisition protocols include three-dimensional T1-weighted magnetization-prepared rapid gradient-echo sequences for volumetric segmentation, T2-weighted and fluid-attenuated inversion recovery (FLAIR) sequences for white-matter hyperintensity and lesion-burden quantification, susceptibility-weighted imaging for microbleed detection, and diffusion-weighted and diffusion tensor imaging for microstructural assessment. Quantitative volumetric analysis is performed by validated automated segmentation pipelines yielding hippocampal volume over a range of from 1500 cubic millimeters to 5000 cubic millimeters per hemisphere; entorhinal cortex volume; basal ganglia structures (caudate, putamen, globus pallidus, substantia nigra) volumes; thalamic nuclei volumes; cortical thickness across the Desikan-Killiany or comparable parcellation atlases; total brain volume; lateral ventricular volume over a range of from 5 milliliters to 100 milliliters; and white-matter hyperintensity volume. Longitudinal volume changes are quantified by tensor-based morphometry, boundary-shift integral, or comparable registration-based methods with intra-subject reproducibility coefficients of from 0.5 percent to 2.0 percent per year. Treatment-attributable preservation or reversal of regional atrophy trajectories relative to a model-predicted untreated trajectory is integrated into the controller as a long-horizon efficacy signal.

Diffusion tensor imaging (DTI) metrics including fractional anisotropy and mean diffusivity are computed at the voxel level and aggregated across white-matter tract atlases (Johns Hopkins University ICBM-DTI-81 atlas, tract-based spatial statistics skeletons) or via fiber-tractography parcellations. Fractional anisotropy ranges from 0 to 1 with white-matter typical values of from 0.4 to 0.8; mean diffusivity ranges from 0.5×10\hat{ }-3 square millimeters per second to 1.5×10\hat{ }-3 square millimeters per second in white matter. Longitudinal decreases in fractional anisotropy or increases in mean diffusivity within disease-relevant tracts (fornix, cingulum, uncinate fasciculus, corticospinal tract, optic radiations) provide microstructural-injury signals incorporated into the composite biomarker score. Magnetic resonance spectroscopy at 3 Tesla or 7 Tesla field strength is acquired with point-resolved spectroscopy (PRESS), stimulated-echo acquisition mode (STEAM), or semi-localization-by-adiabatic-selective-refocusing (sLASER) localization sequences and quantified for N-acetyl-aspartate (NAA), choline-containing compounds (Cho), creatine plus phosphocreatine (Cr+PCr), glutamate, glutamine, gamma-aminobutyric acid (GABA), and myo-inositol. NAA/Cr ratios over a range of from 0.8 to 2.5 serve as neuronal-integrity indices; elevated Cho/Cr reflects membrane turnover; myo-inositol/Cr elevations reflect glial activation. Imaging measurements are acquired at baseline, at from 6 months to 12 months, and at annual intervals thereafter; safety considerations include MRI-compatibility screening for implants, gadolinium-contrast risk assessment when contrast-enhanced sequences are required, and standardized scanner-platform calibration including phantom-based quality control to enable longitudinal and multi-site comparability.

### Positron Emission Tomography Biomarkers

Positron emission tomography (PET) biomarkers are integrated for molecular-target quantification complementing structural MRI. Amyloid-beta PET is performed using radioligands of the florbetapir class (18F-florbetapir, also known as Amyvid), florbetaben class (18F-florbetaben, also known as Neuraceq), and flutemetamol class (18F-flutemetamol, also known as Vizamyl), with quantification by standardized uptake value ratio (SUVR) against whole-cerebellum or cerebellar-cortex reference regions and by Centiloid scaling over a range of from −20 Centiloids to 200 Centiloids. SUVR values for cortical composite regions typically range from 0.9 to 2.5; the Centiloid scale enables harmonization across tracers and sites. Tau PET is performed using flortaucipir class tracers (18F-flortaucipir, also known as Tauvid) and second-generation tracers including 18F-PI-2620, 18F-MK-6240, and 18F-RO-948, with quantification by SUVR in Braak-stage-defined regions of interest (entorhinal cortex, inferior temporal cortex, posterior cingulate, lateral parietal cortex, frontal cortex) ranging from 1.0 to 4.0. Treatment-attributable changes in regional amyloid and tau PET SUVR, including reductions of from 5 Centiloids to 80 Centiloids in amyloid burden and slowing of tau accumulation rates of from 10 percent to 60 percent relative to model-predicted untreated trajectories, are integrated into the feedback controller as definitive molecular-target engagement signals.

Additional PET modalities integrated for movement-disorder, neuroinflammation, synaptic-density, and metabolic monitoring include dopamine transporter PET using 18F-FP-CIT, 11C-CFT, or 123I-FP-CIT single-photon emission tracers in striatal regions of interest, quantified by specific-binding ratios over a range of from 0.5 to 4.0; vesicular monoamine transporter 2 PET using 18F-AV-133 (also known as 18F-florbenazine) for presynaptic monoaminergic neuron integrity; synaptic vesicle glycoprotein 2A PET using 11C-UCB-J for synaptic-density quantification over distribution-volume ratios of from 0.5 to 4.0; translocator protein 18-kilodalton (TSPO) PET using second-generation tracers 11C-PBR28, 18F-DPA-714, or 18F-PBR06 for activated microglia and reactive astrocyte quantification, with binding-potential interpretation stratified by TSPO rs6971 polymorphism (high-, mixed-, and low-affinity binders); and 18F-fluorodeoxyglucose (FDG) PET for regional cerebral glucose metabolic rate, quantified by SUVR or fully quantitative metabolic-rate-of-glucose imaging in disease-relevant networks (posterior cingulate, precuneus, temporoparietal cortex for Alzheimer disease; basal ganglia for Huntington disease; brainstem and cerebellum for atypical parkinsonian syndromes). PET acquisitions are typically performed at baseline, at from 12 months to 18 months, and at from 24 months to 36 months, recognizing the cumulative radiation-dose constraints (typical per-scan effective dose of from 4 millisieverts to 10 millisieverts) and the cost of cyclotron-produced and on-site-radiosynthesized tracers. Safety considerations include radiation-dose accumulation tracking across the longitudinal protocol, contraindications in pregnancy, allergy screening for tracer-vehicle constituents, and standardized cross-site harmonization of scanner-platform calibration and image-reconstruction parameters.

### Functional Neuroimaging Biomarkers

Functional neuroimaging biomarkers are acquired to characterize network-level brain function and quantify treatment-attributable functional changes that may precede or accompany structural changes. Resting-state functional MRI (rs-fMRI) is acquired over scan durations of from 5 minutes to 15 minutes per session at 3 Tesla or 7 Tesla with echo-planar imaging blood-oxygen-level-dependent (BOLD) contrast; preprocessing includes motion correction, slice-timing correction, spatial normalization, nuisance regression for cerebrospinal fluid and white-matter signals and motion parameters, and band-pass temporal filtering over a frequency range of from 0.008 hertz to 0.10 hertz. Connectivity within the default mode network (medial prefrontal cortex, posterior cingulate cortex, precuneus, angular gyrus, hippocampal formation), salience network, frontoparietal executive control network, and disease-relevant networks (sensorimotor for Parkinson disease, language networks for primary progressive aphasias) is quantified by seed-based correlation analysis, independent component analysis, and graph-theoretic metrics including modularity, global efficiency, and characteristic path length. Treatment-attributable preservation or restoration of default-mode-network connectivity, with change magnitudes of from 0.05 to 0.30 in Fisher-transformed correlation coefficients, is incorporated into the composite efficacy score. Task-based fMRI of disease-relevant cognitive networks, including episodic memory encoding for Alzheimer disease, motor sequencing for Parkinson disease, and inhibitory control for Huntington disease, provides target-engagement readouts complementing resting-state metrics.

Arterial spin labeling (ASL) perfusion MRI is incorporated for non-contrast quantification of regional cerebral blood flow over a range of from 20 milliliters per 100 grams per minute to 100 milliliters per 100 grams per minute. Pseudo-continuous ASL with three-dimensional gradient-and-spin-echo readout is the preferred implementation. Regional perfusion deficits in posterior cingulate, precuneus, and temporoparietal cortex for Alzheimer disease and in posterior parietal and occipital cortex for dementia with Lewy bodies provide functional-network signals. Electroencephalography (EEG) is acquired with high-density arrays of from 32 channels to 256 channels at sampling rates of from 250 hertz to 5000 hertz; power-spectral analysis quantifies absolute and relative power in delta (0.5 to 4 hertz), theta (4 to 8 hertz), alpha (8 to 13 hertz), beta (13 to 30 hertz), and gamma (30 to 80 hertz) bands across scalp electrodes and source-localized regions. Functional connectivity is computed via coherence, phase-locking value, weighted phase-lag index, and amplitude-envelope correlation; graph-theoretic network metrics complement direct connectivity measures. Magnetoencephalography (MEG) is acquired with 275-channel to 306-channel whole-head arrays using axial gradiometers, planar gradiometers, or superconducting quantum interference device magnetometers; source localization is performed by beamforming, minimum-norm estimation, or dipole fitting to yield cortical-source time series. The feedback controller incorporates functional-imaging-derived network-integrity scores at protocol intervals of from 3 months to 12 months. Safety considerations include MRI screening, EEG and MEG patient comfort and electrode-impedance management, and standardized acquisition parameters enabling longitudinal comparability.

### Wearable-Device-Derived Digital Biomarkers

Wearable-device-derived digital biomarkers are continuously acquired in the patient's home environment, providing high-temporal-resolution functional measurements complementing episodic clinic-based assessments. Continuous wrist actigraphy is acquired by triaxial accelerometers at sampling rates of from 30 hertz to 100 hertz, with derived metrics including total sleep time, sleep efficiency (target above 85 percent), sleep-onset latency, wake-after-sleep-onset minutes, number of awakenings, rest-activity rhythm amplitude, mesor, acrophase, and interdaily stability and intradaily variability indices. Circadian rhythm fragmentation, quantified by intradaily variability of from 0 to 2, is a prodromal marker for multiple neurodegenerative disorders and is incorporated as a disease-progression biomarker. Gait analysis is performed using inertial measurement units placed at the lumbar or shank position, with derived metrics including cadence (steps per minute), stride length, stride-time variability, gait speed (typically from 0.4 meters per second to 1.6 meters per second), double-support time, asymmetry indices, freezing-of-gait episode frequency and duration, and turning velocity. Speech and language metrics are derived from voice-recording analysis including fundamental frequency variability, jitter, shimmer, harmonics-to-noise ratio, articulation rate, pause-time distributions, and natural-language-processing-derived measures of lexical diversity, syntactic complexity, semantic coherence, and disfluency rates.

Additional wearable-derived biomarkers include electrocardiogram (ECG)-derived heart-rate variability metrics from chest-strap or wrist-worn single-lead ECG monitors, with time-domain measures (standard deviation of normal-to-normal R-R intervals (SDNN), root-mean-square of successive R-R differences (RMSSD)), frequency-domain measures (low-frequency power 0.04 to 0.15 hertz, high-frequency power 0.15 to 0.4 hertz, low-frequency-to-high-frequency ratio), and nonlinear measures (sample entropy, detrended fluctuation analysis alpha exponents) computed over time windows of from 5 minutes to 24 hours. Autonomic-nervous-system dysfunction in alpha-synucleinopathies is reflected in reduced RMSSD and altered low-frequency power. Photoplethysmography (PPG)-derived pulse-wave metrics from wrist-worn optical sensors provide heart rate, pulse-wave amplitude, pulse-arrival time, and continuous oxygen saturation. Continuous skin-temperature monitoring at the wrist or chest provides circadian-temperature rhythm metrics. Activity counts and intensity distributions, quantified as minutes per day in sedentary (less than 1.5 metabolic equivalents (METs)), light (1.5 to 3 METs), moderate (3 to 6 METs), and vigorous (above 6 METs) intensity bins, are derived from accelerometry and serve as overall functional-status indices. The closed-loop controller integrates daily aggregated wearable summaries at weekly review intervals and high-frequency time-series for prespecified safety triggers (e.g., sudden gait-speed drop exceeding 25 percent of personalized baseline, prolonged sleep disruption). Safety considerations include device-skin-tolerability monitoring, battery and charging compliance support, data-loss imputation rules, and patient-privacy safeguards governing continuous in-home sensing.

### Smartphone-Based Assessment

Smartphone-based assessment modules are deployed via a regulated mobile application providing structured cognitive and motor assessments and passive sensing complementing clinic and wearable modalities. Cognitive task batteries delivered through the smartphone include validated working-memory paradigms (n-back, Corsi block tapping adaptations), processing-speed assessments (symbol-digit modalities, choice-reaction-time tasks), executive-function probes (Stroop interference, set-shifting adaptations), episodic-memory assessments (paired-associate learning, narrative-recall adaptations), and language assessments (verbal-fluency, naming, semantic-association tasks). Each task yields normative-referenced scores; intra-individual variability across repeated administration is tracked to disambiguate practice effects, state-dependent fluctuations, and disease-related decline. Home-based motor assessment includes finger-tapping paradigms (taps per 10 seconds, tap-interval variability), gait assessments via accelerometer recording during structured walks, and tremor quantification by smartphone accelerometers held against the affected limb yielding tremor amplitude, peak frequency in the 3 hertz to 7 hertz range, and harmonic-distortion metrics. Voice-based cognitive screening uses brief prompted-speech tasks (counting, picture description, narrative recall) with natural-language-processing extraction of word-finding latency, semantic coherence, and prosodic features. Passive sensing of phone-use patterns (typing speed, typing-error rate, app-engagement duration distributions, screen-on intervals, locomotion radius from GPS-derived location traces) provides ambient behavioral indices.

Ecological momentary assessment (EMA) modules deliver brief in-the-moment self-report surveys at quasi-random intervals throughout waking hours, capturing mood (positive and negative affect scales), symptom severity (pain, fatigue, motor symptoms, hallucinations, anxiety, depression), perceived cognitive function, sleep quality, and medication-adherence status. Survey-completion rates of at least 70 percent over moving 14-day windows are required for valid aggregate scores. Aggregated EMA-derived indices including mean symptom-severity scores, within-day and across-day variability, and event-contingent symptom-symptom association patterns are integrated into the longitudinal feedback record. The smartphone module incorporates within-application feedback to the patient, including visual trend displays, medication-adherence prompts, and clinician-configured personalized goals; engagement gamification elements are deployed within constraints prescribed by the regulatory clearance pathway. Safety considerations include explicit informed consent for passive and active data collection, transparent disclosure of data-flow to caregivers and clinicians, on-device storage encryption with secure cloud-synchronization protocols, options for patient-initiated data-export and data-deletion consistent with applicable privacy regulations, suicide-and-crisis screening with embedded escalation pathways triggering same-day clinical contact for high-severity responses, and accommodations for sensory or motor impairments limiting smartphone use including caregiver-mediated data entry workflows.

### Point-of-Care Immunoassay Platforms

Point-of-care immunoassay platforms are integrated to enable decentralized biomarker quantification at the patient's home, in primary care offices, or at remote clinical sites without requirement for centralized clinical laboratory infrastructure. Lateral-flow strip assays employ colloidal-gold, fluorescent-nanoparticle, or upconverting-phosphor labels with smartphone-camera readers performing quantitative densitometry over dynamic ranges of from 10 picograms per milliliter to 50 nanograms per milliliter; analyte targets include plasma neurofilament light, p-tau181, p-tau217, GFAP, and selected drug concentrations for therapeutic drug monitoring. Microfluidic enzyme-linked immunosorbent assay (ELISA) cartridges integrate sample-preparation, immunocapture, wash, detection, and signal-readout steps within disposable polymer-and-glass cartridges processed by benchtop instruments, achieving sensitivities of from 1 picogram per milliliter to 100 picograms per milliliter with time-to-result of from 15 minutes to 60 minutes. Single-molecule array (Simoa-class) immunoassays employ paramagnetic-bead capture, single-molecule digital detection in arrayed femtoliter wells, and Poisson-distributed counting to achieve sensitivity below 1 femtomolar (0.1 picograms per milliliter for typical protein analytes), enabling reliable quantification of plasma p-tau217, NfL, GFAP, and Aβ42/Aβ40 at concentrations below conventional ELISA detection thresholds.

Electrochemical immunoassay biosensors employ amperometric, potentiometric, or impedimetric detection of antibody-antigen binding events at electrode surfaces functionalized with capture antibodies; signal-amplification by redox-active labels, enzyme-substrate turnover, or nanoparticle-tagged secondary antibodies extends sensitivity into the picomolar regime. Field-effect-transistor (FET) biosensors employing graphene, silicon-nanowire, or carbon-nanotube channels functionalized with capture antibodies provide label-free, real-time analyte quantification with sensitivity below 1 picogram per milliliter for representative protein analytes. Surface-plasmon-resonance (SPR) biosensors quantify antibody-antigen binding by refractive-index changes at gold-film surfaces, providing label-free real-time kinetic readouts and end-point concentration measurements over dynamic ranges spanning four orders of magnitude. The closed-loop controller selects point-of-care platforms based on analyte, required sensitivity, sample matrix, throughput requirements, and economic constraints; central-laboratory confirmatory testing is performed at predefined intervals or when point-of-care readings cross safety thresholds. Safety considerations include device-specific quality control via per-cartridge or per-strip internal calibrators, mandatory operator training and competency documentation, external proficiency testing, traceability of point-of-care results to validated reference methods through bridging studies, regulatory clearance under appropriate jurisdictional pathways, and explicit handling of indeterminate, out-of-range, or sample-failure results within the controller decision logic to prevent erroneous dose adjustments based on unreliable measurements.

### Wearable Continuous Biosensors

Wearable continuous biosensors are integrated to provide high-temporal-resolution physiologic and biochemical monitoring complementing periodic blood and imaging-based biomarker measurements. Interstitial-fluid microneedle-array biosensors employ arrays of from 50 microneedles to 500 microneedles of length from 250 micrometers to 1500 micrometers penetrating the stratum corneum to access dermal interstitial fluid; embedded electrochemical, optical, or affinity-based sensing elements quantify glucose, lactate, electrolytes, cortisol, and selected pharmaceutical analytes at intervals of from 1 minute to 15 minutes over wear durations of from 3 days to 14 days. Sweat-based biosensors integrated into flexible wrist or chest patches employ iontophoretic sweat induction, microfluidic sweat collection, and electrochemical or colorimetric sensing of sodium, chloride, potassium, lactate, glucose, cortisol, and selected drugs of abuse and pharmaceutical analytes. Subcutaneous-implant continuous-monitoring biosensors, deployed via minimally invasive insertion procedures, provide multi-month continuous quantification of glucose, lactate, oxygen tension, and selected pharmaceutical and biomarker analytes; analyte readings are transmitted by near-field communication or Bluetooth low-energy to a paired smartphone or dedicated reader.

Intracranial monitoring implants are incorporated for research contexts and select clinical contexts (e.g., refractory epilepsy patients with concurrently implanted depth electrodes, deep-brain-stimulation device candidates, intracerebral hemorrhage and traumatic brain injury intensive care patients, neuro-oncology patients with extant indwelling devices) in which direct central-compartment monitoring is justified by clinical benefit. Such implants quantify extracellular concentrations of glutamate, gamma-aminobutyric acid, dopamine, glucose, lactate, pyruvate, and pharmaceutical analytes by microdialysis with offline or near-real-time analysis; intraparenchymal oxygen tension by Clark-type electrodes; intracranial pressure by strain-gauge or fiber-optic sensors; and local field potentials and single-unit activity by chronic microelectrode arrays. Sampling intervals span from 30 seconds to 60 minutes depending on modality and analyte. The closed-loop controller integrates continuous-biosensor streams via low-pass filtering, outlier detection, and drift-correction algorithms; high-frequency artifacts arising from motion, temperature transients, or sensor warm-up are rejected by signal-quality flags. Safety considerations include biocompatibility of implanted sensor materials, foreign-body response and fibrotic-encapsulation effects on sensor sensitivity and drift over implant lifetime, infection-risk management at percutaneous interfaces, mandatory monthly sensor-accuracy verification against reference assays, explicit handling of sensor end-of-life, and pre-specified fail-safe behavior in which loss of biosensor signal does not by itself trigger autonomous dose changes but is escalated to clinician review.

### Closed-Loop Control Architectures: Rule-Based

The platform implements a rule-based closed-loop control architecture as the foundational, regulatory-clearance-tractable controller tier. Rule-based control encodes a set of predefined biomarker-threshold-to-action mappings expressed in a declarative rule language readable and editable by clinicians, regulatory reviewers, and prescribing physicians. Representative rules include: if plasma p-tau217 increases more than 25 percent above personalized baseline over consecutive measurements separated by from 4 weeks to 12 weeks, escalate dose by one level within the prescribed dose-titration ladder; if plasma neurofilament light increases more than 50 percent above personalized baseline, pause dose escalation pending clinician review; if Aβ42/Aβ40 ratio normalizes (defined as crossing the assay-specific positivity threshold from positive to negative) and remains normalized across two consecutive measurements, transition from induction to consolidation phase. Tiered dose-escalation rules implement step-wise escalation from a starting dose of from 5 milligrams per day to 50 milligrams per day through up to from 4 escalation steps to 10 escalation steps, with each step requiring confirmation of safety-biomarker stability before progressing. Each rule includes a triggering biomarker or biomarker combination, a temporal-window specification, a comparison operator and threshold, an action specification, and a confirmation or override requirement.

Safety-interlock rules constitute a separate, immutable subset of the rule set that takes precedence over efficacy-driven dose-adjustment rules and that the autonomous controller is constitutionally unable to override. These include: a hard prohibition on autonomous shortening of dose intervals below a per-patient prescribed minimum interval (typically not less than 50 percent of the steady-state regimen interval); a hard maximum dose ceiling per dosing event and per 24-hour cumulative window; a mandatory minimum interval of from 24 hours to 168 hours between consecutive dose escalations; immediate dose-pause triggers on detection of liver-function-test elevations exceeding three times the upper limit of normal, serum creatinine elevations exceeding 50 percent above baseline, hematologic-toxicity grade 3 or 4 events per Common Terminology Criteria for Adverse Events (CTCAE) version 5.0, or new neurologic deficits not attributable to underlying disease progression; and a 24-hour-to-72-hour minimum window between adverse-event report and any further dose change other than dose pause or discontinuation. All rule firings are logged with timestamp, triggering biomarker values, executed action, and clinician notification; rule sets are versioned, signed by responsible clinical authorities, and modifiable only through a documented change-control process with audit trail. The rule-based controller is the default and only fully autonomous controller for outpatient ambulatory use; the more powerful learning-based controllers described in subsequent sections operate in advisory mode unless explicitly authorized for autonomous operation by the responsible clinician within institutional protocols.

### Closed-Loop Control Architectures: Model-Predictive

The platform implements a model-predictive control (MPC) tier built upon individualized pharmacokinetic-pharmacodynamic (PK-PD) models that are updated with the patient's longitudinal monitoring data. The PK component is a multi-compartment model with absorption, central, peripheral, and effect-site compartments parameterized by absorption rate constant, distribution volumes, intercompartmental clearances, and elimination clearance; the PD component links effect-site concentration to biomarker dynamics through pharmacodynamic models including direct-effect models, indirect-response models with stimulation or inhibition of biomarker synthesis or degradation, transit-compartment delay models, and tolerance-and-rebound models where appropriate. Initial parameter estimates derive from population-mean priors obtained from prior clinical-development studies; parameters are updated by Bayesian individual-parameter estimation as each new biomarker measurement becomes available, using the maximum a posteriori or full-posterior-distribution methods. The MPC controller solves a finite-horizon optimization problem at each decision step, computing the dose sequence over a prediction horizon of from 1 month to 12 months that minimizes a cost function combining biomarker deviation from a clinician-prescribed target trajectory, predicted adverse-event probability, dose-burden penalty, and adherence-burden penalty subject to dose-level and dose-interval constraints reflecting the safety-interlock rules.

Two MPC variants are implemented: a population-mean variant in which the controller uses fixed population-typical PK-PD parameters and updates only patient-specific baselines and noise variances, and an individual-personalized variant in which the controller estimates patient-specific PK-PD parameters from each individual's accumulating data. The population-mean variant is appropriate for patients with limited longitudinal data (typically less than 6 months of monitoring) for whom individualized parameter estimates would be unstable; the individual-personalized variant is appropriate once sufficient data accumulates (typically more than from 6 months to 12 months of longitudinal monitoring with from 4 to 8 biomarker timepoints per analyte). The controller automatically transitions from population-mean to individual-personalized variants according to predefined data-sufficiency criteria. Safety considerations include explicit confidence-interval propagation through the model-predictive optimization with dose recommendations marked as low-confidence and routed to clinician review when posterior parameter distributions are wide; sensitivity analysis on model-structural assumptions; per-decision logging of the model state, prediction trajectories, optimization objective values, and chosen action; periodic model-validation tests in which model-predicted biomarker trajectories are compared to subsequent observed biomarker values, with mismatch exceeding pre-specified tolerances triggering controller suspension and human review; and operator-facing model-interpretability visualizations enabling clinicians to inspect and challenge the model's predicted-versus-actual fit before authorizing recommended dose changes.

### Closed-Loop Control Architectures: Reinforcement-Learning

The platform implements a reinforcement-learning (RL) tier suitable for protocols and institutional settings authorized for advanced learning-based decision support. The RL controller treats the patient state (current and historical biomarker measurements, current dose, time since last dose, accumulated dose, demographics, comorbidities, concomitant medications, recent adverse events) as the state vector, the candidate dose adjustments (escalate one level, hold, de-escalate one level, pause, change formulation) as the action space, and a clinician-defined composite reward function as the optimization target. Q-learning and deep-Q-learning controllers are implemented for discrete action spaces, with the Q-function approximated by neural networks of from 2 hidden layers to 8 hidden layers with from 64 units to 1024 units per layer. Policy-gradient methods including REINFORCE, advantage actor-critic (A2C), and proximal policy optimization (PPO) are implemented for continuous-action variants in which dose level and dose interval are continuous decision variables. The reward function is shaped to encode multi-objective optimization across efficacy (biomarker progression toward target), safety (penalty for adverse-event-associated states and for dose levels exceeding individualized safe ranges), adherence (penalty for regimens predicted to produce low adherence), and burden (penalty for excessive monitoring frequency and patient effort).

Recognizing that online exploration of dose policies in human patients is ethically and practically constrained, the RL tier is trained predominantly via offline RL on retrospective clinical and trial datasets using conservative Q-learning (CQL) and behavior-regularized actor-critic methods that explicitly penalize Q-value estimates for state-action pairs poorly supported by the training data, preventing policy extrapolation into regions of the state-action space where the policy has not been validated. Safe RL is implemented through constrained Markov decision process (CMDP) formulations in which expected cumulative cost (representing safety-event probability) is constrained below a pre-specified threshold via Lagrangian relaxation, primal-dual optimization, or constrained policy-optimization methods. The trained RL policy outputs are routed to a mandatory rule-based safety filter (the rule-based controller described above) that overrides any RL recommendation violating safety-interlock rules; the RL policy is further restricted to operate in advisory mode unless and until institutional-review-board-approved and regulatory-cleared protocols authorize autonomous operation. Logging of state, recommended action, executed action, override events, observed outcomes, and reward attributions enables continual policy auditing, fairness analysis across patient subpopulations, and detection of distributional shift requiring policy retraining. Safety considerations include explicit out-of-distribution detection on patient state, mandatory clinician sign-off for actions outside the empirical training distribution, and prohibition on policy updates from any single patient's data without aggregation across diverse populations to prevent overfitting to atypical individual trajectories.

### Closed-Loop Control Architectures: Supervised-Learning Predictors of Treatment Response

The platform implements a supervised-learning predictor tier providing prognostic and treatment-response-prediction outputs that inform but do not autonomously drive dose decisions. Supervised predictors are trained on labeled retrospective and trial datasets in which the input feature vector comprises baseline demographics, genetic risk factors (including apolipoprotein E genotype, polygenic risk scores, and rare pathogenic variants when available), baseline biomarker panels, imaging features, comorbidities, and concomitant medications, and the label is the observed treatment-response outcome over a follow-up window of from 6 months to 36 months. Gradient-boosted decision trees (including extreme gradient boosting and light gradient-boosting machine implementations) are trained for tabular feature predictions, with hyperparameter optimization by Bayesian optimization or grid search and with feature-importance analysis (gain, split, and Shapley additive explanation values) supporting interpretability. Random forests with from 100 trees to 5000 trees provide ensemble baselines with intrinsic out-of-bag generalization estimates and feature-importance rankings. Neural network classifiers including multi-layer perceptrons and one-dimensional convolutional networks process tabular and short time-series inputs respectively. Ensemble methods combining multiple base learners by weighted averaging, stacking, or Bayesian model averaging are deployed for improved generalization.

Transformer-based time-series predictors are trained for forecasting longitudinal biomarker trajectories conditioned on dose-history and covariate-history inputs. The transformer architecture employs from 4 attention heads to 16 attention heads per layer and from 4 layers to 24 layers, with positional encoding adapted for irregular sampling intervals characteristic of clinical longitudinal data. Inputs are tokenized as biomarker-name plus time-stamp plus value tuples, with separate input streams for dose, adverse events, and concomitant medications. Outputs are predicted biomarker trajectories over forecast horizons of from 1 month to 24 months with predictive confidence intervals. The supervised predictor outputs are integrated into the model-predictive controller as initial estimates and into the clinician-facing decision-support interface as forecast visualizations. Predictor model performance is benchmarked by area under the receiver operating characteristic curve, calibration plots, Brier scores for classification tasks, and root-mean-square error and continuous-ranked-probability score for regression and forecasting tasks. Safety considerations include external-cohort validation, subgroup performance analysis (sex, age, race, ethnicity, comorbidity strata, geographic region) with reporting of subgroup performance disparities, calibration monitoring across the deployment lifecycle, formal drift detection with retraining triggers, model-card documentation, and prohibition on use of predictor outputs to deny treatment access to any patient subgroup absent affirmative regulatory and ethical authorization.

### Closed-Loop Control Architectures: Transformer-Based Decision Support

The platform implements a transformer-based clinical decision support tier in which a large language model integrates structured biomarker data, imaging features, electronic health record narrative, and patient-reported outcomes to generate clinician-facing decision-support summaries and dose recommendations. The decision-support model is implemented as a fine-tuned domain-specific large language model with from 7 billion parameters to 200 billion parameters, fine-tuned on de-identified clinical text, biomarker-trajectory annotations, treatment-decision narratives, and clinical practice guidelines through supervised fine-tuning and reinforcement learning from clinician feedback. Retrieval-augmented generation (RAG) is implemented over a curated evidence corpus including peer-reviewed publications, regulatory product labels, clinical practice guidelines, and institutional protocols, with retrieval by dense embedding similarity over a vector database of from 10 million entries to 1 billion entries; retrieved evidence passages are inserted into the model context window and cited in generated recommendations to ensure traceability of evidentiary basis. Chain-of-thought reasoning prompts elicit explicit step-by-step justification chains spanning patient history review, biomarker trajectory interpretation, mechanistic rationale, and evidence-based dose recommendation, enabling clinician inspection and challenge of the reasoning prior to authorization.

Multimodal models integrate structured biomarker tabular data, imaging features (MRI volumetrics, DTI metrics, PET SUVR maps), wearable and smartphone-derived digital biomarker summaries, and patient-reported outcomes through modality-specific encoders fused via cross-attention to a shared transformer trunk. The multimodal model outputs include narrative case summaries, structured biomarker-trajectory plots with annotation, prioritized differential-diagnosis lists for ambiguous trajectories, candidate dose-adjustment options ranked by expected efficacy and adverse-event risk with associated probability estimates, and explicit identification of patient-specific risk factors warranting clinician attention. The decision-support tier operates strictly in advisory mode; all recommendations require clinician review and authorization before execution. Safety considerations include hallucination mitigation via retrieval grounding and citation requirements, mandatory disclosure of model confidence and known limitations, prohibition on generating recommendations for off-label uses outside authorized protocols, explicit refusal pathways when input data are insufficient or out-of-distribution, comprehensive logging of model inputs and outputs, regular bias and fairness audits with reporting of subgroup performance, model-version control with documented validation for each released version, and clinician training on appropriate interpretation of language-model outputs including recognition of common failure modes.

### External Clinical Override and Safety Architecture

The platform implements an external clinical override and safety architecture providing comprehensive human-in-the-loop oversight of all autonomous and advisory controller outputs. A clinician-facing interface accessible through web-browser and dedicated-application clients presents pending dose recommendations with associated biomarker trajectories, model-state visualizations, evidence citations, confidence intervals, and identified safety considerations; clinicians may authorize the recommendation as proposed, modify the recommended dose level or interval within prescribed bounds, veto the recommendation with documented clinical rationale, or escalate to specialist review. Tiered authorization levels are defined: registered-nurse-tier authorization is permitted for routine within-protocol confirmatory authorizations of recommendations falling within pre-specified low-risk envelopes; primary-care-physician-tier authorization is required for dose changes outside the low-risk envelope but within authorized treatment protocols; specialist-physician-tier authorization (neurologist, psychiatrist, or relevant subspecialist) is required for cross-protocol modifications, off-protocol dose adjustments, formulation switches, and treatment discontinuation; institutional-review-tier authorization is required for any deviation from the authorized clinical protocol or for autonomous-mode operation of learning-based controllers.

All controller recommendations, clinician authorization decisions, modifications, vetoes, and resultant administered doses are recorded in an immutable audit trail with timestamp, authenticating-clinician identity, decision rationale, biomarker state at decision time, and controller version identifiers. Audit-trail records are cryptographically signed and stored with redundancy across geographically distributed data centers; records are retrievable on demand for institutional quality review, regulatory inspection, and patient access under applicable health-information-portability frameworks. Mandatory clinical review is performed at defined intervals: weekly for patients in induction phase or with recent adverse events, monthly for patients in stable maintenance with normal biomarker trajectories, and quarterly for patients in long-term stable maintenance with no controller-recommended changes during the prior interval. Mandatory specialist review is performed at from 6-month to 12-month intervals regardless of trajectory status, ensuring expert oversight of long-term treatment decisions. Safety considerations include redundant clinician notification through multiple channels (in-application alerts, email, short-message-service text, telephonic escalation for time-critical events) with documented acknowledgment requirements; backup-clinician on-call rotations ensuring 24/7 coverage for safety-critical alerts; explicit failover behavior in which loss of clinician availability does not result in autonomous treatment continuation outside pre-authorized safe envelopes; and patient-initiated emergency-stop capability through dedicated interface controls and through any treating clinician.

### Safety Interlocks

The platform implements a hierarchical safety-interlock subsystem that constrains all controller-recommended and clinician-authorized actions within predefined safe envelopes regardless of biomarker trajectory or controller confidence. A predefined upper bound on dose level is established at the patient-prescription level by the responsible clinician at the time of regimen initiation, with maximum-dose ceilings ranging from 100 milligrams per dose to 1000 milligrams per dose and from 200 milligrams per 24 hours to 2000 milligrams per 24 hours depending on indication, formulation, and patient-specific tolerability data; controller recommendations exceeding the ceiling are automatically clipped to the ceiling and flagged for clinician review. A predefined minimum dose interval, ranging from 4 hours to 168 hours between consecutive doses, is established to prevent inadvertent overdosing; controller recommendations shortening intervals below the minimum are blocked and routed to clinician review. Mandatory holds on biomarker exceedance are triggered when safety biomarkers cross predefined thresholds: alanine aminotransferase or aspartate aminotransferase elevations above three times the upper limit of normal, total bilirubin above 1.5 times the upper limit of normal, serum creatinine elevations above 50 percent over personalized baseline, platelet count below 75×10\hat{ }9 per liter, absolute neutrophil count below 1.0×10\hat{ }9 per liter, or new electrocardiographic conduction abnormalities trigger automatic dose pause pending clinician review.

Automatic pause is triggered upon any patient-reported, caregiver-reported, or controller-detected adverse event reaching CTCAE grade 3 or higher, with mandatory clinician notification within from 1 hour to 4 hours and mandatory clinical evaluation within from 24 hours to 72 hours before dose resumption. Pharmacist-override interlocks check each controller-recommended dose against the patient's full medication list for known and suspected drug-drug interactions through integration with maintained pharmacotherapy interaction databases; flagged interactions require pharmacist review and explicit override authorization before dose execution. Additional interlocks include: temperature and humidity excursion detection on stored medication supplies with quarantine of supplies exposed outside validated storage envelopes; lot-recall integration with automatic patient notification and dose suspension for affected lots; pregnancy-test interlocks for therapeutics with known or suspected reproductive toxicity, requiring confirmed negative pregnancy test within from 7 days to 14 days prior to each dose escalation in patients of reproductive potential; and contraindication interlocks blocking dose recommendations in the presence of recently documented absolute contraindications. All interlock firings, override requests, override authorizations, and override outcomes are logged in the audit trail with the same immutability and signature requirements as controller decisions. Safety considerations include periodic interlock-coverage audits to identify gaps, simulated-failure testing of interlock behavior under stress conditions, redundant interlock implementations across the controller, the prescribing electronic-health-record interface, and the pharmacy dispensing interface, and prohibition on disabling any safety interlock without documented change-control authorization at the institutional level.

### Data Integration Architecture

The platform implements a comprehensive data integration architecture aggregating biomarker measurements, imaging studies, wearable and smartphone-derived data, clinician annotations, and dispensing records within a secure cloud infrastructure compliant with applicable health-information-privacy and security frameworks. Patient data are encrypted at rest using AES-256-GCM with hardware-security-module-managed keys and encrypted in transit using TLS 1.3 with mutual-authenticated client and server certificates. The architecture implements role-based access control, attribute-based access control, and audit logging of all data accesses with immutable timestamped records of access requestor, accessed records, and access purpose. Compliance with the United States Health Insurance Portability and Accountability Act (HIPAA), the Health Information Technology for Economic and Clinical Health Act (HITECH), the European Union General Data Protection Regulation (GDPR), and other applicable national and regional privacy frameworks is supported by configurable data-residency, data-minimization, data-retention, and patient-rights workflows including data-export, data-correction, and data-deletion processing. Health Level 7 Fast Healthcare Interoperability Resources (HL7 FHIR) standard resources (Observation, MedicationRequest, MedicationAdministration, Patient, Practitioner, ServiceRequest, DiagnosticReport, Condition) are used for interoperability with electronic health records, with bidirectional read-and-write integration to support continuity of care across the patient's clinical care network.

Patient consent management is implemented through a granular consent-capture workflow distinguishing consent for treatment, consent for biomarker data collection, consent for de-identified data use in continuous platform improvement, consent for de-identified data sharing with research partners, and consent for use in regulatory submissions; patient consent records are versioned, signed, and revocable through the patient-facing interface with downstream propagation of revocation to dependent processing systems. Adaptive dosing trajectories are recorded as structured time-series of dose level, dose interval, dose-decision authoring controller or clinician, decision rationale, and resulting biomarker observations, supporting longitudinal analytics and prospective regimen optimization. Data lineage is maintained through a comprehensive provenance system identifying for each derived record the source data, processing algorithms, and processing software versions, enabling reproducibility of all controller and supervised-predictor outputs. Safety considerations include penetration-testing programs, secure-software-development-lifecycle compliance, third-party security audits, breach-notification workflows, secure-decommissioning procedures for end-of-life data, and explicit patient-facing transparency communications detailing data use, sharing, and protection practices in plain language at literacy levels appropriate for the targeted populations.

### Adaptive Dosing Trajectories

The platform supports multi-phase adaptive dosing regimens spanning induction, consolidation, maintenance, and tapering phases with biomarker-informed transition criteria between phases. The induction phase, typically from 4 weeks to 24 weeks in duration, deploys dose-escalation from a starting dose ranging from 5 milligrams per day to 50 milligrams per day toward a target induction dose ranging from 50 milligrams per day to 500 milligrams per day, with escalation conditional on stability of safety biomarkers and absence of dose-limiting toxicity. The consolidation phase, from 12 weeks to 52 weeks, maintains the induction dose while biomarker trajectories are monitored for treatment-response confirmation; favorable biomarker trajectories meeting prespecified response criteria (representative examples: Aβ42/Aβ40 normalization, Centiloid reduction exceeding 20 Centiloids, plasma NfL reduction of at least 25 percent below personalized baseline) authorize transition to maintenance. The maintenance phase, from 12 months to multiple years, deploys reduced dose levels or extended intervals consistent with sustained biomarker control while minimizing cumulative dose exposure and adverse-event burden. The tapering phase is engaged upon achievement of predefined disease-control milestones and consists of step-wise dose reduction of from 10 percent to 25 percent per step at intervals of from 4 weeks to 12 weeks, with each step contingent on continued biomarker stability and reversal-of-tapering pathways triggered by biomarker reactivation.

Long-horizon trajectory optimization is implemented through model-predictive-control horizons of from 12 months to 60 months in which the controller jointly optimizes dose-level and dose-interval trajectories across induction, consolidation, maintenance, and tapering phases under explicit constraints on cumulative dose exposure, predicted adverse-event accumulation, treatment-burden metrics, and patient-stated preferences. Transition-criteria specifications encode the biomarker patterns and durations triggering phase transitions; transition criteria are clinician-configurable within prescribed bounds at the time of regimen initiation and are revisable through documented change-control workflows. Deintensification protocols are engaged upon biomarker normalization defined as biomarker measurements remaining within personalized normalcy bands for from 3 consecutive measurements to 6 consecutive measurements over an observation window of from 6 months to 12 months; deintensification involves controlled stepwise dose reduction with prespecified reinstitution criteria for biomarker reactivation. Safety considerations include explicit modeling of withdrawal and rebound phenomena in the PK-PD models used by the model-predictive controller, mandatory clinician authorization of all phase transitions, biomarker reactivation triggers with automatic re-escalation pathways subject to clinician confirmation, and patient education materials describing the rationale, expected duration, and reversal options for each phase.

### Combination-Therapy Monitoring

The platform supports combination-therapy regimens in which two or more pharmaceutical or biologic agents are co-administered, with independent biomarker monitoring of each component and interaction-aware dose adjustment. For combinations targeting distinct disease mechanisms (representative examples: an amyloid-targeting therapeutic combined with a tau-targeting therapeutic for Alzheimer disease; a synuclein-targeting therapeutic combined with a symptomatic dopaminergic agent for Parkinson disease; an antisense oligonucleotide combined with a small-molecule modifier for Huntington disease), the monitoring subsystem quantifies mechanism-specific biomarkers for each agent and tracks dose-response for each component independently. Interaction-aware dose adjustment is implemented through expanded PK-PD models encoding known and modeled pharmacokinetic and pharmacodynamic interactions including cytochrome P450 and transporter-mediated PK interactions, receptor-level and downstream signaling-pathway PD interactions, and emergent toxicity interactions; the model-predictive controller jointly optimizes the dose trajectories of all combination components subject to per-component and combined-regimen safety constraints. Synergy-quantification metrics including Loewe additivity, Bliss independence, and highest-single-agent excess are computed prospectively from PK-PD model outputs and retrospectively from observed biomarker responses; combinations exhibiting greater than additive efficacy and acceptable additive or sub-additive toxicity are flagged as favorable.

The combination-therapy monitoring architecture incorporates additional safety interlocks specific to combination regimens including: explicit drug-drug interaction checking through pharmacotherapy interaction databases at each dose-recommendation cycle; mandatory clinician authorization for any dose change affecting two or more co-administered components within a 14-day window; explicit identification of overlapping toxicity profiles (representative examples: hepatotoxicity, nephrotoxicity, hematotoxicity, QT prolongation) with conservative dosing strategies when overlap is present; and pre-specified component-prioritization rules dictating which component is held first in the event of biomarker exceedance or adverse-event detection. Combination regimens are documented as integrated treatment plans with shared monitoring schedules, shared adherence-monitoring infrastructure, and shared decision-support outputs distinguishing per-component recommendations from regimen-level recommendations. Safety considerations include the elevated complexity and consequent elevated risk of combination regimens, mandatory pre-treatment review by specialist physicians with combination-therapy expertise, enhanced post-administration monitoring intensity (representative example: doubled biomarker-sampling frequency during the first from 8 weeks to 16 weeks of combination treatment), explicit patient education on the rationale and risks of combination therapy, and clear emergency-stop pathways enabling rapid discontinuation of one or all components in response to suspected adverse drug reaction.

### Pediatric and Special-Population Monitoring

The platform incorporates pediatric-specific and special-population-specific monitoring workflows recognizing the distinct physiologic, developmental, and ethical considerations applicable to children, adolescents, frail elderly patients, pregnant patients, and patients with significant organ-system comorbidities. For pediatric patients, age-adjusted normative ranges are established for circulating, CSF, and imaging biomarkers with reference distributions stratified into age bands of from 0 years to 1 year, from 1 year to 2 years, from 2 years to 6 years, from 6 years to 12 years, and from 12 years to 18 years; the controller applies age-band-specific thresholds for biomarker exceedance and treatment-response evaluation. Weight-based dose scaling is applied at the milligrams-per-kilogram or milligrams-per-square-meter body-surface-area levels with patient weight and height re-measured at intervals of from 1 month to 6 months and dose recommendations automatically re-scaled to account for growth. Growth-corrected biomarker thresholds account for age-and-size-dependent variation in biomarker reference distributions; for example, plasma neurofilament light reference distributions in healthy children differ substantively from adult distributions, and the controller applies pediatric-specific reference distributions when interpreting longitudinal trajectories.

Cognitive-developmental milestones are incorporated as efficacy endpoints in pediatric protocols, with standardized assessments at age-appropriate intervals quantifying expressive and receptive language acquisition, motor-milestone achievement, social-cognitive development, academic achievement trajectories, and adaptive-behavior trajectories. For pediatric neurodevelopmental and neurodegenerative disorders, treatment-attributable preservation or restoration of developmental trajectory relative to expected age-typical curves is integrated as a long-horizon efficacy signal complementing biomarker measurements. For frail-elderly patients, the platform integrates frailty-index assessments, polypharmacy-interaction monitoring, and prospective fall-risk monitoring through wearable-derived gait and balance metrics; dose-escalation rates are reduced and biomarker-exceedance thresholds are tightened for frail-elderly populations. For pregnant patients, dedicated reproductive-toxicology screening workflows, mandatory specialist obstetric co-management, and conservative dose-adjustment policies are engaged with explicit documentation of risk-benefit rationale for each therapeutic decision. For patients with hepatic impairment, renal impairment, or other significant organ-system comorbidities, the PK-PD models incorporate impairment-specific parameter modifications and the controller applies tightened dose-ceiling constraints. Safety considerations specific to special populations include enhanced informed-consent and assent processes for pediatric patients, surrogate-decision-maker integration for cognitively impaired patients, mandatory institutional-review-board oversight for protocol deviations involving vulnerable populations, prospective enrollment of diverse populations in platform validation cohorts to support generalization across demographic and clinical subgroups, and ongoing post-market surveillance reporting stratified by special-population subgroup to detect signal divergence requiring policy updates.

### Expanded Axis 10—Personalization and Cross-Portfolio Integration (Deep Dive)

### Personalization and Cross-Portfolio Integration

### Genome-Indexed Payload Selection

The platform incorporates patient-specific genomic data as a primary input to the payload-selection and dosing decision engine. Genomic input is obtained from one or more of whole-genome sequencing at coverage of from 30× to 60x, whole-exome sequencing at coverage of from 100x to 200x, and targeted pharmacogenomic panel sequencing at coverage of from 500x to 2000x for a curated set of variants comprising CYP2D6, CYP2D6 copy number including duplications and deletions, CYP3A4 and CYP3A5 expressor and nonexpressor alleles, CYP2C9 *2 and *3 reduced-function alleles, CYP2C19 *2, *3, and *17 alleles, UGT1A1 *6 and *28 alleles, SLCO1B1 c.521T>C reduced-transport allele, DPYD *2A, *13, c.2846A>T, and HapB3 alleles, TPMT *2, *3A, *3B, and *3C alleles, HLA-B*57:01, HLA-B*15:02, HLA-B*58:01, HLA-A*31:01, and HLA-DRB1*07:01 alleles. The pharmacogenomic engine maps each detected allele to one or more of dose-adjustment multipliers in a range of from 0.25 to 4.0 relative to a wildtype reference, payload-class avoidance flags, contraindication flags, and enhanced-monitoring flags. Pharmacogenomic data are encoded in Clinical Pharmacogenetics Implementation Consortium star-allele nomenclature and integrated with electronic health record-resident pharmacogenomic decision-support standards comprising HL7 Fast Healthcare Interoperability Resources Genomics Implementation Guide and the National Human Genome Research Institute electronic medical record and genomics network specifications. The genomic input layer further accepts polygenic risk scores derived from a polygenic risk score model of from 100 to 5,000,000 variants and assigns a polygenic-risk-stratified dosing trajectory comprising one or more of dose-titration acceleration in low-risk individuals, dose-titration deceleration in high-risk individuals, and payload-class re-prioritization based on risk-allele burden. The closed-loop feedback channel returns measured pharmacokinetic exposure data and biomarker response data to the genomic decision module, which updates a patient-specific allele-effect-size posterior distribution using Bayesian hierarchical inference with a population-level prior. Regulatory documentation conforms to United States Food and Drug Administration draft and final guidances on clinical pharmacogenomics including ‘Clinical Pharmacogenomics: Premarket Evaluation in Early-Phase Clinical Studies and Recommendations for Labeling’ and European Medicines Agency ‘Guideline on the Use of Pharmacogenetic Methodologies in the Pharmacokinetic Evaluation of Medicinal Products.’

Beyond pharmacogenomic alleles, the genome-indexed payload-selection module accepts disease-predisposing variant calls and disease-modifier variant calls. Variants of primary clinical interest comprise APOE genotype with stratification across APOE *2/*2, *2/*3, *2/*4, *3/*3, *3/*4, and *4/*4 haplotypes for Alzheimer disease risk and lecanemab- and donanemab-class amyloid-related-imaging-abnormality risk; pathogenic and likely-pathogenic variants in APP, PSEN1, and PSEN2 for autosomal-dominant Alzheimer disease; GBA1 N370S, L444P, and other glucocerebrosidase variants for Parkinson disease and Gaucher disease comorbidity; LRRK2 G2019S, R1441C/G/H, and Y1699C variants for LRRK2-Parkinson disease; SNCA point mutations and locus multiplications for synucleinopathy; MAPT exon 10 splice-site and missense variants for frontotemporal dementia and progressive supranuclear palsy spectrum; GRN loss-of-function variants for progranulin-deficient frontotemporal dementia; C9ORF72 hexanucleotide repeat expansions of from 30 to greater than 4,000 repeats for C9-amyotrophic lateral sclerosis and C9-frontotemporal dementia spectrum; SOD1, FUS, TARDBP, ANG, OPTN, UBQLN2, TBK1, and CHCHD10 variants for amyotrophic lateral sclerosis; ATXN1, ATXN2, ATXN3, ATXN7, ATXN8, ATXN10, CACNA1A, TBP, PPP2R2B, ATN1, and HTT CAG-repeat expansions of from 36 to greater than 250 repeats for spinocerebellar ataxias and Huntington disease; FMR1 CGG-repeat expansions for fragile X syndrome and fragile X-associated tremor/ataxia syndrome; and DMPK and CNBP repeat expansions for myotonic dystrophy types 1 and 2. The payload-selection engine consults a variant-payload mapping table that, for each disease-causing variant, prioritizes one or more of allele-selective antisense oligonucleotide cargo, allele-selective small-interfering RNA cargo, CRISPR-Cas-based base-editing or prime-editing cargo, gene-replacement cargo, and small-molecule modulator cargo. Rare-variant-driven indications are addressed through an N-of-1 expansion module that, on detection of a private variant absent from gnomAD aggregations of 807,000 exomes and genomes or present at allele frequency below 1 in 100,000, initiates a regulatory-aligned N-of-1 development workflow comprising patient-specific cargo design, FDA Investigational New Drug expanded-access or individual-patient IND submission, and individualized monitoring plan generation. Modifier-locus signals comprising TMEM106B haplotypes for frontotemporal lobar degeneration, KIF5A and UNC13A variants for amyotrophic lateral sclerosis progression, and BIN1 and CR1 variants for Alzheimer disease modify the dosing trajectory parameters delivered to the closed-loop controller.

The platform interfaces the genome-indexed payload module with the longitudinal closed-loop controller via a structured data-exchange interface that transmits, for each patient encounter, an updated variant-effect posterior distribution, an updated polygenic-risk posterior, an updated pharmacogenomic-derived dose-multiplier vector, and an updated payload-class-permissibility matrix. The controller integrates these inputs with measured plasma drug exposure obtained from dried blood spot sampling at intervals of from 1 to 90 days, measured biomarker response obtained from plasma and cerebrospinal fluid assays at intervals of from 30 to 365 days, and measured digital-biomarker trajectories obtained from continuous wearable monitoring with sampling frequency of from 0.0167 Hz to 1000 Hz. Closed-loop genomic-context-aware dose updates are computed using a Bayesian hierarchical model in which the population-level prior is informed by aggregated pharmacogenomic effect sizes from a reference database of from 10,000 to 10,000,000 genotyped individuals and the patient-specific posterior is updated via Markov chain Monte Carlo inference or variational Bayesian inference with computational latency of less than 10 seconds per update. The clinical utility of genome-indexed payload selection comprises one or more of reduction in payload-class adverse-event rate of from 30 percent to 80 percent relative to genotype-unaware prescribing, reduction in dose-titration time of from 25 percent to 70 percent, and improvement in payload-class response rate of from 15 percent to 60 percent. Regulatory considerations comprise FDA companion-diagnostic clearance pathways under 21 CFR Part 820 and 21 CFR Part 814, in vitro diagnostic device classification, Clinical Laboratory Improvement Amendments-certified laboratory validation for any laboratory-developed test components, and conformity with FDA ‘Use of Real-World Evidence to Support Regulatory Decision-Making for Medical Devices’ and ‘Considerations for the Use of Real-World Data and Real-World Evidence to Support Regulatory Decision-Making for Drug and Biological Products’ guidances.

### Transcriptome-Indexed Personalization

Transcriptomic personalization inputs comprise bulk RNA-sequencing of peripheral blood mononuclear cells at sequencing depth of from 20 million to 100 million paired-end reads per sample, single-cell RNA-sequencing of peripheral blood mononuclear cells at depth of from 50,000 to 500,000 cells per sample with from 20,000 to 100,000 reads per cell, and, where the indication permits and where intrathecal access is being established for therapeutic dosing, single-cell RNA-sequencing of cerebrospinal-fluid-resident leukocytes at depth of from 5,000 to 50,000 cells per sample. Bulk RNA-sequencing yields gene-level expression quantification across approximately 20,000 protein-coding genes, with isoform-level resolution where library preparation comprises full-length transcript capture. Single-cell RNA-sequencing yields cell-type-resolved expression matrices stratified by canonical immune lineages comprising CD4+ T cells with Th1, Th2, Th17, regulatory T cell, and follicular helper T cell subpopulations, CD8+ T cells with naive, effector, effector-memory, central-memory, and exhausted subpopulations, B cells with naive, memory, plasmablast, and plasma cell subpopulations, classical and non-classical monocytes, conventional dendritic cells, plasmacytoid dendritic cells, and natural killer cells. The transcriptomic personalization engine maps gene-expression and cell-population profiles to disease-subtype labels using one or more of supervised classification with elastic-net regularized logistic regression, supervised classification with gradient-boosted decision trees, supervised classification with deep neural network architectures, and unsupervised clustering with uniform manifold approximation and projection followed by Leiden community detection. For Alzheimer disease, the engine resolves from 3 to 7 transcriptomic subtypes comprising amyloid-dominant, tau-dominant, neuroinflammation-dominant, vascular-dominant, and mixed subtypes, with each subtype mapped to differentially-prioritized payload classes. For Parkinson disease, the engine resolves brain-derived versus body-derived subtypes, LRRK2-driven versus GBA-driven versus idiopathic subtypes, and tremor-dominant versus postural-instability-gait-difficulty subtypes. For amyotrophic lateral sclerosis, the engine resolves TDP-43-dominant, FUS-dominant, and SOD1-dominant transcriptomic signatures.

Response-predictor transcriptomic models are constructed using one or more of recursive feature elimination with cross-validation, Boruta feature selection, and L1-regularized regression to identify gene panels of from 50 to 500 genes whose pre-treatment expression predicts post-treatment response with area under the receiver operating characteristic curve of from 0.75 to 0.95 in held-out validation cohorts. Pathway-activity scoring is computed using one or more of single-sample gene set enrichment analysis, gene set variation analysis, Pathway Activity Cell Enrichment, and AUCell, applied to gene sets from the Hallmark collection of 50 cancer hallmark pathways, the Reactome collection of approximately 2,500 human pathways, the Kyoto Encyclopedia of Genes and Genomes collection of approximately 350 human pathways, the Gene Ontology Biological Process collection, and the WikiPathways collection. Pathway-activity scores are integrated as covariates in the closed-loop dosing controller and trigger payload-class re-prioritization when a pathway-activity score crosses a pre-specified threshold corresponding to a shift in pathway activation of from 1.5 to 3.0 standard deviations above or below the patient-specific baseline. The closed-loop integration comprises serial peripheral blood mononuclear cell collections at intervals of from 30 to 180 days, with each collection yielding an updated transcriptomic-subtype posterior probability, an updated response-predictor score, and an updated pathway-activity vector. Clinical utility comprises one or more of subtype-targeted payload deployment yielding response rate improvement of from 20 percent to 70 percent relative to subtype-unaware deployment, early detection of payload-class non-response permitting payload switching within from 30 to 120 days rather than from 6 to 18 months, and identification of off-target inflammation signatures permitting dose reduction or payload-class change before clinically apparent adverse events. Regulatory considerations comprise compliance with the FDA ‘Bioanalytical Method Validation Guidance for Industry,’ the Clinical and Laboratory Standards Institute MM22 guidance on next-generation-sequencing-based clinical assays, and the FDA ‘Use of Public Human Genetic Variant Databases to Support Clinical Validity for Genetic and Genomic-Based In Vitro Diagnostics’ guidance, with single-cell RNA-sequencing assays validated under Clinical Laboratory Improvement Amendments-certified laboratory protocols where deployed as clinical assays.

### Proteome-Indexed Personalization

The platform accepts proteomic personalization inputs comprising plasma proteome profiling using one or more of the Olink Explore HT platform measuring 5,400 proteins per sample with a dynamic range of approximately 10 orders of magnitude, the Olink Explore 3072 platform measuring 2,925 proteins, the SomaScan 11K assay measuring 10,776 protein analytes per sample, the SomaScan 7K assay measuring 7,267 protein analytes, mass-spectrometry-based plasma proteomics using data-independent acquisition workflows quantifying from 3,000 to 7,000 proteins per sample, and immunoassay-based panels for indication-specific protein biomarkers comprising plasma neurofilament light chain, plasma glial fibrillary acidic protein, plasma phosphorylated-tau-181, plasma phosphorylated-tau-217, plasma phosphorylated-tau-231, plasma ubiquitin C-terminal hydrolase L1, plasma amyloid-beta 42/40 ratio, plasma total tau, plasma alpha-synuclein, and plasma TAR DNA-binding protein 43. Where intrathecal access is concurrently established for therapeutic delivery, the personalization layer additionally accepts cerebrospinal fluid proteome profiling using equivalent platforms with sampling volumes of from 0.5 to 5.0 milliliters and processing within from 30 minutes to 4 hours of collection to preserve labile protein species. Pathway-level proteomic signatures are computed using one or more of weighted gene co-expression network analysis applied to protein abundance matrices, hierarchical clustering of protein abundance z-scores, and module-eigenprotein extraction with subsequent correlation to clinical phenotypes. Protein-network-based response prediction is computed using one or more of graph neural network models trained on STRING database protein-protein interaction networks of approximately 19,000 human proteins and approximately 11 million interactions, network propagation algorithms applied to the BioGRID protein-protein interaction database, and prize-collecting Steiner tree algorithms applied to the Pathway Commons composite network. Response-predictor proteomic panels of from 20 to 200 proteins are derived using elastic-net regression, support vector regression, and deep neural network architectures trained on training cohorts of from 200 to 50,000 patients with held-out validation in independent cohorts.

Proteomic data are integrated into the closed-loop dosing controller as a multidimensional input vector comprising protein-abundance z-scores, pathway-activity scores, network-propagation scores, and response-predictor model outputs. Serial plasma proteomic sampling at intervals of from 14 to 180 days yields longitudinal trajectories that are smoothed with one or more of Gaussian-process regression, locally weighted scatterplot smoothing, and Kalman filtering, and the resulting smoothed trajectories are submitted to change-point detection algorithms to identify treatment-response shifts. The closed-loop controller computes a proteome-derived dose adjustment factor of from 0.5 to 2.0 relative to the prior dose, with proteomic-derived adjustments combined with genomic, transcriptomic, metabolomic, and digital-biomarker-derived adjustments using a Bayesian model-averaging framework that weights each modality by its patient-specific posterior predictive accuracy. Clinical utility comprises one or more of early treatment-response detection at from 30 to 90 days post-initiation versus 180 to 540 days for traditional clinical endpoints, identification of subclinical adverse-event signatures permitting prophylactic dose reduction, and stratification of patients into rapid-, intermediate-, and slow-progressor categories with progressor-category-specific dosing trajectories. Regulatory considerations comprise compliance with FDA ‘Biomarker Qualification: Evidentiary Framework’ guidance, conformity to Clinical Proteomic Tumor Analysis Consortium analytical performance standards, validation under Clinical Laboratory Improvement Amendments-certified laboratory protocols, and Letter of Support or Biomarker Qualification submissions to the FDA Biomarker Qualification Program where biomarker-driven dosing decisions are claimed as a labeled feature.

### Metabolome-Indexed Personalization

Metabolomic personalization inputs comprise plasma metabolomics obtained by liquid chromatography coupled to high-resolution mass spectrometry in both untargeted discovery mode quantifying from 5,000 to 50,000 mass-spectral features and targeted quantitative mode quantifying from 200 to 1,500 named metabolites; plasma lipidomics quantifying from 1,000 to 5,000 lipid species across glycerolipid, glycerophospholipid, sphingolipid, sterol, fatty acyl, and prenol lipid classes; gut-microbiome-derived metabolite profiling comprising short-chain fatty acids acetate, propionate, butyrate, valerate, and isobutyrate; primary bile acids cholic acid and chenodeoxycholic acid and secondary bile acids deoxycholic acid, lithocholic acid, ursodeoxycholic acid, and the conjugated taurine and glycine derivatives thereof; tryptophan metabolites comprising kynurenine, kynurenic acid, quinolinic acid, indole-3-acetic acid, indole-3-propionic acid, indole-3-aldehyde, indole-3-lactic acid, and serotonin; and branched-chain amino acids leucine, isoleucine, and valine and their alpha-keto-acid derivatives. Circadian metabolomic state is characterized by sampling at from 2 to 8 time points across a 24-hour cycle with chronotype-stratified time anchoring referenced to dim-light melatonin onset. The personalization engine maps metabolomic profiles to disease-subtype labels, drug-metabolism phenotype labels, and microbiome-metabolic-phenotype labels using machine-learning classifiers analogous to those applied to transcriptomic and proteomic data. Drug-metabolism phenotype inference from endogenous metabolites supplements genotype-based pharmacogenomic prediction by directly measuring CYP2D6 activity via the dextromethorphan-to-dextrorphan ratio, CYP3A4 activity via the 4-beta-hydroxycholesterol-to-cholesterol ratio or the midazolam-to-1-hydroxymidazolam ratio, and CYP2C19 activity via the omeprazole-to-5-hydroxyomeprazole ratio, thereby resolving phenoconversion arising from concomitant medications, inflammation, hepatic disease, or rare uncharacterized alleles.

Microbiome-metabolite axis personalization integrates the gut-microbiome-derived metabolite profile with 16S ribosomal RNA gene sequencing of fecal samples at depth of from 10,000 to 100,000 reads per sample or shotgun metagenomic sequencing of fecal samples at depth of from 5 million to 50 million reads per sample to compute a microbiome-metabolic-state vector that informs dose-scheduling decisions. Specifically, the platform delays dosing of bile-acid-modulating payload classes during periods of dysbiosis-associated bile-acid-pool perturbation, accelerates dosing of butyrate-pathway-modulating payload classes during periods of low butyrate output, and adjusts dosing of indole-pathway-modulating payload classes based on indole-3-propionic acid concentration relative to the patient-specific baseline. Circadian-rhythm-aware dosing time windows are derived from the circadian metabolomic state and are implemented as patient-specific 2-to-6-hour windows during which payload absorption and central-nervous-system exposure are maximized. Closed-loop integration comprises serial metabolomic sampling at intervals of from 7 to 90 days, with each sample yielding an updated metabolomic-state vector that is submitted to the multimodal Bayesian model-averaging framework alongside transcriptomic, proteomic, genomic, and digital-biomarker inputs. Clinical utility comprises one or more of improved oral bioavailability variability reduction of from 25 percent to 60 percent through metabolome-informed timing, reduction in gastrointestinal adverse-event rate of from 30 percent to 70 percent through microbiome-informed payload selection, and improvement in central-nervous-system penetration consistency through chronotype-anchored dosing. Regulatory considerations comprise compliance with FDA draft guidance ‘Microbiome Considerations for Drug Development,’ Metabolomics Standards Initiative analytical reporting standards, mass-spectrometry-based metabolomics validation following the Clinical and Laboratory Standards Institute C62-A guidance, and where metabolomic data inform dosing decisions, validation as either a drug-development tool or a companion diagnostic under the appropriate FDA pathway.

### Patient-Derived Induced Pluripotent Stem Cell Modeling

The platform incorporates patient-derived induced pluripotent stem cell modeling as an ex vivo personalization layer through which payload candidates are screened against a patient's own cellular substrate. Induced pluripotent stem cell reprogramming is performed from peripheral blood mononuclear cells at input of from 1 million to 10 million cells or from dermal fibroblasts at input of from 100,000 to 1,000,000 cells using one or more of episomal reprogramming with Yamanaka factors OCT4, SOX2, KLF4, and L-MYC delivered via episomal plasmids, Sendai virus reprogramming with the CytoTune-iPS 2.0 kit, modified messenger RNA reprogramming with daily transfections over from 14 to 21 days, and microRNA-supplemented reprogramming. Reprogramming yields induced pluripotent stem cell lines at efficiency of from 0.01 percent to 1 percent, with from 3 to 20 clones expanded per patient and karyotyped, copy-number-profiled, pluripotency-marker-validated by immunofluorescence for OCT4, NANOG, SOX2, SSEA-4, and TRA-1-60, and tri-lineage-differentiation-validated to ensure genomic integrity and developmental potential. Differentiation into disease-relevant central nervous system cell types comprises one or more of cortical glutamatergic neuron differentiation via dual-SMAD inhibition followed by neuronal patterning over from 30 to 90 days, ventral midbrain dopaminergic neuron differentiation via floor-plate induction with sonic hedgehog agonists and Wnt activation over from 25 to 75 days, striatal medium spiny neuron differentiation via lateral ganglionic eminence patterning, motor neuron differentiation via caudalization with retinoic acid and ventralization with sonic hedgehog over from 14 to 42 days, GABAergic interneuron differentiation via medial ganglionic eminence patterning, astrocyte differentiation via prolonged neural progenitor culture in FGF and EGF over from 60 to 180 days, microglia-like cell differentiation via mesodermal patterning followed by microglial maturation over from 35 to 70 days, and oligodendrocyte differentiation via PDGF-AA, NT3, and T3 patterning over from 75 to 150 days. Differentiated cells are characterized by single-cell RNA-sequencing, electrophysiology, and immunostaining to confirm cell-type identity and maturation state.

Patient-derived induced pluripotent stem cell screening proceeds in two-dimensional monolayer culture for high-throughput payload screening, three-dimensional organoid culture for tissue-context screening, and assembloid culture for circuit-context screening. Two-dimensional screening is performed in from 96-well to 1536-well format with from 1,000 to 50,000 cells per well, with payload candidates dosed across from 5 to 12 concentration points spanning from 1 nM to 10 microM. Three-dimensional cortical, midbrain, hippocampal, and spinal-cord organoid screening is performed at organoid age of from 30 to 365 days post-induction with payload exposure of from 1 to 21 days. Assembloid models comprise cortico-striatal assembloids fusing cortical glutamatergic-neuron organoids with striatal medium-spiny-neuron organoids for analysis of corticostriatal circuit function, cortico-thalamic assembloids fusing cortical organoids with thalamic organoids for analysis of thalamocortical loop function, cortico-spinal assembloids for analysis of upper- and lower-motor-neuron connectivity in amyotrophic lateral sclerosis screening, and cortico-cortical assembloids for analysis of long-range cortical projections. Readouts comprise one or more of high-content imaging of cell viability, neurite morphology, synaptic-marker density, mitochondrial morphology, and aggregate burden using automated confocal microscopy; multi-electrode-array recording of spontaneous and evoked neuronal activity; calcium imaging using genetically encoded calcium indicators GCaMP6f, GCaMP7f, GCaMP8f, or jGCaMP8; voltage imaging using genetically encoded voltage indicators QuasAr3, Voltron2, or Archon1; single-cell RNA-sequencing for transcriptomic response; and Olink or SomaScan proteomic profiling of conditioned medium. Patient-derived induced pluripotent stem cell screening results are integrated into the personalized payload-ranking module as a patient-specific payload-response prior that is updated by in vivo closed-loop biomarker data. Clinical utility comprises one or more of ex vivo identification of patient-specific non-responder payload classes prior to in vivo exposure, identification of patient-specific synergistic combinations, and ex vivo characterization of patient-specific adverse-event signatures. Regulatory considerations comprise compliance with FDA ‘Considerations for the Design of Early-Phase Clinical Trials of Cellular and Gene Therapy Products’ guidance where induced-pluripotent-stem-cell-derived testing informs first-in-human dose selection, and conformity to International Society for Stem Cell Research guidelines on stem cell research and clinical translation.

### Patient-Derived Organoid Screening

Patient-derived organoid screening platforms comprise cerebral cortical organoids of from 1 to 5 millimeter diameter at age of from 90 to 720 days post-induction containing cortical-plate, sub-plate, and intermediate-zone progenitor layers and supporting glutamatergic neurons, GABAergic interneurons, astrocytes, and oligodendrocyte progenitors; midbrain organoids enriched for ventral mesencephalic dopaminergic neurons with neuromelanin accumulation in cultures of from 120 to 365 days; hippocampal organoids containing dentate-gyrus and Cornu Ammonis subfield identities; spinal-cord organoids containing motor-neuron and interneuron populations along the dorsoventral axis; cerebellar organoids containing Purkinje-cell and granule-cell populations; thalamic and hypothalamic organoids; choroid-plexus organoids producing cerebrospinal-fluid-like secretions; and blood-brain-barrier organoids comprising brain microvascular endothelial cells, pericytes, and astrocytes. Organoid-on-chip platforms incorporate microfluidic vascularization comprising endothelial-cell-lined channels of from 100 to 500 micrometer diameter perfused at shear stress of from 1 to 20 dyne per square centimeter; immune-cell integration comprising patient-derived peripheral blood mononuclear cells or induced-pluripotent-stem-cell-derived microglia introduced at from 1,000 to 100,000 cells per organoid; and biosensor integration for real-time monitoring of oxygen, glucose, lactate, pH, and electrical activity. High-content imaging screens of candidate payload classes are performed at imaging throughput of from 1,000 to 100,000 organoid images per day using one or more of light-sheet microscopy, two-photon microscopy, expansion microscopy with isotropic expansion of from 4-fold to 20-fold, and tissue-clearing-enabled whole-organoid confocal imaging using CLARITY, CUBIC, iDISCO, or SHIELD clearing protocols.

Multi-omic readouts on patient-derived organoid screens comprise spatial transcriptomics at sub-cellular resolution using 10× Genomics Visium HD, 10× Genomics Xenium, NanoString CosMx, Vizgen MERSCOPE, or Resolve Biosciences Molecular Cartography platforms; single-cell and single-nucleus RNA-sequencing at depth of from 10,000 to 200,000 cells per organoid; single-cell ATAC-sequencing for chromatin-accessibility profiling; spatial proteomics using imaging mass cytometry, multiplexed ion-beam imaging, or co-detection by indexing; and metabolomic imaging using matrix-assisted laser desorption/ionization mass-spectrometry imaging. Screening data are integrated into a patient-specific payload-ranking matrix in which each candidate payload is scored across efficacy, off-target, and adverse-event axes, with the resulting matrix submitted to the closed-loop dosing controller as a patient-specific payload prior. The organoid-screening output further informs combination-payload selection by computing pairwise and higher-order synergy scores using Bliss independence, Loewe additivity, highest-single-agent, and zero-interaction-potency models. Clinical utility comprises one or more of patient-stratified payload deployment yielding ex vivo response-rate improvements that translate to in vivo response-rate improvements of from 20 percent to 70 percent, identification of patient-specific resistance mechanisms with sufficient lead time to pre-empt clinical progression, and rational combination-payload selection. Regulatory considerations comprise alignment with FDA ‘Considerations for the Design and Conduct of Externally Controlled Trials for Drug and Biological Products’ where organoid data inform external control arm construction, alignment with FDA ‘Best Practices for Conducting and Reporting Pharmacoepidemiologic Safety Studies Using Electronic Healthcare Data,’ alignment with the FDA Innovative Science and Technology Approaches for New Drugs program for organoid-based predictive models, and compliance with the FDA Modernization Act 2.0 framework supporting non-animal testing methods including organoid and organ-on-chip systems.

### Closed-Loop Adjustment Using Accumulated Longitudinal Monitoring Data

The closed-loop adjustment subsystem accumulates longitudinal monitoring data spanning multi-month and multi-year dosing trajectories at sampling intervals of from 1 minute for continuous physiological sensors to 365 days for annual comprehensive multi-omic profiling. The longitudinal data store maintains for each patient a time-indexed multi-channel record comprising continuous-wearable signals at from 0.0167 Hz to 1000 Hz sampling frequency, intermittent self-administered cognitive and behavioral assessments at from daily to monthly intervals, intermittent biospecimen-derived biomarker measurements at from monthly to annual intervals, intermittent imaging-derived biomarker measurements at from quarterly to annual intervals, and continuous adherence and dosing-event logs. Trend-detection algorithms comprise one or more of Mann-Kendall trend tests applied to time-series segments of from 30 to 365 days, Theil-Sen slope estimators robust to outliers, seasonal-trend decomposition using locally weighted scatterplot smoothing, and structural-time-series models with stochastic level, slope, and seasonal components. Change-point detection algorithms comprise one or more of binary segmentation with the pruned exact linear time method, Bayesian online change-point detection with Gaussian, Student-t, and Poisson observation models, kernel-based change-point detection with radial-basis-function kernels, and Gaussian-process-based change-point detection. Change-point detection sensitivity is tuned to detect biomarker-trajectory shifts corresponding to from 0.5 to 2.0 standard deviations relative to the patient-specific baseline with false-positive rate of from 1 percent to 10 percent per year.

Bayesian updating of patient-specific pharmacokinetic and pharmacodynamic model parameters is performed using one or more of sequential Monte Carlo particle filtering, ensemble Kalman filtering, Markov chain Monte Carlo with adaptive proposal distributions, and variational Bayesian inference with mean-field or full-rank Gaussian variational families. The patient-specific pharmacokinetic model comprises a compartmental structure of from 1 to 5 compartments parameterized by absorption rate constant, distribution rate constants, clearance, and volume of distribution, with each parameter assigned a population-level prior derived from a reference pharmacokinetic database of from 100 to 10,000 individuals and updated by patient-specific drug concentration observations. The patient-specific pharmacodynamic model comprises one or more of direct-effect, biophase-distribution, indirect-response, transit-compartment, and turnover models linking drug concentration to biomarker response with parameters updated by patient-specific biomarker observations. Closed-loop dose adjustment is computed at intervals of from 1 day to 90 days using model-predictive control with a planning horizon of from 30 to 730 days, a cost function combining efficacy, safety, and adherence terms, and a feasibility constraint ensuring that prescribed doses remain within the safe and approved range. Clinical utility comprises one or more of detection of treatment-response shifts at from 30 to 180 days before they would be detected by scheduled clinical visits, anticipatory dose adjustment in response to predicted future trajectory rather than reactive adjustment in response to past trajectory, and individualization of dose-titration trajectory to patient-specific tolerability. Regulatory considerations comprise compliance with FDA guidance on model-informed drug development, FDA guidance on population pharmacokinetics, FDA ‘Software as a Medical Device’ classification for closed-loop dosing algorithms, FDA ‘Marketing Submission Recommendations for a Predetermined Change Control Plan for Artificial Intelligence/Machine Learning-Enabled Device Software Functions,’ and conformity to International Council for Harmonisation E11A pediatric extrapolation principles where pediatric closed-loop adjustment is deployed.

Integration with Personalized Methods-of-Administration Architecture

The platform integrates with the personalized methods-of-administration architecture described in commonly-owned co-pending U.S. Patent Application No. 64/080,301 (‘Personalized Methods of Administration for Central Nervous System Therapeutics’), no priority benefit being claimed to that application. The integration provides, for each patient and each payload class, a personalized administration-route prescription drawn from a route catalog comprising oral solid dosage forms including tablets, capsules, multi-particulate orally-disintegrating tablets, and gastro-retentive formulations; oral liquid dosage forms; sublingual films and tablets; buccal films and tablets; intranasal sprays, gels, and powders; transdermal patches with active or passive enhancement; depot subcutaneous formulations with release durations of from 7 to 365 days; intramuscular depot formulations; intravenous infusion; intrathecal bolus injection; intrathecal continuous infusion via implantable pump; intracerebroventricular bolus injection via Ommaya reservoir; intracerebroventricular continuous infusion; intracerebral convection-enhanced delivery; and intra-arterial infusion with osmotic blood-brain-barrier disruption. Administration-route selection is computed by a route-personalization module that integrates patient-specific factors comprising gastrointestinal absorption capacity from metabolomic and digital-biomarker inputs, swallowing ability from clinical assessment and patient-reported outcomes, hepatic first-pass metabolism capacity from pharmacogenomic and metabolomic inputs, dosing-frequency tolerance from adherence history, caregiver availability for assisted administration, vascular access status, and presence of indwelling intrathecal or intracerebroventricular access devices. The closed-loop controller is administration-route-aware in that pharmacokinetic and pharmacodynamic model parameters are conditioned on the active administration route, and route-switching decisions are made adaptively when oral-route exposure variability exceeds a patient-specific threshold corresponding to from 30 percent to 80 percent coefficient of variation in trough drug concentrations. Clinical utility comprises one or more of preservation of central-nervous-system exposure consistency across disease progression that compromises swallowing, reduction in dosing burden through depot or implant route transitions, and rescue of non-response attributable to oral absorption failure. Regulatory considerations comprise compliance with FDA guidance on bioequivalence and dosage-form-substitution standards, FDA combination-product classification under 21 CFR Part 3 where device-mediated routes are employed, and supplemental New Drug Application or efficacy supplement filings as new routes are added to the labeled indications.

Integration with Genome-Indexed Therapeutic Subscription and Multi-Decade Financing Architecture

The platform integrates with the genome-indexed therapeutic subscription and multi-decade financing architecture described in commonly-owned co-pending application family DVB-063 (‘Genome-Indexed Therapeutic Subscription and Multi-Decade Financing’), no priority benefit being claimed to that application family. The integration enables subscription-based dosing economics in which the patient or payer commits to a multi-year therapeutic relationship with payment structured as an annuity rather than a per-prescription transaction, with subscription pricing actuarially derived from the patient-specific genomic risk profile, predicted lifetime dose schedule, and predicted lifetime clinical-outcome trajectory. Payer alignment is achieved through outcome-linked pricing in which a portion of subscription revenue of from 5 percent to 50 percent is contingent on achievement of pre-specified clinical-outcome milestones comprising one or more of biomarker-trajectory improvement of from 20 percent to 70 percent relative to the predicted untreated trajectory, functional-outcome milestone achievement on the Clinical Dementia Rating-Sum of Boxes, Movement Disorder Society-Unified Parkinson's Disease Rating Scale, ALS Functional Rating Scale-Revised, or other indication-appropriate clinical outcome assessment, time-to-event endpoints comprising time to nursing-home placement or time to assisted-living transition of from 12 to 120 months extension, and survival endpoints of from 6 to 60 months extension. The closed-loop controller interfaces with the financing module by providing predicted dose trajectories that inform actuarial pricing models and by providing measured outcome trajectories that trigger outcome-contingent payment flows. Clinical utility comprises one or more of patient access to multi-decade personalized therapeutic regimens without per-dose financial barriers, alignment of manufacturer incentives with long-term patient outcomes, and removal of dose-escalation cost barriers that otherwise compromise adherence. Regulatory considerations comprise Centers for Medicare and Medicaid Services value-based payment arrangement frameworks under the Medicaid Best Price Final Rule, the Inflation Reduction Act drug price negotiation framework, FDA marketing-claim substantiation requirements for outcome-linked pricing claims, and state-law insurance and risk-bearing entity classifications.

Integration with Integrated Peptide Therapeutic Platform Architecture

The platform integrates with the integrated peptide therapeutic platform architecture described in commonly-owned co-pending application family DVB-070 (‘Integrated Peptide Therapeutic Platform’), no priority benefit being claimed to that application family. The integration provides peptide-cargo-specific delivery enhancements drawn from a peptide-delivery enhancement catalog comprising N-terminal capping with acetyl, pyroglutamyl, or fatty acyl groups for amino-peptidase resistance; C-terminal amidation for carboxypeptidase resistance; non-natural amino acid substitution including D-amino acids, beta-amino acids, N-methylated amino acids, and alpha-methyl amino acids for proteolytic resistance; cyclization via head-to-tail, head-to-side-chain, side-chain-to-side-chain, or stapling chemistries for conformational restriction and proteolytic resistance; lipidation with palmitoyl, stearoyl, or fatty-diacid conjugates for plasma-albumin-binding-mediated half-life extension; PEGylation with linear or branched polyethylene glycol of molecular weight from 5 kilodaltons to 80 kilodaltons; XTEN or PASylation polymer fusion; Fc-fusion for FcRn-mediated half-life extension; transferrin or albumin genetic fusion; and incorporation of unnatural amino acids enabling site-specific bioconjugation via copper-free click chemistry, oxime ligation, or sortase-mediated ligation. The peptide-delivery enhancement selection module computes a patient-specific and payload-specific enhancement vector based on the patient's proteolytic capacity from metabolomic and proteomic inputs, the patient's renal clearance capacity from creatinine clearance and proteomic kidney-injury markers, and the patient's immunogenicity risk profile from HLA genotype. Closed-loop integration provides peptide-specific exposure monitoring through plasma peptide concentration measurement and anti-drug-antibody monitoring, with antibody-positive patients triggered into an alternative-enhancement payload-switch workflow. Clinical utility comprises one or more of extension of peptide half-life from minutes-to-hours in unmodified form to days-to-weeks in optimally-modified form, reduction in immunogenicity-related loss of efficacy of from 30 percent to 80 percent, and rescue of patients who develop neutralizing antibodies to first-line peptide payloads. Regulatory considerations comprise compliance with FDA ‘Immunogenicity Testing of Therapeutic Protein Products’ and ‘Immunogenicity Assessment for Therapeutic Protein Products’ guidances, conformity to International Council for Harmonisation S6 (R1) preclinical safety evaluation principles for biotechnology-derived pharmaceuticals, and post-marketing immunogenicity surveillance commitments.

Integration with Reversible Blood-Brain-Barrier-Penetrating Peptide Delivery Platform

The platform integrates with the reversible blood-brain-barrier-penetrating peptide delivery platform described in commonly-owned co-pending application family DVB-016 (‘Reversible Blood-Brain-Barrier-Penetrating Neurogenic Peptide Platform’), no priority benefit being claimed to that application family. The integration provides blood-brain-barrier shuttle peptide modality compatibility comprising covalent, non-covalent, and genetic-fusion conjugation of payload cargoes to shuttle peptides drawn from a shuttle-peptide catalog comprising angiopep-2 and angiopep-derivatives targeting low-density lipoprotein receptor-related protein 1; ApoB and ApoE peptides targeting low-density lipoprotein receptor-related proteins; melanotransferrin-derived peptides targeting p97; transferrin-receptor-binding peptides including B6, T7, and 7E peptides; rabies-virus-glycoprotein-derived RVG29 peptide targeting nicotinic acetylcholine receptor; GLUT1-binding peptides targeting glucose transporter 1; insulin-receptor-binding peptides; nicotinic-acetylcholine-receptor-binding peptides; and enzyme-activated peptides whose blood-brain-barrier transcytosis activity is gated by central-nervous-system-localized proteases. Shuttle-peptide selection is computed by a shuttle-selection module that integrates patient-specific factors comprising blood-brain-barrier-receptor expression inferred from plasma proteomic and cerebrospinal fluid proteomic inputs, transferrin saturation status, glucose homeostasis status, and indication-specific blood-brain-barrier-receptor-modulation patterns. Reversibility of blood-brain-barrier penetration is implemented through enzyme-activated peptide architectures in which transcytosis-promoting moieties are masked by central-nervous-system-protease-cleavable linkers, ensuring that systemic blood-brain-barrier opening is bounded in time and space and that off-target peripheral exposure minimized. Closed-loop integration provides shuttle-peptide-exposure monitoring through quantitative plasma shuttle-peptide measurement, central-nervous-system-exposure surrogate measurement via paired serum and cerebrospinal fluid sampling where intrathecal access is available, and adverse-event monitoring for blood-brain-barrier-opening-related events including amyloid-related imaging abnormalities and seizure risk. Clinical utility comprises one or more of central-nervous-system delivery enhancement of from 5-fold to 100-fold for shuttle-conjugated payloads versus unconjugated payloads, reduction in systemic-exposure burden for equivalent central-nervous-system exposure, and rescue of patients with shuttle-receptor-expression deficiency through shuttle-class switching. Regulatory considerations comprise FDA ‘Drug Products, Including Biological Products, that Contain Nanomaterials’ guidance, blood-brain-barrier-targeting agent characterization standards, and combination-product-classification considerations where shuttle-payload conjugates qualify as combination products.

### Portfolio-Level Integration Across Neurocore Biotechnologies LLC Patent Family

The platform is architected for modular interoperation with the broader Neurocore Biotechnologies LLC patent portfolio, with interoperation specifications defining data-exchange interfaces, mechanism-of-action complementarities, and combination-deployment protocols. Interoperation with the magnetically guided self-powered neural nanochip platform described in commonly-owned application DVB-001 enables payload-loaded nanochip deployment at magnetically-targeted central-nervous-system loci with self-powered chronic residence times of from 30 to 3,650 days, with the present oral platform providing the systemically-administered priming and supplementation regimen surrounding the implanted nanochip. Interoperation with the CRISPR plus DNA origami blood-brain-barrier nanorobot described in commonly-owned application family DVB-003 enables genome-editing payload deployment to selected central-nervous-system loci, with the present oral platform providing pharmacogenomically-personalized small-molecule and peptide-cargo regimens that synergize with or substitute for genome-editing interventions. Interoperation with the tetrahedral DNA origami tri-pathway protein degradation platform described in commonly-owned application family DVB-014 enables PROTAC-, ATTEC-, and LYTAC-mediated proteinopathy degradation, with the present oral platform providing personalized cargo-selection across the from 300 to 500 protein-degradation indications mapped therein. Interoperation with the self-amplifying circular-RNA platform described in commonly-owned application family DVB-013 enables sustained protein-replacement-therapy deployment over from 14 to 28 days per dose with personalized cargo-selection across protein-deficiency indications. Interoperation with the sequential-release glymphatic-widening platform described in commonly-owned application family DVB-012 enables glymphatic-augmentation-paired proteinopathy clearance with personalized aquaporin-4-modulator and nanobody pairing. Interoperation with the reversible epigenetic-editing platform described in commonly-owned application family DVB-011 enables dCas9-DNMT3A, dCas9-TET1, and dCas9-p300-mediated epigenetic reprogramming with personalized locus selection and reversibility-trajectory programming. Interoperation with the cell-fate reprogramming nanorobot platform described in commonly-owned application family DVB-010 enables in vivo partial reprogramming with personalized OSK-cargo dosing and rejuvenation-trajectory monitoring. Interoperation with the blood-brain-barrier-penetrating gene-therapy platform described in commonly-owned application family DVB-009 enables AAV-, lentiviral-, and non-viral gene-therapy cargo deployment with personalized capsid and promoter selection. The portfolio-integration controller computes patient-specific multi-modality regimens combining selections from the foregoing platforms using a multi-objective optimization framework that maximizes predicted efficacy subject to safety, cost, and combination-tractability constraints, with the resulting regimens deployed under coordinated longitudinal-monitoring schedules.

### Genotype-Stratified Clinical-Trial Design

The platform supports genotype-stratified clinical-trial designs comprising N-of-1 trial designs using genome-indexed enrollment, basket trial designs spanning multiple indications unified by shared molecular targets, and master-protocol architectures spanning multiple investigational payloads and multiple indications. N-of-1 trial designs are implemented for ultra-rare-variant-driven indications using a within-patient crossover structure of from 2 to 8 alternating active and placebo periods of from 14 to 365 days each, with patient-specific endpoint selection driven by patient-specific biomarker and functional-outcome trajectories. Basket trials enroll patients across multiple indications who share a molecular feature including a shared variant, a shared pathway dysregulation, or a shared transcriptomic subtype, and randomize across investigational payloads with allocation ratios of from 1:1 to 1:5 active-to-control. Adaptive Bayesian dose-finding designs employ continual reassessment methods, escalation with overdose control, model-assisted designs including Bayesian optimal interval and modified toxicity probability interval, and machine-learning-augmented designs in which historical-trial data inform priors. Biomarker-stratified randomization ensures balanced allocation of biomarker-positive and biomarker-negative patients across treatment arms, and biomarker-driven enrichment designs restrict enrollment to biomarker-positive patients where prior evidence justifies enrichment. Master-protocol architectures permit simultaneous investigation of multiple payloads across multiple indications under a single regulatory umbrella, with shared control arms, shared infrastructure, and shared statistical analysis plans. Closed-loop integration provides real-time enrollment-stratification updates as new patient-specific genomic and transcriptomic data become available, real-time interim-analysis updates triggering arm-dropping and arm-adding decisions, and real-time safety-monitoring updates. Clinical utility comprises one or more of acceleration of payload-evaluation timelines of from 25 percent to 60 percent relative to non-adaptive single-indication trials, reduction in sample-size requirements of from 30 percent to 70 percent through informative-prior incorporation, and identification of biomarker-defined responder subpopulations that would be missed in unstratified trials. Regulatory considerations comprise compliance with FDA ‘Adaptive Designs for Clinical Trials of Drugs and Biologics’ guidance, FDA ‘Master Protocols: Efficient Clinical Trial Design Strategies to Expedite Development of Oncology Drugs and Biologics’ guidance, FDA ‘Enrichment Strategies for Clinical Trials to Support Determination of Effectiveness of Human Drugs and Biological Products’ guidance, FDA ‘Demonstrating Substantial Evidence of Effectiveness for Human Drug and Biological Products’ guidance, and pre-submission and Type B meetings with the Center for Drug Evaluation and Research for master-protocol architectures.

### Decentralized Clinical-Trial Elements

The platform supports decentralized clinical-trial elements comprising at-home dosing with telemedicine oversight, wearable-device data collection, and remote biomarker sampling. At-home dosing is implemented for oral and self-administered subcutaneous payload classes through a patient-facing smart-dispenser device that integrates with the closed-loop dosing controller and that records, for each dosing event, the time of dispensation, the dose dispensed, the patient identity confirmed by biometric authentication comprising one or more of fingerprint, face-recognition, and voice-recognition modalities, and a post-dose confirmation video uploaded for telemedicine review. Telemedicine oversight is conducted via synchronous video visits at intervals of from 14 to 180 days, asynchronous secure-messaging follow-up at intervals of from 1 to 30 days, and continuous passive monitoring through wearable-device data streams. Wearable-device data collection comprises one or more of triaxial accelerometry at from 25 Hz to 200 Hz for activity, gait, and tremor quantification; photoplethysmography for heart rate, heart rate variability, and oxygen saturation; continuous electrocardiography for arrhythmia detection; electrodermal activity for autonomic-arousal quantification; skin temperature for circadian-rhythm and febrile-event detection; global positioning system data for life-space quantification; smartphone keystroke dynamics for cognitive and motor function inference; and voice and speech biomarkers from passive smartphone microphone sampling. Remote biomarker sampling comprises dried blood spot collection on Whatman 903 or equivalent collection cards with shipment to a central laboratory for quantification of plasma drug concentrations, plasma neurofilament light chain, plasma glial fibrillary acidic protein, plasma phosphorylated-tau, and other indication-appropriate analytes; volumetric absorptive microsampling using Mitra or Hamilton VAMP devices; saliva collection for cortisol, alpha-amylase, and select drug analytes; and home urine collection for metabolomic profiling. Closed-loop integration ingests decentralized-trial data through encrypted, HIPAA-compliant, 21 CFR Part 11-compliant data pipelines with audit trails, source-data verification, and electronic signature provisions. Clinical utility comprises one or more of expansion of trial accessibility to patients in non-research-center geographies, reduction in patient travel and time burdens of from 50 percent to 90 percent, improved enrollment diversity, and richer longitudinal data densities. Regulatory considerations comprise compliance with FDA ‘Conducting Clinical Trials With Decentralized Elements’ guidance, FDA ‘Digital Health Technologies for Remote Data Acquisition in Clinical Investigations’ guidance, FDA 21 CFR Part 11 guidance, International Council for Harmonisation E6 (R3) Good Clinical Practice, and applicable state-by-state telemedicine licensure regulations.

### Real-World Evidence Collection Post-Approval

The platform supports systematic real-world evidence collection in the post-approval setting comprising pragmatic trials, registry-based long-term follow-up, insurance-claims-data analysis, and patient-reported outcomes integration. Pragmatic trials are designed under the PRECIS-2 framework with intentional reduction of explanatory-trial restrictions across eligibility, recruitment, setting, organization, flexibility of delivery, flexibility of adherence, follow-up, primary outcome, and primary analysis domains, with target enrollment of from 1,000 to 100,000 patients per pragmatic trial across from 10 to 1,000 community-practice sites. Registry-based long-term follow-up is conducted via patient-condition-specific registries with prospective enrollment of all initiated patients, structured follow-up data collection at intervals of from 90 to 365 days for from 5 to 30 years post-initiation, and linkage to electronic health record, claims, and vital-status data sources. Insurance-claims-data analysis is performed against payer-specific and aggregated claims databases comprising Medicare fee-for-service and Medicare Advantage, Medicaid, commercial-payer aggregations, and integrated-delivery-network electronic health record plus claims linkages, with analytic methods comprising propensity-score matching, propensity-score weighting, instrumental-variable analysis, target-trial emulation, and high-dimensional propensity-score adjustment. Patient-reported outcomes integration is performed via validated patient-reported outcome measures including the Patient-Reported Outcomes Measurement Information System instruments, indication-specific instruments such as the Quality of Life in Alzheimer's Disease, Parkinson's Disease Questionnaire-39, ALS Assessment Questionnaire-40, EQ-5D-5L, and SF-36, with patient-reported outcome data collected at intervals of from 30 to 365 days. Closed-loop integration channels real-world evidence findings back to the personalization controller as updated population-level priors for pharmacokinetic, pharmacodynamic, and outcome models, and back to the regulatory and labeling functions as post-marketing safety and effectiveness signals. Clinical utility comprises one or more of generation of comparative-effectiveness evidence supporting payer coverage decisions, identification of rare adverse events at incidence rates of from 1 in 1,000 to 1 in 1,000,000 patient-years, identification of effectiveness in subpopulations excluded from pre-approval trials, and refinement of dosing recommendations in the post-approval setting. Regulatory considerations comprise compliance with the FDA Real-World Evidence Program framework, FDA ‘Considerations for the Use of Real-World Data and Real-World Evidence to Support Regulatory Decision-Making for Drug and Biological Products’ guidance, FDA ‘Real-World Data: Assessing Electronic Health Records and Medical Claims Data’ guidance, the 21st Century Cures Act real-world-evidence provisions, and equivalent European Medicines Agency ‘Guideline on Registry-Based Studies’ provisions.

### Companion Diagnostic Integration

The platform integrates payload-class-specific companion diagnostics, multiplex biomarker panels, imaging-based companion diagnostics, and digital companion diagnostics. Payload-class-specific companion diagnostics are developed in parallel with payload-development programs and submitted to the FDA via the in vitro diagnostic premarket approval pathway under 21 CFR Part 814 or, where applicable, the 510 (k) clearance pathway under 21 CFR Part 807, with co-development conducted under FDA ‘In Vitro Companion Diagnostic Devices’ guidance and FDA ‘Principles for Codevelopment of an In Vitro Companion Diagnostic Device with a Therapeutic Product’ guidance. Multiplex biomarker panels integrate from 5 to 500 analytes per panel and are deployed as orthogonal personalization inputs to the closed-loop controller, with multiplex panels validated under Clinical Laboratory Improvement Amendments-certified laboratory protocols and, where claimed as companion diagnostics, under FDA in vitro diagnostic regulatory pathways. Imaging-based companion diagnostics comprise one or more of amyloid positron emission tomography using florbetapir-F18, florbetaben-F18, and flutemetamol-F18 tracers with Centiloid quantification, tau positron emission tomography using flortaucipir-F18, MK-6240-F18, and PI-2620-F18 tracers with regional standardized-uptake-value-ratio quantification, dopamine-transporter single-photon emission computed tomography using ioflupane-1123 for Parkinson disease and Lewy-body dementia, neuroinflammation positron emission tomography using translocator-protein ligands, and synaptic-density positron emission tomography using SV2A ligands. Magnetic-resonance-imaging-based companion diagnostics comprise volumetric hippocampal and whole-brain measurement, diffusion-tensor imaging of white-matter tracts, resting-state functional magnetic resonance imaging of default-mode and salience networks, arterial-spin-labeling perfusion imaging, and magnetic resonance spectroscopy of neurometabolites. Digital companion diagnostics comprise validated cognitive-assessment instruments deployed via smartphone and tablet at intervals of from 1 to 90 days, validated motor-function instruments including digital finger tapping, gait analysis, and tremor quantification, validated speech and language instruments, and composite digital-biomarker scores derived from passive smartphone and wearable data. Closed-loop integration channels companion-diagnostic outputs as patient-specific posterior updates to the personalization controller, with companion-diagnostic-driven payload-switching decisions implemented at intervals of from 30 to 365 days. Clinical utility comprises one or more of payload-class precision-matching yielding response-rate improvements of from 25 percent to 75 percent, early detection of treatment failure permitting timely payload switching, and reduction of unnecessary exposure to non-effective payloads. Regulatory considerations comprise FDA in vitro companion diagnostic co-development pathway compliance, FDA ‘Software as a Medical Device’ classification for digital companion diagnostics, FDA ‘Clinical Decision Support Software’ guidance applicability, and Centers for Medicare and Medicaid Services coverage determinations for companion diagnostics under National Coverage Determinations and Local Coverage Determinations.

### Adaptive Dosing Across the Lifespan

The platform implements adaptive dosing across the human lifespan comprising pediatric dosing scaling rules, geriatric dose adjustments, pregnancy and lactation dosing where applicable, and end-of-life palliative dosing. Pediatric dosing scaling is implemented using one or more of allometric scaling with exponents of 0.75 for clearance and 1.0 for volume of distribution, body-surface-area scaling, age-dependent maturation functions for cytochrome P450 enzymes, uridine diphosphate glucuronosyltransferase enzymes, and transporter proteins, and physiologically-based pharmacokinetic modeling with age-stratified organ-volume, blood-flow, and enzyme-expression parameters. Pediatric dose-titration is bounded by safety thresholds derived from indication-specific pediatric clinical trials and is conditioned on pediatric blood-brain-barrier-permeability development. Geriatric dose adjustments are implemented for the geriatric population aged from 65 years to greater than 100 years and address age-associated decline in glomerular filtration rate of approximately 1 milliliter per minute per 1.73 square meters per year of age beyond age 40, hepatic blood flow decline of approximately 0.5 percent to 1 percent per year, cytochrome P450 enzyme activity decline of variable magnitude across isoforms, plasma albumin decline, body-composition shifts toward increased adipose and decreased lean mass, and polypharmacy-associated drug-drug interaction risk that increases monotonically with concomitant medication count. Pregnancy and lactation dosing, where the payload class is approved for use in pregnancy under FDA Pregnancy and Lactation Labeling Rule classifications, is implemented with adjustments for pregnancy-associated increases in plasma volume, glomerular filtration rate, hepatic enzyme induction or inhibition, and protein binding alterations, with closed-loop monitoring intensified to weekly or biweekly intervals and with lactation transfer monitored through paired maternal-plasma and breast-milk drug concentration assays. End-of-life palliative dosing is implemented through transition protocols that prioritize symptom control, comfort, and reduction in dosing burden, with closed-loop monitoring transitioned from disease-modification optimization to symptom-control and quality-of-life optimization. Clinical utility comprises one or more of safe pediatric label-extension permitting pediatric patient access, optimized geriatric dosing reducing adverse-event rates of from 30 percent to 70 percent relative to age-unadjusted dosing, evidence-based dosing recommendations in pregnancy and lactation that minimize fetal and infant exposure risk while maintaining maternal therapeutic benefit, and humane end-of-life dosing that respects patient autonomy. Regulatory considerations comprise compliance with the Pediatric Research Equity Act, the Best Pharmaceuticals for Children Act, FDA ‘General Clinical Pharmacology Considerations for Pediatric Studies of Drugs, Including Biological Products’ guidance, FDA ‘Pregnancy, Lactation, and Reproductive Potential: Labeling for Human Prescription Drug and Biological Products’ guidance, and Centers for Medicare and Medicaid Services hospice and palliative-care coverage determinations.

### Cultural and Demographic Personalization

The platform implements cultural and demographic personalization comprising adherence-pattern personalization, pill-burden minimization for high-comorbidity populations, multi-language patient-facing interfaces, and health-literacy-stratified communication. Adherence-pattern personalization is based on machine-learning models trained on longitudinal medication-event-monitoring-system data, smart-dispenser data, pharmacy-refill data, and electronic-health-record-derived medication-reconciliation data, with patient-specific adherence-risk scores updated at intervals of from 7 to 90 days and adherence-support interventions targeted to high-risk patients comprising one or more of automated reminder systems with patient-specified timing and modality, pharmacist outreach, caregiver-engagement protocols, and packaging modifications including blister packs, weekly pill organizers, and multi-dose adherence packaging. Pill-burden minimization for high-comorbidity populations comprises one or more of fixed-dose combination formulations integrating multiple payload classes into a single dosage form, long-acting formulations extending dosing intervals from daily to weekly, monthly, or longer, depot formulations administered at intervals of from 30 to 365 days, and implant formulations with release durations of from 90 to 3,650 days. Multi-language patient-facing interfaces support patient-facing applications, instructional videos, consent documents, and labeling in from 20 to 100 languages with locale-specific cultural adaptation, with translation quality assured through professional translation, back-translation, and cognitive-debriefing protocols compliant with International Society for Pharmacoeconomics and Outcomes Research good-practice guidelines for cross-cultural translation. Health-literacy-stratified communication uses validated health-literacy screening instruments including the Newest Vital Sign, the Rapid Estimate of Adult Literacy in Medicine, and the Short Test of Functional Health Literacy in Adults to stratify patients across from 3 to 5 health-literacy strata, with stratum-specific patient-facing communication materials authored at reading levels of from 4th-grade to 12th-grade equivalence, and with visual-supplementation including pictograms, video demonstrations, and augmented-reality medication-identification aids for low-literacy strata. Closed-loop integration channels adherence-pattern data, communication-engagement data, and patient-reported-outcome data back to the personalization controller, which adjusts dosing schedules, formulations, and communication modalities to maximize patient-specific adherence and engagement. Clinical utility comprises one or more of improvement in adherence rates of from 15 percent to 60 percent relative to non-personalized communication, expansion of access to non-English-speaking and low-health-literacy populations historically excluded from advanced therapeutic regimens, and reduction in disparities in therapeutic outcomes across demographic strata. Regulatory considerations comprise compliance with FDA ‘Diversity Plans to Improve Enrollment of Participants From Underrepresented Racial and Ethnic Populations in Clinical Trials’ guidance, FDA Patient-Focused Drug Development guidance series, Health and Human Services Office of Civil Rights language-access regulations under Title VI of the Civil Rights Act, and the Affordable Care Act Section 1557 anti-discrimination provisions.

### Drug-Drug Interaction Management

The platform implements drug-drug interaction management comprising population-pharmacokinetic-model-based drug-drug interaction prediction, concomitant-medication-aware dose adjustment, reverse drug-drug interaction analysis, and cytochrome P450 substrate, inhibitor, and inducer interaction handling. Population-pharmacokinetic-model-based prediction is implemented using physiologically-based pharmacokinetic models implemented in Simcyp, GastroPlus, PK-Sim, or analogous platforms with patient-specific parameters comprising organ volumes, blood flows, plasma protein concentrations, and cytochrome P450, uridine diphosphate glucuronosyltransferase, and transporter expression levels conditioned on patient genotype, age, sex, organ-function status, and pregnancy status where applicable. Concomitant-medication-aware dose adjustment is implemented via real-time interrogation of the patient's electronic-health-record medication list and pharmacy-claims medication-fill history, with concomitant medications cross-referenced against drug-drug interaction databases including the University of Washington Drug Interaction Database, DrugBank, Lexicomp, and Micromedex, and with platform-specific drug-drug interaction tables curated for each payload class. Reverse drug-drug interaction analysis evaluates the effect of the platform-administered payload on the pharmacokinetics of co-administered drugs through pre-clinical in vitro and in vivo cytochrome P450 inhibition and induction assays following FDA ‘In Vitro Drug Interaction Studies’ guidance and Clinical Pharmacology Drug-Drug Interaction studies following FDA ‘Clinical Drug Interaction Studies’ guidance, with reverse drug-drug interaction findings incorporated into the platform's prescribing-decision-support module and labeled in the prescribing information. Cytochrome P450 substrate, inhibitor, and inducer interaction handling stratifies the payload portfolio across the principal cytochrome P450 isoforms including CYP1A2, CYP2B6, CYP2C8, CYP2C9, CYP2C19, CYP2D6, CYP2E1, CYP3A4, and CYP3A5, with patient-specific cytochrome P450 phenotype derived from genotype, endogenous metabolite ratios, and concomitant medication context. Closed-loop integration provides drug-drug-interaction-aware dose adjustment with adjustment magnitudes of from 25 percent to 400 percent relative to interaction-free dosing, drug-drug-interaction-aware monitoring intensification with monitoring-frequency increases of from 2-fold to 10-fold during interaction periods, and drug-drug-interaction-aware payload-switching decisions where interaction severity precludes safe co-administration. Clinical utility one or more comprises of reduction in drug-drug-interaction-attributable adverse events of from 40 percent to 80 percent, prevention of subtherapeutic exposure attributable to inducer co-administration, and rational guidance on co-administration of high-prevalence concomitant medications including statins, oral anticoagulants, antiepileptic drugs, antidepressants, antipsychotics, opioids, and proton-pump inhibitors. Regulatory considerations comprise compliance with FDA ‘Clinical Drug Interaction Studies—Cytochrome P450 Enzyme- and Transporter-Mediated Drug Interactions’ guidance, FDA ‘In Vitro Drug Interaction Studies—Cytochrome P450 Enzyme- and Transporter-Mediated Drug Interactions’ guidance, European Medicines Agency ‘Guideline on the Investigation of Drug Interactions,’ and labeled drug-drug interaction sections in the United States Prescribing Information conforming to 21 CFR 201.57.

### Environmental and Microbiome-Based Personalization

The platform implements environmental and microbiome-based personalization comprising gut-microbiome-aware dose scheduling, circadian-rhythm-aware dosing time windows, diet-aware co-administration guidance, and environmental-toxin-exposure-stratified risk profiles. Gut-microbiome-aware dose scheduling integrates 16S ribosomal RNA gene sequencing and shotgun metagenomic sequencing of fecal samples at intervals of from 30 to 365 days with patient-specific computation of microbiome-derived drug-metabolism capacity comprising bacterial cytochrome P450 homolog abundance, bacterial beta-glucuronidase abundance influencing enterohepatic recirculation, bacterial azoreductase and nitroreductase abundance, bile-acid-modifying enzyme abundance, and aromatic-amino-acid-decarboxylase abundance influencing levodopa and related catecholamine-precursor bioavailability. Dose scheduling is shifted forward or backward by from 1 to 12 hours and dose magnitude is adjusted by from 10 percent to 50 percent in response to microbiome-derived metabolic-capacity shifts. Circadian-rhythm-aware dosing time windows are derived from patient-specific chronotype assessment using the Munich ChronoType Questionnaire, dim-light melatonin onset measurement where available, and actigraphy-derived sleep-wake-cycle estimation, with dosing scheduled within patient-specific 2-to-6-hour windows aligned to chronotype-anchored peak central-nervous-system penetration or to nadir adverse-event-sensitivity windows. Diet-aware co-administration guidance is generated based on patient-specific dietary patterns inferred from dietary-recall data, food-frequency questionnaires, plasma-metabolomic dietary biomarkers, and where deployed, continuous-glucose-monitor-derived postprandial responses, with patient-specific guidance issued regarding fasting versus fed administration, high-fat versus low-fat-meal co-administration, grapefruit-juice avoidance for cytochrome-P450-3A4-substrate payloads, calcium-containing co-administration avoidance for cation-chelating payloads, and tyramine-restricted-diet observance for monoamine-oxidase-inhibitor payloads. Environmental-toxin-exposure-stratified risk profiles integrate residential geocoded air-quality data from Environmental Protection Agency monitoring networks, occupational-history-derived toxicant-exposure inventories, and biomarker-based toxicant-exposure measurements where indicated, with risk profiles informing payload-class avoidance flags for patients with high cumulative exposure to neurotoxic solvents, pesticides, heavy metals, or other documented neurotoxicants. Closed-loop integration channels environmental, microbiome, circadian, and dietary inputs into the multimodal Bayesian model-averaging framework alongside genomic, transcriptomic, proteomic, metabolomic, and digital-biomarker inputs. Clinical utility comprises one or more of improvement in oral bioavailability consistency of from 25 percent to 60 percent through microbiome- and diet-aware scheduling, reduction in circadian-mismatch-attributable adverse events of from 30 percent to 70 percent through chronotype-aligned dosing, and avoidance of toxicant-exposure-amplified adverse events in vulnerable populations. Regulatory considerations comprise compliance with FDA draft guidance on microbiome considerations for drug development, FDA ‘Food-Effect Bioavailability and Fed Bioequivalence Studies’ guidance, FDA ‘Assessing the Effects of Food on Drugs in INDs and NDAs-Clinical Pharmacology Considerations’ guidance, Environmental Protection Agency Integrated Risk Information System reference doses where toxicant exposure thresholds inform dosing, and Occupational Safety and Health Administration permissible-exposure-limit frameworks where occupational toxicant exposure is documented.

### Consolidated Closed-Loop Multimodal Personalization Architecture

The foregoing genomic, transcriptomic, proteomic, metabolomic, induced-pluripotent-stem-cell-derived, organoid-derived, longitudinal-monitoring-derived, methods-of-administration, financing, peptide-platform, blood-brain-barrier-shuttle, portfolio-integration, clinical-trial-design, decentralized-trial, real-world-evidence, companion-diagnostic, lifespan-adaptive, cultural-and-demographic, drug-drug-interaction, and environmental-and-microbiome-based personalization layers are integrated into a unified multimodal Bayesian model-averaging framework that computes, for each patient and at each closed-loop update epoch, a posterior distribution over candidate payload-class selections, dose magnitudes, dose-timing schedules, administration routes, monitoring schedules, and combination-regimen specifications. Each personalization layer contributes a marginal likelihood and a posterior weight to the model-averaging computation, with weights updated continuously based on each layer's per-patient predictive accuracy on held-out longitudinal outcomes. The unified controller dispatches dosing decisions to the patient-facing dispensation device, the clinician-facing decision-support interface, the pharmacy electronic prescribing interface, and the patient's electronic health record, and dispatches monitoring schedules to the patient-facing data-collection applications, the wearable-device data pipelines, and the central laboratory ordering systems. Updates to the controller's internal models are propagated through a continuous-learning pipeline subject to a predetermined change control plan filed with the FDA under the ‘Marketing Submission Recommendations for a Predetermined Change Control Plan for Artificial Intelligence/Machine Learning-Enabled Device Software Functions’ framework. The unified personalization architecture is interoperable with the broader Neurocore Biotechnologies LLC patent portfolio as set forth in the foregoing sections and is extensible to additional personalization layers and additional commonly-owned therapeutic platforms as they are developed and as their integration interfaces are specified. The clinical utility of the consolidated architecture comprises one or more of integrated multi-modality precision-medicine deployment, multi-decade longitudinal optimization of therapeutic outcomes, alignment of therapeutic, diagnostic, financing, and care-delivery infrastructure around the individual patient, and provision of a portfolio-spanning infrastructure for ongoing innovation in personalized central-nervous-system therapeutics. Regulatory considerations comprise the totality of guidances, frameworks, and statutes enumerated in the preceding sections, with overarching coordination through pre-submission and Type B meetings with the relevant FDA Centers, alignment with the European Medicines Agency Innovation Task Force, and engagement with global regulatory authorities including the United

Kingdom Medicines and Healthcare products Regulatory Agency, Health Canada, the Japanese Pharmaceuticals and Medical Devices Agency, and the Australian Therapeutic Goods Administration for international harmonization.

### Best-Mode Embodiments Across Representative Disease Verticals

The following best-mode embodiments illustrate the practice of the invention across five representative disease verticals. The embodiments are prophetic in nature; specific compositions, protocols, and outcomes have not been reduced to experimental practice as of the priority date and are provided to enable persons of ordinary skill in the art to practice the invention across the full claimed scope.

### Best Mode 1—Oral Alzheimer's Disease Therapy Via BACE1-Targeted Nanostructure

In a best mode for treating early-stage Alzheimer's disease in patients identified by amyloid-beta or phosphorylated-tau biomarker elevation, the hybrid nanostructure comprises (i) an outer shell of a virus-like-particle architecture providing gastrointestinal stability across pH 1 through pH 8 and intestinal protease challenge; (ii) an inner DNA origami core with internal cavity sized to retain a beta-secretase 1 enzyme inhibitor of molecular weight below 1,500 daltons through intercalation into double-helical regions of the inner core; (iii) M-cell-targeting peptide ligands of 10 to 14 amino acid residues on the outer shell exterior; (iv) blood-brain-barrier-crossing peptide ligands of 5 to 35 amino acid residues binding the transferrin receptor and the nicotinic acetylcholine receptor on the inner core exterior; and (v) a pH-responsive cytosine-rich inner-core element that releases the inhibitor upon endosomal acidification following central-nervous-system uptake. Oral administration once daily over a 24-week induction phase followed by a twice-weekly maintenance phase is contemplated.

### Best Mode 2—Oral Parkinson's Disease Disease-Modifying Therapy Via Alpha-Synuclein-Targeted Antibody Fragment

In a best mode for treating early Parkinson's disease, the hybrid nanostructure encapsulates an alpha-synuclein-binding antibody fragment of from 25 to 50 kilodaltons in molecular weight within an inner DNA origami core, with a virus-like-particle outer shell providing gastrointestinal protection, M-cell-targeting peptides on the outer shell exterior, low-density lipoprotein receptor-related protein 1-binding peptide ligands on the inner core for blood-brain-barrier crossing, and an enzyme-responsive linker releasing the antibody fragment selectively in regions of elevated cathepsin B activity associated with lysosomal dysfunction. Daily oral dosing over 52 weeks is contemplated, with alpha-synuclein-conformer-specific plasma biomarker monitoring at 4-week intervals.

### Best Mode 3—Oral Glioblastoma Adjunct Therapy Via Co-Loaded Cytotoxic and Immune-Checkpoint-Inhibitor Modalities

In a best mode for adjunctive therapy of newly diagnosed glioblastoma, the hybrid nanostructure co-encapsulates (a) a DNA-intercalating cytotoxic small molecule and (b) an immune-checkpoint-inhibitor antibody fragment, with matrix-metalloproteinase-cleavable peptide linkers selectively releasing both payloads within the proteolytic glioblastoma microenvironment. The composition is administered orally twice daily during the post-resection, post-radiation maintenance phase, in conjunction with standard-of-care temozolomide or its successor regimens.

### Best Mode 4—Oral Multiple-Sclerosis Disease-Modifying Therapy Via Anti-Inflammatory Cargo

In a best mode for relapsing-remitting multiple sclerosis, the hybrid nanostructure carries an anti-CD20 antibody-fragment payload or an equivalent B-cell-depleting modality, with the outer shell providing gastrointestinal protection, M-cell targeting on the outer shell exterior, transferrin-receptor and nicotinic-acetylcholine-receptor dual blood-brain-barrier-crossing ligands on the inner core, and pH-responsive intracellular release elements. Weekly oral dosing for 24 weeks with maintenance at extended intervals thereafter is contemplated, with magnetic-resonance-imaging lesion-burden and plasma neurofilament light chain monitoring at 12-week intervals.

Best Mode 5—Oral Lysosomal Storage Disease Enzyme Replacement Therapy with Central Nervous System Involvement

In a best mode for enzyme replacement therapy of a lysosomal storage disorder with central nervous system involvement, the hybrid nanostructure carries a recombinant lysosomal enzyme payload of approximately 50 to 100 kilodaltons within an inner protein nanocage core with matched internal cavity size, surrounded by a stabilizing outer shell, with cation-independent mannose-6-phosphate-receptor-targeting glycan modifications on the inner core for lysosomal targeting following central-nervous-system uptake, and pH-responsive release elements that trigger enzyme release upon lysosomal acidification. Bi-weekly oral dosing with quarterly substrate-accumulation biomarker monitoring is contemplated, replacing current parenteral enzyme-replacement-therapy regimens.

### PROPHETIC EXAMPLE DISCLAIMER

Certain embodiments and examples disclosed herein, including specific compositions, protocols, dosing regimens, and outcome projections, are prophetic in nature and have not yet been reduced to experimental practice. Such prophetic disclosures are provided to enable persons of ordinary skill in the art to practice the invention across the full claimed scope. Actual experimental validation may yield outcomes that vary from prophetic projections.

### Section R—Prophetic Examples

The following examples are prophetic and describe experiments and embodiments that have not necessarily been performed. All performance values represent predictions based on published literature for the individual component technologies cited throughout this specification and are expressed using terms such as “expected,” “predicted,” “anticipated,” and “would.” These examples are provided to illustrate the manner of making and using the claimed invention and to demonstrate enablement; actual experimental results may differ. Each component operation recited below is supported by the corresponding validated method described in the Detailed Description above.

### Prophetic Example 1—DNA Origami Core Assembly and Characterization

A tubular DNA origami inner core suitable for encapsulation within a virus-like-particle outer shell is assembled from a single-stranded DNA scaffold of approximately 7,000 to 7,500 nucleotides combined with a pool of approximately 200 staple oligonucleotides (each from 32 to 48 nucleotides), of which a subset carries amine-modified handles for subsequent conjugation to transferrin receptor-binding and nicotinic acetylcholine receptor-binding peptide ligands. The mixture is thermally annealed and purified by polyethylene glycol precipitation, then stabilized by oligolysine coating. The target geometry is a hollow tube of approximately 70 nanometers in length and 40 nanometers in outer diameter. Based on published literature, assembly yield of 70 to 85 percent is expected; agarose gel electrophoresis is expected to show a single dominant band; and the coated origami is expected to exhibit less than 10 percent degradation after 1 hour of exposure to 10 units per milliliter DNase I and less than 30 percent after 24 hours.

### Prophetic Example 2—P22 Virus-Like Particle Production and Characterization

Virus-like particles suitable for inner-core encapsulation are produced by expressing P22 bacteriophage coat protein in Escherichia coli with appropriate scaffold protein, with induction by isopropyl-beta-D-thiogalactopyranoside, followed by lysis, clarification, polyethylene-glycol precipitation, sucrose-cushion ultracentrifugation, and size-exclusion chromatography. A yield of 10 to 50 milligrams of virus-like particles per liter of culture is expected, with hydrodynamic diameter of 60 to 68 nanometers, polydispersity index below 0.15, and retention of structural integrity across pH 2.0 to 10.0 with greater than 90 percent intact particles after 2 hours at pH 2.0.

### Prophetic Example 3—Hybrid Nanostructure Assembly-Inner Core into Outer Shell

Hybrid nanostructures are assembled by electrostatic-driven co-assembly. Pre-formed virus-like particles are disassembled into coat protein dimers in a high-pH, high-ionic-strength buffer, the coat protein dimers are purified by size exclusion chromatography, mixed with stabilized inner cores at controlled stoichiometry in an assembly buffer, incubated, and purified by sucrose-gradient ultracentrifugation. An encapsulation efficiency of 50 to 80 percent is expected, with core-shell structures observable by transmission electron microscopy and a hybrid band that co-stains for both nucleic-acid and protein components by agarose gel electrophoresis. If electrostatic co-assembly is unsuccessful, a scaffold-protein-mediated loading approach using a scaffold protein fused to a nucleic-acid-binding domain provides a validated alternative.

### Prophetic Example 4—M-Cell-Targeting Peptide Ligand Conjugation

Gastrointestinal-targeting peptide ligands of 10 to 14 amino acid residues are conjugated to the exterior of purified hybrid nanostructures by reaction of an N-hydroxysuccinimide-activated ligand-polyethylene-glycol species at approximately 50-fold molar excess relative to nanostructure with surface-exposed primary amines, in a phosphate buffer for 2 hours, followed by quenching and removal of unreacted ligand. A conjugation density of 40 to 80 ligands per nanostructure is expected, with 5- to 10-fold enhancement of M-cell binding relative to non-targeted controls in functional binding assays.

### Prophetic Example 5—In Vitro Gastrointestinal Stability Assessment

Hybrid nanostructures (1 milligram per milliliter) are incubated in United States Pharmacopeia simulated gastric fluid and simulated intestinal fluid at 37 degrees Celsius. Based on published literature for the constituent shell and core technologies, expected structural integrity in simulated gastric fluid is approximately 98 percent at 0.5 hours, 95 percent at 1 hour, 90 percent at 2 hours, and 80 percent at 4 hours, with cargo retention tracking closely. In simulated intestinal fluid, expected structural integrity is approximately 98, 95, 88, and 80 percent at 1, 2, 4, and 6 hours, while targeting peptide retention is expected to decline more rapidly, to approximately 90, 80, 65, and 50 percent over the same intervals, identifying proteolytic peptide degradation as the limiting factor that may be mitigated by D-amino acid substitution, cyclization, or polyethylene-glycol shielding.

### Prophetic Example 6—In Vitro M-Cell Transcytosis Assessment

M-cell transcytosis is assessed in a validated Caco-2/Raji B lymphocyte co-culture model. Fluorophore-labeled hybrid nanostructures (10\hat{ }10 particles per milliliter) are applied to the apical chamber and fluorescence is quantified in the basolateral chamber over time, with non-targeted hybrids and free-label as controls. Based on published M-cell-targeting nanoparticle data, M-cell-targeted hybrids in the Caco-2/Raji model are expected to achieve cumulative transcytosis of 3 to 6 percent of applied dose at 4 hours and 8 to 15 percent at 24 hours, compared with 0.5 to 1.5 percent and 1.5 to 3 percent for non-targeted hybrids.

### Prophetic Example 7—In Vitro Blood-Brain Barrier Transcytosis Assessment

Blood-brain barrier transcytosis is assessed using the hCMEC/D3 human brain microvascular endothelial cell model. Fluorophore-labeled inner cores or assembled hybrids are applied apically and basolateral fluorescence is quantified over 24 hours for dual-targeted, single-targeted, and non-targeted constructs. Expected cumulative transcytosis at 24 hours is 0.5 to 1.5 percent for non-targeted, 2 to 5 percent for single-receptor-targeted, 3 to 6 percent for alternative single-receptor-targeted, and 5 to 10 percent for dual-targeted constructs.

### Prophetic Example 8—In Vivo Pharmacokinetics in Mouse

Oral M-cell-targeted hybrids, oral non-targeted hybrids, intravenous inner cores, and oral unprotected inner cores are administered to C57BL/6 mice, with oral dosing at 5 milligrams per kilogram by gavage and intravenous dosing at 1 milligram per kilogram, followed by blood and tissue sampling over 24 hours. Oral M-cell-targeted hybrids are expected to show a time to maximum concentration of 2 to 4 hours, a maximum concentration of 2 to 8 micrograms per milliliter, and an oral bioavailability of approximately 3 to 10 percent. Expected brain accumulation from oral targeted dosing is approximately 0.2 to 0.5 percent of the oral dose.

### Prophetic Example 9—Doxorubicin Payload Loading and Cellular Cytotoxicity

A model small-molecule chemotherapeutic agent is loaded into the inner core through intercalation into double-helical regions of the inner-core nucleic acid. Loading capacity of 2,000 to 4,000 drug molecules per inner core at 70 to 90 percent efficiency is expected, with pH-responsive release of less than 20 percent at physiological pH 7.4 over 24 hours but 60 to 80 percent at endosomal pH 5.5 within 4 hours. Cellular half-maximal inhibitory concentration is expected to be 2- to 5-fold lower than free drug.

### Prophetic Example 10—Oral mRNA Delivery for Pediatric Lysosomal Storage Disease

A messenger RNA payload encoding a deficient lysosomal enzyme is loaded into a structured inner core through hybridization to internal capture sequences, with cationic polymer condensation of the messenger RNA prior to encapsulation. The composition is administered orally to pediatric patients with central-nervous-system-involving lysosomal storage disease, achieving transient hepatic and central-nervous-system enzyme expression sufficient to clear accumulating substrate at multi-week intervals. Expected substrate reduction is 30 to 60 percent at 4 weeks and 50 to 80 percent at 12 weeks based on published mRNA enzyme-replacement preclinical data.

### Prophetic Example 11—Oral CRISPR-Cas9 Ribonucleoprotein Delivery for Familial Alzheimer's Disease

A clustered regularly interspaced short palindromic repeats associated nuclease ribonucleoprotein complex configured to introduce a protective genetic edit at a disease-modifying locus is loaded into a large-cavity inner core. The composition is administered orally to patients carrying high-risk alleles, achieving partial central-nervous-system editing of target cells with prophetic editing efficiency of 1 to 5 percent per dose and cumulative editing of 5 to 15 percent over 12 weeks of weekly dosing.

### Prophetic Example 12—Oral Anti-Tau Antibody Fragment Therapy with Tau-217 Monitoring

An anti-tau antibody fragment is delivered orally as recited in Best Mode 2, with plasma phosphorylated-tau-217 monitoring at 4-week intervals serving as a pharmacodynamic readout. Closed-loop dosing logic adjusts dose interval from weekly to bi-weekly to monthly depending on biomarker trajectory, with a defined safety floor preventing dosing intervals shorter than weekly and an external clinical override available at every adjustment.

### Prophetic Example 13-Oral Self-Amplifying RNA Vaccine for Tau Immunotherapy

A self-amplifying RNA payload encoding a tau-derived immunogen is delivered orally for active immunization against pathological tau species. The composition is administered as a three-dose primary series followed by annual boosters, with expected anti-tau IgG titer development of 1:1,000 to 1:10,000 after the third dose.

### Prophetic Example 14—Oral Anti-Synuclein Aptamer Therapy

A DNA aptamer ligand selected for high-affinity binding to pathological alpha-synuclein oligomers serves as the therapeutic payload, delivered orally as described, achieving central-nervous-system aptamer concentrations sufficient to interfere with synuclein aggregation and propagation.

### Prophetic Example 15—Oral Neurotrophic Peptide Therapy for Ischemic Stroke Recovery

A growth-factor-mimetic peptide of approximately 20 amino acid residues with neurotrophic activity is delivered orally beginning at 24 to 72 hours after ischemic stroke onset, throughout the subacute and chronic recovery phases, with weekly functional assessment and dosing-interval adjustment based on motor recovery trajectories.

### Prophetic Example 16—Oral Antiseizure Gene-Therapy Cargo for Drug-Resistant Epilepsy

A nucleic-acid payload encoding neuropeptide-Y or galanin under tissue-specific regulatory control is delivered orally to patients with drug-resistant focal epilepsy. Closed-loop dosing logic incorporates ambulatory electroencephalography seizure-detection metrics from a wearable device, adjusting subsequent dosing to reduce seizure frequency.

### Prophetic Example 17—Oral Mood-Disorder Therapy with Closed-Loop Wearable Integration

A rapid-acting antidepressant peptide payload is delivered orally on an as-needed basis triggered by a wearable-device-derived depression-symptom score. The closed-loop architecture authorizes dosing only when both the symptom score and a defined physiologic marker exceed predefined thresholds, with mandatory physician review of each authorized dose.

### Prophetic Example 18—Oral Anti-Inflammatory Cargo for Chronic Neuroinflammation

An anti-cytokine antibody-fragment payload targeting tumor-necrosis-factor-alpha or interleukin-6 is delivered orally for chronic neuroinflammatory conditions, with monthly plasma neurofilament light chain monitoring serving as the pharmacodynamic readout.

### Prophetic Example 19—Oral Microbiome-Modulating Therapy with Gut-Brain Axis Targeting

A bacteriophage-derived therapeutic payload selectively modulating gut microbiota implicated in central nervous system disease is delivered using an outer shell adapted to colonic release, with biomarkers including plasma trimethylamine-N-oxide and lipopolysaccharide-binding-protein monitored as gut-brain axis readouts.

### Prophetic Example 20—Oral Mitochondrial-Targeted Therapy for Friedreich's Ataxia

A mitochondrial-targeting peptide-payload conjugate is delivered orally, with the peptide moiety facilitating subcellular targeting to mitochondria following central-nervous-system uptake. Quarterly cardiac and neurologic assessment guides long-term dosing adjustment.

### Prophetic Example 21—Oral Anti-Aging-Pathway Modulator for Cognitive Maintenance

A senolytic or autophagy-inducing payload is delivered orally on an intermittent dosing schedule, with cognitive performance tracked via a smartphone-based assessment battery and dosing adjusted to maintain performance above defined thresholds.

### Prophetic Example 22—Oral Combination Therapy for Glioblastoma-Cytotoxic Plus Immune-Checkpoint Plus Vascular-Normalizing Triple Cargo

A triple-payload composition co-encapsulates a cytotoxic small molecule, an immune-checkpoint-inhibitor antibody fragment, and a vascular-normalizing agent, with sequential release controlled by orthogonal stimulus-responsive linkers releasing the vascular-normalizing agent first to enhance subsequent payload distribution.

### Prophetic Example 23—Oral Pediatric Formulation for Rett Syndrome

An MECP2-targeted nucleic-acid therapy is delivered orally to pediatric patients with Rett syndrome, in a pediatric-friendly orally disintegrating tablet formulation with weight-based dose scaling and quarterly functional outcome monitoring.

### Prophetic Example 24—Oral Anti-Prion Therapy

An anti-prion antibody fragment or small-molecule prion-aggregation inhibitor is delivered orally to patients with familial prion disease, with quarterly clinical and magnetic-resonance-imaging assessment.

### Prophetic Example 25—Oral Spinal-Cord-Injury Recovery Therapy

A pro-axon-regenerative peptide payload is delivered orally during the acute and subacute phases following spinal cord injury, with weekly functional assessment.

### Prophetic Example 26—Oral Therapy for Substance Use Disorder

A neurochemical-pathway-modulating payload addressing reward-circuit dysfunction in substance use disorder is delivered orally on a daily or weekly schedule, with objective biomarker monitoring of substance use and craving-related neuroendocrine markers.

### Prophetic Example 27—Oral Neuropsychiatric Therapy with AI-Driven Closed-Loop Dosing

A neuropsychiatric payload is delivered orally with closed-loop dosing controlled by a machine-learning model trained on the patient's historical response data and current wearable-derived state metrics, with mandatory clinical oversight and external override.

### Prophetic Example 28—Manufacturing Embodiment-Continuous-Flow Hybrid Assembly at Commercial Scale

A continuous-flow manufacturing embodiment is described in which inner-core folding, payload loading, outer-shell co-assembly, and ligand conjugation occur in sequential flow reactors with in-line characterization at each step, enabling production of multi-gram batches with consistent quality attributes.

### Prophetic Example 29—Personalization Embodiment-Patient-Derived Organoid-Indexed Payload Selection

A patient-derived organoid model of the patient's disease tissue is used to screen candidate payload species and combinations, with the highest-efficacy combination selected for incorporation into a personalized hybrid nanostructure formulation for that patient.

### Prophetic Example 30—Closed-Loop Architecture-Genome-Indexed Long-Horizon Therapeutic Regimen

A multi-year therapeutic regimen is constructed in which payload selection, dose level, and dosing interval are initialized from the patient's genome and proteome profiles and then adjusted longitudinally over the course of therapy using monthly biomarker measurements, with mandatory annual clinical review and external override.

What is claimed is:

1. A hybrid biomimetic nanostructure composition for oral delivery of central nervous system therapeutics across dual biological barriers, the composition comprising: (a) a protective outer shell of outer diameter of from 20 nanometers to 200 nanometers configured to protect the nanostructure during gastrointestinal transit by resisting degradation at a pH of from 1.0 to 3.0 for at least 30 minutes at 37 degrees Celsius and by resisting degradation by intestinal proteases under physiological conditions for at least 1 hour at 37 degrees Celsius; (b) a structured inner core encapsulated within the protective outer shell, the structured inner core configured to organize and controllably release a therapeutic payload and defining an internal cavity having a diameter of from 5 nanometers to 60 nanometers; (c) at least one therapeutic payload of molecular weight of from 100 daltons to 200 kilodaltons associated with the structured inner core; (d) at least one M-cell targeting moiety conjugated to or associated with an exterior surface of the protective outer shell and configured to mediate transcytosis across intestinal microfold-cell epithelium; (e) at least one blood-brain barrier transcytosis moiety conjugated to or associated with the structured inner core and configured to mediate receptor-mediated transcytosis across brain capillary endothelium; and (f) at least one pH-responsive or stimulus-responsive element configured to trigger conformational change or payload release in response to a defined pH transition between physiological pH and endosomal pH, an enzymatic stimulus, a redox stimulus, or an externally applied physical stimulus.

2. The composition of claim 1, wherein the at least one therapeutic payload is selected from the group consisting of a small molecule drug, a peptide therapeutic, a protein therapeutic, an antibody, an antibody fragment, a bispecific T-cell engager, a designed ankyrin-repeat protein, a monobody, an affibody, an anticalin, a messenger ribonucleic acid, a self-amplifying ribonucleic acid, a circular ribonucleic acid, a small interfering ribonucleic acid, a short hairpin ribonucleic acid, a microRNA mimic, a microRNA inhibitor, an antisense oligonucleotide, an aptamer, a clustered regularly interspaced short palindromic repeats associated nuclease ribonucleoprotein complex, a base editor ribonucleoprotein, a prime editor ribonucleoprotein, a molecular glue degrader, a proteolysis-targeting chimera, an autophagy-targeting chimera, a lysosome-targeting chimera, and a combination of any two or more of the foregoing.

3. The composition of claim 1, wherein the protective outer shell is selected from the group consisting of a virus-like particle assembled from coat protein subunits derived from a bacteriophage selected from P22, MS2, Qβ, PP7, AP205, T4, T7, lambda, and phi-29 and functional equivalents thereof, a virus-like particle assembled from coat protein subunits derived from a plant virus selected from cowpea chlorotic mottle virus, cowpea mosaic virus, brome mosaic virus, and tobacco mosaic virus, an engineered protein nanocage selected from ferritin, apoferritin, encapsulin, lumazine synthase, and heat-shock-protein nanocages, a lipid bilayer shell selected from a liposome, a lipid nanoparticle, a solid lipid nanoparticle, and a nanostructured lipid carrier, a polymer shell selected from poly(lactic-co-glycolic acid), polylactic acid, polycaprolactone, chitosan, alginate, gelatin, casein, silk fibroin, and biodegradable copolymers thereof, an inorganic shell selected from mesoporous silica and calcium phosphate and hydroxyapatite, and a biomimetic membrane coating derived from a red blood cell membrane, a platelet membrane, a leukocyte membrane, a mesenchymal stem cell membrane, or an exosome membrane, in each case substantially devoid of any viral genomic nucleic acid that would render the protective outer shell infectious.

4. The composition of claim 1, wherein the structured inner core is selected from the group consisting of a deoxyribonucleic acid origami nanostructure formed by folding a single-stranded deoxyribonucleic acid scaffold of from 1,000 to 50,000 nucleotides with a plurality of complementary staple oligonucleotides into a predetermined three-dimensional shape, a ribonucleic acid origami nanostructure, a peptide nanofiber core, a peptide-amphiphile core, a protein nanocage interior, a dendrimer-based core, a mesoporous inorganic core, a polymeric micelle core, and a hydrogel core, and combinations of two or more of the foregoing arranged hierarchically.

5. The composition of claim 1, wherein the at least one M-cell targeting moiety comprises a peptide ligand of from 5 to 30 amino acid residues identified by phage display screening for binding to a microfold-cell apical surface, an antibody fragment specific for glycoprotein 2 or beta-1 integrin, a carbohydrate moiety that binds an M-cell-expressed lectin, or an aptamer ligand selected for M-cell binding, and wherein the at least one blood-brain barrier transcytosis moiety comprises a peptide ligand of from 5 to 35 amino acid residues configured to bind a receptor selected from the transferrin receptor, the insulin receptor, the insulin-like growth factor 1 receptor, the low-density lipoprotein receptor-related protein 1, the nicotinic acetylcholine receptor, and the leptin receptor with affinity below 1 micromolar, or an antibody fragment, an engineered binder, an aptamer, or a natural ligand of any of the foregoing receptors.

6. The composition of claim 1, wherein the at least one pH-responsive or stimulus-responsive element is selected from the group consisting of a pH-responsive cytosine-rich oligonucleotide element of from 20 to 30 nucleotides configured to undergo conformational change at pH below 6.5, a triplex deoxyribonucleic acid element configured to undergo pH-dependent transition, a hydrazone or acetal or beta-thiopropionate pH-sensitive linker, an enteric polymer system configured to dissolve at a pH above a defined threshold in the range of pH 5.5 to pH 7.0, a peptide linker cleavable by cathepsin B or by a matrix metalloproteinase, a disulfide bond cleavable by intracellular glutathione, a thioketal or phenylboronic ester reactive-oxygen-species-responsive element, a photocleavable linker activated by ultraviolet, visible, or near-infrared light, a thermosensitive element activated above 40 degrees Celsius, an ultrasound-responsive element, and a logic-gated combination of two or more of the foregoing requiring conjunction of multiple stimulus inputs to trigger payload release.

7. The composition of claim 1, further comprising a stealth coating selected from poly(ethylene glycol) of molecular weight from 1,000 to 20,000 daltons, polysarcosine, poly(carboxybetaine), poly(sulfobetaine), and poly(2-methacryloyloxyethyl phosphorylcholine), conjugated to an exterior surface of the protective outer shell at a surface coverage of from 5 to 50 percent of available conjugation sites, and configured to be shed in response to a defined stimulus encountered along the dual-barrier transit path.

8. A method of treating a neurological, neurodegenerative, neuroinflammatory, neuropsychiatric, neurodevelopmental, or neuro-oncologic condition in a patient in need thereof, the method comprising: (i) orally administering to the patient a therapeutically effective amount of the composition of claim 1; (ii) allowing the composition to traverse an intestinal epithelium by microfold-cell-mediated transcytosis directed by the at least one M-cell targeting moiety; (iii) allowing the composition to enter systemic circulation; (iv) allowing the composition to cross a blood-brain barrier by receptor-mediated transcytosis directed by the at least one blood-brain barrier transcytosis moiety; (v) allowing release of the therapeutic payload in central nervous system tissue under the control of the at least one stimulus-responsive element; and (vi) thereby treating the condition.

9. The method of claim 8, wherein the condition is selected from the group consisting of Alzheimer's disease, Parkinson's disease, Huntington's disease, amyotrophic lateral sclerosis, frontotemporal dementia, Lewy body dementia, multiple system atrophy, progressive supranuclear palsy, spinocerebellar ataxia, multiple sclerosis, neuromyelitis optica spectrum disorder, autoimmune encephalitis, glioblastoma multiforme, diffuse midline glioma, medulloblastoma, ependymoma, central nervous system metastasis of an extracranial primary tumor, major depressive disorder including treatment-resistant subtypes, bipolar disorder, schizophrenia, generalized anxiety disorder, obsessive-compulsive disorder, post-traumatic stress disorder, substance use disorder, autism spectrum disorder, Rett syndrome, Angelman syndrome, Dravet syndrome, fragile X syndrome, a lysosomal storage disease with central nervous system involvement, ischemic stroke, hemorrhagic stroke, traumatic brain injury, spinal cord injury, status epilepticus, and drug-resistant focal epilepsy.

10. The method of claim 8, wherein the oral administering is performed according to a dosing schedule selected from once daily, twice daily, three times daily, once weekly, twice weekly, bi-weekly, monthly, and an as-needed schedule triggered by a biomarker or wearable-device signal, over a treatment period of at least four weeks.

11. The method of claim 8, wherein the composition is administered in an oral dosage form selected from a hard gelatin capsule, a hydroxypropyl methylcellulose capsule, an oral tablet, an orally disintegrating tablet, an oral liquid suspension, an oral powder for reconstitution, and a dissolvable oral film, in each case bearing an enteric coating that is stable at a pH below 5.5 and dissolves at a pH above 6.0.

12. The method of claim 8, further comprising selecting the patient for treatment on the basis of at least one biomarker selected from an amyloid positron emission tomography signal, a tau positron emission tomography signal, a cerebrospinal fluid amyloid or tau biomarker level, a plasma neurofilament light chain level, a plasma phosphorylated tau level, a plasma glial fibrillary acidic protein level, a magnetic resonance imaging volumetric measurement, a genetic risk variant, a transcriptomic profile, and a patient-derived organoid drug-response readout.

13. The method of claim 8, further comprising monitoring the patient during treatment by measurement of at least one biomarker selected from the biomarkers recited in claim 12 and at least one wearable-derived digital biomarker selected from continuous actigraphy, gait analysis, speech and language metrics, sleep architecture, heart-rate variability, and cognitive task performance, at intervals of from one week to one month.

14. The method of claim 8, further comprising co-administering to the patient a second therapeutic agent by a route of administration different from the oral administration of the composition of claim 1, wherein the composition and the second therapeutic agent target different pathological mechanisms of the condition or address different temporal phases of the condition.

15. A closed-loop oral therapeutic delivery system for treatment of a central nervous system condition, the system comprising: (i) the composition of claim 1; (ii) at least one biomarker detection element configured to detect at least one therapeutic response indicator selected from a circulating biomarker measurable in peripheral blood, a cerebrospinal fluid biomarker, an imaging biomarker, and a wearable-device-derived digital biomarker; (iii) at least one feedback element coupling the biomarker detection element to a dosing-adjustment logic that modulates a subsequent dose level, a subsequent dosing interval, or a selection among alternative formulations of the composition of claim 1; and (iv) at least one external control element configured to override the closed-loop dosing-adjustment logic and to halt or modify dosing in response to a clinical input from a physician or in response to a safety interlock condition.

16. The system of claim 15, wherein the at least one biomarker detection element comprises a point-of-care immunoassay device selected from a lateral-flow strip, a microfluidic enzyme-linked immunosorbent assay device, a single-molecule array immunoassay device, an electrochemical biosensor, a field-effect-transistor biosensor, and a wearable continuous biosensor.

17. The system of claim 15, wherein the feedback element comprises a rules-based controller applying predefined biomarker thresholds, a model-predictive controller employing a pharmacokinetic-pharmacodynamic patient model, a reinforcement-learning controller, a supervised-learning predictor of treatment response trained on patient cohort data, or a transformer-based decision support model.

18. The system of claim 15, further comprising a wearable device or a subcutaneous implant operatively coupled to the biomarker detection element and configured to provide continuous or near-continuous data streams to the feedback element.

19. The system of claim 15, wherein the external control element comprises a clinician interface configured to authorize, modify, or veto dosing decisions issued by the feedback element, and at least one safety interlock that prevents autonomous dose escalation beyond a predefined safety bound or autonomous shortening of dosing interval below a predefined minimum interval.

20. The system of claim 15, further comprising a personalization element configured to initialize the feedback element from one or more of a patient genome profile, a patient transcriptome profile, a patient proteome profile, a patient metabolome profile, and a patient-derived organoid drug-response screening result, and to adjust the feedback element over time using accumulated longitudinal response data.
